(19) United States
(12) Patent Application Publication
(10) Pub. No.: US 2026/0207782 A1
(43) Pub. Date: Jul. 23, 2026

(54) METHODS AND COMPOSITIONS FOR ALTERING MECP2 EXPRESSION

(71) Applicant: Emugen Therapeutics LLC, Woburn, MA (US)

(72) Inventors: Jonathan J. Wilde, Waltham, MA (US); Tobias Kaiser, Boston, MA (US); Xian Gao, Belmont, MA (US); Olga Brevnova, Somerville, MA (US); Tomomi Aida, Arlington, MA (US); Yuanyuan Hou, Cambridge, MA (US); Tyler Brown, Somerville, MA (US)

(21) Appl. No.: 19/636,491

(22) Filed: Apr. 1, 2026

Related U.S. Application Data
(63) Continuation of application No. PCT/US24/50790, filed on Oct. 10, 2024.
(60) Provisional application No. 63/589,505, filed on Oct. 11, 2023.

(51) Int. Cl. A61K 48/00; A61K 31/711; A61K 38/17; A61P 25/28; C12N 15/113; C12N 15/86

(52) U.S. Cl. CPC A61K 48/005; A61K 31/711; A61K 38/1709; A61P 25/28; C12N 15/113; C12N 15/86; C12N 2310/11; C12N 2310/14; C12N 2320/33; C12N 2750/14143; C12N 2830/42; C12N 2830/50

(57) ABSTRACT

Provided herein, inter alia, are expression systems capable of modulating expression of MECP2. In embodiments, the expression systems silence the expression of endogenous MECP2 and induce expression of recombinant MECP2. The expression system provided herein may be useful for treating a genetic disorder such as Rett Syndrome.

### CROSS-REFERENCE

This application is a continuation of International Patent Application No. PCT/US2024/050790 filed Oct. 10, 2024, which claims the benefit of U.S. Provisional Application No. 63/589,505, filed Oct. 11, 2023, which application is incorporated herein by reference.

### INCORPORATION BY REFERENCE OF SEQUENCE LISTING

The present application is being filed along with a Sequence Listing in electronic format. The Sequence Listing is provided as a file entitled 062692-505001WO.xml, created Oct. 9, 2024, which is 376,599 bytes in size. The information in the electronic format of the Sequence Listing is incorporated by reference in its entirety.

### BACKGROUND

Rett Syndrome is an example of an X-linked neurodevelopmental disorder that may be caused by haploinsufficiency of a gene encoding methyl CpG binding protein 2 (MECP2 or Mecp2). The disease, which is almost exclusively seen in females, may include symptoms such as intellectual disability, speech impairment, stereotyped hand movements, breathing abnormalities, and increased mortality. A similar neurodevelopmental disorder that affects both sexes, MECP2 duplication syndrome, may be caused by increased levels of MECP2. In females with Rett Syndrome, tissues may exhibit mosaic expression of MECP2 due to random X-inactivation, leading to approximately 50% of cells expressing normal levels of MECP2 and 50% of cells completely lacking MECP2. This may pose a problem for gene therapies using gene replacement, since overexpression of MECP2 can be toxic, but some cells maintain normal expression of MECP2. As such, it would be useful for a gene therapy for Rett Syndrome to not only restore MECP2 expression in cells lacking MECP2, but also avoid overexpression in all cells, especially those that express wild-type levels of the gene. Provided herein, inter alia, are solutions to these and other problems in the art.

### SUMMARY

Disclosed herein, in some embodiments, are expression systems for altering gene expression, comprising: a silencing module deoxyribonucleic acid (DNA) sequence comprising: a first promoter sequence, an exonic splicing silencer (ESS) nucleic acid sequence, an antisense nucleic acid sequence that targets an MECP2 ribonucleic acid (RNA), an Sm binding site sequence, a 3′ hairpin sequence, and a 3′ terminator sequence, wherein the silencing module encodes a modified U7 small nuclear RNA (snRNA) that silences or reduces endogenous MECP2 protein expression; and an MECP2 synthesis module DNA sequence comprising: a second promoter sequence, a 5′ untranslated region (UTR) sequence, a nucleic acid coding sequence (CDS) encoding MECP2, and a 3′ UTR sequence, wherein the MECP2 synthesis module encodes a recombinant messenger RNA (mRNA) that generates MECP2 protein. Disclosed herein, in some embodiments, are dual RNA systems for altering gene expression, comprising: a U7 small nuclear RNA (snRNA) silencing module comprising: an exonic splicing silencer (ESS) nucleic acid sequence, an antisense nucleic acid sequence that binds an MECP2 ribonucleic acid (RNA), an Sm binding site sequence, and a 3′ hairpin sequence; and an MECP2 messenger RNA (mRNA) synthesis module comprising: a 5′ untranslated region (UTR) sequence, a nucleic acid coding sequence (CDS) encoding MECP2, and a 3′ UTR sequence; wherein the U7 snRNA silencing module silences or reduces endogenous MECP2 protein expression, and the MECP2 mRNA synthesis module generates MECP2 protein. Disclosed herein, in some embodiments, are systems for altering gene expression, comprising: a silencing module comprising an exonic splicing silencer (ESS) nucleic acid sequence coupled with an antisense nucleic acid sequence that targets an endogenous MECP2 ribonucleic acid (RNA); and a synthesis module comprising a nucleic acid coding sequence (CDS) that encodes a recombinant version of the MECP2 RNA. In some embodiments, the synthesis module comprises an RNA molecule. In some embodiments, the RNA molecule of the synthesis module comprise a messenger RNA (mRNA). In some embodiments, the synthesis module comprises a DNA molecule. In some embodiments, the DNA molecule of the synthesis module encodes an mRNA. In some embodiments, the synthesis module further comprises a synthesis module promoter sequence, or wherein the synthesis module is encoded by a nucleic acid comprising the synthesis module promoter sequence. In some embodiments, the synthesis module promoter sequence comprises a mouse or human promoter sequence. In some embodiments, the synthesis module promoter comprises a ubiquitous promoter. In some embodiments, the synthesis module promoter comprises a weak promoter. In some embodiments, the weak promoter drives expression of mRNA molecules at a rate no greater than does an endogenous MECP2 promoter. In some embodiments, the synthesis module promoter sequence comprises a promoter sequence of a −Ubc promoter, a PGK promoter, or an EF1a-core promoter. In some embodiments, the synthesis module promoter sequence is at least 80%, at least 85%, at least 90%, at least 95%, at least 98%, at least 99%, or 100% identical to a promoter sequence in Table 1. In some embodiments, the synthesis module promoter sequence is 5′ or upstream relative to the CDS. In some embodiments, the synthesis module further comprises an SV40 intron sequence. In some embodiments, the SV40 intron sequence is at least 80%, at least 85%, at least 90%, at least 95%, at least 98%, at least 99%, or 100% identical to an SV40 intron sequence in Table 1. In some embodiments, the SV40 intron sequence is 3′ or downstream of the synthesis module promoter sequence. In some embodiments, the SV40 intron sequence is 5′ or upstream relative to the CDS. In some embodiments, the synthesis module further comprises a 5′ untranslated region (UTR) sequence of the MECP2 RNA. In some embodiments, the 5′ UTR sequence is at least 80%, at least 85%, at least 90%, at least 95%, at least 98%, at least 99%, or 100% identical to a 5′ UTR sequence in Table 1. In some embodiments, the synthesis module further comprises a Kozak sequence. In some embodiments, the Kozak sequence is at least 80%, at least 85%, at least 90%, at least 95%, at least 98%, at least 99%, or 100% identical to 5′-CGGAAAATG-3′. In some embodiments, the Kozak sequence is at least 80%, at least 85%, at least 90%, at least 95%, at least 98%, at least 99%, or 100% identical to 5′-GCCNCCATG-3′ where N is A, T, C, or G, and ATG is a start codon. In some embodiments, the Kozak sequence is at least 80%, at least 85%, at least 90%, at least 95%, at least 98%, at least 99%, or 100% identical to 5′-GCCNCCAUG-3′ where N is A, U, C, or G, and AUG is a start codon. In some embodiments, the 5′ UTR sequence or the Kozak sequence is upstream or 5′ relative to the CDS within the synthesis module. In some embodiments, the CDS comprises exons interspaced with an intron. In some embodiments, the CDS comprises exons adjacent to one another without an intervening intron. In some embodiments, the CDS comprises exons 1, 3 and 4 of MECP2. In some embodiments, the CDS excludes exon 2 of MECP2. In some embodiments, the CDS encodes an e1 isoform of MECP2. In some embodiments, the CDS sequence is at least 80%, at least 85%, at least 90%, at least 95%, at least 98%, at least 99%, or 100% identical to a CDS sequence in Table 1. In some embodiments, the CDS comprises exon 2 of MECP2. In some embodiments, the CDS encodes an e2 isoform of MECP2. In some embodiments, the CDS comprises exons 1 and 3 of MECP2 with an intron between exons 1 and 3. In some embodiments, the CDS comprises an intron fragment sequence between exon 1 and 3. In some embodiments, the intron fragment sequence between exon 1 and 3 comprises a MECP2 intron 1 fragment sequence. In some embodiments, the MECP2 intron 1 fragment sequence is at least 80%, at least 85%, at least 90%, at least 95%, at least 98%, at least 99%, or 100% identical to an intron 1 fragment sequence in Table 1. In some embodiments, the CDS comprises exons 1 and 3 of MECP2 without an intron between exons 1 and 3. In some embodiments, the CDS comprises exons 3 and 4 of MECP2 with an intron between exons 3 and 4. In some embodiments, the CDS comprises an intron fragment sequence between exon 3 and 4. In some embodiments, the intron fragment sequence between exon 3 and 4 comprises a MECP2 intron 3 fragment sequence. In some embodiments, the MECP2 intron 3 fragment sequence is at least 80%, at least 85%, at least 90%, at least 95%, at least 98%, at least 99%, or 100% identical to an intron 3 fragment sequence in Table 1. In some embodiments, the CDS comprises exons 3 and 4 of MECP2 without an intron between exons 3 and 4. In some embodiments, the synthesis module further comprises a 3′ untranslated region (UTR) sequence of the MECP2 RNA. In some embodiments, the 3′ UTR sequence comprises an endogenous MECP2 3′ UTR sequence or fragment thereof. In some embodiments, the 3′ UTR sequence is downstream or 3′ relative to the promoter sequence, the SV40 intron sequence, the 5′ UTR sequence, the Kozak sequence, or the CDS within the synthesis module. In some embodiments, the synthesis module comprises a polyA signal sequence. In some embodiments, the 3′ UTR sequence comprises the polyA signal sequence. In some embodiments, the polyA signal sequence comprises a −bGH signal sequence, a SV40 signal sequence, or a hGH polyA signal sequence. In some embodiments, the silencing module reduces an MECP2 (e.g. MECP2 mRNA or protein) measurement in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%, relative to a baseline MECP2 measurement. In some embodiments, the synthesis module increases an MECP2 (e.g. MECP2 mRNA or protein) measurement in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, or at least 250%, relative to a baseline MECP2 measurement. In some embodiments, contact or expression of the system with a cell or cell population results in an MECP2 (e.g. MECP2 mRNA or protein) measurement between 1× and 2× relative to a control. In some embodiments, the 3′ UTR sequence or polyA signal sequence is at least 80%, at least 85%, at least 90%, at least 95%, at least 98%, at least 99%, or 100% identical to a 3′ UTR or polyA sequence in Table 1. In some embodiments, the silencing module comprises an RNA molecule. In some embodiments, the RNA molecule of the silencing module comprises a modified U7 small nuclear RNA (snRNA). In some embodiments, the modified U7 snRNA comprises a U7 core sequence at least 80%, at least 85%, at least 90%, at least 95%, at least 98%, at least 99%, or 100% identical to a U7 core sequence in Table 1. In some embodiments, the silencing module comprises a DNA molecule. In some embodiments, the DNA molecule of the silencing module encodes a modified U7 snRNA. In some embodiments, the DNA molecule of the silencing module comprises an arrayed series of silencing modules. In some embodiments, the silencing module further comprises a silencing module promoter sequence, or wherein the silencing module is encoded by a nucleic acid comprising the silencing module promoter sequence. In some embodiments, the silencing module promoter sequence comprises a human promoter sequence. In some embodiments, the silencing module promoter sequence comprises a mouse promoter sequence. In some embodiments, the silencing module promoter comprises a U7 snRNA promoter sequence. In some embodiments, the silencing module promoter sequence comprises a U1 snRNA promoter sequence. In some embodiments, the silencing module promoter sequence comprises a mouse U7 snRNA (“Mm U7”) promoter sequence, a human U7 snRNA (“Hs U7”) promoter sequence, a mouse U1a1 (“mu1a1” or “Mm U1a1”) promoter sequence, or a human U1-1 (“HU1” or “Hs U1-1”) promoter sequence, or a fragment or combination of fragments thereof. In some embodiments, the silencing module promoter sequence comprises a U7 snRNA promoter sequence having a distal sequence element (DSE) replaced with a DSE of a U1-1 or U1a1 promoter sequence. In some embodiments, the silencing module promoter sequence comprises a mouse U7 promoter sequence having a proximal sequence element (PSE) replaced with a PSE of a U1-1 or U1a1 promoter sequence. In some embodiments, the silencing module promoter sequence is at least 80%, at least 85%, at least 90%, at least 95%, at least 98%, at least 99%, or 100% identical to a promoter sequence in Table 1. In some embodiments, the silencing module promoter sequence is at least 80%, at least 85%, at least 90%, at least 95%, at least 98%, at least 99%, or 100% identical to a promoter sequence in Table 8. In some embodiments, the silencing module promoter sequence is 5′ or upstream relative to the ESS nucleic acid sequence or the antisense nucleic acid sequence. In some embodiments, the synthesis module is operatively coupled to the synthesis module promoter. In some embodiments, the silencing module is operatively coupled to the silencing module promoter. In some embodiments, the ESS recruits a protein factor or group of factors that reduce or silence splicing of the endogenous MECP2 RNA. In some embodiments, the ESS nucleic acid sequence comprises a nucleic acid sequence at least 80%, at least 85%, at least 90%, at least 95%, at least 98%, at least 99%, or at least 100% identical to ATGATAGGGACTTAGGGTGA (SEQ ID NO: 240). In some embodiments, the ESS nucleic acid sequence comprises a nucleic acid sequence at least 80%, at least 85%, at least 90%, at least 95%, at least 98%, at least 99%, or at least 100% identical to TTTGTTCCGTGGGTGGTTTA (SEQ ID NO: 241). In some embodiments, the ESS nucleic acid sequence comprises a nucleic acid sequence at least 80%, at least 85%, at least 90%, at least 95%, at least 98%, at least 99%, or at least 100% identical to TGGGGGGAGGTAGGTAGGTA (SEQ ID NO: 242). In some embodiments, the antisense nucleic acid sequence targets a targeted region of the endogenous MECP2 RNA. In some embodiments, the antisense nucleic acid sequence binds to the targeted region. In some embodiments, the antisense nucleic acid sequence is fully reverse complementary or partially reverse complementary (e.g. at least 90% reverse complementary) to the targeted region. In some embodiments, the antisense nucleic acid sequence is at least 80%, at least 85%, at least 90%, at least 95%, at least 98%, at least 99%, or at least 100% reverse complementary to the targeted region. In some embodiments, the targeted region is within an intron of the endogenous MECP2 RNA. In some embodiments, the intron comprises an intron between exons 1 and 3 of MECP2, or an intron between exons 3 and 4 of MECP2. In some embodiments, the targeted region is within an exon of the endogenous MECP2 RNA. In some embodiments, the antisense nucleic acid sequence targets an alternatively spliced exon of the endogenous MECP2 RNA. In some embodiments, the targeted region is within 100 nucleotides of an intron/exon junction. In some embodiments, the antisense nucleic acid sequence is 10-60 nucleotides in length. In some embodiments, the antisense nucleic acid sequence comprises a nucleic acid sequence at least 80%, at least 85%, at least 90%, at least 95%, at least 98%, at least 99%, or at least 100% identical to an antisense nucleic acid sequence in Table 2. In some embodiments, the antisense nucleic acid sequence is 3′ or downstream relative to the ESS nucleic acid sequence within the silencing module. In some embodiments, the silencing module further comprises an Sm binding site sequence. In some embodiments, the Sm binding site sequence comprises AAUUUGUCUAG (SEQ ID NO: 243) or AAUUUUUGGAG (SEQ ID NO: 244; smOPT). In some embodiments, the Sm binding site sequence is 3′ or downstream relative to the silencing module promoter sequence, the ESS nucleic acid sequence, or the antisense nucleic acid sequence within the silencing module. In some embodiments, the silencing module further comprises a hairpin sequence. In some embodiments, the hairpin sequence comprises a U7 small nuclear RNA (snRNA) 3′ hairpin sequence. In some embodiments, the hairpin sequence is 3′ or downstream relative to the silencing module promoter sequence, the ESS nucleic acid sequence, the antisense nucleic acid sequence, or the Sm binding site sequence within the silencing module. In some embodiments, the silencing module further comprises a terminator sequence. In some embodiments, the terminator sequence comprises mouse or human terminator sequence. In some embodiments, the terminator sequence comprises a U7 snRNA terminator sequence. In some embodiments, the terminator sequence comprises a U1 terminator sequence. In some embodiments, the terminator sequence comprises a Mm U7 terminator sequence, a Hs U7 terminator sequence, a mu1a1 terminator sequence, or a HU1 terminator sequence, or a fragment or combination of fragments thereof. In some embodiments, the terminator sequence comprises a U7 snRNA terminator sequence having a distal sequence element (DSE) replaced with a DSE of a U1-1 or U1a1 terminator sequence. In some embodiments, the terminator sequence comprises a mouse U7 snRNA terminator sequence having a proximal sequence element (PSE) replaced with a PSE of a U1-1 or U1a1 terminator sequence. In some embodiments, the terminator sequence is 3′ or downstream relative to the silencing module promoter sequence, the ESS nucleic acid sequence, the antisense nucleic acid sequence, the Sm binding site sequence, or the hairpin sequence. In some embodiments, the silencing module terminator sequence is at least 80%, at least 85%, at least 90%, at least 95%, at least 98%, at least 99%, or 100% identical to a promoter sequence in Table 1. In some embodiments, the silencing module terminator sequence is at least 80%, at least 85%, at least 90%, at least 95%, at least 98%, at least 99%, or 100% identical to a terminator sequence in Table 9. Some embodiments include one or more deoxyribonucleic acid (DNA) molecules comprising the synthesis module and the silencing module. In some embodiments, the system comprises an expression cassette. In some embodiments, the expression cassette encodes the synthesis module and the silencing module. In some embodiments, the synthesis module is upstream or 5′ relative to the silencing module. In some embodiments, the synthesis module is downstream or 3′ relative to the silencing module. In some embodiments, the components are combined together within a single nucleic acid. In some embodiments, some components are separated among multiple nucleic acids. In some embodiments, the synthesis module and the silencing module are included in separate nucleic acids. Disclosed herein, in some embodiments, are pharmaceutical compositions comprising a system described herein, and a pharmaceutically acceptable carrier. Disclosed herein, in some embodiments, are viruses comprising a system described herein. In some embodiments, the virus comprises an adeno-associated virus (AAV). Disclosed herein, in some embodiments, are cells comprising a system described herein. In some embodiments, the cell comprises a neural cell. Disclosed herein, in some embodiments, are methods, comprising administering the pharmaceutical composition or virus to a subject.

Disclosed herein, in some embodiments, are methods of treating a genetic disease in a subject, comprising: administering a system described herein to the subject. In some embodiments, the subject has been identified as having a genetic disease prior to the treatment. In some embodiments, the genetic disease is associated with haploinsufficiency of the endogenous MECP2 RNA. In some embodiments, the genetic disease is associated with tissue mosaic expression of the endogenous MECP2 RNA. In some embodiments, the genetic disease comprises Rett syndrome. Disclosed herein, in some embodiments, are methods, comprising: suppressing protein expression of an endogenous MECP2 RNA in a first cell expressing the endogenous MECP2 RNA; and synthesizing or enhancing protein expression of a recombinant version of the MECP2 RNA in a second cell that otherwise does not express the endogenous MECP2 RNA, or that expresses the endogenous MECP2 RNA at a low level. Some embodiments include synthesizing or enhancing protein expression of the recombinant version of the MECP2 RNA in the first cell. In some embodiments, said suppressing is performed upon contacting the first cell with a silencing module described herein. In some embodiments, suppressing protein expression comprises suppressing endogenous MECP2 protein expression by at least 10%. In some embodiments, said synthesizing or enhancing recombinant MECP2 protein expression is performed upon contacting the second cell with a synthesis module described herein. In some embodiments, enhancing recombinant MECP2 protein expression comprises enhancing protein expression by at least 10%. In some embodiments, the low level of expression of the endogenous MECP2 RNA in the second cell comprises an undetectable level, comprises a level below a desired level, comprises a level lower than a wild type cell, or comprises an expression lower than that of the first cell. In some embodiments, the low level of expression of the endogenous MECP2 RNA in the second cell comprises a level at least 10% lower than that of the first cell. In some embodiments, the silencing module and the synthesis module are encoded together in a nucleic acid construct. In some embodiments, the silencing module and the synthesis module are encoded in separate nucleic acid constructs. In some embodiments, the nucleic acid construct or the separate nucleic acid constructs are delivered to the cells using one or more viral vectors. In some embodiments, the one or more viral vectors comprise an AAV vector.

### BRIEF DESCRIPTION OF THE DRAWINGS

FIG. 1 is a schematic of a nucleic acid vector encoding RNAs for simultaneous depletion of endogenous MECP2 and expression of a regulated MECP2 transgene.

FIG. 2 is a schematic of an engineered 3′ UTR including of an array of fragments from an endogenous human MECP2 3′ UTR.

FIG. 3 is a plot showing experimental results where candidate U7 constructs were screened in quadruplicate and sorted by their combined rank within each experiment and their overall reduction of Mecp2 levels.

FIG. 4 is a plot showing experimental results where U7 candidates 1 and 2 were transfected into Neuro-2a cells and their ability to reduce Mecp2 levels was confirmed by qRT-PCR.

FIG. 5 is a plot showing experimental results where co-expression of U7 candidate 1 or 2 with an Mecp2 transgene containing fragments of intron 1 and 3 of Mecp2 reduces total Mecp2 expression in Neuro-2a cells.

FIG. 6 is a plot showing dose-dependent reduction of Mecp2 RNA levels in mouse primary cortical neurons cultured with different amounts of AAV expressing U7 candidate 1.

FIG. 7 is a plot showing that delivery of AAV expressing either U7 candidate 1 or 2 to adult C57Bl/6J mice reduced the level of MECP2 protein across transduced cells. The plot includes groups of 3 bar graphs. In each group, the left bar corresponds to U7-Scram, the middle bar corresponds to U7-01, and the right bar corresponds to U7-02.

FIG. 8 includes images of immunohistochemistry for MECP2, showing reduced MECP2 protein levels in animals injected with AAVs expressing Ubc-Mecp2 along with either U7 candidate 1 or 2 compared to animals expressing only Ubc-Mecp2.

FIG. 9 is a plot including immunohistochemistry results showing reduced MECP2 protein levels in animals injected with AAVs expressing Ubc-Mecp2 along with either U7 candidate 1 or 2 compared to animals expressing only Ubc-Mecp2

FIG. 10 is a plot of results where total MECP2 protein levels across thalamic cells were normalized in wildtype female mice injected with Ubc-Mecp2-U7-01 or Ubc-Mecp2-U7-02 compared to animals expressing Ubc-Mecp2 with a scrambled U7 cassette.

FIG. 11 is a violin plot of MECP2 protein expression levels across thalamic cells in female heterozygous Mecp2 mutant mice, showing normalization of MECP2 protein expression patterns in animals injected with Ubc-Mecp2-U7-01 or Ubc-Mecp2-U7-02 compared to animals expressing Ubc-Mecp2 with a scrambled U7 cassette.

FIG. 12 is a violin plot showing the per-cell levels of MECP2 protein in the cortex of wild-type mice injected systemically at P28 with the stated doses of either unregulated vector or U7 candidate 2.

FIG. 13 is a violin plot showing the per-cell levels of MECP2 protein in the thalamus of wild-type mice injected systemically at P28 with the stated doses of either unregulated vector or U7 candidate 2.

FIG. 14 is a log-transformed violin plot showing the per-cell levels of MECP2 protein in the thalamus of wild-type and Mecp2^{+/-} mice injected systemically at P28 with the stated dose of either unregulated vector or U7 candidate 2.

FIG. 15 is a Kaplan-Meier plot showing the survival curves of saline or U7 candidate 2-injected male mice of the noted genotypes and doses. Animals were injected systemically at P28.

FIG. 16 is a plot showing weekly phenotypic “Bird” scores, a commonly used scoring system to assess phenotypic progression in mouse models of Rett Syndrome. Mice of the stated genotypes were given IV injection of saline or virus at the described doses at P28.

FIG. 17 is a Kaplan-Meier plot showing the survival curves of saline or U7 candidate 2-injected male mice of the noted genotypes and doses. Animals were injected systemically at P14.

FIG. 18 is a plot showing weekly phenotypic “Bird” scores, a commonly used scoring system to assess phenotypic progression in mouse models of Rett Syndrome. Mice of the stated genotypes were given IV injection of saline or virus at the described doses at P14.

FIG. 19 is a plot showing the weekly body weights of wild-type and heterozygous Mecp2 mutant female mice injected with saline or the described doses of AAV at P28.

FIG. 20 is quantification of total MECP2 protein levels in differentiated ReNcell CX cultures with two candidate U7 constructs. Cells were transduced with 1e10 vg AAV, which gives ~70% transduction efficiency, or treated with formulation buffer for 1-week before analysis.

FIG. 21 is a schematic of the fully humanized therapeutic vector.

FIG. 22 is quantification of total MECP2 protein levels in differentiated ReNcell CX cultures treated with formulation buffer, the mouse therapeutic vector used in mouse studies, or one of two different humanized therapeutic candidate vectors corresponding to the U7 sequences used in FIG. 21. Cells were transduced with the stated doses of AAV or treated with formulation buffer for 1-week before analysis.

### DETAILED DESCRIPTION

An approach was developed that is useful for avoiding toxic overexpression of MECP2 by depleting endogenous MECP2 in cells expressing from the wildtype X chromosome, while simultaneously expressing wildtype MECP2 under the control of a constitutively expressed promoter and endogenous regulatory elements. The approach may include a gene therapy approach useful for treating Rett Syndrome without toxic transgene overexpression including multiple functional units. The approach may include a silencing module and a synthesis module. The silencing module may make use of a modified U7 small nuclear RNA (snRNA) that silences or reduces endogenous MECP2 protein expression. The synthesis module may use a recombinant MECP2 gene or MECP2 mRNA to produce MECP2 protein.

This disclosure includes a therapy in which expression of a target gene is both knocked down and increased within a cell. Due to random X-inactivation, cells in Rett syndrome patients may have either some MECP2 or none. AAV may be delivered to multiple cells and in the cells that express MECP2 the endogenous transcript is suppressed and then replaced by expression from the AAV. In some embodiments, in cells that lack MECP2, no knockdown of endogenous MECP2 occurs, but expression of MECP2 from the AAV occurs.

Disclosed herein, in some embodiments, are systems for altering methyl CpG binding protein 2 (MECP2) expression. The system may include a dual system for reducing endogenous MECP2, while expressing recombinant MECP2. This may be done in in separate cells, or both in the same cell, depending for example on the level of endogenous MECP2 expression in the cell. Some embodiments include an RNA such as a modified U7 small nuclear RNA (snRNA) that reduces endogenous MECP2 expression. Some embodiments include an RNA that encodes the recombinant MECP2. Some embodiments include an expression construct encoding the RNAs. Also included are methods of using the system to modify MECP2 expression. The method may be used to treat a disorder associated with abnormal MECP2 expression such as Rett syndrome or MECP2 duplication syndrome.

Some embodiments relate to a system for altering gene expression. The system may include a silencing module. The silencing module may include an exonic splicing silencer (ESS) nucleic acid sequence. The ESS may be coupled with an antisense nucleic acid sequence targeting an endogenous target ribonucleic acid (RNA). The system may include a synthesis module. The synthesis module may include a nucleic acid coding sequence (CDS). The CDS may encode a recombinant version of the target RNA. Some embodiments relate to a system for altering gene expression, comprising: a silencing module comprising an exonic splicing silencer (ESS) nucleic acid sequence coupled with an antisense nucleic acid sequence targeting an endogenous target ribonucleic acid (RNA); and a synthesis module comprising a nucleic acid coding sequence (CDS) that encodes a recombinant version of the target RNA. An example of a target RNA may include an RNA encoding MECP2. For the system provided herein, in embodiments, the components are combined together within a single nucleic acid. For example, the components may be in a single viral vector or plasmid. In embodiments, the components are in separate nucleic acids. In embodiments, some components are separated among multiple nucleic acids. In embodiments, the synthesis module and the silencing module are included in separate nucleic acids. For example, the synthesis module may be in a first expression cassette and the silencing module may be in a second expression cassette. The system may include an expression construct. The system may include RNAs such as a U7 sRNA and an mRNA. The RNAs may be encoded by an expression construct. The system may be included in a method such as a method of treatment. An example of a disease that may be treated may include Rett syndrome. Another example of a disease that may be treated include Mecp2 duplication syndrome.

Described herein are compositions and methods for treating a neurodevelopmental disorder in a subject in need thereof. The compositions and methods described herein address an unmet need for safe and effective treatment of neurodevelopmental disorders. The neurodevelopmental disorder may be genetically caused, such as by a mutation in MECP2 (e.g. resulting in MECP2 haploinsufficiency). Described herein are compositions and methods for treating disorders such as Rett syndrome or MECP2 duplication syndrome in a subject in need thereof. The compositions and methods described herein address an unmet need for safe and effective treatment of disorders such as Rett syndrome and MECP2 duplication syndrome.

The compositions and methods provided herein may improve upon previous methods and systems. Some previous systems are described at Sinnett et al, Brain, 2021 Nov. 29; 144(10):3005-3019; Gadalla et al, Mol Therapy, 2017 Apr. 22:5:180-190; Sinnett et al, Mol Therapy, 2017 Apr. 19:5:106-115; and Luoni et al, eLife, 2020 Mar. 24:9:e52629, which references are incorporated by reference in their entirety.

In some embodiments, contact or expression of the system with a cell or cell population results in a target (e.g. protein or RNA, such as a MECP2 protein or RNA) measurement of at least 0.5×, at least 0.6×, at least 0.7×, at least 0.8×, at least 0.9×, at least 1×, at least 1.1×, at least 1.2×, at least 1.3×, at least 1.4×, at least 1.5×, at least 1.6×, at least 1.7×, at least 1.8×, at least 1.9×, at least 2×, at least 2.1×, at least 2.2×, at least 2.3×, at least 2.4×, or at least 2.5×, relative to a control. In some embodiments, contact or expression of the system with a cell or cell population results in a target (e.g. protein or RNA, such as a MECP2 protein or RNA) measurement of less than 0.5×, less than 0.6×, less than 0.7×, less than 0.8×, less than 0.9×, less than 1×, less than 1.1×, less than 1.2×, less than 1.3×, less than 1.4×, less than 1.5×, less than 1.6×, less than 1.7×, less than 1.8×, less than 1.9×, less than 2×, less than 2.1×, less than 2.2×, less than 2.3×, less than 2.4×, or less than 2.5×, relative to a control. In some embodiments, contact or expression of the system with a cell or cell population results in a target (e.g. protein or RNA, such as a MECP2 protein or RNA) measurement between 0.5× and 2.5× relative to a control. In some embodiments, contact or expression of the system with a cell or cell population results in a target (e.g. protein or RNA, such as a MECP2 protein or RNA) measurement between 1× and 2× relative to a control. The control may be or include a cell or cell population not contacted with the system. The control may be or include a cell or cell population that does not express the system.

### Expression Systems

Provided herein, inter alia, are expression systems. The expression system may be capable of silencing expression of an endogenous target such as MECP2, and synthesizing or enhancing expression of a recombinant version of MECP2. The expression systems provided herein including embodiments thereof may include a modified U7 small nuclear RNA (snRNA) capable of specifically targeting an endogenous MECP2 messenger RNA (mRNA) or portion thereof to increase or decrease splicing of one or more exons in the endogenous MECP2 mRNA (e.g. pre-mRNA), thereby inhibiting or downregulating expression of endogenous MECP2. For example, the expression system may include an exonic splicing silencer (ESS) sequence capable of downregulating or inhibiting splicing or inducing exon skipping and an antisense nucleic acid sequence targeting an endogenous MECP2 ribonucleic acid (RNA), thereby silencing expression of endogenous MECP2. The expression systems provided herein including embodiments thereof may further include a MECP2 synthesis module allowing expressing of a recombinant MECP2. For example, the MECP2 synthesis module may include a nucleic acid coding sequence that encodes MECP2 or a recombinant MECP2 having at least 80% identity to a wild type MECP2. The expression system may be or include a DNA construct that encodes one or more RNAs.

In embodiments, silencing expression of a gene refers to inhibiting or downregulating expression levels of the gene. For example, silencing expression of a gene includes inhibiting or downregulating pre-mRNA processing. In another example, silencing expression of a gene includes downregulating or inhibiting production of a protein encoded by the gene. In embodiments, the expression system decreases expression of an endogenous gene at least 10%, 20%, 30%, 40%, 50%, 60%, 70%, 80%, 85%, 90%, 91%, 92% 93%, 94%, 95%, 96%, 97%, 98%, 99% or 100% relative to expression of the endogenous gene in the absence of the expression system. For example, the expression system may decrease the level of an endogenous MECP2 protein at least 10%, 20%, 30%, 40%, 50%, 60%, 70%, 80%, 85%, 90%, 91%, 92% 93%, 94%, 95%, 96%, 97%, 98%, 99% or 100% relative to the level of endogenous MECP2 protein in the absence of the expression system.

Some embodiments include synthesizing MECP2 protein (e.g. from an mRNA encoding the MECP2 protein). The MECP2 mRNA may be encoded by a synthetic construct. In embodiments, synthesizing a recombinant version of a MECP2 gene or a recombinant MECP2 mRNA includes to expressing MECP2 from a nucleic acid exogenous to the cell. In embodiments, the expression system provided herein produces a recombinant MECP2 protein (e.g. MECP2 protein produced from the recombinant mRNA), wherein the level of the recombinant MECP2 protein is at least 20% of the level of an endogenous MECP2 protein in a healthy cell (e.g. in a cell that does not have Rett syndrome). In some embodiments, the level of the recombinant version is at least 10%, at least 20%, at least 30%, at least 40%, at least 50%, at least 60%, at least 70%, at least 80%, at least 90%, at least 100%, at least 110%, or at least 120% of the level in a healthy cell. In some embodiments, the level of the recombinant version is no greater than 10%, no greater than 20%, no greater than 30%, no greater than 40%, no greater than 50%, no greater than 60%, no greater than 70%, no greater than 80%, no greater than 90%, no greater than 100%, no greater than 110%, or no greater than 120% of the level in a healthy cell.

The MECP2 protein may be recombinant. In some embodiments, the MECP2 protein is considered recombinant because it is produced from a recombinant nucleic acid, or is produced from an mRNA encoded by a recombinant nucleic acid. In some embodiments, the MECP2 protein is further considered recombinant in that it includes a modification relative to an endogenous MECP2. In embodiments, the recombinant MECP2 protein produced by the recombinant mRNA has at least 80% sequence identity to an endogenous MECP2 protein. In embodiments, the recombinant MECP2 protein produced by the recombinant mRNA has at least 85% sequence identity to an endogenous MECP2 protein. In embodiments, the recombinant MECP2 protein produced by the recombinant mRNA has at least 90% sequence identity to an endogenous MECP2 protein. In embodiments, the recombinant MECP2 protein produced by the recombinant mRNA has at least 91% sequence identity to an endogenous MECP2 protein. In embodiments, the recombinant MECP2 protein produced by the recombinant mRNA has at least 92% sequence identity to an endogenous MECP2 protein. In embodiments, the recombinant MECP2 protein produced by the recombinant mRNA has at least 93% sequence identity to an endogenous MECP2 protein. In embodiments, the recombinant MECP2 protein produced by the recombinant mRNA has at least 94% sequence identity to an endogenous MECP2 protein. In embodiments, the recombinant MECP2 protein produced by the recombinant mRNA has at least 95% sequence identity to an endogenous MECP2 protein. In embodiments, the recombinant MECP2 protein produced by the recombinant mRNA has at least 96% sequence identity to an endogenous MECP2 protein. In embodiments, the recombinant MECP2 protein produced by the recombinant mRNA has at least 97% sequence identity to an endogenous MECP2 protein. In embodiments, the recombinant MECP2 protein produced by the recombinant mRNA has at least 98% sequence identity to an endogenous MECP2 protein. In embodiments, the recombinant MECP2 protein produced by the recombinant mRNA has at least 99% sequence identity to an endogenous MECP2 protein. In embodiments, the recombinant MECP2 protein produced by the recombinant mRNA has includes the sequence of an endogenous MECP2 protein. In embodiments, the recombinant MECP2 protein produced by the recombinant mRNA has is the sequence of an endogenous MECP2 protein. In some embodiments, the recombinant MECP2 has less than 100%, less than 99%, less than 98%, less than 97%, less than 96%, less than 95%, less than 94%, less than 93%, less than 92%, less than 91%, less than 90%, or less than 85%, sequence identity to the endogenous MECP2 protein.

In some aspects is provided an expression system. The system may be useful for altering gene expression. The system may include a silencing module. The silencing module may include a silencing module deoxyribonucleic acid (DNA) sequence. The silencing module may include a first promoter sequence. The silencing module may include an exonic splicing silencer (ESS) nucleic acid sequence. The silencing module may include an antisense nucleic acid sequence targeting an endogenous MECP2 ribonucleic acid (RNA). The silencing module may include an Sm binding site sequence. The silencing module may include a 3′ hairpin sequence. The silencing module may include a 3′ terminator sequence. The system may include an MECP2 synthesis module. The synthesis module may include a synthesis module DNA sequence. The synthesis module may include a second promoter sequence. The synthesis module may include a 5′ untranslated region (UTR) sequence. The synthesis module may include a nucleic acid coding sequence (CDS) encoding MECP2. The synthesis module may include a 3′ UTR sequence. In some aspects, the silencing module encodes a modified U7 small nuclear RNA (snRNA) that silences or reduces endogenous MECP2 protein expression. In some aspects, the MECP2 synthesis module encodes a recombinant messenger RNA (mRNA) that generates recombinant MECP2 protein.

In an aspect is provided an expression system for altering gene expression, including: a silencing module deoxyribonucleic acid (DNA) sequence including: a first promoter sequence, an exonic splicing silencer (ESS) nucleic acid sequence, an antisense nucleic acid sequence targeting an endogenous MECP2 ribonucleic acid (RNA), an Sm binding site sequence, a 3′ hairpin sequence, and a 3′ terminator sequence; and an MECP2 synthesis module DNA sequence including: a second promoter sequence, a 5′ untranslated region (UTR) sequence, a nucleic acid coding sequence (CDS) encoding MECP2, and a 3′ UTR sequence; wherein the silencing module encodes a modified U7 small nuclear RNA (snRNA) that silences or reduces endogenous MECP2 protein expression, and the MECP2 synthesis module encodes a recombinant messenger RNA (mRNA) that generates recombinant MECP2 protein.

The term “snRNA” and “small nuclear RNA” may include RNA molecules typically involved in processing other RNA molecules (e.g. pre-mRNA, histone RNA, etc.). For example, snRNA may play a role in splicing pre-mRNA molecules. For example, snRNA are capable of binding and/or recruiting proteins involved in RNA processing. The snRNA/protein complex may be referred to as a small nuclear ribonucleoprotein (snRNP). For example, an snRNA may bind to one or more Sm proteins, which may typically regulate and are involved in pre-mRNA processing. In embodiments, the snRNA is between about 40 and 200 nt in length. In embodiments, the snRNA is U7 snRNA.

The terms “U7 small nuclear snRNA” and “U7 snRNA” may refer to an RNA molecule that may form part of the small nuclear ribonucleoprotein complex (U7 snRNP), or may play a role in processing of mRNAs. A “modified U7 small nuclear snRNA” or “modified snRNA” may refer to a U7 snRNA that includes one or more sequence modifications to affect splicing of a target RNA such as an MECP2 RNA. The modified snRNA may also include a backbone modification. In embodiments, the U7 snRNA is modified to target a portion of an mRNA such as an MECP2 mRNA. In embodiments, the modified U7 snRNA may target or bind to an endogenous MECP2 RNA (e.g. an endogenous MECP2 pre-mRNA, or an endogenous MECP2 mRNA). In embodiments, the U7 snRNA is modified to be at least partially complementary to the endogenous MECP2 RNA (e.g. to an exon of the MECP2 RNA). In embodiments, modification of the U7 snRNA sequence inhibits binding of splicing factors. Thus, in embodiments, the modified U7 snRNA does not bind splicing factors. In embodiments, modification of the U7 snRNA allows recruitment of splicing proteins capable of splicing an endogenous MECP2 RNA (e.g. MECP2 pre-mRNA).

An “exonic splicing silencer” or “ESS” sequence may refer to a nucleic acid sequence that may inhibit or downregulate splicing of an endogenous target RNA (e.g. target pre-mRNA or target mRNA). In embodiments, the ESS inhibits or downregulates splicing of an endogenous target pre-mRNA by inhibiting binding or recruitment of one or more components of the splicing complex to the endogenous target RNA. In embodiments, the ESS inhibits or downregulates splicing of an endogenous target pre-mRNA by inducing exon skipping. Thus, in embodiments, the ESS inhibits or downregulates splicing of an endogenous target RNA. An example of an ESS sequence may include 5′-ATGATAGGGACTTAGGGTGA-3′ (SEQ ID NO: 240), 5′-TTTGTTCCGTGGGTGGTTTA-3′ (SEQ ID NO: 241), or 5′-TGGGGGGAGGTAGGTAGGTA-3′ (SEQ ID NO: 242). The ESS sequence may be at least 90%, 91%, 92%, 93%, 94%, 95%, 96%, 97%, 98%, or 99%, identical to any of the aforesaid ESS sequences or a reverse complement thereof. Ts may be replaced with Us when the sequences is an RNA sequence (as opposed to, for example, a DNA sequence).

An antisense nucleic acid sequence targeting an MECP2 ribonucleic acid (RNA) may include a nucleic acid sequence capable of recruiting the silencing module DNA sequence to the MECP2 RNA or a portion thereof. In embodiments, the antisense nucleic acid sequence targeting the endogenous MECP2 RNA includes a sequence at least partially complementary to a portion of the endogenous MECP2 RNA. In embodiments, the antisense nucleic acid sequence targeting the endogenous MECP2 RNA includes a sequence at least partially complementary to a splice site of the endogenous MECP2 RNA.

An “Sm binding site sequence” may include a sequence that binds to or recruits one or more proteins involved in splicing RNA. In embodiments, the Sm binding site sequence binds to or recruits one or more Sm proteins. An “Sm protein” may include one or more proteins found in the spliceosomal small nuclear ribonucleoprotein complex. In embodiments, the Sm binding site binds to or recruits one or more of SmB/B0, SmD3, SmE, SmF and SmG. In embodiments, the Sm binding site sequence binds to or recruit Lsm proteins (e.g. Lsm10, Lsm11). In embodiments, the Sm binding site sequence does not bind to or recruit Lsm proteins (e.g. Lsm10, Lsm11). In embodiments, the Sm binding site binds or recruits one or more pre-mRNA spliceosome proteins.

The terms “hairpin sequence”, “stem loop sequence”, “hairpin loop sequence” may be interchangeable, and may refer to a region of an RNA oligonucleotide (e.g. RNA stem loop oligonucleotide) that that includes two nucleotide sequences that base pair to form a double-stranded (e.g. RNA double-helix) structure (e.g. the stem) with a non-base paired structure (e.g. the loop) at one end of the double-stranded structure. The double-stranded structure (e.g. stem) in the RNA stem loop may be referred to as an “RNA double-helix”. In embodiments, the hairpin sequence may form a portion of the modified U7 snRNA. In embodiments, the hairpin sequence may increase stability of the modified U7 small snRNA. In embodiments, the hairpin sequence is located 3′ of the antisense nucleic acid sequence targeting an endogenous MECP2 RNA.

The terms “terminator sequence” or “terminator” may include a sequence that regulates RNA production. For example, a terminator sequence may signal the end of an RNA molecule. In embodiments, the terminator sequence refers to a sequence at the 3′ end of the snRNA silencing module. In embodiments, the terminator sequence regulates stability of an RNA molecule. For example, the terminator sequence may increase stability of the U7 snRNA.

### MECP2

Some embodiments relate to a methyl CpG binding protein 2 (MECP2) protein. For example, a method may be directed at reducing or increasing expression of MECP2. Some examples of MECP2 protein sequences are included at UniProt.org under accession numbers P51608 (human) and Q9Z2D6 (mouse), as last updated as of the effective filing date. The MECP2 protein may include the amino acid sequence of SEQ ID NO: 364. The MECP2 protein may include an amino acid sequence at least 85%, at least 90%, at least 91%, at least 92%, at least 93%, at least 94%, at least 95%, at least 96%, at least 97%, at least 98%, or at least 99% identical to SEQ ID NO: 364.

A MECP2 protein may be encoded by an MECP2 RNA. An MECP2 RNA may be included in or encoded by an MECP2 synthesis module. An MECP2 RNA may be targeted by a silencing module. Some examples of MECP2 mRNA sequences may included sequences found at ncbi.nlm.nih.gov at NCBI Reference Sequence: NM_001110792, NM_004992, NM_001316337, NM_001369391, or NM_001369392, as last updated as of the effective filing date.

There are multiple possible isoforms of MECP2. One example utilizes exons 1, 3, and 4 (where 2 is spliced out) and one example utilizes 1, 2, 3, and 4. In the second isoform an alternative translation start site at the 3′ end of exon 2 is used instead of the one located in exon 1, so there is an extended 5′ UTR. This isoform is almost exclusively expressed outside of the CNS during embryonic development. Its utilization is then shut off in most/all tissues after birth. Examples of exon 1 are provided as SEQ ID NO: 365 (mouse) and 368 (human). Examples of exon 3 are provided as SEQ ID NO: 366 (mouse) and 369 (human). Examples of exon 4 are provided as SEQ ID NO: 367 (mouse) and 370 (human).

### Systems for Altering MECP2 Expression

In some aspects is provided a system for altering gene expression. The system may include one or more RNAs, such as a U7 RNA or silencing module, and an RNA synthesis module. The system may be encoded by an expression system or an expression construct herein.

In some aspects is provided a system for altering gene expression. The system may include a dual RNA system including. The system may include a silencing module. The silencing module may include a U7 small nuclear RNA (snRNA) silencing module. The silencing module may include an exonic splicing silencer (ESS) nucleic acid sequence. The silencing module may include an antisense nucleic acid sequence. The antisense nucleic acid sequence may bind an endogenous MECP2 ribonucleic acid (RNA). The silencing module may include an Sm binding site sequence. The silencing module may include a 3′ hairpin sequence. The system may include a synthesis module. The synthesis module may include an MECP2 messenger RNA (mRNA) synthesis module. The synthesis module may include a 5′ untranslated region (UTR) sequence. The synthesis module a nucleic acid coding sequence (CDS). The CDS may encode encoding an MECP2 protein (e.g. a recombinant MECP2 protein). The synthesis module may include a 3′ UTR. In some aspects, the U7 snRNA silencing module silences or reduces an endogenous target protein expression. In some aspects, the target mRNA synthesis module generates a recombinant version of the target protein.

In some aspects is provided a dual RNA system for altering gene expression, including: a U7 small nuclear RNA (snRNA) silencing module including: an exonic splicing silencer (ESS) nucleic acid sequence, an antisense nucleic acid sequence that binds an endogenous MECP2 ribonucleic acid (RNA), an Sm binding site sequence, and a 3′ hairpin sequence; and an MECP2 messenger RNA (mRNA) synthesis module including: a 5′ untranslated region (UTR) sequence, a nucleic acid coding sequence (CDS) encoding MECP2, and a 3′ UTR sequence; wherein the U7 snRNA silencing module silences or reduces endogenous MECP2 protein expression, and the MECP2 mRNA synthesis module generates recombinant MECP2 protein.

Provided herein, inter alia, are systems for modulating expression levels of a MECP2 in a cell. For example, systems provided herein including embodiments thereof are contemplated to be useful for a producing a recombinant version of a MECP2 protein in a cell, wherein the expression level of the recombinant version of MECP2 protein is at least 20%, 30%, 40%, 50%, 60%, 70%, 80%, 85%, 90%, 91%, 92%, 93%, 95%, 95%, 96%, 97%, 98%, 99%, or 100% of the expression level of the endogenous MECP2 protein in a healthy cell (e.g. a cell without Rett syndrome). In embodiments, the expression level of the recombinant MECP2 gene is at least 0.2-fold, 0.3-fold, 0.4-fold, 0.5-fold, 0.6-fold, 0.7-fold, 0.8-fold, 0.9-fold, or 1-fold of the expression level of the endogenous MECP2 gene in a healthy cell (e.g. a cell without a genetic disorder, a cell without Rett syndrome). In embodiments, the expression level of the recombinant MECP2 protein is less than 4-fold, 3-fold, 2-fold, 1.8-fold, 1.6-fold, 1.5-fold, 1.4-fold, 1.3-fold, 1.2-fold, 1.1-fold, 1-fold, 0.9-fold, 0.8-fold, 0.7-fold, 0.6-fold, or 0.5-fold than the expression level of the endogenous MECP2 protein in a healthy cell (e.g. a cell without Rett syndrome).

Thus, in an aspect is provided a system for altering gene expression, including: a silencing module including an exonic splicing silencer (ESS) nucleic acid sequence coupled with an antisense nucleic acid sequence targeting an endogenous MECP2 ribonucleic acid (RNA); and a synthesis module including a nucleic acid coding sequence (CDS) that encodes a recombinant version of the MECP2 RNA. In embodiments, the endogenous MECP2 RNA includes an mRNA splice site.

For the system provided herein, in embodiments, the expression level of the recombinant MECP2 protein is between about 0.2-fold to about 4-fold of the expression level of the endogenous MECP2 protein in a healthy cell (e.g. a cell without Rett syndrome). In embodiments, the expression level of the recombinant MECP2 protein is between about 0.3-fold to about 4-fold of the expression level of the endogenous MECP2 protein in a healthy cell (e.g. a cell without Rett syndrome). In embodiments, the expression level of the recombinant MECP2 protein is between about 0.4-fold to about 4-fold of the expression level of the endogenous MECP2 protein in a healthy cell (e.g. a cell without Rett syndrome). In embodiments, the expression level of the recombinant MECP2 protein is between about 0.5-fold to about 4-fold of the expression level of the endogenous MECP2 protein in a healthy cell (e.g. a cell without Rett syndrome). In embodiments, the expression level of the recombinant MECP2 protein is between about 0.6-fold to about 4-fold of the expression level of the endogenous MECP2 protein in a healthy cell (e.g. a cell without Rett syndrome). In embodiments, the expression level of the recombinant MECP2 protein is between about 0.7-fold to about 4-fold of the expression level of the endogenous MECP2 protein in a healthy cell (e.g. a cell without Rett syndrome). In embodiments, the expression level of the recombinant MECP2 protein is between about 0.8-fold to about 4-fold of the expression level of the endogenous MECP2 protein in a healthy cell (e.g. a cell without Rett syndrome). In embodiments, the expression level of the recombinant MECP2 protein is between about 0.9-fold to about 4-fold of the expression level of the endogenous MECP2 protein in a healthy cell (e.g. a cell without Rett syndrome). In embodiments, the expression level of the recombinant MECP2 protein is between about 1-fold to about 4-fold of the expression level of the endogenous MECP2 protein in a healthy cell (e.g. a cell without Rett syndrome).

In embodiments, the expression level of the recombinant MECP2 protein is between about 1.1-fold to about 4-fold of the expression level of the endogenous MECP2 protein in a healthy cell (e.g. a cell without Rett syndrome). In embodiments, the expression level of the recombinant MECP2 protein is between about 1.2-fold to about 4-fold of the expression level of the endogenous MECP2 protein in a healthy cell (e.g. a cell without Rett syndrome). In embodiments, the expression level of the recombinant MECP2 protein is between about 1.3-fold to about 4-fold of the expression level of the endogenous MECP2 protein in a healthy cell (e.g. a cell without Rett syndrome). In embodiments, the expression level of the recombinant MECP2 protein is between about 1.4-fold to about 4-fold of the expression level of the endogenous MECP2 protein in a healthy cell (e.g. a cell without Rett syndrome). In embodiments, the expression level of the recombinant MECP2 protein is between about 1.5-fold to about 4-fold of the expression level of the endogenous MECP2 protein in a healthy cell (e.g. a cell without Rett syndrome). In embodiments, the expression level of the recombinant MECP2 protein is between about 1.6-fold to about 4-fold of the expression level of the endogenous MECP2 protein in a healthy cell (e.g. a cell without Rett syndrome). In embodiments, the expression level of the recombinant MECP2 protein is between about 1.7-fold to about 4-fold of the expression level of the endogenous MECP2 protein in a healthy cell (e.g. a cell without Rett syndrome). In embodiments, the expression level of the recombinant MECP2 protein is between about 1.8-fold to about 4-fold of the expression level of the endogenous MECP2 protein in a healthy cell (e.g. a cell without Rett syndrome). In embodiments, the expression level of the recombinant MECP2 protein is between about 1.9-fold to about 4-fold of the expression level of the endogenous MECP2 protein in a healthy cell (e.g. a cell without Rett syndrome). In embodiments, the expression level of the recombinant MECP2 protein is between about 2-fold to about 4-fold of the expression level of the endogenous MECP2 protein in a healthy cell (e.g. a cell without Rett syndrome). In embodiments, the expression level of the recombinant MECP2 protein is between about 2.1-fold to about 4-fold of the expression level of the endogenous MECP2 protein in a healthy cell (e.g. a cell without Rett syndrome). In embodiments, the expression level of the recombinant MECP2 protein is between about 2.2-fold to about 4-fold of the expression level of the endogenous MECP2 protein in a healthy cell (e.g. a cell without Rett syndrome). In embodiments, the expression level of the recombinant MECP2 protein is between about 2.3-fold to about 4-fold of the expression level of the endogenous MECP2 protein in a healthy cell (e.g. a cell without Rett syndrome). In embodiments, the expression level of the recombinant MECP2 protein is between about 2.4-fold to about 4-fold of the expression level of the endogenous MECP2 protein in a healthy cell (e.g. a cell without Rett syndrome). In embodiments, the expression level of the recombinant MECP2 protein is between about 2.5-fold to about 4-fold of the expression level of the endogenous MECP2 protein in a healthy cell (e.g. a cell without Rett syndrome). In embodiments, the expression level of the recombinant MECP2 protein is between about 2.6-fold to about 4-fold of the expression level of the endogenous MECP2 protein in a healthy cell (e.g. a cell without Rett syndrome). In embodiments, the expression level of the recombinant MECP2 protein is between about 2.7-fold to about 4-fold of the expression level of the endogenous MECP2 protein in a healthy cell (e.g. a cell without Rett syndrome). In embodiments, the expression level of the recombinant MECP2 protein is between about 2.8-fold to about 4-fold of the expression level of the endogenous MECP2 protein in a healthy cell (e.g. a cell without Rett syndrome). In embodiments, the expression level of the recombinant MECP2 protein is between about 2.9-fold to about 4-fold of the expression level of the endogenous MECP2 protein in a healthy cell (e.g. a cell without Rett syndrome). In embodiments, the expression level of the recombinant MECP2 protein is between about 3-fold to about 4-fold of the expression level of the endogenous MECP2 protein in a healthy cell (e.g. a cell without Rett syndrome). In embodiments, the expression level of the recombinant MECP2 protein is between about 3.1-fold to about 4-fold of the expression level of the endogenous MECP2 protein in a healthy cell (e.g. a cell without Rett syndrome). In embodiments, the expression level of the recombinant MECP2 protein is between about 3.2-fold to about 4-fold of the expression level of the endogenous MECP2 protein in a healthy cell (e.g. a cell without Rett syndrome). In embodiments, the expression level of the recombinant MECP2 protein is between about 3.3-fold to about 4-fold of the expression level of the endogenous MECP2 protein in a healthy cell (e.g. a cell without Rett syndrome). In embodiments, the expression level of the recombinant MECP2 protein is between about 3.4-fold to about 4-fold of the expression level of the endogenous MECP2 protein in a healthy cell (e.g. a cell without Rett syndrome). In embodiments, the expression level of the recombinant MECP2 protein is between about 3.5-fold to about 4-fold of the expression level of the endogenous MECP2 protein in a healthy cell (e.g. a cell without Rett syndrome). In embodiments, the expression level of the recombinant MECP2 protein is between about 3.6-fold to about 4-fold of the expression level of the endogenous MECP2 protein in a healthy cell (e.g. a cell without Rett syndrome). In embodiments, the expression level of the recombinant MECP2 protein is between about 3.7-fold to about 4-fold of the expression level of the endogenous MECP2 protein in a healthy cell (e.g. a cell without Rett syndrome). In embodiments, the expression level of the recombinant MECP2 protein is between about 3.8-fold to about 4-fold of the expression level of the endogenous MECP2 protein in a healthy cell (e.g. a cell without Rett syndrome). In embodiments, the expression level of the recombinant MECP2 protein is between about 3.9-fold to about 4-fold of the expression level of the endogenous MECP2 protein in a healthy cell (e.g. a cell without Rett syndrome). In embodiments, the expression level of the recombinant MECP2 protein is about 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, or 4-fold of the expression level of the endogenous MECP2 protein in a healthy cell (e.g. a cell without a genetic disorder, a cell without Rett Syndrome). In embodiments, the expression level of the recombinant MECP2 protein is no greater than 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, or 4-fold of the expression level of the endogenous MECP2 protein in a healthy cell (e.g. a cell without a genetic disorder, a cell without Rett Syndrome). In embodiments, the expression level of the recombinant MECP2 protein is no greater than the expression level of the endogenous MECP2 protein in a healthy cell (e.g. a cell without a genetic disorder, a cell without Rett Syndrome).

The systems provided herein are further capable of silencing (e.g. inhibiting, decreasing, or downregulating) the expression level of the endogenous MECP2 gene in a cell. In embodiments, the system decreases the expression level of the endogenous MECP2 gene at least 20%, 30%, 40%, 50%, 60%, 70%, 80%, 85%, 90%, 91%, 92%, 93%, 95%, 95%, 96%, 97%, 98%, 99%, or 100% compared to the expression level of the endogenous MECP2 gene in the absence of the system. In embodiments, the system decreases the expression level of the endogenous MECP2 gene wherein the expression level of the endogenous MECP2 gene in a cell is undetectable. In embodiments, the expression level of the endogenous MECP2 gene is measured by the level of endogenous MECP2 protein produced by the endogenous MECP2 gene.

In embodiments, the system decreases the expression level of the endogenous MECP2 gene from about 20% to about 100% compared to the expression level of the endogenous MECP2 gene in the absence of the system. In embodiments, the system decreases the expression level of the endogenous MECP2 gene from about 25% to about 100% compared to the expression level of the endogenous MECP2 gene in the absence of the system. In embodiments, the system decreases the expression level of the endogenous MECP2 gene from about 30% to about 100% compared to the expression level of the endogenous MECP2 gene in the absence of the system. In embodiments, the system decreases the expression level of the endogenous MECP2 gene from about 35% to about 100% compared to the expression level of the endogenous MECP2 gene in the absence of the system. In embodiments, the system decreases the expression level of the endogenous MECP2 gene from about 40% to about 100% compared to the expression level of the endogenous MECP2 gene in the absence of the system. In embodiments, the system decreases the expression level of the endogenous MECP2 gene from about 45% to about 100% compared to the expression level of the endogenous MECP2 gene in the absence of the system. In embodiments, the system decreases the expression level of the endogenous MECP2 gene from about 50% to about 100% compared to the expression level of the endogenous MECP2 gene in the absence of the system. In embodiments, the system decreases the expression level of the endogenous MECP2 gene from about 55% to about 100% compared to the expression level of the endogenous MECP2 gene in the absence of the system. In embodiments, the system decreases the expression level of the endogenous MECP2 gene from about 60% to about 100% compared to the expression level of the endogenous MECP2 gene in the absence of the system. In embodiments, the system decreases the expression level of the endogenous MECP2 gene from about 65% to about 100% compared to the expression level of the endogenous MECP2 gene in the absence of the system. In embodiments, the system decreases the expression level of the endogenous MECP2 gene from about 70% to about 100% compared to the expression level of the endogenous MECP2 gene in the absence of the system. In embodiments, the system decreases the expression level of the endogenous MECP2 gene from about 75% to about 100% compared to the expression level of the endogenous MECP2 gene in the absence of the system. In embodiments, the system decreases the expression level of the endogenous MECP2 gene from about 80% to about 100% compared to the expression level of the endogenous MECP2 gene in the absence of the system. In embodiments, the system decreases the expression level of the endogenous MECP2 gene from about 85% to about 100% compared to the expression level of the endogenous MECP2 gene in the absence of the system. In embodiments, the system decreases the expression level of the endogenous MECP2 gene from about 90% to about 100% compared to the expression level of the endogenous MECP2 gene in the absence of the system. In embodiments, the system decreases the expression level of the endogenous MECP2 gene from about 95% to about 100% compared to the expression level of the endogenous MECP2 gene in the absence of the system.

In embodiments, the system decreases the expression level of the endogenous MECP2 gene from about 20% to about 95% compared to the expression level of the endogenous MECP2 gene in the absence of the system. In embodiments, the system decreases the expression level of the endogenous MECP2 gene from about 20% to about 90% compared to the expression level of the endogenous MECP2 gene in the absence of the system. In embodiments, the system decreases the expression level of the endogenous MECP2 gene from about 20% to about 85% compared to the expression level of the endogenous MECP2 gene in the absence of the system. In embodiments, the system decreases the expression level of the endogenous MECP2 gene from about 20% to about 80% compared to the expression level of the endogenous MECP2 gene in the absence of the system. In embodiments, the system decreases the expression level of the endogenous MECP2 gene from about 20% to about 75% compared to the expression level of the endogenous MECP2 gene in the absence of the system. In embodiments, the system decreases the expression level of the endogenous MECP2 gene from about 20% to about 70% compared to the expression level of the endogenous MECP2 gene in the absence of the system. In embodiments, the system decreases the expression level of the endogenous MECP2 gene from about 20% to about 65% compared to the expression level of the endogenous MECP2 gene in the absence of the system. In embodiments, the system decreases the expression level of the endogenous MECP2 gene from about 20% to about 60% compared to the expression level of the endogenous MECP2 gene in the absence of the system. In embodiments, the system decreases the expression level of the endogenous MECP2 gene from about 20% to about 55% compared to the expression level of the endogenous MECP2 gene in the absence of the system. In embodiments, the system decreases the expression level of the endogenous MECP2 gene from about 20% to about 50% compared to the expression level of the endogenous MECP2 gene in the absence of the system. In embodiments, the system decreases the expression level of the endogenous MECP2 gene from about 20% to about 45% compared to the expression level of the endogenous MECP2 gene in the absence of the system. In embodiments, the system decreases the expression level of the endogenous MECP2 gene from about 20% to about 40% compared to the expression level of the endogenous MECP2 gene in the absence of the system. In embodiments, the system decreases the expression level of the endogenous MECP2 gene from about 20% to about 35% compared to the expression level of the endogenous MECP2 gene in the absence of the system. In embodiments, the system decreases the expression level of the endogenous MECP2 gene from about 20% to about 30% compared to the expression level of the endogenous MECP2 gene in the absence of the system. In embodiments, the system decreases the expression level of the endogenous MECP2 gene from about 20% to about 25% compared to the expression level of the endogenous MECP2 gene in the absence of the system. In embodiments, the system decreases the expression level of the endogenous MECP2 gene about 20%, 25%, 30%, 35%, 40%, 45%, 50%, 55%, 60%, 65%, 70%, 75%, 80%, 85%, 90%, 95%, or 100% compared to the expression level of the endogenous MECP2 gene in the absence of the system. In embodiments, the system decreases the expression level of the endogenous MECP2 gene at least about 20%, 25%, 30%, 35%, 40%, 45%, 50%, 55%, 60%, 65%, 70%, 75%, 80%, 85%, 90%, 95%, or 100% compared to the expression level of the endogenous MECP2 gene in the absence of the system.

In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant version of the MECP2 gene) of the MECP2 gene is about the expression level of the endogenous MECP2 gene in a healthy cell (e.g. a cell without Rett syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant version of the MECP2 gene) of the MECP2 gene is less than 4-fold, 3-fold, 2-fold, 1.8-fold, 1.6-fold, 1.5-fold, 1.4-fold, 1.3-fold, 1.2-fold, 1.1-fold, 1-fold, 0.9-fold, 0.8-fold, 0.7-fold, 0.6-fold, or 0.5-fold of the expression level of the endogenous MECP2 gene in a healthy cell (e.g. a cell without Rett syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant version of the MECP2 gene) of the MECP2 gene is at least 0.2-fold, 0.3-fold, 0.4-fold, 0.5-fold, 0.6-fold, 0.7-fold, 0.8-fold, 0.9-fold, or 1-fold of the expression level of the endogenous MECP2 gene in a healthy cell (e.g. a cell without Rett syndrome). In embodiments, the total expression level (including the endogenous and the recombinant versions of the MECP2 gene) of the MECP2 gene is at least 20%, 30%, 40%, 50%, 60%, 70%, 80%, 85%, 90%, 91%, 92%, 93%, 94%, 95%, 96%, 97%, 98%, 99% or 100% of the expression level of the endogenous MECP2 gene in a healthy cell (e.g. a cell without Rett syndrome).

In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.2-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.3-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.4-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.5-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.6-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.7-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.8-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 0.9-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.1-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.2-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.3-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.4-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.5-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.6-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.7-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.8-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 1.9-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.1-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.2-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.3-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.4-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.5-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.6-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.7-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.8-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 2.9-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.1-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.2-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.3-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.4-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.5-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.6-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.7-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.8-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is from about 3.9-fold to about 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene is about 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, or 4-fold of the expression level of the endogenous MECP2 gene in a healthy cell (e.g. a cell without Rett Syndrome). In embodiments, the system silences (e.g. downregulates, decreases, or inhibits) the expression level of an endogenous MECP2 gene in a cell and expresses a recombinant version of the MECP2 gene in the cell, wherein the total expression level (including the endogenous and the recombinant versions of the MECP2 gene) of the MECP2 gene less than about 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, or 4-fold of the expression level of the endogenous MECP2 gene in a healthy cell (e.g. a cell without Rett Syndrome).

### Synthesis Module

For the system provided herein, in embodiments, the synthesis module includes an RNA molecule. In embodiments, the RNA molecule includes a messenger RNA (mRNA). In embodiments, the mRNA encodes MECP2 or a fragment thereof. In embodiments, the mRNA of the synthesis module does not include an mRNA splice site. In embodiments, the mRNA of the synthesis module excludes an mRNA splice site.

In embodiments, the synthesis module includes a DNA molecule. In embodiments, the DNA molecule encodes MECP2 or a fragment thereof. In embodiments, the DNA molecule of the synthesis module encodes an mRNA. In embodiments, the mRNA encodes MECP2 or a fragment thereof. In embodiments, the mRNA does not include an mRNA splice site.

Some embodiments of a synthesis module include any one or all of the following:
a weak, ubiquitous promoter (e.g. −Ubc, PGK, EF1a-core);
   (In some embodiments, the promoter is strengthened via a SV40 intron)
An endogenous MECP2 5′ UTR and Kozak sequence;
   (In some embodiments, the vector lacks the MECP2 5′ UTR and/or uses a standard Kozak sequence (GCC(RCC)ATG, where R is any nucleotide and ATG is the start codon))
A full-length MECP2 coding sequence consisting of exons 1, 3, and 4;
   (In some embodiments, this contains endogenous intron fragments between exons 1 and 3 or 3 and 4); and
An engineered 3′ UTR consisting of multiple fragments of the endogenous MECP2 3′ UTR and the distal MECP2 polyA signal
   (In some embodiments, the vector lacks this element and only contains a standard polyA signal (e.g. −bGH, SV40, hGH)).

In some embodiments, the synthesis module increases a target measurement (e.g. protein or RNA, such as a MECP2 protein or RNA) in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, or at least 250%, relative to a baseline target measurement. In some embodiments, the synthesis module increases a target measurement (e.g. protein or RNA, such as a MECP2 protein or RNA) in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 90%, less than 100%, less than 110%, less than 120%, less than 130%, less than 140%, less than 150%, less than 160%, less than 170%, less than 180%, less than 190%, less than 200%, less than 210%, less than 220%, less than 230%, less than 240%, or less than 250%, relative to a baseline target measurement.

### Synthesis Module Promoter

For the system provided herein, in embodiments, the synthesis module further includes a synthesis module promoter sequence, or is encoded by a nucleic acid including the synthesis module promoter sequence. In embodiments, the synthesis module includes a synthesis module promoter sequence. In embodiments, the synthesis module is encoded by a nucleic acid including the synthesis module promoter sequence.

In embodiments, the synthesis module promoter sequence includes a mouse or human promoter sequence. In embodiments, the synthesis module promoter sequence includes a mouse promoter sequence. In embodiments, the synthesis module promoter sequence includes a human promoter sequence.

In embodiments, the synthesis module promoter includes a ubiquitous promoter. The term “ubiquitous promoter” refers to a promoter that may be active in a wide range cellular conditions. For example, a ubiquitous promoter may allow continuous expression of a gene in a cell. In embodiments, a ubiquitous promoter allows expression of a gene in a variety of cells or in multiple stages of the cell cycle. In embodiments, a ubiquitous promoter is active in a wide range of tissue types. In embodiments, the ubiquitous promoter allows for constitutive expression of the recombinant version of the MECP2 RNA. In embodiments, the recombinant version of the MECP2 RNA may be expressed in a wide range of cells. In embodiments, the recombinant version of the MECP2 RNA may be expressed during multiple phases of the cell cycle.

In embodiments, the synthesis module promoter includes a weak promoter. For example, a weak promoter may regulate transcription of a recombinant version of the MECP2 gene wherein the expression level of the recombinant version of the MECP2 gene is no greater than the expression level of the endogenous MECP2 gene in a healthy cell (e.g. a cell without Rett syndrome). In embodiments, the weak promoter drives expression of mRNA molecules at a rate no greater than does an endogenous MECP2 promoter.

In embodiments, the weak promoter drives expression of mRNA molecules at 10%, 20%, 30% 40%, 50%, 60%, 70%, 80%, 85%, 90%, 91%, 92%, 93%, 94%, 95%, 96%, 97%, 98%, 99%, or 100% of the rate as does an endogenous promoter. In embodiments, the endogenous promoter is a MECP2 promoter.

In embodiments, the weak promoter drives expression of mRNA molecules at less than 2-fold, 1.8-fold, 1.6-fold, 1.4-fold, 1.2-fold, 1-fold, 0.8-fold, 0.6-fold, 0.4-fold, or 0.2-fold of the rate as does an endogenous promoter. In embodiments, the endogenous promoter is an endogenous MECP2 promoter.

In embodiments, the synthesis module promoter sequence includes a promoter sequence of a Ubc promoter, a PGK promoter, or an EF1a-core promoter. In embodiments, the synthesis module promoter sequence includes a Ubc promoter sequence, a PGK promoter sequence, or an EF1a-core promoter sequence. In embodiments, the synthesis module promoter sequence includes a Ubc promoter sequence. In embodiments, the synthesis module promoter sequence includes a PGK promoter sequence. In embodiments, the synthesis module promoter sequence includes an EF1a-core promoter sequence. In embodiments, the synthesis module promoter sequence is a Ubc promoter sequence. In embodiments, the synthesis module promoter sequence is a PGK promoter sequence. In embodiments, the synthesis module promoter sequence is an EF1a-core promoter sequence.

In embodiments, the synthesis module promoter sequence is at least 90% identical to a promoter sequence set forth in Table 1. In embodiments, the synthesis module promoter sequence is at least 91% identical to a promoter sequence set forth in Table 1. In embodiments, the synthesis module promoter sequence is at least 92% identical to a promoter sequence set forth in Table 1. In embodiments, the synthesis module promoter sequence is at least 93% identical to a promoter sequence set forth in Table 1. In embodiments, the synthesis module promoter sequence is at least 94% identical to a promoter sequence set forth in Table 1. In embodiments, the synthesis module promoter sequence is at least 94% identical to a promoter sequence set forth in Table 1. In embodiments, the synthesis module promoter sequence is at least 96% identical to a promoter sequence set forth in Table 1. In embodiments, the synthesis module promoter sequence is at least 97% identical to a promoter sequence set forth in Table 1. In embodiments, the synthesis module promoter sequence is at least 98% identical to a promoter sequence set forth in Table 1. In embodiments, the synthesis module promoter sequence is at least 99% identical to a promoter sequence set forth in Table 1. In embodiments, the synthesis module promoter sequence includes a promoter sequence set forth in Table 1. In embodiments, the synthesis module promoter sequence is a promoter sequence set forth in Table 1. In some embodiments, the synthesis module promoter sequence is at least 80%, at least 85%, at least 90%, at least 95%, at least 98%, at least 99%, or 100% identical to a promoter sequence in Table 1 or Table 8.

In embodiments, the synthesis promoter sequence is at least 90% identical to SEQ ID NO: 1, SEQ ID NO:2, SEQ ID NO:3, SEQ ID NO:19, SEQ ID NO:20, SEQ ID NO:21, or SEQ ID NO:22. In embodiments, the synthesis promoter sequence is at least 90% identical to SEQ ID NO:1. In embodiments, the synthesis promoter sequence is at least 90% identical to SEQ ID NO:2. In embodiments, the synthesis promoter sequence is at least 90% identical to SEQ ID NO:3. In embodiments, the synthesis promoter sequence is at least 90% identical to SEQ ID NO:19. In embodiments, the synthesis promoter sequence is at least 90% identical to SEQ ID NO:20. In embodiments, the synthesis promoter sequence is at least 90% identical to SEQ ID NO:21. In embodiments, the synthesis promoter sequence is at least 90% identical to SEQ ID NO:22. In embodiments, the synthesis promoter sequence includes SEQ ID NO: 1. In embodiments, the synthesis promoter sequence includes SEQ ID NO:2. In embodiments, the synthesis promoter sequence includes SEQ ID NO:3. In embodiments, the synthesis promoter sequence includes SEQ ID NO: 19. In embodiments, the synthesis promoter sequence includes SEQ ID NO:20. In embodiments, the synthesis promoter sequence includes SEQ ID NO:21. In embodiments, the synthesis promoter sequence includes SEQ ID NO:22.

In embodiments, the synthesis module promoter sequence is 5′ or upstream relative to the nucleic acid coding sequence (CDS). In embodiments, the synthesis module promoter sequence is 5′ relative to the nucleic acid coding sequence (CDS). In embodiments, the synthesis module promoter sequence is upstream relative to the nucleic acid coding sequence (CDS).

Some include a weak, ubiquitous promoter (e.g. −Ubc, PGK, EF1a-core). In some embodiments, the promoter is strengthened via a SV40 intron.

### SV40 Intron

The synthesis module provided herein including embodiments thereof may further include one or more nucleic acid sequences that increases or upregulates expression levels of a recombinant MECP2 gene (e.g. recombinant version of the MECP2 RNA, recombinant MECP2). In embodiments, the synthesis module includes a nucleic acid sequence that increases the stability of the recombinant MECP2 gene (e.g. recombinant version of the MECP2 RNA, recombinant MECP2). In embodiments, the synthesis module includes a nucleic acid sequence that regulates processing of the recombinant MECP2 gene (e.g. recombinant version of the MECP2 RNA, recombinant MECP2).

In embodiments, the synthesis module further includes an SV40 intron sequence. In embodiments, the SV40 intron sequence is at least 90% identical to an SV40 intron sequence set forth in Table 1. In embodiments, the SV40 intron sequence is at least 910% identical to an SV40 intron sequence set forth in Table 1. In embodiments, the SV40 intron sequence is at least 92% identical to an SV40 intron sequence set forth in Table 1. In embodiments, the SV40 intron sequence is at least 93% identical to an SV40 intron sequence set forth in Table 1. In embodiments, the SV40 intron sequence is at least 94% identical to an SV40 intron sequence set forth in Table 1. In embodiments, the SV40 intron sequence is at least 95% identical to an SV40 intron sequence set forth in Table 1. In embodiments, the SV40 intron sequence is at least 96% identical to an SV40 intron sequence set forth in Table 1. In embodiments, the SV40 intron sequence is at least 97% identical to an SV40 intron sequence set forth in Table 1. In embodiments, the SV40 intron sequence is at least 98% identical to an SV40 intron sequence set forth in Table 1. In embodiments, the SV40 intron sequence is at least 99% identical to an SV40 intron sequence set forth in Table 1. In embodiments, the SV40 intron sequence includes an SV40 intron sequence set forth in Table 1. In embodiments, the SV40 intron sequence is an SV40 intron sequence set forth in Table 1.

In embodiments, the SV40 intron sequence is at least 80% identical to SEQ ID NO:4. In embodiments, the SV40 intron sequence is at least 85% identical to SEQ ID NO:4. In embodiments, the SV40 intron sequence is at least 90% identical to SEQ ID NO:4. In embodiments, the SV40 intron sequence is at least 91% identical to SEQ ID NO:4. In embodiments, the SV40 intron sequence is at least 92% identical to SEQ ID NO:4. In embodiments, the SV40 intron sequence is at least 93% identical to SEQ ID NO:4. In embodiments, the SV40 intron sequence is at least 94% identical to SEQ ID NO:4. In embodiments, the SV40 intron sequence is at least 95% identical to SEQ ID NO:4. In embodiments, the SV40 intron sequence is at least 96% identical to SEQ ID NO:4. In embodiments, the SV40 intron sequence is at least 97% identical to SEQ ID NO:4. In embodiments, the SV40 intron sequence is at least 98% identical to SEQ ID NO:4. In embodiments, the SV40 intron sequence is at least 99% identical to SEQ ID NO:4. In embodiments, the SV40 intron sequence includes SEQ ID NO:4. In embodiments, the SV40 intron sequence is SEQ ID NO:4.

In embodiments, the SV40 intron sequence is 3′ or downstream of the synthesis module promoter sequence. In embodiments, the SV40 intron sequence is 3′ of the synthesis module promoter sequence. In embodiments, the SV40 intron sequence is downstream of the synthesis module promoter sequence.

In embodiments, SV40 intron sequence is 5′ or upstream relative to the CDS. In embodiments, SV40 intron sequence is 5′ to the CDS. In embodiments, SV40 intron sequence is upstream relative to the CDS.

### 5′ UTR and Kozak Sequence

The system provided herein may in embodiments include one or more nucleic acid sequences capable of regulating expression of the recombinant MECP2 (e.g. recombinant version of the MECP2 RNA, recombinant MECP2). For example, the system module may include a sequence capable of regulating translation of the recombinant MECP2 RNA. Thus, in embodiments, the synthesis module further includes a 5′ untranslated region (UTR) sequence of the MECP2 RNA.

In embodiments, the 5′ UTR sequence is at least 90% identical to a 5′ UTR sequence set forth in Table 1. In embodiments, the 5′ UTR sequence is at least 91% identical to a 5′ UTR sequence set forth in Table 1. In embodiments, the 5′ UTR sequence is at least 92% identical to a 5′ UTR sequence set forth in Table 1. In embodiments, the 5′ UTR sequence is at least 93% identical to a 5′ UTR sequence set forth in Table 1. In embodiments, the 5′ UTR sequence is at least 94% identical to a 5′ UTR sequence set forth in Table 1. In embodiments, the 5′ UTR sequence is at least 95% identical to a 5′ UTR sequence set forth in Table 1. In embodiments, the 5′ UTR sequence is at least 96% identical to a 5′ UTR sequence set forth in Table 1. In embodiments, the 5′ UTR sequence is at least 97% identical to a 5′ UTR sequence set forth in Table 1. In embodiments, the 5′ UTR sequence is at least 98% identical to a 5′ UTR sequence set forth in Table 1. In embodiments, the 5′ UTR sequence is at least 99% identical to a 5′ UTR sequence set forth in Table 1. In embodiments, the 5′ UTR sequence includes a 5′ UTR sequence set forth in Table 1. In embodiments, the 5′ UTR sequence is a 5′ UTR sequence set forth in Table 1.

In embodiments, the synthesis module further includes a Kozak sequence. In embodiments, the Kozak sequence is at least 90% identical to 5′-GCCNCCATG-3′ where N is A, T, C, or G, and ATG is a start codon; or wherein the Kozak sequence is at least 90% identical to 5′-GCCNCCAUG-3′ where N is A, U, G, or C, and AUG is a start codon. In embodiments, the Kozak sequence is at least 90% identical to 5′-GCCNCCATG-3′ where N is A, T, C, or G, and ATG is a start codon. In embodiments, the Kozak sequence is at least 90% identical to 5′-GCCNCCAUG-3′ where N is A, U, C, or G, and AUG is a start codon. In embodiments, the Kozak sequence is an endogenous Kozak sequence. For example, the Kozak sequence may be a naturally-occurring Kozak sequence found in the endogenous MECP2 gene. In embodiments, the Kozak sequence is at least 90% identical to 5′-CGGAAAATG-3′. “Kozak sequence” as used herein refers to a nucleic acid sequence in mRNA that directs proteins involved in translation to the translation initiation site. For example, the Kozak sequence may direct the pre-initiation complex and ribosome to the translation initiation site. In embodiments, the Kozak sequence is involved in ribosome assembly.

In embodiments, the 5′ UTR sequence or the Kozak sequence is upstream or 5′ relative to the CDS within the synthesis module. In embodiments, the 5′ UTR sequence is upstream or 5′ relative to the CDS within the synthesis module. In embodiments, the 5′ UTR sequence is upstream relative to the CDS within the synthesis module. In embodiments, the 5′ UTR sequence is 5′ relative to the CDS within the synthesis module. In embodiments, the Kozak sequence is upstream or 5′ relative to the CDS within the synthesis module. In embodiments, the Kozak sequence is upstream relative to the CDS within the synthesis module. In embodiments, the Kozak sequence is 5′ relative to the CDS within the synthesis module.

Some embodiments include an endogenous MECP2 5′ UTR and Kozak sequence. In some embodiments, the vector lacks the MECP2 5′ UTR and/or uses a standard Kozak sequence (GCC(RCC)ATG, where R is any nucleotide and ATG is the start codon).

### Nucleic Acid Coding Sequence

For the system provided herein, in embodiments, the nucleic acid coding sequence (CDS) includes introns and exons. In embodiments, the CDS includes exons interspaced with an intron. In embodiments, the CDS includes exons without introns. Some embodiments include or encode an open reading frame (ORF) of an MECP2 mRNA.

In some embodiments, the CDS excludes 1 or more exons or introns of a target gene. In embodiments, the CDS does not include introns. Thus, in embodiments, the CDS includes exons adjacent to one another without an intervening intron.

In embodiments, the CDS includes exons 1, 3 and 4 of MECP2. In embodiments, the CDS excludes exon 2 of MECP2. In embodiments, the CDS encodes an e1 isoform of MECP2. In embodiments, the e1 isoform of MECP2 includes exons 1, 3, and 4 of MECP2 or fragments thereof. In embodiments, the e1 isoform of MECP2 does not include exon 2 of MECP2.

In some embodiments, the CDS includes exon 1 of MECP2. An example of an MECP2 exon 1 sequence is included as SEQ ID NO: 365. An exon of a target may include a sequence at least 80%, at least 85%, at least 90%, at least 95%, at least 98%, at least 99%, or 100% identical to SEQ ID NO: 365. An example of an MECP2 exon 1 sequence is included as SEQ ID NO: 368. An exon of a target may include a sequence at least 80%, at least 85%, at least 90%, at least 95%, at least 98%, at least 99%, or 100% identical to SEQ ID NO: 368.

In some embodiments, the CDS includes exon 3 of MECP2. An example of an MECP2 exon 3 sequence is included as SEQ ID NO: 366. An exon of a target may include a sequence at least 80%, at least 85%, at least 90%, at least 95%, at least 98%, at least 99%, or 100% identical to SEQ ID NO: 366. An example of an MECP2 exon 3 sequence is included as SEQ ID NO: 369. An exon of a target may include a sequence at least 80%, at least 85%, at least 90%, at least 95%, at least 98%, at least 99%, or 100% identical to SEQ ID NO: 369.

In some embodiments, the CDS includes exon 4 of MECP2. An example of an MECP2 exon 4 sequence is included as SEQ ID NO: 367. An exon of a target may include a sequence at least 80%, at least 85%, at least 90%, at least 95%, at least 98%, at least 99%, or 100% identical to SEQ ID NO: 367. An example of an MECP2 exon 4 sequence is included as SEQ ID NO: 370. An exon of a target may include a sequence at least 80%, at least 85%, at least 90%, at least 95%, at least 98%, at least 99%, or 100% identical to SEQ ID NO: 370.

In embodiments, the CDS sequence is at least 90% identical to a CDS sequence set forth in Table 1. In embodiments, the CDS sequence is at least 91% identical to a CDS sequence set forth in Table 1. In embodiments, the CDS sequence is at least 92% identical to a CDS sequence set forth in Table 1. In embodiments, the CDS sequence is at least 93% identical to a CDS sequence set forth in Table 1. In embodiments, the CDS sequence is at least 94% identical to a CDS sequence set forth in Table 1. In embodiments, the CDS sequence is at least 95% identical to a CDS sequence set forth in Table 1. In embodiments, the CDS sequence is at least 96% identical to a CDS sequence set forth in Table 1. In embodiments, the CDS sequence is at least 97% identical to a CDS sequence set forth in Table 1. In embodiments, the CDS sequence is at least 98% identical to a CDS sequence set forth in Table 1. In embodiments, the CDS sequence is at least 99% identical to a CDS sequence set forth in Table 1. In embodiments, the CDS sequence includes a CDS sequence set forth in Table 1. In embodiments, the CDS sequence is a CDS sequence set forth in Table 1.

In embodiments, the CDS sequence is at least 90% identical to SEQ ID NO: 6. In embodiments, the CDS sequence is at least 91% identical to SEQ ID NO: 6. In embodiments, the CDS sequence is at least 92% identical to SEQ ID NO: 6. In embodiments, the CDS sequence is at least 93% identical to SEQ ID NO: 6. In embodiments, the CDS sequence is at least 94% identical to SEQ ID NO: 6. In embodiments, the CDS sequence is at least 95% identical to SEQ ID NO: 6. In embodiments, the CDS sequence is at least 96% identical to SEQ ID NO: 6. In embodiments, the CDS sequence is at least 97% identical to SEQ ID NO: 6. In embodiments, the CDS sequence is at least 98% identical to SEQ ID NO: 6. In embodiments, the CDS sequence is at least 99% identical to SEQ ID NO: 6. In embodiments, the CDS sequence includes SEQ ID NO: 6. In embodiments, the CDS sequence is SEQ ID NO: 6.

In embodiments, the CDS sequence is at least 90% identical to SEQ ID NO: 7. In embodiments, the CDS sequence is at least 91% identical to SEQ ID NO: 7. In embodiments, the CDS sequence is at least 92% identical to SEQ ID NO: 7. In embodiments, the CDS sequence is at least 93% identical to SEQ ID NO: 7. In embodiments, the CDS sequence is at least 94% identical to SEQ ID NO: 7. In embodiments, the CDS sequence is at least 95% identical to SEQ ID NO: 7. In embodiments, the CDS sequence is at least 96% identical to SEQ ID NO: 7. In embodiments, the CDS sequence is at least 97% identical to SEQ ID NO: 7. In embodiments, the CDS sequence is at least 98% identical to SEQ ID NO: 7. In embodiments, the CDS sequence is at least 99% identical to SEQ ID NO: 7. In embodiments, the CDS sequence includes SEQ ID NO: 7. In embodiments, the CDS sequence is SEQ ID NO: 7.

In some embodiments, the CDS includes exon 2 of MECP2. In some embodiments, the CDS comprises MECP2 exons 2, 3 and 4. In some embodiments, the CDS encodes an e2 isoform of MECP2. In some embodiments, the CDS does not include exon 1 of MECP2.

In embodiments, the CDS includes exons 1 and 3 of MECP2 with an intron between exons 1 and 3. In embodiments, the CDS includes an intron fragment sequence between exon 1 and 3. In embodiments, the intron fragment sequence between exon 1 and 3 includes a MECP2 intron 1 fragment sequence.

In embodiments, the MECP2 intron 1 fragment sequence is at least 90% identical to an intron 1 fragment sequence set forth in Table 1. In embodiments, the MECP2 intron 1 fragment sequence is at least 910% identical to an intron 1 fragment sequence set forth in Table 1. In embodiments, the MECP2 intron 1 fragment sequence is at least 92% identical to an intron 1 fragment sequence set forth in Table 1. In embodiments, the MECP2 intron 1 fragment sequence is at least 93% identical to an intron 1 fragment sequence set forth in Table 1. In embodiments, the MECP2 intron 1 fragment sequence is at least 94% identical to an intron 1 fragment sequence set forth in Table 1. In embodiments, the MECP2 intron 1 fragment sequence is at least 95% identical to an intron 1 fragment sequence set forth in Table 1. In embodiments, the MECP2 intron 1 fragment sequence is at least 96% identical to an intron 1 fragment sequence set forth in Table 1. In embodiments, the MECP2 intron 1 fragment sequence is at least 97% identical to an intron 1 fragment sequence set forth in Table 1. In embodiments, the MECP2 intron 1 fragment sequence is at least 98% identical to an intron 1 fragment sequence set forth in Table 1. In embodiments, the MECP2 intron 1 fragment sequence is at least 99% identical to an intron 1 fragment sequence set forth in Table 1. In embodiments, the MECP2 intron 1 fragment sequence includes an intron 1 fragment sequence set forth in Table 1. In embodiments, the MECP2 intron 1 fragment sequence is an intron 1 fragment sequence set forth in Table 1.

In embodiments, the CDS includes exons 1 and 3 of MECP2 without an intron between exons 1 and 3. In embodiments, the CDS includes exons 3 and 4 of MECP2 with an intron between exons 3 and 4. In embodiments, the CDS includes an intron fragment sequence between exon 3 and 4. In embodiments, the intron fragment sequence between exon 3 and 4 includes a MECP2 intron 3 fragment sequence.

In embodiments, the MECP2 intron 3 fragment sequence is at least 90% identical to an intron 3 fragment sequence set forth in Table 1. In embodiments, the MECP2 intron 3 fragment sequence is at least 910% identical to an intron 3 fragment sequence set forth in Table 1. In embodiments, the MECP2 intron 3 fragment sequence is at least 92% identical to an intron 3 fragment sequence set forth in Table 1. In embodiments, the MECP2 intron 3 fragment sequence is at least 93% identical to an intron 3 fragment sequence set forth in Table 1. In embodiments, the MECP2 intron 3 fragment sequence is at least 94% identical to an intron 3 fragment sequence set forth in Table 1. In embodiments, the MECP2 intron 3 fragment sequence is at least 95% identical to an intron 3 fragment sequence set forth in Table 1. In embodiments, the MECP2 intron 3 fragment sequence is at least 96% identical to an intron 3 fragment sequence set forth in Table 1. In embodiments, the MECP2 intron 3 fragment sequence is at least 97% identical to an intron 3 fragment sequence set forth in Table 1. In embodiments, the MECP2 intron 3 fragment sequence is at least 98% identical to an intron 3 fragment sequence set forth in Table 1. In embodiments, the MECP2 intron 3 fragment sequence is at least 99% identical to an intron 3 fragment sequence set forth in Table 1. In embodiments, the MECP2 intron 3 fragment sequence includes an intron 3 fragment sequence set forth in Table 1. In embodiments, the MECP2 intron 3 fragment sequence is an intron 3 fragment sequence set forth in Table 1.

In embodiments, the intron fragment sequence includes a nucleic acid sequence at least 90% identical to SEQ ID NO: 8. In embodiments, the intron fragment sequence includes a nucleic acid sequence at least 910% identical to SEQ ID NO: 8. In embodiments, the intron fragment sequence includes a nucleic acid sequence at least 92% identical to SEQ ID NO: 8. In embodiments, the intron fragment sequence includes a nucleic acid sequence at least 93% identical to SEQ ID NO: 8. In embodiments, the intron fragment sequence includes a nucleic acid sequence at least 94% identical to SEQ ID NO: 8. In embodiments, the intron fragment sequence includes a nucleic acid sequence at least 95% identical to SEQ ID NO: 8. In embodiments, the intron fragment sequence includes a nucleic acid sequence at least 96% identical to SEQ ID NO: 8. In embodiments, the intron fragment sequence includes a nucleic acid sequence at least 97% identical to SEQ ID NO: 8. In embodiments, the intron fragment sequence includes a nucleic acid sequence at least 98% identical to SEQ ID NO: 8. In embodiments, the intron fragment sequence includes a nucleic acid sequence at least 99% identical to SEQ ID NO: 8. In embodiments, the intron fragment sequence includes SEQ ID NO: 8. In embodiments, the intron fragment sequence is SEQ ID NO: 8.

In embodiments, the intron fragment sequence includes a nucleic acid sequence at least 90% identical to SEQ ID NO: 9. In embodiments, the intron fragment sequence includes a nucleic acid sequence at least 91% identical to SEQ ID NO: 9. In embodiments, the intron fragment sequence includes a nucleic acid sequence at least 92% identical to SEQ ID NO: 9. In embodiments, the intron fragment sequence includes a nucleic acid sequence at least 93% identical to SEQ ID NO: 9. In embodiments, the intron fragment sequence includes a nucleic acid sequence at least 94% identical to SEQ ID NO: 9. In embodiments, the intron fragment sequence includes a nucleic acid sequence at least 95% identical to SEQ ID NO: 9. In embodiments, the intron fragment sequence includes a nucleic acid sequence at least 96% identical to SEQ ID NO: 9. In embodiments, the intron fragment sequence includes a nucleic acid sequence at least 97% identical to SEQ ID NO: 9. In embodiments, the intron fragment sequence includes a nucleic acid sequence at least 98% identical to SEQ ID NO: 9. In embodiments, the intron fragment sequence includes a nucleic acid sequence at least 99% identical to SEQ ID NO: 9. In embodiments, the intron fragment sequence includes SEQ ID NO: 9. In embodiments, the intron fragment sequence is SEQ ID NO: 9.

In embodiments, the intron fragment sequence includes a nucleic acid sequence at least 90% identical to SEQ ID NO: 10. In embodiments, the intron fragment sequence includes a nucleic acid sequence at least 91% identical to SEQ ID NO: 10. In embodiments, the intron fragment sequence includes a nucleic acid sequence at least 92% identical to SEQ ID NO: 10. In embodiments, the intron fragment sequence includes a nucleic acid sequence at least 93% identical to SEQ ID NO: 10. In embodiments, the intron fragment sequence includes a nucleic acid sequence at least 94% identical to SEQ ID NO: 10. In embodiments, the intron fragment sequence includes a nucleic acid sequence at least 95% identical to SEQ ID NO: 10. In embodiments, the intron fragment sequence includes a nucleic acid sequence at least 96% identical to SEQ ID NO: 10. In embodiments, the intron fragment sequence includes a nucleic acid sequence at least 97% identical to SEQ ID NO: 10. In embodiments, the intron fragment sequence includes a nucleic acid sequence at least 98% identical to SEQ ID NO: 10. In embodiments, the intron fragment sequence includes a nucleic acid sequence at least 99% identical to SEQ ID NO: 10. In embodiments, the intron fragment sequence includes SEQ ID NO: 10. In embodiments, the intron fragment sequence is SEQ ID NO: 10.

In embodiments, the intron fragment sequence includes a nucleic acid sequence at least 90% identical to SEQ ID NO: 11. In embodiments, the intron fragment sequence includes a nucleic acid sequence at least 91% identical to SEQ ID NO: 11. In embodiments, the intron fragment sequence includes a nucleic acid sequence at least 92% identical to SEQ ID NO: 11. In embodiments, the intron fragment sequence includes a nucleic acid sequence at least 93% identical to SEQ ID NO: 11. In embodiments, the intron fragment sequence includes a nucleic acid sequence at least 94% identical to SEQ ID NO: 11. In embodiments, the intron fragment sequence includes a nucleic acid sequence at least 95% identical to SEQ ID NO: 11. In embodiments, the intron fragment sequence includes a nucleic acid sequence at least 96% identical to SEQ ID NO: 11. In embodiments, the intron fragment sequence includes a nucleic acid sequence at least 97% identical to SEQ ID NO: 11. In embodiments, the intron fragment sequence includes a nucleic acid sequence at least 98% identical to SEQ ID NO: 11. In embodiments, the intron fragment sequence includes a nucleic acid sequence at least 99% identical to SEQ ID NO: 11. In embodiments, the intron fragment sequence includes SEQ ID NO: 11. In embodiments, the intron fragment sequence is SEQ ID NO: 11.

In embodiments, the intron fragment sequence includes a nucleic acid sequence at least 90% identical to SEQ ID NO: 12. In embodiments, the intron fragment sequence includes a nucleic acid sequence at least 91% identical to SEQ ID NO: 12. In embodiments, the intron fragment sequence includes a nucleic acid sequence at least 92% identical to SEQ ID NO: 12. In embodiments, the intron fragment sequence includes a nucleic acid sequence at least 93% identical to SEQ ID NO: 12. In embodiments, the intron fragment sequence includes a nucleic acid sequence at least 94% identical to SEQ ID NO: 12. In embodiments, the intron fragment sequence includes a nucleic acid sequence at least 95% identical to SEQ ID NO: 12. In embodiments, the intron fragment sequence includes a nucleic acid sequence at least 96% identical to SEQ ID NO: 12. In embodiments, the intron fragment sequence includes a nucleic acid sequence at least 97% identical to SEQ ID NO: 12. In embodiments, the intron fragment sequence includes a nucleic acid sequence at least 98% identical to SEQ ID NO: 12. In embodiments, the intron fragment sequence includes a nucleic acid sequence at least 99% identical to SEQ ID NO: 12. In embodiments, the intron fragment sequence includes SEQ ID NO: 12. In embodiments, the intron fragment sequence is SEQ ID NO: 12.

In embodiments, the intron fragment sequence includes a nucleic acid sequence at least 90% identical to SEQ ID NO: 13. In embodiments, the intron fragment sequence includes a nucleic acid sequence at least 91% identical to SEQ ID NO: 13. In embodiments, the intron fragment sequence includes a nucleic acid sequence at least 92% identical to SEQ ID NO: 13. In embodiments, the intron fragment sequence includes a nucleic acid sequence at least 93% identical to SEQ ID NO: 13. In embodiments, the intron fragment sequence includes a nucleic acid sequence at least 94% identical to SEQ ID NO: 13. In embodiments, the intron fragment sequence includes a nucleic acid sequence at least 95% identical to SEQ ID NO: 13. In embodiments, the intron fragment sequence includes a nucleic acid sequence at least 96% identical to SEQ ID NO: 13. In embodiments, the intron fragment sequence includes a nucleic acid sequence at least 97% identical to SEQ ID NO: 13. In embodiments, the intron fragment sequence includes a nucleic acid sequence at least 98% identical to SEQ ID NO: 13. In embodiments, the intron fragment sequence includes a nucleic acid sequence at least 99% identical to SEQ ID NO: 13. In embodiments, the intron fragment sequence includes SEQ ID NO: 13. In embodiments, the intron fragment sequence is SEQ ID NO: 13.

In embodiments, the intron fragment sequence includes a nucleic acid sequence at least 90% identical to SEQ ID NO: 14. In embodiments, the intron fragment sequence includes a nucleic acid sequence at least 910% identical to SEQ ID NO: 14. In embodiments, the intron fragment sequence includes a nucleic acid sequence at least 92% identical to SEQ ID NO: 14. In embodiments, the intron fragment sequence includes a nucleic acid sequence at least 93% identical to SEQ ID NO: 14. In embodiments, the intron fragment sequence includes a nucleic acid sequence at least 94% identical to SEQ ID NO: 14. In embodiments, the intron fragment sequence includes a nucleic acid sequence at least 95% identical to SEQ ID NO: 14. In embodiments, the intron fragment sequence includes a nucleic acid sequence at least 96% identical to SEQ ID NO: 14. In embodiments, the intron fragment sequence includes a nucleic acid sequence at least 97% identical to SEQ ID NO: 14. In embodiments, the intron fragment sequence includes a nucleic acid sequence at least 98% identical to SEQ ID NO: 14. In embodiments, the intron fragment sequence includes a nucleic acid sequence at least 99% identical to SEQ ID NO: 14. In embodiments, the intron fragment sequence includes SEQ ID NO: 14. In embodiments, the intron fragment sequence is SEQ ID NO: 14.

In embodiments, the intron fragment sequence includes a nucleic acid sequence at least 90% identical to SEQ ID NO: 15. In embodiments, the intron fragment sequence includes a nucleic acid sequence at least 91% identical to SEQ ID NO: 15. In embodiments, the intron fragment sequence includes a nucleic acid sequence at least 92% identical to SEQ ID NO: 15. In embodiments, the intron fragment sequence includes a nucleic acid sequence at least 93% identical to SEQ ID NO: 15. In embodiments, the intron fragment sequence includes a nucleic acid sequence at least 94% identical to SEQ ID NO: 15. In embodiments, the intron fragment sequence includes a nucleic acid sequence at least 95% identical to SEQ ID NO: 15. In embodiments, the intron fragment sequence includes a nucleic acid sequence at least 96% identical to SEQ ID NO: 15. In embodiments, the intron fragment sequence includes a nucleic acid sequence at least 97% identical to SEQ ID NO: 15. In embodiments, the intron fragment sequence includes a nucleic acid sequence at least 98% identical to SEQ ID NO: 15. In embodiments, the intron fragment sequence includes a nucleic acid sequence at least 99% identical to SEQ ID NO: 15. In embodiments, the intron fragment sequence includes SEQ ID NO: 15. In embodiments, the intron fragment sequence is SEQ ID NO: 15.

In embodiments, the CDS includes exons 3 and 4 of MECP2 without an intron between exons 3 and 4.

Some embodiments include a full-length MECP2 coding sequence comprising exons 1, 3, and 4 of an MECP2 RNA. Some embodiments include a full-length MECP2 coding sequence consisting of exons 1, 3, and 4 of an MECP2 RNA. In some embodiments, this contains endogenous intron fragments between exons 1 and 3 or 3 and 4.

### 3′ UTR

The expression system provided herein in embodiments may include a sequence that regulates expression of the recombinant MECP2 (e.g. recombinant version of the MECP2 RNA, recombinant MECP2). For example, the expression system may include a sequence that regulates translation or post-translational modifications of the recombinant MECP2 RNA. The expression sequence may include a sequence that regulates stability of the recombinant MECP2 RNA. Thus, in embodiments, the synthesis module further comprises a 3′ untranslated region (UTR) sequence of the MECP2 RNA. In embodiments, the 3′ UTR sequence includes an endogenous MECP2 3′ UTR sequence or fragment thereof.

In embodiments, the 3′UTR includes multiple fragments of an endogenous MECP2 3′UTR sequence. For example, the 3′UTR may include multiple fragments of an endogenous MECP2 3′UTR sequence, each including endogenous miRNA binding sites.

In embodiments, the 3′ UTR sequence is downstream or 3′ relative to the promoter sequence, the SV40 intron sequence, the 5′ UTR sequence, the Kozak sequence, or the CDS within the synthesis module. In embodiments, the 3′ UTR sequence is downstream or 3′ relative to the promoter sequence within the synthesis module. In embodiments, the 3′ UTR sequence is downstream or 3′ relative to the SV40 intron sequence within the synthesis module. In embodiments, the 3′ UTR sequence is downstream or 3′ relative to the 5′ UTR sequence within the synthesis module. In embodiments, the 3′ UTR sequence is downstream or 3′ relative to the Kozak sequence within the synthesis module. In embodiments, the 3′ UTR sequence is downstream or 3′ relative to the CDS within the synthesis module.

In embodiments, the synthesis module includes a polyA signal sequence. In embodiments, the 3′ UTR sequence includes the polyA signal sequence. In embodiments, the synthesis module further includes a sequence that regulates polyadenylation and/or termination of the target RNA. In embodiments, the polyA signal sequence includes a −bGH signal sequence, a SV40 signal sequence, or a hGH polyA signal sequence. In embodiments, the polyA signal sequence includes a −bGH signal sequence. In embodiments, the polyA signal sequence includes a SV40 signal sequence. In embodiments, the polyA signal sequence includes a hGH polyA signal sequence.

In embodiments, the 3′ UTR sequence or polyA signal sequence is at least 90% identical to a 3′ UTR or polyA sequence set forth in Table 1. In embodiments, the 3′ UTR sequence or polyA signal sequence is at least 91% identical to a 3′ UTR or polyA sequence set forth in Table 1. In embodiments, the 3′ UTR sequence or polyA signal sequence is at least 92% identical to a 3′ UTR or polyA sequence set forth in Table 1. In embodiments, the 3′ UTR sequence or polyA signal sequence is at least 93% identical to a 3′ UTR or polyA sequence set forth in Table 1. In embodiments, the 3′ UTR sequence or polyA signal sequence is at least 94% identical to a 3′ UTR or polyA sequence set forth in Table 1. In embodiments, the 3′ UTR sequence or polyA signal sequence is at least 95% identical to a 3′ UTR or polyA sequence set forth in Table 1. In embodiments, the 3′ UTR sequence or polyA signal sequence is at least 96% identical to a 3′ UTR or polyA sequence set forth in Table 1. In embodiments, the 3′ UTR sequence or polyA signal sequence is at least 97% identical to a 3′ UTR or polyA sequence set forth in Table 1. In embodiments, the 3′ UTR sequence or polyA signal sequence is at least 98% identical to a 3′ UTR or polyA sequence set forth in Table 1. In embodiments, the 3′ UTR sequence or polyA signal sequence is at least 99% identical to a 3′ UTR or polyA sequence set forth in Table 1. In embodiments, the 3′ UTR sequence or polyA signal sequence includes a 3′ UTR or polyA sequence set forth in Table 1. In embodiments, the 3′ UTR sequence or polyA signal sequence is a 3′ UTR or polyA sequence set forth in Table 1.

Some embodiments include an engineered 3′ UTR comprising multiple fragments of an endogenous MECP2 3′ UTR. Some embodiments include a polyA signal (e.g. a distal MECP2 polyA signal). Some embodiments include an engineered 3′ UTR consisting of multiple fragments of the endogenous MECP2 3′ UTR and the distal MECP2 polyA signal. In some embodiments, the vector lacks this element and only contains a standard polyA signal (e.g. −bGH, SV40, hGH).

### Silencing Module

Some embodiments relate to a silencing module. The silencing module may include a system for reducing nucleic acid expression by modifying splicing of the nucleic acid, which may include: an engineered U7 snRNA comprising an antisense nucleic acid sequence that targets an alternatively spliced region of an RNA encoding a MECP2 protein. In some embodiments, the engineered U7 snRNA further comprises an ESS nucleic acid sequence. In some embodiments, the engineered U7 snRNA does not comprise an ESS nucleic acid sequence.

Disclosed herein, in some embodiments, are recombinant small nuclear RNAs (snRNAs) or snRNA sequences. Disclosed herein, in some embodiments, are modified or recombinant U7 small nuclear RNAs (snRNAs) or modified or recombinant U7 snRNA sequences. A modified or recombinant U7 snRNA, or a modified or recombinant U7 snRNA sequence may be included as part of a system, or may be used in a method herein. Disclosed herein, in some embodiments, are systems that include a modified or recombinant U7 snRNA or that include a modified or recombinant U7 snRNA sequence. The modified or recombinant U7 snRNA may be or include an engineered U7 snRNA. Terms such as modified, recombinant, and engineered may be used interchangeably herein. The U7 snRNA sequence may be useful for modifying nucleic acid splicing. In some embodiments, the U7 snRNA sequence may include an exonic splicing silencer (ESS) nucleic acid sequence. Some embodiments do not include an ESS nucleic acid sequence. In some embodiments, the U7 snRNA sequence may include an antisense nucleic acid sequence that targets an alternatively spliced region of a ribonucleic acid (RNA). In some embodiments, the targeted RNA may encode a MECP2 protein. The U7 snRNA sequence may include a smOPT sequence. The U7 snRNA sequence may include a hairpin.

Described herein, in some embodiments, are methods or systems that affect splicing of a target RNA such as an MECP2 RNA. A target RNA may be referred to as a targeted RNA. A target RNA may include a targeted region. A targeted region may bind with or be bound by an antisense nucleic acid sequence. A targeted region may include an exon sequence. The targeted region may include an exon of an MECP2 mRNA. A targeted region may include a splice junction of an exon (e.g. an intron/exon junction). A targeted region may include a region near an exon such as an intron sequence. A targeted region may include an intron sequence. A targeted region may exclude an intron. A targeted region may exclude an exon sequence. A targeted region may include part of an intron sequence. A targeted region may include part of an exon sequence. A targeted region may exclude part of an intron sequence. A targeted region may exclude part of an exon sequence. A targeted region may encompass both a region near an exon and at least part of the exon.

A targeted region may include a sequence or region within an mRNA sequence of a NCBI Reference Sequence selected from the group consisting of: NM_001110792, NM_004992, NM_001316337, NM_001369391, and NM_001369392.

For the system provided herein, in embodiments, the silencing module includes an RNA molecule. In embodiments, the RNA molecule of the silencing module includes a modified U7 small nuclear RNA (snRNA). In embodiments, the modified U7 snRNA includes a U7 core sequence at least 90% identical to a U7 core sequence set forth in Table 1. In embodiments, the modified U7 snRNA includes a U7 core sequence at least 910% identical to a U7 core sequence set forth in Table 1. In embodiments, the modified U7 snRNA includes a U7 core sequence at least 92% identical to a U7 core sequence set forth in Table 1. In embodiments, the modified U7 snRNA includes a U7 core sequence at least 93% identical to a U7 core sequence set forth in Table 1. In embodiments, the modified U7 snRNA includes a U7 core sequence at least 94% identical to a U7 core sequence set forth in Table 1. In embodiments, the modified U7 snRNA includes a U7 core sequence at least 95% identical to a U7 core sequence set forth in Table 1. In embodiments, the modified U7 snRNA includes a U7 core sequence at least 96% identical to a U7 core sequence set forth in Table 1. In embodiments, the modified U7 snRNA includes a U7 core sequence at least 97% identical to a U7 core sequence set forth in Table 1. In embodiments, the modified U7 snRNA includes a U7 core sequence at least 98% identical to a U7 core sequence set forth in Table 1. In embodiments, the modified U7 snRNA includes a U7 core sequence at least 99% identical to a U7 core sequence set forth in Table 1. In embodiments, the modified U7 snRNA includes a U7 core sequence set forth in Table 1. In embodiments, the modified U7 snRNA is a U7 core sequence set forth in Table 1.

For the system provided herein, in embodiments, the silencing module includes a DNA molecule. In embodiments, the DNA molecule of the silencing module encodes a modified U7 snRNA. In embodiments, the DNA molecule of the silencing module includes a single silencing module. In embodiments, the DNA molecule of the silencing module includes an arrayed series of silencing modules. For example, the DNA molecule of the silencing module may include multiple silencing modules. In embodiments, the DNA molecule of the silencing module may include 1, 2, 3, 4, 5, 6, 8, 8, 9, or 10 silencing modules. In embodiments, the DNA molecule of the silencing module includes 4 silencing modules.

In embodiments, the silencing module includes an arrayed series of modified U7 snRNAs. In some embodiments, the array includes multiple U7 modules. Each module may include a targeting sequence. The targeting sequences of multiple modules may be the same. The targeting sequences of some modules may be different.

For the system provided herein, in embodiments, the silencing module includes an siRNA targeting an endogenous MECP2 RNA. Thus, in embodiments, the antisense nucleic acid sequence targeting an endogenous MECP2 RNA is an siRNA. In embodiments, the silencing module does not include a U7 snRNA or a modified U7 snRNA. A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene (e.g. when expressed in the same cell as the gene or target gene). The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

Disclosed herein, in some embodiments, is a system for modifying nucleic acid splicing, comprising: an exonic splicing silencer (ESS) nucleic acid sequence; and an antisense nucleic acid sequence that targets a region of a ribonucleic acid (RNA) encoding MECP2. The region may include an exon or a region near an exon. Targeting an RNA such as an MECP2 RNA may include binding or being reverse complementary to the RNA.

A modified U7 snRNA may include, in the following order from 5′ to 3′: (1) an optional exonic splicing silencer, (2) a targeting sequence (e.g. an antisense sequence) complementary to a region of a MECP2 mRNA, (4) a smOPT sequence, and (5) a hairpin sequence.

In some embodiments, a modified U7 snRNA includes a U7 core sequence, or an aspect of a U7 core sequence. In some embodiments, a recombinant U7 snRNA includes a U7 core sequence, or an aspect of a U7 core sequence. An example of a U7 core sequence is included in Table 1. For the U7 core sequence shown in Table 1, an antisense sequence is in parentheses, a smOPT is in in upper case, and a hairpin is in brackets. The antisense sequence may be or include a targeting sequence herein.

Some embodiments of a silencing module include a U7 module to suppress splicing of endogenous MECP2 transcripts. In some embodiments, the U7 module contains or includes a single U7 expression cassette or an array. In some embodiments, the U7 module contains or includes a single U7 expression cassette. In some embodiments, the U7 module contains or includes an array of U7 expression cassettes. The U7 module may contain or include U7 regulatory sequences such as a promoter and a 3′ element. The U7 module may contain or include an altered regulatory sequence. The U7 module may contain or include an altered promoter from another small RNA or an engineered promoter sequence. The U7 module may contain or include an altered 3′ element from another small RNA or an engineered 3′ element.

In some embodiments, the silencing module reduces a target (e.g. protein or RNA, such as a MECP2 protein or RNA) measurement in a cell or population of cells by at least 50%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%, relative to a baseline target measurement. In some embodiments, the silencing module reduces a target (e.g. protein or RNA, such as a MECP2 protein or RNA) measurement in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, or less than 80%, relative to a baseline target measurement.

### Silencing Module Promoter

For the system provided herein, in embodiments, the silencing module further includes a silencing module promoter sequence, or the silencing module is encoded by a nucleic acid including the silencing module promoter sequence. In embodiments, the silencing module further includes a silencing module promoter sequence. In embodiments, the silencing module is encoded by a nucleic acid including the silencing module promoter sequence.

The promoter may be operably connected with an ESS sequence, a U7 targeting sequence, a Sm binding site, or a U7 3′ hairpin, or a combination thereof. For example, the promoter may be operably connected with an ESS sequence, a U7 targeting sequence, a Sm binding site, and a U7 3′ hairpin within an expression construct. In embodiments, the silencing module promoter sequence includes a human promoter sequence. In embodiments, the silencing module promoter sequence includes a mouse promoter sequence.

In embodiments, the silencing module promoter includes a U7 snRNA promoter sequence. In embodiments, the silencing module promoter sequence includes a U1 snRNA promoter sequence. In embodiments, the silencing module promoter sequence includes a mouse U7 snRNA (“Mm U7”) promoter sequence, a human U7 snRNA (“Hs U7”) promoter sequence, a mouse U1a1 (“mu1a1” or “Mm U1a1”) promoter sequence, or a human U1-1 (“HU1” or “Hs U1-1”) promoter sequence, or a fragment or combination of fragments thereof. In embodiments, the silencing module promoter sequence includes a mouse U7 snRNA promoter sequence or a fragment thereof. In embodiments, the silencing module promoter sequence includes a human U7 snRNA promoter sequence or a fragment thereof. In embodiments, the silencing module promoter sequence includes a mouse U1a1 promoter sequence or a fragment thereof. In embodiments, the silencing module promoter sequence includes a human U1-1 promoter sequence or a fragment thereof.

In embodiments, the silencing module promoter sequence includes a U7 snRNA promoter sequence having a distal sequence element (DSE) replaced with a DSE of a U1-1 or U1a1 promoter sequence. In embodiments, the DSE is replaced with a DSE of a U1-1 promoter sequence. In embodiments, the DSE is replaced with a DSE of a U1a1 promoter sequence.

For the modified Mm U7 promoter in Table 1 (SEQ ID NO: 8), a U7 distal sequence element has been replaced with that of human U1-1 and a U7 proximal sequence element has been replaced with that of mouse U1a1. In embodiments, the silencing module promoter sequence includes a mouse U7 promoter sequence having a proximal sequence element (PSE) replaced with a PSE of a U1-1 or U1a1 promoter sequence. In embodiments, the PSE is replaced with a PSE of a U1-1 promoter sequence. In embodiments, the PSE is replaced with a PSE of a U1a1 promoter sequence.

In embodiments, the silencing module promoter sequence is at least 90% identical to a promoter sequence set forth in Table 1. In embodiments, the silencing module promoter sequence is at least 91% identical to a promoter sequence set forth in Table 1. In embodiments, the silencing module promoter sequence is at least 92% identical to a promoter sequence set forth in Table 1. In embodiments, the silencing module promoter sequence is at least 93% identical to a promoter sequence set forth in Table 1. In embodiments, the silencing module promoter sequence is at least 94% identical to a promoter sequence set forth in Table 1. In embodiments, the silencing module promoter sequence is at least 95% identical to a promoter sequence set forth in Table 1. In embodiments, the silencing module promoter sequence is at least 96% identical to a promoter sequence set forth in Table 1. In embodiments, the silencing module promoter sequence is at least 97% identical to a promoter sequence set forth in Table 1. In embodiments, the silencing module promoter sequence is at least 98% identical to a promoter sequence set forth in Table 1. In embodiments, the silencing module promoter sequence is at least 99% identical to a promoter sequence set forth in Table 1. In embodiments, the silencing module promoter sequence includes a promoter sequence set forth in Table 1. In embodiments, the silencing module promoter sequence is a promoter sequence set forth in Table 1. In some embodiments, the silencing module promoter sequence is at least 80%, at least 85%, at least 90%, at least 95%, at least 98%, at least 99%, or 100% identical to a promoter sequence in Table 1.

In embodiments, the silencing module promoter sequence is at least 90% identical to a promoter sequence set forth in Table 8. In embodiments, the silencing module promoter sequence is at least 91% identical to a promoter sequence set forth in Table 8. In embodiments, the silencing module promoter sequence is at least 92% identical to a promoter sequence set forth in Table 8. In embodiments, the silencing module promoter sequence is at least 93% identical to a promoter sequence set forth in Table 8. In embodiments, the silencing module promoter sequence is at least 94% identical to a promoter sequence set forth in Table 8. In embodiments, the silencing module promoter sequence is at least 95% identical to a promoter sequence set forth in Table 8. In embodiments, the silencing module promoter sequence is at least 96% identical to a promoter sequence set forth in Table 8. In embodiments, the silencing module promoter sequence is at least 97% identical to a promoter sequence set forth in Table 8. In embodiments, the silencing module promoter sequence is at least 98% identical to a promoter sequence set forth in Table 8. In embodiments, the silencing module promoter sequence is at least 99% identical to a promoter sequence set forth in Table 8. In embodiments, the silencing module promoter sequence includes a promoter sequence set forth in Table 8. In embodiments, the silencing module promoter sequence is a promoter sequence set forth in Table 8. In some embodiments, the silencing module promoter sequence is at least 80%, at least 85%, at least 90%, at least 95%, at least 98%, at least 99%, or 100% identical to a promoter sequence in Table 8.

In embodiments, the silencing module promoter sequence is 5′ or upstream relative to the ESS nucleic acid sequence or the antisense nucleic acid sequence.

In embodiments, the silencing module is operatively coupled to the silencing module promoter. In embodiments, the silencing module is operatively coupled to the synthesis module promoter. In embodiments, the synthesis module is operatively coupled to the synthesis module promoter. In embodiments, the synthesis module is operatively coupled to the silencing module promoter.

### Exonic Splicing Silencer (ESS)

Described herein, in some embodiments, are exonic splicing silencer (ESS) sequences. An ESS nucleic acid sequence is a sequence capable of enhancing splicing suppression. An ESS may be or include a 10-20 nt sequence at a 5′ terminus of an snRNA (e.g. engineered snRNA) capable of enhancing splicing suppression. The ESS may be included in a modified or recombinant snRNA or U7 snRNA. The ESS may recruit a protein factor that reduces splicing of the RNA encoding an MECP2 protein. An ESS sequence may refer to an ESS or to a sequence that encodes an ESS. An ESS may include a short region of an exon and is a cis-regulatory element (CREs). CREs are regions of non-coding DNA which regulate transcription of neighboring genes. CREs may include components of genetic regulatory networks that control the timing and the amount that a specific gene is expressed. An ESS may be bound by a negatively acting factor such as a heterogeneous ribonucleoprotein (hnRNP).

In embodiments, the ESS recruits a protein factor or group of factors that reduce or silence splicing of the endogenous MECP2 RNA. In embodiments, the ESS nucleic acid sequence includes a nucleic acid sequence at least 90% identical to ATGATAGGGACTTAGGGTGA (SEQ ID NO: 240), at least 90% identical to TTTGTTCCGTGGGTGGTTTA (SEQ ID NO: 241), or at least 90% identical to TGGGGGGAGGTAGGTAGGTA (SEQ ID NO: 242). In embodiments, the ESS nucleic acid sequence includes a nucleic acid sequence at least 90% identical to SEQ ID NO: 240. In embodiments, the ESS nucleic acid sequence includes a nucleic acid sequence at least 91% identical to SEQ ID NO: 240. In embodiments, the ESS nucleic acid sequence includes a nucleic acid sequence at least 92% identical to SEQ ID NO: 240. In embodiments, the ESS nucleic acid sequence includes a nucleic acid sequence at least 93% identical to SEQ ID NO: 240. In embodiments, the ESS nucleic acid sequence includes a nucleic acid sequence at least 94% identical to SEQ ID NO: 240. In embodiments, the ESS nucleic acid sequence includes a nucleic acid sequence at least 95% identical to SEQ ID NO: 240. In embodiments, the ESS nucleic acid sequence includes a nucleic acid sequence at least 96% identical to SEQ ID NO: 240. In embodiments, the ESS nucleic acid sequence includes a nucleic acid sequence at least 97% identical to SEQ ID NO: 240. In embodiments, the ESS nucleic acid sequence includes a nucleic acid sequence at least 98% identical to SEQ ID NO: 240. In embodiments, the ESS nucleic acid sequence includes a nucleic acid sequence at least 99% identical to SEQ ID NO: 240. In embodiments, the ESS nucleic acid sequence includes SEQ ID NO: 240. In embodiments, the ESS nucleic acid sequence is SEQ ID NO: 240.

In embodiments, the ESS nucleic acid sequence includes a nucleic acid sequence at least 90% identical to SEQ ID NO: 241. In embodiments, the ESS nucleic acid sequence includes a nucleic acid sequence at least 91% identical to SEQ ID NO: 241. In embodiments, the ESS nucleic acid sequence includes a nucleic acid sequence at least 92% identical to SEQ ID NO: 241. In embodiments, the ESS nucleic acid sequence includes a nucleic acid sequence at least 93% identical to SEQ ID NO: 241. In embodiments, the ESS nucleic acid sequence includes a nucleic acid sequence at least 94% identical to SEQ ID NO: 241. In embodiments, the ESS nucleic acid sequence includes a nucleic acid sequence at least 95% identical to SEQ ID NO: 241. In embodiments, the ESS nucleic acid sequence includes a nucleic acid sequence at least 96% identical to SEQ ID NO: 241. In embodiments, the ESS nucleic acid sequence includes a nucleic acid sequence at least 97% identical to SEQ ID NO: 241. In embodiments, the ESS nucleic acid sequence includes a nucleic acid sequence at least 98% identical to SEQ ID NO: 241. In embodiments, the ESS nucleic acid sequence includes a nucleic acid sequence at least 99% identical to SEQ ID NO: 241. In embodiments, the ESS nucleic acid sequence includes SEQ ID NO: 241. In embodiments, the ESS nucleic acid sequence is SEQ ID NO: 241.

In embodiments, the ESS nucleic acid sequence includes a nucleic acid sequence at least 90% identical to SEQ ID NO: 242. In embodiments, the ESS nucleic acid sequence includes a nucleic acid sequence at least 91% identical to SEQ ID NO: 242. In embodiments, the ESS nucleic acid sequence includes a nucleic acid sequence at least 92% identical to SEQ ID NO: 242. In embodiments, the ESS nucleic acid sequence includes a nucleic acid sequence at least 93% identical to SEQ ID NO: 242. In embodiments, the ESS nucleic acid sequence includes a nucleic acid sequence at least 94% identical to SEQ ID NO: 242. In embodiments, the ESS nucleic acid sequence includes a nucleic acid sequence at least 95% identical to SEQ ID NO: 242. In embodiments, the ESS nucleic acid sequence includes a nucleic acid sequence at least 96% identical to SEQ ID NO: 242. In embodiments, the ESS nucleic acid sequence includes a nucleic acid sequence at least 97% identical to SEQ ID NO: 242. In embodiments, the ESS nucleic acid sequence includes a nucleic acid sequence at least 98% identical to SEQ ID NO: 242. In embodiments, the ESS nucleic acid sequence includes a nucleic acid sequence at least 99% identical to SEQ ID NO: 242. In embodiments, the ESS nucleic acid sequence includes SEQ ID NO: 242. In embodiments, the ESS nucleic acid sequence is SEQ ID NO: 242.

### U7 Targeting Sequence

Described herein, in some embodiments, are targeting nucleic acid sequences such as snRNA targeting sequences or U7 targeting sequences. A targeting nucleic acid sequence may be or include an antisense nucleic acid sequence. A U7 targeting nucleic acid sequence may be or include a U7 antisense nucleic acid sequence. An snRNA targeting nucleic acid sequence may be or include a snRNA antisense nucleic acid sequence. Described herein, in some embodiments, are antisense nucleic acid sequences such as snRNA antisense sequences or U7 antisense sequences. An antisense sequence may be referred to as a targeting sequence. The antisense nucleic acid sequence may be included in a modified or recombinant snRNA or U7 snRNA. The antisense nucleic acid sequence may target (e.g. bind or be reverse complementary to) a target RNA such as an RNA encoding an MECP2 protein. The antisense nucleic acid sequence may bind to the target RNA. In some embodiments, an antisense nucleic acid sequence is encoded by a DNA sequence (e.g. a DNA expression construct).

For the system provided herein, in embodiments the antisense nucleic acid sequence targets a targeted region of the endogenous MECP2 RNA. In embodiments, the antisense nucleic acid sequence binds to the targeted region of the endogenous MECP2 RNA. In embodiments, the antisense nucleic acid sequence is fully reverse complementary or partially reverse complementary (e.g. at least 90% reverse complementary) to the targeted region. For example, in embodiments, the antisense nucleic acid is at least partially complementary to the targeted region of the endogenous MECP2 RNA.

In embodiments, the targeted region is within an intron of the endogenous MECP2 RNA. In embodiments, the endogenous MECP2 RNA is an MECP2 mRNA. In embodiments, the targeted region is within an intron of MECP2 mRNA. In embodiments, the intron includes an intron between exons 1 and 3 of MECP2, or an intron between exons 3 and 4 of MECP2. In embodiments, the targeted region is at least partially complementary to a splice site of the endogenous MECP2 RNA.

In embodiments, the intron includes a nucleic acid sequence at least 90% identical to SEQ ID NO: 8. In embodiments, the intron includes a nucleic acid sequence at least 91% identical to SEQ ID NO: 8. In embodiments, the intron includes a nucleic acid sequence at least 92% identical to SEQ ID NO: 8. In embodiments, the intron includes a nucleic acid sequence at least 93% identical to SEQ ID NO: 8. In embodiments, the intron includes a nucleic acid sequence at least 94% identical to SEQ ID NO: 8. In embodiments, the intron includes a nucleic acid sequence at least 95% identical to SEQ ID NO: 8. In embodiments, the intron includes a nucleic acid sequence at least 96% identical to SEQ ID NO: 8. In embodiments, the intron includes a nucleic acid sequence at least 97% identical to SEQ ID NO: 8. In embodiments, the intron includes a nucleic acid sequence at least 98% identical to SEQ ID NO: 8. In embodiments, the intron includes a nucleic acid sequence at least 99% identical to SEQ ID NO: 8. In embodiments, the intron includes SEQ ID NO: 8.

In embodiments, the intron includes a nucleic acid sequence at least 90% identical to SEQ ID NO: 9. In embodiments, the intron includes a nucleic acid sequence at least 91% identical to SEQ ID NO: 9. In embodiments, the intron includes a nucleic acid sequence at least 92% identical to SEQ ID NO: 9. In embodiments, the intron includes a nucleic acid sequence at least 93% identical to SEQ ID NO: 9. In embodiments, the intron includes a nucleic acid sequence at least 94% identical to SEQ ID NO: 9. In embodiments, the intron includes a nucleic acid sequence at least 95% identical to SEQ ID NO: 9. In embodiments, the intron includes a nucleic acid sequence at least 96% identical to SEQ ID NO: 9. In embodiments, the intron includes a nucleic acid sequence at least 97% identical to SEQ ID NO: 9. In embodiments, the intron includes a nucleic acid sequence at least 98% identical to SEQ ID NO: 9. In embodiments, the intron includes a nucleic acid sequence at least 99% identical to SEQ ID NO: 9. In embodiments, the intron includes SEQ ID NO: 9.

In embodiments, the intron includes a nucleic acid sequence at least 90% identical to SEQ ID NO: 10. In embodiments, the intron includes a nucleic acid sequence at least 91% identical to SEQ ID NO: 10. In embodiments, the intron includes a nucleic acid sequence at least 92% identical to SEQ ID NO: 10. In embodiments, the intron includes a nucleic acid sequence at least 93% identical to SEQ ID NO: 10. In embodiments, the intron includes a nucleic acid sequence at least 94% identical to SEQ ID NO: 10. In embodiments, the intron includes a nucleic acid sequence at least 95% identical to SEQ ID NO: 10. In embodiments, the intron includes a nucleic acid sequence at least 96% identical to SEQ ID NO: 10. In embodiments, the intron includes a nucleic acid sequence at least 97% identical to SEQ ID NO: 10. In embodiments, the intron includes a nucleic acid sequence at least 98% identical to SEQ ID NO: 10. In embodiments, the intron includes a nucleic acid sequence at least 99% identical to SEQ ID NO: 10. In embodiments, the intron includes SEQ ID NO: 10.

In embodiments, the intron includes a nucleic acid sequence at least 90% identical to SEQ ID NO: 11. In embodiments, the intron includes a nucleic acid sequence at least 91% identical to SEQ ID NO: 11. In embodiments, the intron includes a nucleic acid sequence at least 92% identical to SEQ ID NO: 11. In embodiments, the intron includes a nucleic acid sequence at least 93% identical to SEQ ID NO: 11. In embodiments, the intron includes a nucleic acid sequence at least 94% identical to SEQ ID NO: 11. In embodiments, the intron includes a nucleic acid sequence at least 95% identical to SEQ ID NO: 11. In embodiments, the intron includes a nucleic acid sequence at least 96% identical to SEQ ID NO: 11. In embodiments, the intron includes a nucleic acid sequence at least 97% identical to SEQ ID NO: 11. In embodiments, the intron includes a nucleic acid sequence at least 98% identical to SEQ ID NO: 11. In embodiments, the intron includes a nucleic acid sequence at least 99% identical to SEQ ID NO: 11. In embodiments, the intron includes SEQ ID NO: 11.

In embodiments, the intron includes a nucleic acid sequence at least 90% identical to SEQ ID NO: 12. In embodiments, the intron includes a nucleic acid sequence at least 91% identical to SEQ ID NO: 12. In embodiments, the intron includes a nucleic acid sequence at least 92% identical to SEQ ID NO: 12. In embodiments, the intron includes a nucleic acid sequence at least 93% identical to SEQ ID NO: 12. In embodiments, the intron includes a nucleic acid sequence at least 94% identical to SEQ ID NO: 12. In embodiments, the intron includes a nucleic acid sequence at least 95% identical to SEQ ID NO: 12. In embodiments, the intron includes a nucleic acid sequence at least 96% identical to SEQ ID NO: 12. In embodiments, the intron includes a nucleic acid sequence at least 97% identical to SEQ ID NO: 12. In embodiments, the intron includes a nucleic acid sequence at least 98% identical to SEQ ID NO: 12. In embodiments, the intron includes a nucleic acid sequence at least 99% identical to SEQ ID NO: 12. In embodiments, the intron includes SEQ ID NO: 12.

In embodiments, the intron includes a nucleic acid sequence at least 90% identical to SEQ ID NO: 13. In embodiments, the intron includes a nucleic acid sequence at least 91% identical to SEQ ID NO: 13. In embodiments, the intron includes a nucleic acid sequence at least 92% identical to SEQ ID NO: 13. In embodiments, the intron includes a nucleic acid sequence at least 93% identical to SEQ ID NO: 13. In embodiments, the intron includes a nucleic acid sequence at least 94% identical to SEQ ID NO: 13. In embodiments, the intron includes a nucleic acid sequence at least 95% identical to SEQ ID NO: 13. In embodiments, the intron includes a nucleic acid sequence at least 96% identical to SEQ ID NO: 13. In embodiments, the intron includes a nucleic acid sequence at least 97% identical to SEQ ID NO: 13. In embodiments, the intron includes a nucleic acid sequence at least 98% identical to SEQ ID NO: 13. In embodiments, the intron includes a nucleic acid sequence at least 99% identical to SEQ ID NO: 13. In embodiments, the intron includes SEQ ID NO: 13.

In embodiments, the intron includes a nucleic acid sequence at least 90% identical to SEQ ID NO: 14. In embodiments, the intron includes a nucleic acid sequence at least 91% identical to SEQ ID NO: 14. In embodiments, the intron includes a nucleic acid sequence at least 92% identical to SEQ ID NO: 14. In embodiments, the intron includes a nucleic acid sequence at least 93% identical to SEQ ID NO: 14. In embodiments, the intron includes a nucleic acid sequence at least 94% identical to SEQ ID NO: 14. In embodiments, the intron includes a nucleic acid sequence at least 95% identical to SEQ ID NO: 14. In embodiments, the intron includes a nucleic acid sequence at least 96% identical to SEQ ID NO: 14. In embodiments, the intron includes a nucleic acid sequence at least 97% identical to SEQ ID NO: 14. In embodiments, the intron includes a nucleic acid sequence at least 98% identical to SEQ ID NO: 14. In embodiments, the intron includes a nucleic acid sequence at least 99% identical to SEQ ID NO: 14. In embodiments, the intron includes SEQ ID NO: 14.

In embodiments, the intron includes a nucleic acid sequence at least 90% identical to SEQ ID NO: 15. In embodiments, the intron includes a nucleic acid sequence at least 91% identical to SEQ ID NO: 15. In embodiments, the intron includes a nucleic acid sequence at least 92% identical to SEQ ID NO: 15. In embodiments, the intron includes a nucleic acid sequence at least 93% identical to SEQ ID NO: 15. In embodiments, the intron includes a nucleic acid sequence at least 94% identical to SEQ ID NO: 15. In embodiments, the intron includes a nucleic acid sequence at least 95% identical to SEQ ID NO: 15. In embodiments, the intron includes a nucleic acid sequence at least 96% identical to SEQ ID NO: 15. In embodiments, the intron includes a nucleic acid sequence at least 97% identical to SEQ ID NO: 15. In embodiments, the intron includes a nucleic acid sequence at least 98% identical to SEQ ID NO: 15. In embodiments, the intron includes a nucleic acid sequence at least 99% identical to SEQ ID NO: 15. In embodiments, the intron includes SEQ ID NO: 15.

The antisense nucleic acid sequence may bind or target a target RNA. A target RNA or MECP2 RNA may be or include an MECP2 mRNA. An MECP2 mRNA may be or include an MECP2 pre-mRNA. For example, a target MECP2 RNA may include a MECP2 pre-mRNA. A pre-mRNA may include an mRNA before splicing, or before splicing is completed. Whan an mRNA has fully undergone splicing, it may be referred to as a mature mRNA.

In some embodiments, the target RNA or MECP2 RNA comprises a mammalian MECP2 RNA. In some embodiments, the MECP2 RNA comprises a primate MECP2 RNA. In some embodiments, the MECP2 RNA comprises a human MECP2 RNA. In some embodiments, the MECP2 RNA comprises a rodent or mouse MECP2 RNA.

A target RNA such as a target MECP2 RNA may include a targeted region. In embodiments, the targeted region is within an exon of the endogenous MECP2 RNA. In embodiments, the targeted region is within an exon of an endogenous MECP2 mRNA. In embodiments, the antisense nucleic acid sequence targets an alternatively spliced exon of the endogenous MECP2 RNA. In embodiments, the alternatively spliced exon includes an exon 2 of MECP2.

In embodiments, the targeted region is within a 5′ half or 5′ end of an intron or exon of the endogenous MECP2 RNA. For example, in embodiments, the targeted region may be closer to the 5′ end of an intron of the endogenous MECP2 RNA. In embodiments, the targeted region includes the 5′ end of an intron of the endogenous MECP2 RNA. In embodiments, the targeted region may be closer to the 5′ end of an exon of the endogenous MECP2 RNA. In embodiments, the targeted region includes the 5′ end of an exon of the endogenous MECP2 RNA.

In embodiments, the targeted region is within a 3′ half or 3′ end of an intron or exon of the endogenous MECP2 RNA. For example, in embodiments, the targeted region may be closer to the 3′ end of an intron of the endogenous MECP2 RNA. In embodiments, the targeted region includes the 3′ end of an intron of the endogenous MECP2 RNA. In embodiments, the targeted region may be closer to the 3′ end of an exon of the endogenous MECP2 RNA. In embodiments, the targeted region includes the 3′ end of an exon of the endogenous MECP2 RNA.

In embodiments, the targeted region is within 100 nucleotides of an intron/exon junction. In some embodiments, the targeted region is within 10, 20, 30, 40, 50, 60, 70, 80, 90, or 100 nucleotides of an intron/exon junction. In some embodiments, the targeted region is not within 10, 20, 30, 40, 50, 60, 70, 80, 90, or 100 nucleotides of an intron/exon junction.

In embodiments, the targeted region of the endogenous MECP2 RNA (e.g. MECP2 RNA) includes an intron-exon junction of the endogenous RNA. The intron-exon junction refers to the boundary between an intron and exon and includes the splice site that separates the intron and the exon upon pre-mRNA splicing. In embodiments, the targeted region of the endogenous MECP2 RNA is within 0 nt and 150 nt of an intron-exon junction. In embodiments, the targeted region of the endogenous MECP2 RNA is within 10 nt and 150 nt of an intron-exon junction. In embodiments, the targeted region of the endogenous MECP2 RNA is within 20 nt and 150 nt of an intron-exon junction. In embodiments, the targeted region of the endogenous MECP2 RNA is within 30 nt and 150 nt of an intron-exon junction. In embodiments, the targeted region of the endogenous MECP2 RNA is within 40 nt and 150 nt of an intron-exon junction. In embodiments, the targeted region of the endogenous MECP2 RNA is within 40 nt and 150 nt of an intron-exon junction. In embodiments, the targeted region of the endogenous MECP2 RNA is within 60 nt and 150 nt of an intron-exon junction. In embodiments, the targeted region of the endogenous MECP2 RNA is within 70 nt and 150 nt of an intron-exon junction. In embodiments, the targeted region of the endogenous MECP2 RNA is within 80 nt and 150 nt of an intron-exon junction. In embodiments, the targeted region of the endogenous MECP2 RNA is within 90 nt and 150 nt of an intron-exon junction. In embodiments, the targeted region of the endogenous MECP2 RNA is within 100 nt and 150 nt of an intron-exon junction. In embodiments, the targeted region of the endogenous MECP2 RNA is within 110 nt and 150 nt of an intron-exon junction. In embodiments, the targeted region of the endogenous MECP2 RNA is within 120 nt and 150 nt of an intron-exon junction. In embodiments, the targeted region of the endogenous MECP2 RNA is within 130 nt and 150 nt of an intron-exon junction. In embodiments, the targeted region of the endogenous MECP2 RNA is within 140 nt and 150 nt of an intron-exon junction.

In embodiments, the targeted region of the endogenous MECP2 RNA is within 0 nt and 140 nt of an intron-exon junction. In embodiments, the targeted region of the endogenous MECP2 RNA is within 0 nt and 130 nt of an intron-exon junction. In embodiments, the targeted region of the endogenous MECP2 RNA is within 0 nt and 120 nt of an intron-exon junction. In embodiments, the targeted region of the endogenous MECP2 RNA is within 0 nt and 110 nt of an intron-exon junction. In embodiments, the targeted region of the endogenous MECP2 RNA is within 0 nt and 100 nt of an intron-exon junction. In embodiments, the targeted region of the endogenous MECP2 RNA is within 0 nt and 90 nt of an intron-exon junction. In embodiments, the targeted region of the endogenous MECP2 RNA is within 0 nt and 80 nt of an intron-exon junction. In embodiments, the targeted region of the endogenous MECP2 RNA is within 0 nt and 70 nt of an intron-exon junction. In embodiments, the targeted region of the endogenous MECP2 RNA is within 0 nt and 60 nt of an intron-exon junction. In embodiments, the targeted region of the endogenous MECP2 RNA is within 0 nt and 50 nt of an intron-exon junction. In embodiments, the targeted region of the endogenous MECP2 RNA is within 0 nt and 40 nt of an intron-exon junction. In embodiments, the targeted region of the endogenous MECP2 RNA is within 0 nt and 30 nt of an intron-exon junction. In embodiments, the targeted region of the endogenous MECP2 RNA is within 0 nt and 20 nt of an intron-exon junction.

In embodiments, the targeted region of the endogenous MECP2 RNA is within 0 nt, 10 nt, 20 nt, 30 nt, 40 nt, 50 nt, 60 nt, 70 nt, 80 nt, 90 nt, 100 nt, 110 nt, 120 nt, 130 nt, 140 nt, or 150 of an intron-exon junction. In embodiments, the targeted region of the endogenous MECP2 RNA includes an intro-exon junction.

A targeted region may be or comprise a length of nucleotides. For example, a targeted region may be about 10, about 15, about 20, about 25, about 30, about 35, about 40, about 45, about 50, about 60, about 70, about 80, about 90, about 100, about 110, about 120, about 130, about 140, or about 150 nucleotides in length, or a range of lengths defined by any 2 of the aforementioned lengths. In some embodiments, the length is at least 10 nucleotides. In some embodiments, the length is at least 15 nucleotides. In some embodiments, the length is at least 20 nucleotides. In some embodiments, the length is at least 25 nucleotides. In some embodiments, the length is at least 30 nucleotides. In some embodiments, the length is at least 35 nucleotides. In some embodiments, the length is at least 40 nucleotides. In some embodiments, the length is at least 45 nucleotides. In some embodiments, the length is at least 50 nucleotides. In some embodiments, the length is at least 60 nucleotides. In some embodiments, the length is at least 70 nucleotides. In some embodiments, the length is at least 80 nucleotides. In some embodiments, the length is at least 90 nucleotides. In some embodiments, the length is at least 100 nucleotides. In some embodiments, the length is at least 110 nucleotides. In some embodiments, the length is at least 120 nucleotides. In some embodiments, the length is at least 130 nucleotides. In some embodiments, the length is at least 140 nucleotides. In some embodiments, the length is at least 150 nucleotides. In some embodiments, the length is less than 15 nucleotides. In some embodiments, the length is less than 20 nucleotides. In some embodiments, the length is less than 25 nucleotides. In some embodiments, the length is less than 30 nucleotides. In some embodiments, the length is less than 35 nucleotides. In some embodiments, the length is less than 40 nucleotides. In some embodiments, the length is less than 45 nucleotides. In some embodiments, the length is less than 50 nucleotides. In some embodiments, the length is less than 60 nucleotides. In some embodiments, the length is less than 70 nucleotides. In some embodiments, the length is less than 80 nucleotides. In some embodiments, the length is less than 90 nucleotides. In some embodiments, the length is less than 100 nucleotides. In some embodiments, the length is less than 110 nucleotides. In some embodiments, the length is less than 120 nucleotides. In some embodiments, the length is less than 130 nucleotides. In some embodiments, the length is less than 140 nucleotides. In some embodiments, the length is less than 150 nucleotides.

In embodiments, the antisense nucleic acid sequence (e.g. antisense nucleic acid sequence targeting an MECP2 RNA such as an endogenous MECP2 RNA) is 10-60 nucleotides in length. In embodiments, the antisense nucleic acid sequence (e.g. antisense nucleic acid sequence targeting an MECP2 RNA such as an endogenous MECP2 RNA) is 15-60 nucleotides in length. In embodiments, the antisense nucleic acid sequence (e.g. antisense nucleic acid sequence targeting an MECP2 RNA such as an endogenous MECP2 RNA) is 20-60 nucleotides in length. In embodiments, the antisense nucleic acid sequence (e.g. antisense nucleic acid sequence targeting an MECP2 RNA such as an endogenous MECP2 RNA) is 25-60 nucleotides in length. In embodiments, the antisense nucleic acid sequence (e.g. antisense nucleic acid sequence targeting an MECP2 RNA such as an endogenous MECP2 RNA) is 30-60 nucleotides in length. In embodiments, the antisense nucleic acid sequence (e.g. antisense nucleic acid sequence targeting an MECP2 RNA such as an endogenous MECP2 RNA) is 35-60 nucleotides in length. In embodiments, the antisense nucleic acid sequence (e.g. antisense nucleic acid sequence targeting an MECP2 RNA such as an endogenous MECP2 RNA) is 40-60 nucleotides in length. In embodiments, the antisense nucleic acid sequence (e.g. antisense nucleic acid sequence targeting an MECP2 RNA such as an endogenous MECP2 RNA) is 45-60 nucleotides in length. In embodiments, the antisense nucleic acid sequence (e.g. antisense nucleic acid sequence targeting an MECP2 RNA such as an endogenous MECP2 RNA) is 50-60 nucleotides in length. In embodiments, the antisense nucleic acid sequence (e.g. antisense nucleic acid sequence targeting an MECP2 RNA such as an endogenous MECP2 RNA) is 55-60 nucleotides in length.

In embodiments, the antisense nucleic acid sequence (e.g. antisense nucleic acid sequence targeting an MECP2 RNA such as an endogenous MECP2 RNA) is 10-55 nucleotides in length. In embodiments, the antisense nucleic acid sequence (e.g. antisense nucleic acid sequence targeting an MECP2 RNA such as an endogenous MECP2 RNA) is 10-50 nucleotides in length. In embodiments, the antisense nucleic acid sequence (e.g. antisense nucleic acid sequence targeting an MECP2 RNA such as an endogenous MECP2 RNA) is 10-45 nucleotides in length. In embodiments, the antisense nucleic acid sequence (e.g. antisense nucleic acid sequence targeting an MECP2 RNA such as an endogenous MECP2 RNA) is 10-40 nucleotides in length. In embodiments, the antisense nucleic acid sequence (e.g. antisense nucleic acid sequence targeting an MECP2 RNA such as an endogenous MECP2 RNA) is 10-35 nucleotides in length. In embodiments, the antisense nucleic acid sequence (e.g. antisense nucleic acid sequence targeting an MECP2 RNA such as an endogenous MECP2 RNA) is 10-30 nucleotides in length. In embodiments, the antisense nucleic acid sequence (e.g. antisense nucleic acid sequence targeting an MECP2 RNA such as an endogenous MECP2 RNA) is 10-25 nucleotides in length. In embodiments, the antisense nucleic acid sequence (e.g. antisense nucleic acid sequence targeting an MECP2 RNA such as an endogenous MECP2 RNA) is 10-20 nucleotides in length. In embodiments, the antisense nucleic acid sequence (e.g. antisense nucleic acid sequence targeting an MECP2 RNA such as an endogenous MECP2 RNA) is 10-15 nucleotides in length. In embodiments, the antisense nucleic acid sequence (e.g. antisense nucleic acid sequence targeting an MECP2 RNA such as an endogenous MECP2 RNA) is about 10, 15, 20, 25, 30, 35, 40, 45, 50, 55, or 60 nucleotides in length, or a range defined by any two of the aforementioned numbers of nucleotides in length.

In some embodiments, the nucleic acid targeting sequence targets or binds exon 1 of MECP2. In some embodiments, the nucleic acid targeting sequence targets or binds SEQ ID NO: 365. In some embodiments, the nucleic acid targeting sequence targets or binds a sequence at least 80%, at least 85%, at least 90%, at least 95%, at least 98%, at least 99%, or 100% identical to SEQ ID NO: 365. In some embodiments, the nucleic acid targeting sequence targets or binds SEQ ID NO: 368. In some embodiments, the nucleic acid targeting sequence targets or binds a sequence at least 80%, at least 85%, at least 90%, at least 95%, at least 98%, at least 99%, or 100% identical to SEQ ID NO: 368.

In some embodiments, the nucleic acid targeting sequence targets or binds exon 3 of MECP2. In some embodiments, the nucleic acid targeting sequence targets or binds SEQ ID NO: 366. In some embodiments, the nucleic acid targeting sequence targets or binds a sequence at least 80%, at least 85%, at least 90%, at least 95%, at least 98%, at least 99%, or 100% identical to SEQ ID NO: 366. In some embodiments, the nucleic acid targeting sequence targets or binds SEQ ID NO: 369. In some embodiments, the nucleic acid targeting sequence targets or binds a sequence at least 80%, at least 85%, at least 90%, at least 95%, at least 98%, at least 99%, or 100% identical to SEQ ID NO: 369.

In some embodiments, the nucleic acid targeting sequence targets or binds exon 4 of MECP2. In some embodiments, the nucleic acid targeting sequence targets or binds SEQ ID NO: 367. In some embodiments, the nucleic acid targeting sequence targets or binds a sequence at least 80%, at least 85%, at least 90%, at least 95%, at least 98%, at least 99%, or 100% identical to SEQ ID NO: 367. In some embodiments, the nucleic acid targeting sequence targets or binds SEQ ID NO: 370. In some embodiments, the nucleic acid targeting sequence targets or binds a sequence at least 80%, at least 85%, at least 90%, at least 95%, at least 98%, at least 99%, or 100% identical to SEQ ID NO: 370.

In embodiments, the antisense nucleic acid sequence includes a nucleic acid sequence at least 90% identical to an antisense nucleic acid sequence set forth in Table 2. In embodiments, the antisense nucleic acid sequence includes a nucleic acid sequence at least 91% identical to an antisense nucleic acid sequence set forth in Table 2. In embodiments, the antisense nucleic acid sequence includes a nucleic acid sequence at least 92% identical to an antisense nucleic acid sequence set forth in Table 2. In embodiments, the antisense nucleic acid sequence includes a nucleic acid sequence at least 93% identical to an antisense nucleic acid sequence set forth in Table 2. In embodiments, the antisense nucleic acid sequence includes a nucleic acid sequence at least 94% identical to an antisense nucleic acid sequence set forth in Table 2. In embodiments, the antisense nucleic acid sequence includes a nucleic acid sequence at least 95% identical to an antisense nucleic acid sequence set forth in Table 2. In embodiments, the antisense nucleic acid sequence includes a nucleic acid sequence at least 96% identical to an antisense nucleic acid sequence set forth in Table 2. In embodiments, the antisense nucleic acid sequence includes a nucleic acid sequence at least 97% identical to an antisense nucleic acid sequence set forth in Table 2. In embodiments, the antisense nucleic acid sequence includes a nucleic acid sequence at least 98% identical to an antisense nucleic acid sequence set forth in Table 2. In embodiments, the antisense nucleic acid sequence includes a nucleic acid sequence at least 99% identical to an antisense nucleic acid sequence set forth in Table 2. In embodiments, the antisense nucleic acid sequence includes an antisense nucleic acid sequence set forth in Table 2. In embodiments, the antisense nucleic acid sequence is an antisense nucleic acid sequence set forth in Table 2.

In embodiments, the antisense nucleic acid sequence is at least 90% identical, at least 95% identical, or 100% identical to SEQ ID NO: 30. In embodiments, the antisense nucleic acid sequence is at least 90% identical, at least 95% identical, or 100% identical, at least 95% identical, or 100% identical to SEQ ID NO: 31. In embodiments, the antisense nucleic acid sequence is at least 90% identical, at least 95% identical, or 100% identical to SEQ ID NO: 32. In embodiments, the antisense nucleic acid sequence is at least 90% identical, at least 95% identical, or 100% identical to SEQ ID NO: 33. In embodiments, the antisense nucleic acid sequence is at least 90% identical, at least 95% identical, or 100% identical to SEQ ID NO: 34. In embodiments, the antisense nucleic acid sequence is at least 90% identical, at least 95% identical, or 100% identical to SEQ ID NO: 35. In embodiments, the antisense nucleic acid sequence is at least 90% identical, at least 95% identical, or 100% identical to SEQ ID NO: 36. In embodiments, the antisense nucleic acid sequence is at least 90% identical, at least 95% identical, or 100% identical to SEQ ID NO: 37. In embodiments, the antisense nucleic acid sequence is at least 90% identical, at least 95% identical, or 100% identical to SEQ ID NO: 38. In embodiments, the antisense nucleic acid sequence is at least 90% identical, at least 95% identical, or 100% identical to SEQ ID NO: 39. In embodiments, the antisense nucleic acid sequence is at least 90% identical, at least 95% identical, or 100% identical to SEQ ID NO: 40. In embodiments, the antisense nucleic acid sequence is at least 90% identical, at least 95% identical, or 100% identical to SEQ ID NO: 41. In embodiments, the antisense nucleic acid sequence is at least 90% identical, at least 95% identical, or 100% identical to SEQ ID NO: 42. In embodiments, the antisense nucleic acid sequence is at least 90% identical, at least 95% identical, or 100% identical to SEQ ID NO: 43. In embodiments, the antisense nucleic acid sequence is at least 90% identical, at least 95% identical, or 100% identical to SEQ ID NO: 44. In embodiments, the antisense nucleic acid sequence is at least 90% identical, at least 95% identical, or 100% identical to SEQ ID NO: 45. In embodiments, the antisense nucleic acid sequence is at least 90% identical, at least 95% identical, or 100% identical to SEQ ID NO: 46. In embodiments, the antisense nucleic acid sequence is at least 90% identical, at least 95% identical, or 100% identical to SEQ ID NO: 47. In embodiments, the antisense nucleic acid sequence is at least 90% identical, at least 95% identical, or 100% identical to SEQ ID NO: 48. In embodiments, the antisense nucleic acid sequence is at least 90% identical, at least 95% identical, or 100% identical to SEQ ID NO: 49. In embodiments, the antisense nucleic acid sequence is at least 90% identical, at least 95% identical, or 100% identical to SEQ ID NO: 50. In embodiments, the antisense nucleic acid sequence is at least 90% identical, at least 95% identical, or 100% identical to SEQ ID NO: 51. In embodiments, the antisense nucleic acid sequence is at least 90% identical, at least 95% identical, or 100% identical to SEQ ID NO: 52. In embodiments, the antisense nucleic acid sequence is at least 90% identical, at least 95% identical, or 100% identical to SEQ ID NO: 53. In embodiments, the antisense nucleic acid sequence is at least 90% identical, at least 95% identical, or 100% identical to SEQ ID NO: 54. In embodiments, the antisense nucleic acid sequence is at least 90% identical, at least 95% identical, or 100% identical to SEQ ID NO: 55. In embodiments, the antisense nucleic acid sequence is at least 90% identical, at least 95% identical, or 100% identical to SEQ ID NO: 56. In embodiments, the antisense nucleic acid sequence is at least 90% identical, at least 95% identical, or 100% identical to SEQ ID NO: 57. In embodiments, the antisense nucleic acid sequence is at least 90% identical, at least 95% identical, or 100% identical to SEQ ID NO: 58. In embodiments, the antisense nucleic acid sequence is at least 90% identical, at least 95% identical, or 100% identical to SEQ ID NO: 59. In embodiments, the antisense nucleic acid sequence is at least 90% identical, at least 95% identical, or 100% identical to SEQ ID NO: 60. In embodiments, the antisense nucleic acid sequence is at least 90% identical, at least 95% identical, or 100% identical to SEQ ID NO: 61. In embodiments, the antisense nucleic acid sequence is at least 90% identical, at least 95% identical, or 100% identical to SEQ ID NO: 62. In embodiments, the antisense nucleic acid sequence is at least 90% identical, at least 95% identical, or 100% identical to SEQ ID NO: 63. In embodiments, the antisense nucleic acid sequence is at least 90% identical, at least 95% identical, or 100% identical to SEQ ID NO: 64. In embodiments, the antisense nucleic acid sequence is at least 90% identical, at least 95% identical, or 100% identical to SEQ ID NO: 65. In embodiments, the antisense nucleic acid sequence is at least 90% identical, at least 95% identical, or 100% identical to SEQ ID NO: 66. In embodiments, the antisense nucleic acid sequence is at least 90% identical, at least 95% identical, or 100% identical to SEQ ID NO: 67. In embodiments, the antisense nucleic acid sequence is at least 90% identical, at least 95% identical, or 100% identical to SEQ ID NO: 68. In embodiments, the antisense nucleic acid sequence is at least 90% identical, at least 95% identical, or 100% identical to SEQ ID NO: 69. In embodiments, the antisense nucleic acid sequence is at least 90% identical, at least 95% identical, or 100% identical to SEQ ID NO: 70. In embodiments, the antisense nucleic acid sequence is at least 90% identical, at least 95% identical, or 100% identical to SEQ ID NO: 71. In embodiments, the antisense nucleic acid sequence is at least 90% identical, at least 95% identical, or 100% identical to SEQ ID NO: 72. In embodiments, the antisense nucleic acid sequence is at least 90% identical, at least 95% identical, or 100% identical to SEQ ID NO: 73. In embodiments, the antisense nucleic acid sequence is at least 90% identical, at least 95% identical, or 100% identical to SEQ ID NO: 74. In embodiments, the antisense nucleic acid sequence is at least 90% identical, at least 95% identical, or 100% identical to SEQ ID NO: 75. In embodiments, the antisense nucleic acid sequence is at least 90% identical, at least 95% identical, or 100% identical to SEQ ID NO: 76. In embodiments, the antisense nucleic acid sequence is at least 90% identical, at least 95% identical, or 100% identical to SEQ ID NO: 76. In embodiments, the antisense nucleic acid sequence is at least 90% identical, at least 95% identical, or 100% identical to SEQ ID NO: 77. In embodiments, the antisense nucleic acid sequence is at least 90% identical, at least 95% identical, or 100% identical to SEQ ID NO: 78. In embodiments, the antisense nucleic acid sequence is at least 90% identical, at least 95% identical, or 100% identical to SEQ ID NO: 79. In embodiments, the antisense nucleic acid sequence is at least 90% identical, at least 95% identical, or 100% identical to SEQ ID NO: 80. In embodiments, the antisense nucleic acid sequence is at least 90% identical, at least 95% identical, or 100% identical to SEQ ID NO: 81. In embodiments, the antisense nucleic acid sequence is at least 90% identical, at least 95% identical, or 100% identical to SEQ ID NO: 82. In embodiments, the antisense nucleic acid sequence is at least 90% identical, at least 95% identical, or 100% identical to SEQ ID NO: 83. In embodiments, the antisense nucleic acid sequence is at least 90% identical, at least 95% identical, or 100% identical to SEQ ID NO: 84. In embodiments, the antisense nucleic acid sequence is at least 90% identical, at least 95% identical, or 100% identical to SEQ ID NO: 84. In embodiments, the antisense nucleic acid sequence is at least 90% identical, at least 95% identical, or 100% identical to SEQ ID NO: 85. In embodiments, the antisense nucleic acid sequence is at least 90% identical, at least 95% identical, or 100% identical to SEQ ID NO: 86. In embodiments, the antisense nucleic acid sequence is at least 90% identical, at least 95% identical, or 100% identical to SEQ ID NO: 87. In embodiments, the antisense nucleic acid sequence is at least 90% identical, at least 95% identical, or 100% identical to SEQ ID NO: 88. In embodiments, the antisense nucleic acid sequence is at least 90% identical, at least 95% identical, or 100% identical to SEQ ID NO: 89. In embodiments, the antisense nucleic acid sequence is at least 90% identical, at least 95% identical, or 100% identical to SEQ ID NO: 90. In embodiments, the antisense nucleic acid sequence is at least 90% identical, at least 95% identical, or 100% identical to SEQ ID NO: 91. In embodiments, the antisense nucleic acid sequence is at least 90% identical, at least 95% identical, or 100% identical to SEQ ID NO: 92. In embodiments, the antisense nucleic acid sequence is at least 90% identical, at least 95% identical, or 100% identical to SEQ ID NO: 93. In embodiments, the antisense nucleic acid sequence is at least 90% identical, at least 95% identical, or 100% identical to SEQ ID NO: 94. In embodiments, the antisense nucleic acid sequence is at least 90% identical, at least 95% identical, or 100% identical to SEQ ID NO: 95. In embodiments, the antisense nucleic acid sequence is at least 90% identical, at least 95% identical, or 100% identical to SEQ ID NO: 96. In embodiments, the antisense nucleic acid sequence is at least 90% identical, at least 95% identical, or 100% identical to SEQ ID NO: 97. In embodiments, the antisense nucleic acid sequence is at least 90% identical, at least 95% identical, or 100% identical to SEQ ID NO: 98. In embodiments, the antisense nucleic acid sequence is at least 90% identical, at least 95% identical, or 100% identical to SEQ ID NO: 99. In embodiments, the antisense nucleic acid sequence is at least 90% identical, at least 95% identical, or 100% identical to SEQ ID NO: 100. In embodiments, the antisense nucleic acid sequence is at least 90% identical, at least 95% identical, or 100% identical to SEQ ID NO: 101. In embodiments, the antisense nucleic acid sequence is at least 90% identical, at least 95% identical, or 100% identical to SEQ ID NO: 102. In embodiments, the antisense nucleic acid sequence is at least 90% identical, at least 95% identical, or 100% identical to SEQ ID NO: 103. In embodiments, the antisense nucleic acid sequence is at least 90% identical, at least 95% identical, or 100% identical to SEQ ID NO: 104. In embodiments, the antisense nucleic acid sequence is at least 90% identical, at least 95% identical, or 100% identical to SEQ ID NO: 105. In embodiments, the antisense nucleic acid sequence is at least 90% identical, at least 95% identical, or 100% identical to SEQ ID NO: 106. In embodiments, the antisense nucleic acid sequence is at least 90% identical, at least 95% identical, or 100% identical to SEQ ID NO: 107. In embodiments, the antisense nucleic acid sequence is at least 90% identical, at least 95% identical, or 100% identical to SEQ ID NO: 108. In embodiments, the antisense nucleic acid sequence is at least 90% identical, at least 95% identical, or 100% identical to SEQ ID NO: 109. In embodiments, the antisense nucleic acid sequence is at least 90% identical, at least 95% identical, or 100% identical to SEQ ID NO: 110. In embodiments, the antisense nucleic acid sequence is at least 90% identical, at least 95% identical, or 100% identical to SEQ ID NO: 111. In embodiments, the antisense nucleic acid sequence is at least 90% identical, at least 95% identical, or 100% identical to SEQ ID NO: 112. In embodiments, the antisense nucleic acid sequence is at least 90% identical, at least 95% identical, or 100% identical to SEQ ID NO: 113. In embodiments, the antisense nucleic acid sequence is at least 90% identical, at least 95% identical, or 100% identical to SEQ ID NO: 114. In embodiments, the antisense nucleic acid sequence is at least 90% identical, at least 95% identical, or 100% identical to SEQ ID NO: 115. In embodiments, the antisense nucleic acid sequence is at least 90% identical, at least 95% identical, or 100% identical to SEQ ID NO: 116. In embodiments, the antisense nucleic acid sequence is at least 90% identical, at least 95% identical, or 100% identical to SEQ ID NO: 117. In embodiments, the antisense nucleic acid sequence is at least 90% identical, at least 95% identical, or 100% identical to SEQ ID NO: 118. In embodiments, the antisense nucleic acid sequence is at least 90% identical, at least 95% identical, or 100% identical to SEQ ID NO: 119. In embodiments, the antisense nucleic acid sequence is at least 90% identical, at least 95% identical, or 100% identical to SEQ ID NO: 120. In embodiments, the antisense nucleic acid sequence is at least 90% identical, at least 95% identical, or 100% identical to SEQ ID NO: 121. In embodiments, the antisense nucleic acid sequence is at least 90% identical, at least 95% identical, or 100% identical to SEQ ID NO: 126. In embodiments, the antisense nucleic acid sequence is at least 90% identical, at least 95% identical, or 100% identical to SEQ ID NO: 127. In embodiments, the antisense nucleic acid sequence is at least 90% identical, at least 95% identical, or 100% identical to SEQ ID NO: 128. In embodiments, the antisense nucleic acid sequence is at least 90% identical, at least 95% identical, or 100% identical to SEQ ID NO: 129. In embodiments, the antisense nucleic acid sequence is at least 90% identical, at least 95% identical, or 100% identical to SEQ ID NO: 130. In embodiments, the antisense nucleic acid sequence is at least 90% identical, at least 95% identical, or 100% identical to SEQ ID NO: 131. In embodiments, the antisense nucleic acid sequence is at least 90% identical, at least 95% identical, or 100% identical to SEQ ID NO: 132. In embodiments, the antisense nucleic acid sequence is at least 90% identical, at least 95% identical, or 100% identical to SEQ ID NO: 133. In embodiments, the antisense nucleic acid sequence is at least 90% identical, at least 95% identical, or 100% identical to SEQ ID NO: 134. In embodiments, the antisense nucleic acid sequence is at least 90% identical, at least 95% identical, or 100% identical to SEQ ID NO: 135. In embodiments, the antisense nucleic acid sequence is at least 90% identical, at least 95% identical, or 100% identical to SEQ ID NO: 136. In embodiments, the antisense nucleic acid sequence is at least 90% identical, at least 95% identical, or 100% identical to SEQ ID NO: 137. In embodiments, the antisense nucleic acid sequence is at least 90% identical, at least 95% identical, or 100% identical to SEQ ID NO: 138. In embodiments, the antisense nucleic acid sequence is at least 90% identical, at least 95% identical, or 100% identical to SEQ ID NO: 139. In embodiments, the antisense nucleic acid sequence is at least 90% identical, at least 95% identical, or 100% identical to SEQ ID NO: 140. In embodiments, the antisense nucleic acid sequence is at least 90% identical, at least 95% identical, or 100% identical to SEQ ID NO: 141. In embodiments, the antisense nucleic acid sequence is at least 90% identical, at least 95% identical, or 100% identical to SEQ ID NO: 142. In embodiments, the antisense nucleic acid sequence is at least 90% identical, at least 95% identical, or 100% identical to SEQ ID NO: 143. In embodiments, the antisense nucleic acid sequence is at least 90% identical, at least 95% identical, or 100% identical to SEQ ID NO: 144. In embodiments, the antisense nucleic acid sequence is at least 90% identical, at least 95% identical, or 100% identical to SEQ ID NO: 145. In embodiments, the antisense nucleic acid sequence is at least 90% identical, at least 95% identical, or 100% identical to SEQ ID NO: 146. In embodiments, the antisense nucleic acid sequence is at least 90% identical, at least 95% identical, or 100% identical to SEQ ID NO: 147. In embodiments, the antisense nucleic acid sequence is at least 90% identical, at least 95% identical, or 100% identical to SEQ ID NO: 148. In embodiments, the antisense nucleic acid sequence is at least 90% identical, at least 95% identical, or 100% identical to SEQ ID NO: 149. In embodiments, the antisense nucleic acid sequence is at least 90% identical, at least 95% identical, or 100% identical to SEQ ID NO: 150. In embodiments, the antisense nucleic acid sequence is at least 90% identical, at least 95% identical, or 100% identical to SEQ ID NO: 151. In embodiments, the antisense nucleic acid sequence is at least 90% identical, at least 95% identical, or 100% identical to SEQ ID NO: 152. In embodiments, the antisense nucleic acid sequence is at least 90% identical, at least 95% identical, or 100% identical to SEQ ID NO: 153. In embodiments, the antisense nucleic acid sequence is at least 90% identical, at least 95% identical, or 100% identical to SEQ ID NO: 154. In embodiments, the antisense nucleic acid sequence is at least 90% identical, at least 95% identical, or 100% identical to SEQ ID NO: 155. In embodiments, the antisense nucleic acid sequence is at least 90% identical, at least 95% identical, or 100% identical to SEQ ID NO: 156. In embodiments, the antisense nucleic acid sequence is at least 90% identical, at least 95% identical, or 100% identical to SEQ ID NO: 157. In embodiments, the antisense nucleic acid sequence is at least 90% identical, at least 95% identical, or 100% identical to SEQ ID NO: 158. In embodiments, the antisense nucleic acid sequence is at least 90% identical, at least 95% identical, or 100% identical to SEQ ID NO: 159. In embodiments, the antisense nucleic acid sequence is at least 90% identical, at least 95% identical, or 100% identical to SEQ ID NO: 160. In embodiments, the antisense nucleic acid sequence is at least 90% identical, at least 95% identical, or 100% identical to SEQ ID NO: 161. In embodiments, the antisense nucleic acid sequence is at least 90% identical, at least 95% identical, or 100% identical to SEQ ID NO: 162. In embodiments, the antisense nucleic acid sequence is at least 90% identical, at least 95% identical, or 100% identical to SEQ ID NO: 163. In embodiments, the antisense nucleic acid sequence is at least 90% identical, at least 95% identical, or 100% identical to SEQ ID NO: 164. In embodiments, the antisense nucleic acid sequence is at least 90% identical, at least 95% identical, or 100% identical to SEQ ID NO: 165. In embodiments, the antisense nucleic acid sequence is at least 90% identical, at least 95% identical, or 100% identical to SEQ ID NO: 166. In embodiments, the antisense nucleic acid sequence is at least 90% identical, at least 95% identical, or 100% identical to SEQ ID NO: 167. In embodiments, the antisense nucleic acid sequence is at least 90% identical, at least 95% identical, or 100% identical to SEQ ID NO: 168. In embodiments, the antisense nucleic acid sequence is at least 90% identical, at least 95% identical, or 100% identical to SEQ ID NO: 169. In embodiments, the antisense nucleic acid sequence is at least 90% identical, at least 95% identical, or 100% identical to SEQ ID NO: 170. In embodiments, the antisense nucleic acid sequence is at least 90% identical, at least 95% identical, or 100% identical to SEQ ID NO: 171. In embodiments, the antisense nucleic acid sequence is at least 90% identical, at least 95% identical, or 100% identical to SEQ ID NO: 172. In embodiments, the antisense nucleic acid sequence is at least 90% identical, at least 95% identical, or 100% identical to SEQ ID NO: 173. In embodiments, the antisense nucleic acid sequence is at least 90% identical, at least 95% identical, or 100% identical to SEQ ID NO: 174. In embodiments, the antisense nucleic acid sequence is at least 90% identical, at least 95% identical, or 100% identical to SEQ ID NO: 175. In embodiments, the antisense nucleic acid sequence is at least 90% identical, at least 95% identical, or 100% identical to SEQ ID NO: 176. In embodiments, the antisense nucleic acid sequence is at least 90% identical, at least 95% identical, or 100% identical to SEQ ID NO: 176. In embodiments, the antisense nucleic acid sequence is at least 90% identical, at least 95% identical, or 100% identical to SEQ ID NO: 177. In embodiments, the antisense nucleic acid sequence is at least 90% identical, at least 95% identical, or 100% identical to SEQ ID NO: 178. In embodiments, the antisense nucleic acid sequence is at least 90% identical, at least 95% identical, or 100% identical to SEQ ID NO: 179. In embodiments, the antisense nucleic acid sequence is at least 90% identical, at least 95% identical, or 100% identical to SEQ ID NO: 180. In embodiments, the antisense nucleic acid sequence is at least 90% identical, at least 95% identical, or 100% identical to SEQ ID NO: 181. In embodiments, the antisense nucleic acid sequence is at least 90% identical, at least 95% identical, or 100% identical to SEQ ID NO: 182. In embodiments, the antisense nucleic acid sequence is at least 90% identical, at least 95% identical, or 100% identical to SEQ ID NO: 183. In embodiments, the antisense nucleic acid sequence is at least 90% identical, at least 95% identical, or 100% identical to SEQ ID NO: 184. In embodiments, the antisense nucleic acid sequence is at least 90% identical, at least 95% identical, or 100% identical to SEQ ID NO: 184. In embodiments, the antisense nucleic acid sequence is at least 90% identical, at least 95% identical, or 100% identical to SEQ ID NO: 185. In embodiments, the antisense nucleic acid sequence is at least 90% identical, at least 95% identical, or 100% identical to SEQ ID NO: 186. In embodiments, the antisense nucleic acid sequence is at least 90% identical, at least 95% identical, or 100% identical to SEQ ID NO: 187. In embodiments, the antisense nucleic acid sequence is at least 90% identical, at least 95% identical, or 100% identical to SEQ ID NO: 188. In embodiments, the antisense nucleic acid sequence is at least 90% identical, at least 95% identical, or 100% identical to SEQ ID NO: 189. In embodiments, the antisense nucleic acid sequence is at least 90% identical, at least 95% identical, or 100% identical to SEQ ID NO: 190. In embodiments, the antisense nucleic acid sequence is at least 90% identical, at least 95% identical, or 100% identical to SEQ ID NO: 191. In embodiments, the antisense nucleic acid sequence is at least 90% identical, at least 95% identical, or 100% identical to SEQ ID NO: 192. In embodiments, the antisense nucleic acid sequence is at least 90% identical, at least 95% identical, or 100% identical to SEQ ID NO: 193. In embodiments, the antisense nucleic acid sequence is at least 90% identical, at least 95% identical, or 100% identical to SEQ ID NO: 194. In embodiments, the antisense nucleic acid sequence is at least 90% identical, at least 95% identical, or 100% identical to SEQ ID NO: 195. In embodiments, the antisense nucleic acid sequence is at least 90% identical, at least 95% identical, or 100% identical to SEQ ID NO: 196. In embodiments, the antisense nucleic acid sequence is at least 90% identical, at least 95% identical, or 100% identical to SEQ ID NO: 197. In embodiments, the antisense nucleic acid sequence is at least 90% identical, at least 95% identical, or 100% identical to SEQ ID NO: 198. In embodiments, the antisense nucleic acid sequence is at least 90% identical, at least 95% identical, or 100% identical to SEQ ID NO: 199. In embodiments, the antisense nucleic acid sequence is at least 90% identical, at least 95% identical, or 100% identical to SEQ ID NO: 200. In embodiments, the antisense nucleic acid sequence is at least 90% identical, at least 95% identical, or 100% identical to SEQ ID NO: 201. In embodiments, the antisense nucleic acid sequence is at least 90% identical, at least 95% identical, or 100% identical to SEQ ID NO: 202. In embodiments, the antisense nucleic acid sequence is at least 90% identical, at least 95% identical, or 100% identical to SEQ ID NO: 203. In embodiments, the antisense nucleic acid sequence is at least 90% identical, at least 95% identical, or 100% identical to SEQ ID NO: 204. In embodiments, the antisense nucleic acid sequence is at least 90% identical, at least 95% identical, or 100% identical to SEQ ID NO: 205. In embodiments, the antisense nucleic acid sequence is at least 90% identical, at least 95% identical, or 100% identical to SEQ ID NO: 206. In embodiments, the antisense nucleic acid sequence is at least 90% identical, at least 95% identical, or 100% identical to SEQ ID NO: 207. In embodiments, the antisense nucleic acid sequence is at least 90% identical, at least 95% identical, or 100% identical to SEQ ID NO: 208. In embodiments, the antisense nucleic acid sequence is at least 90% identical, at least 95% identical, or 100% identical to SEQ ID NO: 209. In embodiments, the antisense nucleic acid sequence is at least 90% identical, at least 95% identical, or 100% identical to SEQ ID NO: 210. In embodiments, the antisense nucleic acid sequence is at least 90% identical, at least 95% identical, or 100% identical to SEQ ID NO: 211. In embodiments, the antisense nucleic acid sequence is at least 90% identical, at least 95% identical, or 100% identical to SEQ ID NO: 212. In embodiments, the antisense nucleic acid sequence is at least 90% identical, at least 95% identical, or 100% identical to SEQ ID NO: 213. In embodiments, the antisense nucleic acid sequence is at least 90% identical, at least 95% identical, or 100% identical to SEQ ID NO: 214. In embodiments, the antisense nucleic acid sequence is at least 90% identical, at least 95% identical, or 100% identical to SEQ ID NO: 215. In embodiments, the antisense nucleic acid sequence is at least 90% identical, at least 95% identical, or 100% identical to SEQ ID NO: 216. In embodiments, the antisense nucleic acid sequence is at least 90% identical, at least 95% identical, or 100% identical to SEQ ID NO: 217.

In embodiments, the antisense nucleic acid sequence includes SEQ ID NO: 30. In embodiments, the antisense nucleic acid sequence includes SEQ ID NO: 31. In embodiments, the antisense nucleic acid sequence includes SEQ ID NO: 32. In embodiments, the antisense nucleic acid sequence includes SEQ ID NO: 33. In embodiments, the antisense nucleic acid sequence includes SEQ ID NO: 34. In embodiments, the antisense nucleic acid sequence includes SEQ ID NO: 35. In embodiments, the antisense nucleic acid sequence includes SEQ ID NO: 36. In embodiments, the antisense nucleic acid sequence includes SEQ ID NO: 37. In embodiments, the antisense nucleic acid sequence includes SEQ ID NO: 38. In embodiments, the antisense nucleic acid sequence includes SEQ ID NO: 39. In embodiments, the antisense nucleic acid sequence includes SEQ ID NO: 40. In embodiments, the antisense nucleic acid sequence includes SEQ ID NO: 41. In embodiments, the antisense nucleic acid sequence includes SEQ ID NO: 42. In embodiments, the antisense nucleic acid sequence includes SEQ ID NO: 43. In embodiments, the antisense nucleic acid sequence includes SEQ ID NO: 44. In embodiments, the antisense nucleic acid sequence includes SEQ ID NO: 45. In embodiments, the antisense nucleic acid sequence includes SEQ ID NO: 46. In embodiments, the antisense nucleic acid sequence includes SEQ ID NO: 47. In embodiments, the antisense nucleic acid sequence includes SEQ ID NO: 48. In embodiments, the antisense nucleic acid sequence includes SEQ ID NO: 49. In embodiments, the antisense nucleic acid sequence includes SEQ ID NO: 50. In embodiments, the antisense nucleic acid sequence includes SEQ ID NO: 51. In embodiments, the antisense nucleic acid sequence includes SEQ ID NO: 52. In embodiments, the antisense nucleic acid sequence includes SEQ ID NO: 53. In embodiments, the antisense nucleic acid sequence includes SEQ ID NO: 54. In embodiments, the antisense nucleic acid sequence includes SEQ ID NO: 55. In embodiments, the antisense nucleic acid sequence includes SEQ ID NO: 56. In embodiments, the antisense nucleic acid sequence includes SEQ ID NO: 57. In embodiments, the antisense nucleic acid sequence includes SEQ ID NO: 58. In embodiments, the antisense nucleic acid sequence includes SEQ ID NO: 59. In embodiments, the antisense nucleic acid sequence includes SEQ ID NO: 60. In embodiments, the antisense nucleic acid sequence includes SEQ ID NO: 61. In embodiments, the antisense nucleic acid sequence includes SEQ ID NO: 62. In embodiments, the antisense nucleic acid sequence includes SEQ ID NO: 63. In embodiments, the antisense nucleic acid sequence includes SEQ ID NO: 64. In embodiments, the antisense nucleic acid sequence includes SEQ ID NO: 65. In embodiments, the antisense nucleic acid sequence includes SEQ ID NO: 66. In embodiments, the antisense nucleic acid sequence includes SEQ ID NO: 67. In embodiments, the antisense nucleic acid sequence includes SEQ ID NO: 68. In embodiments, the antisense nucleic acid sequence includes SEQ ID NO: 69. In embodiments, the antisense nucleic acid sequence includes SEQ ID NO: 70. In embodiments, the antisense nucleic acid sequence includes SEQ ID NO: 71. In embodiments, the antisense nucleic acid sequence includes SEQ ID NO: 72. In embodiments, the antisense nucleic acid sequence includes SEQ ID NO: 73. In embodiments, the antisense nucleic acid sequence includes SEQ ID NO: 74. In embodiments, the antisense nucleic acid sequence includes SEQ ID NO: 75. In embodiments, the antisense nucleic acid sequence includes SEQ ID NO: 76. In embodiments, the antisense nucleic acid sequence includes SEQ ID NO: 76. In embodiments, the antisense nucleic acid sequence includes SEQ ID NO: 77. In embodiments, the antisense nucleic acid sequence includes SEQ ID NO: 78. In embodiments, the antisense nucleic acid sequence includes SEQ ID NO: 79. In embodiments, the antisense nucleic acid sequence includes SEQ ID NO: 80. In embodiments, the antisense nucleic acid sequence includes SEQ ID NO: 81. In embodiments, the antisense nucleic acid sequence includes SEQ ID NO: 82. In embodiments, the antisense nucleic acid sequence includes SEQ ID NO: 83. In embodiments, the antisense nucleic acid sequence includes SEQ ID NO: 84. In embodiments, the antisense nucleic acid sequence includes SEQ ID NO: 84. In embodiments, the antisense nucleic acid sequence includes SEQ ID NO: 85. In embodiments, the antisense nucleic acid sequence includes SEQ ID NO: 86. In embodiments, the antisense nucleic acid sequence includes SEQ ID NO: 87. In embodiments, the antisense nucleic acid sequence includes SEQ ID NO: 88. In embodiments, the antisense nucleic acid sequence includes SEQ ID NO: 89. In embodiments, the antisense nucleic acid sequence includes SEQ ID NO: 90. In embodiments, the antisense nucleic acid sequence includes SEQ ID NO: 91. In embodiments, the antisense nucleic acid sequence includes SEQ ID NO: 92. In embodiments, the antisense nucleic acid sequence includes SEQ ID NO: 93. In embodiments, the antisense nucleic acid sequence includes SEQ ID NO: 94. In embodiments, the antisense nucleic acid sequence includes SEQ ID NO: 95. In embodiments, the antisense nucleic acid sequence includes SEQ ID NO: 96. In embodiments, the antisense nucleic acid sequence includes SEQ ID NO: 97. In embodiments, the antisense nucleic acid sequence includes SEQ ID NO: 98. In embodiments, the antisense nucleic acid sequence includes SEQ ID NO: 99. In embodiments, the antisense nucleic acid sequence includes SEQ ID NO: 100. In embodiments, the antisense nucleic acid sequence includes SEQ ID NO: 101. In embodiments, the antisense nucleic acid sequence includes SEQ ID NO: 102. In embodiments, the antisense nucleic acid sequence includes SEQ ID NO: 103. In embodiments, the antisense nucleic acid sequence includes SEQ ID NO: 104. In embodiments, the antisense nucleic acid sequence includes SEQ ID NO: 105. In embodiments, the antisense nucleic acid sequence includes SEQ ID NO: 106. In embodiments, the antisense nucleic acid sequence includes SEQ ID NO: 107. In embodiments, the antisense nucleic acid sequence includes SEQ ID NO: 108. In embodiments, the antisense nucleic acid sequence includes SEQ ID NO: 109. In embodiments, the antisense nucleic acid sequence includes SEQ ID NO: 110. In embodiments, the antisense nucleic acid sequence includes SEQ ID NO: 111. In embodiments, the antisense nucleic acid sequence includes SEQ ID NO: 112. In embodiments, the antisense nucleic acid sequence includes SEQ ID NO: 113. In embodiments, the antisense nucleic acid sequence includes SEQ ID NO: 114. In embodiments, the antisense nucleic acid sequence includes SEQ ID NO: 115. In embodiments, the antisense nucleic acid sequence includes SEQ ID NO: 116. In embodiments, the antisense nucleic acid sequence includes SEQ ID NO: 117. In embodiments, the antisense nucleic acid sequence includes SEQ ID NO: 118. In embodiments, the antisense nucleic acid sequence includes SEQ ID NO: 119. In embodiments, the antisense nucleic acid sequence includes SEQ ID NO: 120. In embodiments, the antisense nucleic acid sequence includes SEQ ID NO: 121. In embodiments, the antisense nucleic acid sequence includes SEQ ID NO: 126. In embodiments, the antisense nucleic acid sequence includes SEQ ID NO: 127. In embodiments, the antisense nucleic acid sequence includes SEQ ID NO: 128. In embodiments, the antisense nucleic acid sequence includes SEQ ID NO: 129. In embodiments, the antisense nucleic acid sequence includes SEQ ID NO: 130. In embodiments, the antisense nucleic acid sequence includes SEQ ID NO: 131. In embodiments, the antisense nucleic acid sequence includes SEQ ID NO: 132. In embodiments, the antisense nucleic acid sequence includes SEQ ID NO: 133. In embodiments, the antisense nucleic acid sequence includes SEQ ID NO: 134. In embodiments, the antisense nucleic acid sequence includes SEQ ID NO: 135. In embodiments, the antisense nucleic acid sequence includes SEQ ID NO: 136. In embodiments, the antisense nucleic acid sequence includes SEQ ID NO: 137. In embodiments, the antisense nucleic acid sequence includes SEQ ID NO: 138. In embodiments, the antisense nucleic acid sequence includes SEQ ID NO: 139. In embodiments, the antisense nucleic acid sequence includes SEQ ID NO: 140. In embodiments, the antisense nucleic acid sequence includes SEQ ID NO: 141. In embodiments, the antisense nucleic acid sequence includes SEQ ID NO: 142. In embodiments, the antisense nucleic acid sequence includes SEQ ID NO: 143. In embodiments, the antisense nucleic acid sequence includes SEQ ID NO: 144. In embodiments, the antisense nucleic acid sequence includes SEQ ID NO: 145. In embodiments, the antisense nucleic acid sequence includes SEQ ID NO: 146. In embodiments, the antisense nucleic acid sequence includes SEQ ID NO: 147. In embodiments, the antisense nucleic acid sequence includes SEQ ID NO: 148. In embodiments, the antisense nucleic acid sequence includes SEQ ID NO: 149. In embodiments, the antisense nucleic acid sequence includes SEQ ID NO: 150. In embodiments, the antisense nucleic acid sequence includes SEQ ID NO: 151. In embodiments, the antisense nucleic acid sequence includes SEQ ID NO: 152. In embodiments, the antisense nucleic acid sequence includes SEQ ID NO: 153. In embodiments, the antisense nucleic acid sequence includes SEQ ID NO: 154. In embodiments, the antisense nucleic acid sequence includes SEQ ID NO: 155. In embodiments, the antisense nucleic acid sequence includes SEQ ID NO: 156. In embodiments, the antisense nucleic acid sequence includes SEQ ID NO: 157. In embodiments, the antisense nucleic acid sequence includes SEQ ID NO: 158. In embodiments, the antisense nucleic acid sequence includes SEQ ID NO: 159. In embodiments, the antisense nucleic acid sequence includes SEQ ID NO: 160. In embodiments, the antisense nucleic acid sequence includes SEQ ID NO: 161. In embodiments, the antisense nucleic acid sequence includes SEQ ID NO: 162. In embodiments, the antisense nucleic acid sequence includes SEQ ID NO: 163. In embodiments, the antisense nucleic acid sequence includes SEQ ID NO: 164. In embodiments, the antisense nucleic acid sequence includes SEQ ID NO: 165. In embodiments, the antisense nucleic acid sequence includes SEQ ID NO: 166. In embodiments, the antisense nucleic acid sequence includes SEQ ID NO: 167. In embodiments, the antisense nucleic acid sequence includes SEQ ID NO: 168. In embodiments, the antisense nucleic acid sequence includes SEQ ID NO: 169. In embodiments, the antisense nucleic acid sequence includes SEQ ID NO: 170. In embodiments, the antisense nucleic acid sequence includes SEQ ID NO: 171. In embodiments, the antisense nucleic acid sequence includes SEQ ID NO: 172. In embodiments, the antisense nucleic acid sequence includes SEQ ID NO: 173. In embodiments, the antisense nucleic acid sequence includes SEQ ID NO: 174. In embodiments, the antisense nucleic acid sequence includes SEQ ID NO: 175. In embodiments, the antisense nucleic acid sequence includes SEQ ID NO: 176. In embodiments, the antisense nucleic acid sequence includes SEQ ID NO: 176. In embodiments, the antisense nucleic acid sequence includes SEQ ID NO: 177. In embodiments, the antisense nucleic acid sequence includes SEQ ID NO: 178. In embodiments, the antisense nucleic acid sequence includes SEQ ID NO: 179. In embodiments, the antisense nucleic acid sequence includes SEQ ID NO: 180. In embodiments, the antisense nucleic acid sequence includes SEQ ID NO: 181. In embodiments, the antisense nucleic acid sequence includes SEQ ID NO: 182. In embodiments, the antisense nucleic acid sequence includes SEQ ID NO: 183. In embodiments, the antisense nucleic acid sequence includes SEQ ID NO: 184. In embodiments, the antisense nucleic acid sequence includes SEQ ID NO: 184. In embodiments, the antisense nucleic acid sequence includes SEQ ID NO: 185. In embodiments, the antisense nucleic acid sequence includes SEQ ID NO: 186. In embodiments, the antisense nucleic acid sequence includes SEQ ID NO: 187. In embodiments, the antisense nucleic acid sequence includes SEQ ID NO: 188. In embodiments, the antisense nucleic acid sequence includes SEQ ID NO: 189. In embodiments, the antisense nucleic acid sequence includes SEQ ID NO: 190. In embodiments, the antisense nucleic acid sequence includes SEQ ID NO: 191. In embodiments, the antisense nucleic acid sequence includes SEQ ID NO: 192. In embodiments, the antisense nucleic acid sequence includes SEQ ID NO: 193. In embodiments, the antisense nucleic acid sequence includes SEQ ID NO: 194. In embodiments, the antisense nucleic acid sequence includes SEQ ID NO: 195. In embodiments, the antisense nucleic acid sequence includes SEQ ID NO: 196. In embodiments, the antisense nucleic acid sequence includes SEQ ID NO: 197. In embodiments, the antisense nucleic acid sequence includes SEQ ID NO: 198. In embodiments, the antisense nucleic acid sequence includes SEQ ID NO: 199. In embodiments, the antisense nucleic acid sequence includes SEQ ID NO: 200. In embodiments, the antisense nucleic acid sequence includes SEQ ID NO: 201. In embodiments, the antisense nucleic acid sequence includes SEQ ID NO: 202. In embodiments, the antisense nucleic acid sequence includes SEQ ID NO: 203. In embodiments, the antisense nucleic acid sequence includes SEQ ID NO: 204. In embodiments, the antisense nucleic acid sequence includes SEQ ID NO: 205. In embodiments, the antisense nucleic acid sequence includes SEQ ID NO: 206. In embodiments, the antisense nucleic acid sequence includes SEQ ID NO: 207. In embodiments, the antisense nucleic acid sequence includes SEQ ID NO: 208. In embodiments, the antisense nucleic acid sequence includes SEQ ID NO: 209. In embodiments, the antisense nucleic acid sequence includes SEQ ID NO: 210. In embodiments, the antisense nucleic acid sequence includes SEQ ID NO: 211. In embodiments, the antisense nucleic acid sequence includes SEQ ID NO: 212. In embodiments, the antisense nucleic acid sequence includes SEQ ID NO: 213. In embodiments, the antisense nucleic acid sequence includes SEQ ID NO: 214. In embodiments, the antisense nucleic acid sequence includes SEQ ID NO: 215. In embodiments, the antisense nucleic acid sequence includes SEQ ID NO: 216. In embodiments, the antisense nucleic acid sequence includes SEQ ID NO: 217.

A antisense nucleic acid sequence may include a sequence at least 80%, at least 85%, at least 90%, at least 95%, at least 98%, at least 99%, or 100% identical to SEQ ID NO: 141. A antisense nucleic acid sequence may include a sequence at least 80%, at least 85%, at least 90%, at least 95%, at least 98%, at least 99%, or 100% identical to SEQ ID NO: 191. A antisense nucleic acid sequence may include a sequence at least 80%, at least 85%, at least 90%, at least 95%, at least 98%, at least 99%, or 100% identical to SEQ ID NO: 250. A antisense nucleic acid sequence may include a sequence at least 80%, at least 85%, at least 90%, at least 95%, at least 98%, at least 99%, or 100% identical to SEQ ID NO: 251. A antisense nucleic acid sequence may include a sequence at least 80%, at least 85%, at least 90%, at least 95%, at least 98%, at least 99%, or 100% identical to SEQ ID NO: 252. A antisense nucleic acid sequence may include a sequence at least 80%, at least 85%, at least 90%, at least 95%, at least 98%, at least 99%, or 100% identical to SEQ ID NO: 253. A antisense nucleic acid sequence may include a sequence at least 80%, at least 85%, at least 90%, at least 95%, at least 98%, at least 99%, or 100% identical to SEQ ID NO: 254. A antisense nucleic acid sequence may include a sequence at least 80%, at least 85%, at least 90%, at least 95%, at least 98%, at least 99%, or 100% identical to SEQ ID NO: 255. A antisense nucleic acid sequence may include a sequence at least 80%, at least 85%, at least 90%, at least 95%, at least 98%, at least 99%, or 100% identical to SEQ ID NO: 256. A antisense nucleic acid sequence may include a sequence at least 80%, at least 85%, at least 90%, at least 95%, at least 98%, at least 99%, or 100% identical to SEQ ID NO: 257. A antisense nucleic acid sequence may include a sequence at least 80%, at least 85%, at least 90%, at least 95%, at least 98%, at least 99%, or 100% identical to SEQ ID NO: 258. A antisense nucleic acid sequence may include a sequence at least 80%, at least 85%, at least 90%, at least 95%, at least 98%, at least 99%, or 100% identical to SEQ ID NO: 259. A antisense nucleic acid sequence may include a sequence at least 80%, at least 85%, at least 90%, at least 95%, at least 98%, at least 99%, or 100% identical to SEQ ID NO: 260. A antisense nucleic acid sequence may include a sequence at least 80%, at least 85%, at least 90%, at least 95%, at least 98%, at least 99%, or 100% identical to SEQ ID NO: 261. A antisense nucleic acid sequence may include a sequence at least 80%, at least 85%, at least 90%, at least 95%, at least 98%, at least 99%, or 100% identical to SEQ ID NO: 262. A antisense nucleic acid sequence may include a sequence at least 80%, at least 85%, at least 90%, at least 95%, at least 98%, at least 99%, or 100% identical to SEQ ID NO: 263. A antisense nucleic acid sequence may include a sequence at least 80%, at least 85%, at least 90%, at least 95%, at least 98%, at least 99%, or 100% identical to SEQ ID NO: 264. A antisense nucleic acid sequence may include a sequence at least 80%, at least 85%, at least 90%, at least 95%, at least 98%, at least 99%, or 100% identical to SEQ ID NO: 265. A antisense nucleic acid sequence may include a sequence at least 80%, at least 85%, at least 90%, at least 95%, at least 98%, at least 99%, or 100% identical to SEQ ID NO: 266. A antisense nucleic acid sequence may include a sequence at least 80%, at least 85%, at least 90%, at least 95%, at least 98%, at least 99%, or 100% identical to SEQ ID NO: 267. A antisense nucleic acid sequence may include a sequence at least 80%, at least 85%, at least 90%, at least 95%, at least 98%, at least 99%, or 100% identical to SEQ ID NO: 268. A antisense nucleic acid sequence may include a sequence at least 80%, at least 85%, at least 90%, at least 95%, at least 98%, at least 99%, or 100% identical to SEQ ID NO: 269. A antisense nucleic acid sequence may include a sequence at least 80%, at least 85%, at least 90%, at least 95%, at least 98%, at least 99%, or 100% identical to SEQ ID NO: 270. A antisense nucleic acid sequence may include a sequence at least 80%, at least 85%, at least 90%, at least 95%, at least 98%, at least 99%, or 100% identical to SEQ ID NO: 271. A antisense nucleic acid sequence may include a sequence at least 80%, at least 85%, at least 90%, at least 95%, at least 98%, at least 99%, or 100% identical to SEQ ID NO: 272. A antisense nucleic acid sequence may include a sequence at least 80%, at least 85%, at least 90%, at least 95%, at least 98%, at least 99%, or 100% identical to SEQ ID NO: 273. A antisense nucleic acid sequence may include a sequence at least 80%, at least 85%, at least 90%, at least 95%, at least 98%, at least 99%, or 100% identical to SEQ ID NO: 274. A antisense nucleic acid sequence may include a sequence at least 80%, at least 85%, at least 90%, at least 95%, at least 98%, at least 99%, or 100% identical to SEQ ID NO: 275. A antisense nucleic acid sequence may include a sequence at least 80%, at least 85%, at least 90%, at least 95%, at least 98%, at least 99%, or 100% identical to SEQ ID NO: 276. A antisense nucleic acid sequence may include a sequence at least 80%, at least 85%, at least 90%, at least 95%, at least 98%, at least 99%, or 100% identical to SEQ ID NO: 277. A antisense nucleic acid sequence may include a sequence at least 80%, at least 85%, at least 90%, at least 95%, at least 98%, at least 99%, or 100% identical to SEQ ID NO: 278. A antisense nucleic acid sequence may include a sequence at least 80%, at least 85%, at least 90%, at least 95%, at least 98%, at least 99%, or 100% identical to SEQ ID NO: 279. A antisense nucleic acid sequence may include a sequence at least 80%, at least 85%, at least 90%, at least 95%, at least 98%, at least 99%, or 100% identical to SEQ ID NO: 280.

In embodiments, the antisense nucleic acid sequence is 3′ or downstream relative to the ESS nucleic acid sequence within the silencing module.

smOPT

Described herein, in some embodiments, are systems that include a Sm binding site. The Sm binding site may be included in a modified or recombinant snRNA or U7 snRNA. Sm proteins may bind to a U7 snRNA via the Sm binding site. Once the Sm proteins bind the U7 snRNA in the cytoplasm they may bind to a pre-mRNA and regulate splicing. In some embodiments, the system contains a Sm-like binding site. A modified or recombinant U7 snRNA sequence may include a Sm binding site. A system may encode a modified or recombinant U7 snRNA sequence that includes a Sm binding site.

For the system provided herein, in embodiments, the silencing module includes a sequence capable of binding or recruiting one or more proteins capable of splicing RNA. In embodiments, the silencing module further includes an Sm binding site sequence. The term “Sm binding site sequence” refers to a nucleic acid sequence typically found in U snRNA capable of binding and/or recruiting Sm proteins. Binding of Sm proteins to U snRNA results in formation of the snRNP complex, which is typically involved in RNA processing. In embodiments, U7 specific proteins (e.g. Lsm10 and Lsm11) to the bind to a Sm binding site of U7 snRNA to form the U7 snRNP, allowing histone RNA processing.

In embodiments, the Sm binding site sequence includes AAUUUGUCUAG (SEQ ID NO:243) or AAUUUUUGGAG (SEQ ID NO: 244; smOPT). In embodiments, the Sm binding site sequence includes SEQ ID NO: 243. In embodiments, the Sm binding site sequence is SEQ ID NO: 243. In embodiments, the Sm binding site sequence includes SEQ ID NO: 244. In embodiments, the Sm binding site sequence is SEQ ID NO: 244.

In embodiments, the Sm binding site sequence includes an smOPT sequence. In embodiments, the smOPT sequence includes SEQ ID NO: 244. The term “smOPT sequence” as used herein refers to a nucleic acid sequence that binds and/or recruits proteins involved in non-histone RNA splicing. Thus, in embodiments, the smOPT sequence does not bind and/or recruit spliceosomal proteins specifically involved in histone processing. In embodiments, the smOPT sequence does not bind to proteins that specifically bind to U7 snRNA. In embodiments, the smOPT sequence binds and/or recruits proteins involved in non-histone mRNA processing. In embodiments, the smOPT sequence does not bind and/or recruit Lsm10 or Lsm11. In embodiments, the smOPT sequence does not bind and/or recruit Lsm10. In embodiments, the smOPT sequence does not bind and/or recruit Lsm11.

In embodiments, the smOPT sequence includes a consensus sequence found in a variety of snRNA. In embodiments, the consensus sequence includes SEQ ID NO: 244. The smOPT sequence may bind to proteins, forming a structure including a hepameric protein core.

In embodiments, the Sm binding site sequence is 3′ or downstream relative to the silencing module promoter sequence, the ESS nucleic acid sequence, or the antisense nucleic acid sequence within the silencing module. In embodiments, the Sm binding site sequence is 3′ or downstream relative to the silencing module promoter sequence. In embodiments, the Sm binding site sequence is 3′ or downstream relative to the ESS nucleic acid sequence. In embodiments, the Sm binding site sequence is 3′ or downstream relative to the antisense nucleic acid sequence within the silencing module.

### U7 3′ Hairpin

Described herein, in some embodiments, are systems that include a hairpin sequence. The hairpin sequence may be included in a modified or recombinant snRNA or U7 snRNA. The hairpin sequence may include a U7 hairpin sequence. The U7 hairpin sequence may be a 3′ U7 hairpin sequence. A modified or recombinant U7 snRNA sequence may include the hairpin sequence. A system may encode a modified or recombinant U7 snRNA sequence that includes a hairpin sequence.

For the system provided herein, in embodiments, the silencing module further includes a hairpin sequence. In embodiments, the hairpin sequence includes a U7 small nuclear RNA (snRNA) 3′ hairpin sequence. For example, the hairpin sequence may be a hairpin sequence found in naturally occurring U7 snRNA. In embodiments, the hairpin sequence is 3′ or downstream relative to the silencing module promoter sequence, the ESS nucleic acid sequence, the antisense nucleic acid sequence, or the Sm binding site sequence within the silencing module. In embodiments, the hairpin sequence is 3′ or downstream relative to the silencing module promoter sequence within the silencing module. In embodiments, the hairpin sequence is 3′ or downstream relative to the ESS nucleic acid sequence within the silencing module. In embodiments, the hairpin sequence is 3′ or downstream relative to the antisense nucleic acid sequence within the silencing module. In embodiments, the hairpin sequence is 3′ or downstream relative to the Sm binding site sequence within the silencing module.

### 3′ Terminator Sequence

For the system provided herein, in embodiments, the silencing module further includes a terminator sequence. In embodiments, the terminator sequence includes a mouse or human terminator sequence. In embodiments, the terminator sequence includes a mouse terminator sequence. In embodiments, the terminator sequence includes a human terminator sequence.

In some embodiments, the terminator sequence may include a nucleic acid sequence. In some embodiments, the terminator sequence may include a nucleic acid sequence identical to a terminator sequence in Table 1. In some embodiments, the terminator sequence may include a nucleic acid sequence at least 99% identical to a terminator sequence in Table 1. In some embodiments, the terminator sequence may include a nucleic acid sequence at least 98% identical to a terminator sequence in Table 1. In some embodiments, the terminator sequence may include a nucleic acid sequence at least 97% identical to a terminator sequence in Table 1. In some embodiments, the terminator sequence may include a nucleic acid sequence at least 96% identical to a terminator sequence in Table 1. In some embodiments, the terminator sequence may include a nucleic acid sequence at least 95% identical to a terminator sequence in Table 1. In some embodiments, the terminator sequence may include a nucleic acid sequence at least 94% identical to a terminator sequence in Table 1. In some embodiments, the terminator sequence may include a nucleic acid sequence at least 93% identical to a terminator sequence in Table 1. In some embodiments, the terminator sequence may include a nucleic acid sequence at least 92% identical to a terminator sequence in Table 1. In some embodiments, the terminator sequence may include a nucleic acid sequence at least 910% identical to a terminator sequence in Table 1. In some embodiments, the terminator sequence may include a nucleic acid sequence at least 90% identical to a terminator sequence in Table 1. In some embodiments, the terminator sequence may include a nucleic acid sequence at least 85% identical to a terminator sequence in Table 1. In some embodiments, the terminator sequence may include a nucleic acid sequence at least 80% identical to a terminator sequence in Table 1. In some embodiments, the terminator sequence may include a nucleic acid sequence at least 75% identical to a terminator sequence in Table 1. In some embodiments, the terminator sequence may include a nucleic acid sequence at least 70% identical to a terminator sequence in Table 1. In some embodiments, the silencing module terminator sequence is at least 80%, at least 85%, at least 90%, at least 95%, at least 98%, at least 99%, or 100% identical to a promoter sequence in Table 1.

In some embodiments, the terminator sequence may include a nucleic acid sequence. In some embodiments, the terminator sequence may include a nucleic acid sequence identical to a terminator sequence in Table 9. In some embodiments, the terminator sequence may include a nucleic acid sequence at least 99% identical to a terminator sequence in Table 9. In some embodiments, the terminator sequence may include a nucleic acid sequence at least 98% identical to a terminator sequence in Table 9. In some embodiments, the terminator sequence may include a nucleic acid sequence at least 97% identical to a terminator sequence in Table 9. In some embodiments, the terminator sequence may include a nucleic acid sequence at least 96% identical to a terminator sequence in Table 9. In some embodiments, the terminator sequence may include a nucleic acid sequence at least 95% identical to a terminator sequence in Table 9. In some embodiments, the terminator sequence may include a nucleic acid sequence at least 94% identical to a terminator sequence in Table 9. In some embodiments, the terminator sequence may include a nucleic acid sequence at least 93% identical to a terminator sequence in Table 9. In some embodiments, the terminator sequence may include a nucleic acid sequence at least 92% identical to a terminator sequence in Table 9. In some embodiments, the terminator sequence may include a nucleic acid sequence at least 910% identical to a terminator sequence in Table 9. In some embodiments, the terminator sequence may include a nucleic acid sequence at least 90% identical to a terminator sequence in Table 9. In some embodiments, the terminator sequence may include a nucleic acid sequence at least 85% identical to a terminator sequence in Table 9. In some embodiments, the terminator sequence may include a nucleic acid sequence at least 80% identical to a terminator sequence in Table 9. In some embodiments, the terminator sequence may include a nucleic acid sequence at least 75% identical to a terminator sequence in Table 9. In some embodiments, the terminator sequence may include a nucleic acid sequence at least 70% identical to a terminator sequence in Table 9. In some embodiments, the silencing module terminator sequence is at least 80%, at least 85%, at least 90%, at least 95%, at least 98%, at least 99%, or 100% identical to a terminator sequence in Table 9.

The terminator sequence may be operably connected with an ESS sequence, a U7 targeting sequence, a Sm binding site, or a U7 3′ hairpin, or a combination thereof. For example, the terminator sequence may be operably connected with an ESS sequence, a U7 targeting sequence, a Sm binding site, and a U7 3′ hairpin within an expression construct. In embodiments, the terminator sequence includes a U7 snRNA terminator sequence.

In embodiments, the terminator sequence includes a U1 terminator sequence. In embodiments, the terminator sequence includes a Mm U7 terminator sequence, a Hs U7 terminator sequence, a mu1a1 terminator sequence, or a HU1 terminator sequence, or a fragment or combination of fragments thereof. In embodiments, the terminator sequence includes a Mm U7 terminator sequence or a fragment thereof. In embodiments, the terminator sequence includes a Hs U7 terminator sequence or a fragment thereof. In embodiments, the terminator sequence includes a mu1a1 terminator sequence or a fragment thereof. In embodiments, the terminator sequence includes a HU1 terminator sequence or a fragment thereof.

In embodiments, the terminator sequence includes a U7 snRNA terminator sequence having a distal sequence element (DSE) replaced with a DSE of a U1-1 or U1a1 terminator sequence. In embodiments, the terminator sequence includes a U7 snRNA terminator sequence having a DSE replaced with a DSE of a U1-1 terminator sequence. In embodiments, the terminator sequence includes a U7 snRNA terminator sequence having a DSE replaced with a DSE of a U1a1 terminator sequence. “Distal sequence element” or “DSE” refers to a nucleic acid sequence that regulates expression of snRNA gene. A distal sequence element may, in embodiments, be found upstream of an snRNA promoter. The DSE may include one or more binding sites for a transcription factor and/or one or more proteins that activate transcription of the snRNA.

In embodiments, the terminator sequence includes a mouse U7 snRNA terminator sequence having a proximal sequence element (PSE) replaced with a PSE of a U1-1 or U1a1 terminator sequence. In embodiments, the terminator sequence includes a mouse U7 snRNA terminator sequence having a PSE replaced with a PSE of a U1-1 terminator sequence. In embodiments, the terminator sequence includes a mouse U7 snRNA terminator sequence having a PSE replaced with a PSE of U1a1 terminator sequence. “Proximal sequence element” or “PSE” refers to a sequence typically found in snRNA genes that regulate expressing of the snRNA gene. The PSE is typically found in RNA Pol II and RNA Pol III transcribed snRNA genes. In embodiments, the PSE includes a PSE-binding transcription factor (PTF) binding site.

In embodiments, the terminator sequence is 3′ or downstream relative to the silencing module promoter sequence, the ESS nucleic acid sequence, the antisense nucleic acid sequence, the Sm binding site sequence, or the hairpin sequence.

In embodiments, the terminator sequence is 3′ or downstream relative to the silencing module promoter sequence, the ESS nucleic acid sequence, the antisense nucleic acid sequence, the Sm binding site sequence, or the hairpin sequence. In embodiments, the terminator sequence is 3′ or downstream relative to the silencing module promoter sequence. In embodiments, the terminator sequence is 3′ or downstream relative to the ESS nucleic acid sequence. In embodiments, the terminator sequence is 3′ or downstream relative to the antisense nucleic acid sequence. In embodiments, the terminator sequence is 3′ or downstream relative to the hairpin sequence.

### Expression Construct

In an aspect is provided an expression cassette (e.g. expression vector) including the system provided herein including embodiments thereof. In embodiments, the expression cassette is a viral vector or a plasmid. In embodiments, the expression cassette is a viral vector. Expression cassettes contemplated to include, but are not limited to, viral vectors based on various viral sequences as well as those contemplated for eukaryotic target cells or prokaryotic target cells. The “target cells” may refer to the cells where the expression vector is transfected and the nucleotide sequence encoding the protein is expressed. In embodiments, the target cells are neural cells.

In embodiments, the system includes one or more deoxyribonucleic acid (DNA) molecules including the synthesis module and the silencing module.

In embodiments, the system includes an expression cassette. In embodiments, the expression cassette encodes the synthesis module and the silencing module. In embodiments, the expression cassette encodes the synthesis module. In embodiments, the expression cassette encodes the silencing module.

In embodiments, the synthesis module is upstream or 5′ relative to the silencing module. In embodiments, the synthesis module is downstream or 3′ relative to the silencing module.

Described herein, in some embodiments, are expression constructs. An expression construct may include an expression cassette. The expression construct may be DNA. The expression construct may encode an RNA system described herein. The expression construct may encode an RNA such as a modified U7 snRNA that targets MECP2, or may include or encode an MECP2 coding sequence. The expression construct may encode an ESS sequence, a U7 targeting sequence, a Sm binding site, or a U7 3′ hairpin, or a combination thereof. For example, an expression construct may encode an ESS sequence, a U7 targeting sequence, a Sm binding site, and a U7 3′ hairpin, which may be operably connected to a promoter within the expression construct. The expression construct may include an MECP2 coding sequence, which may be operably connected to a promoter (e.g. a different promoter). The expression construct may be or include a viral vector. The expression construct may be included in a composition herein. The expression construct may be included in a virus or viral delivery agent.

In some embodiments, the system may include an expression cassette. In some embodiments, the expression cassette may include a promoter. In some embodiments, the expression cassette may encode an exonic splicing sequence. In some embodiments, the expression cassette may encode a U7 targeting sequence. In some embodiments, the expression cassette may encode a smOPT sequence. In some embodiments, the expression cassette may encode a U7 3′ hairpin structure. In some embodiments, the expression cassette may include a 3′ terminator sequence. In some embodiments, the expression cassette may include or encode a combination of two or more, or all of the following: a promoter, an exonic splicing sequence, a U7 targeting sequence that targets an MECP2 mRNA, a smOPT, a U7 3′ hairpin structure, and a 3′ terminator sequence.

In some embodiments, an expression cassette may include a promoter. In some embodiments, the expression cassette may include an MECP2 coding sequence. In some embodiments, the expression cassette may include a 3′ terminator sequence. In some embodiments, the expression cassette may include or encode a combination of two or more, or all of the following: a promoter, an MECP2 coding sequence, and a 3′ terminator sequence. An expression construct may include multiple expression cassettes.

Some embodiments include an arrayed series of modified U7 snRNAs. In some embodiments, the array includes multiple U7 modules. Each module may encode a targeting sequence. The targeting sequences of multiple modules may be the same as each other. The targeting sequences of some modules may be different from each other.

An expression construct may encode multiple engineered or modified snRNAs. For example, an expression construct may encode 1, 2, 3, 4, 5, 6, 7, 8, 9, or 10 copies of a recombinant U7 snRNA. In some embodiments, the expression construct encodes 1 copy of a recombinant U7 snRNA. In some embodiments, the expression construct encodes 2 copies of the recombinant U7 snRNA. In some embodiments, the expression construct encodes 3 copies of the recombinant U7 snRNA. In some embodiments, the expression construct encodes 4 copies of the recombinant U7 snRNA. In some embodiments, the expression construct encodes 5 copies of the recombinant U7 snRNA. In some embodiments, the expression construct encodes 6 copies of the recombinant U7 snRNA. In some embodiments, the expression construct encodes 7 copies of the recombinant U7 snRNA. In some embodiments, the expression construct encodes 8 copies of the recombinant U7 snRNA. In some embodiments, the expression construct encodes 9 copies of the recombinant U7 snRNA. In some embodiments, the expression construct encodes 10 copies of the recombinant U7 snRNA. In some embodiments, all 1, 2, 3, 4, 5, 6, 7, 8, 9, or 10 copies of a recombinant U7 snRNA sequence are operably linked to one or more promoters. For example, multiple copies of the recombinant U7 snRNA sequence may be operably linked to a single promoter. In some embodiments, all 1, 2, 3, 4, 5, 6, 7, 8, 9, or 10 copies of a recombinant U7 snRNA sequence are operably linked to one or more 3′ terminator sequences.

Some embodiments include an arrayed series of MECP2 coding sequences. In some embodiments, the array includes multiple MECP2 coding sequences. Each module may encode a MECP2 coding sequence. The MECP2 coding sequences of multiple modules may be the same as each other. The MECP2 coding sequences of some modules may be different.

An expression construct may encode or include multiple coding sequences. For example, an expression construct may include 1, 2, 3, 4, 5, 6, 7, 8, 9, or 10 copies of a MECP2 coding sequence. In some embodiments, the expression construct includes 1 copy of a MECP2 coding sequence. In some embodiments, the expression construct includes 2 copies of the MECP2 coding sequence. In some embodiments, the expression construct includes 3 copies of the MECP2 coding sequence. In some embodiments, the expression construct includes 4 copies of the MECP2 coding sequence. In some embodiments, the expression construct includes 5 copies of the MECP2 coding sequence. In some embodiments, the expression construct includes 6 copies of the MECP2 coding sequence. In some embodiments, the expression construct includes 7 copies of the MECP2 coding sequence. In some embodiments, the expression construct includes 8 copies of the MECP2 coding sequence. In some embodiments, the expression construct includes 9 copies of the MECP2 coding sequence. In some embodiments, the expression construct includes 10 copies of the MECP2 coding sequence. In some embodiments, all 1, 2, 3, 4, 5, 6, 7, 8, 9, or 10 copies of a MECP2 coding sequence sequence are operably linked to one or more promoters. For example, multiple copies of the MECP2 coding sequence may be operably linked to a single promoter. In some embodiments, all 1, 2, 3, 4, 5, 6, 7, 8, 9, or 10 copies of a MECP2 coding sequence are operably linked to one or more 3′ terminator sequences.

A modified U7 snRNA may be expressed from a U7 cassette. A functional U7 cassette may include, in the following order from 5′ to 3′: (1) a promoter, (2) an optional exonic splicing silencer, (3) a targeting sequence complementary to a region of a target mRNA (e.g. MECP2 mRNA), (4) an smOPT sequence, (5) a hairpin sequence, and (6) a 3′ termination signal. The expressed functional U7 snRNA consists of, in the following order from 5′ to 3′: (1) an optional exonic splicing silencer, (2) a targeting sequence complementary to a region of target mRNA (e.g. MECP2 mRNA), (3) an smOPT sequence, and (4) a hairpin sequence.

### Pharmaceutical Compositions

Disclosed herein, in some embodiments, are compositions. The composition may be a pharmaceutical composition. The composition may include a silencing module. The composition may include an RNA such as recombinant or modified U7 snRNA sequences described herein, or an expression construct. The composition may include an expression construct encoding a recombinant or modified U7 snRNA. The composition may include a synthesis module. The composition may include an MECP2 mRNA comprising an MECP2 coding sequence. The composition may include an expression construct comprising or encoding an MECP2 coding sequence. The composition may include a viral vector.

The compositions provided herein are contemplated to be effective for treating diseases. For example, the compositions provided herein are effective gene therapies for diseases including Rett's diseases. The compositions provided herein include systems provided herein, and expression cassettes provided herein. Thus, in an aspect is provided a pharmaceutical composition including a system provided herein including embodiments thereof and a pharmaceutically acceptable carrier.

The compositions are suitable for formulation and administration in vitro or in vivo. In embodiments, the pharmaceutical composition further includes a pharmaceutically acceptable carrier or excipient. Suitable carriers and excipients and their formulations are known in the art and described, e.g., in Remington: The Science and Practice of Pharmacy, 21st Edition, David B. Troy, ed., Lippicott Williams & Wilkins (2005), which is incorporated herein in its entirety and for all purposes.

Described herein, in some embodiments, the components of the system may be combined together within a single nucleic acid. In some embodiments, the components are separated among multiple nucleic acids.

In some embodiments, a pharmaceutical composition comprising the system and a pharmaceutically acceptable carrier is described. A composition may include a carrier such as a pharmaceutically acceptable carrier. Examples of carriers may include a solution such as water, a buffer, or saline, or a lipid composition.

The composition may include a delivery agent such as a virus, liposome, or nanoparticle. In some embodiments, the composition includes a delivery agent. In some embodiments, the delivery agent includes a virus. In some embodiments, the delivery agent includes a liposome. In some embodiments, the delivery agent includes a nanoparticle.

In an aspect is provided a virus including the system provided herein including embodiments thereof. In embodiments, the virus includes an adeno-associated virus (AAV). A virus may include the nucleic acid system. In some embodiments, the virus is a parvovirus. An example of a parvovirus may include a dependoparvovirus. An example of a dependoparvovirus may include an adeno-associated virus (AAV). In some embodiments, the virus may be an adeno-associated virus (AAV). In some embodiments, the AAV is a self-complementary AAV. In some embodiments, the AAV is a single-strand AAV. In some embodiments, the AAV may be serotype AAV1. In some embodiments, the AAV may be serotype AAV2. In some embodiments, the AAV may be serotype AAV4. In some embodiments, the AAV may be serotype AAV5. In some embodiments, the AAV may be serotype AAV6. In some embodiments, the AAV may be serotype AAV7. In some embodiments, the AAV may be serotype AAV8. In some embodiments, the AAV may be serotype AAV9.

In some embodiments, the, AAV may express one U7 cassette. Gene cassettes may include small mobile elements, consisting of a single gene and a recombination site, which may be integrated into larger elements called integrons. Several gene cassettes can be inserted into the same integrin forming a tandem array where cassettes can be expressed together. In some embodiments, the AAV may express an array of two U7 cassettes. In some embodiments, the AAV may express an array of three U7 cassettes. In some embodiments, the AAV may express an array of four U7 cassettes. In some embodiments, the AAV may express an array of five U7 cassettes. In some embodiments, the AAV may express at least one U7 cassette. In some embodiments, the AAV may express an array of at least two U7 cassettes. In some embodiments, the AAV may express an array of at least three U7 cassettes. In some embodiments, the AAV may express an array of at least four U7 cassettes. In some embodiments, the AAV may express an array of at least five U7 cassettes. In some embodiments, the AAV may express at most one U7 cassette. In some embodiments, the AAV may express an array of at most two U7 cassettes. In some embodiments, the AAV may express an array of at most three U7 cassettes. In some embodiments, the AAV may express an array of at most four U7 cassettes. In some embodiments, the AAV may express an array of at most five U7 cassettes.

In some embodiments, the AAV may express an array of two MECP2 coding sequences. In some embodiments, the AAV may express an array of three MECP2 coding sequences. In some embodiments, the AAV may express an array of four MECP2 coding sequences. In some embodiments, the AAV may express an array of five MECP2 coding sequences. In some embodiments, the AAV may express at least one MECP2 coding sequence. In some embodiments, the AAV may express an array of at least two MECP2 coding sequences. In some embodiments, the AAV may express an array of at least three MECP2 coding sequences. In some embodiments, the AAV may express an array of at least four MECP2 coding sequences. In some embodiments, the AAV may express an array of at least five MECP2 coding sequences. In some embodiments, the AAV may express at most one MECP2 coding sequence. In some embodiments, the AAV may express an array of at most two MECP2 coding sequences. In some embodiments, the AAV may express an array of at most three MECP2 coding sequences. In some embodiments, the AAV may express an array of at most four MECP2 coding sequences. In some embodiments, the AAV may express an array of at most five MECP2 coding sequences.

### Cells

In an aspect is provided a cell including the system provided herein including embodiments thereof. In embodiments, the cell includes an expression cassette comprising the system.

In embodiments, the cell includes a brain cell. In embodiments, the cell includes a neural cell. In embodiments, the cell includes a neuron. In embodiments, the cell includes a cerebrum cell.

In embodiments, the cell is a healthy cell. In embodiments, the cell is a Rett Syndrome cell. In embodiments, the cell has a lower expression level of MECP2 compared to the expression level of MECP2 in a healthy cell.

In some embodiments, a cell comprises the system (e.g. a synthesis module and a silencing module). For example, the system may be delivered to a cell. The system may be delivered to the cell in vivo. The system may be delivered to the cell in vitro. The system may be administered to a subject, and thereby enter a cell of the subject.

In some embodiments, a tissue or biofluid of a subject comprises the system (e.g. modified U7 snRNA). For example, the system may be delivered to a tissue or biofluid. The system may be delivered to the tissue or biofluid in vivo. The system may be delivered to the tissue or biofluid in vitro. The system may be administered to a subject, and thereby enter a tissue or biofluid of the subject. In some embodiments, the tissue includes neural tissue. In some embodiments, the tissue includes brain tissue. In some embodiments, the tissue includes cerebral tissue. In some embodiments, the tissue includes nerve tissue. In some embodiments, the biofluid includes blood. In some embodiments, the biofluid includes serum. In some embodiments, the biofluid includes serum.

### Methods of Treatment

Described herein, in some embodiments, are methods. The method may include administering a composition or system described herein. The method may include delivering a composition or system described herein in a cell. The method may include expressing a composition or system described herein in a cell. The cell may be in vivo (e.g. in a living body). The cell may be in vitro. The method may include a method of treatment. The method may affect MECP2 expression in a subject, or in a cell of a subject.

As described throughout the specification, the system provided herein is contemplated to be effective for treating genetic disorders (e.g. Rett Syndrome) in a subject. Thus, in an aspect is provided a method of treating a disease in a subject, including administering the system provided herein including embodiments thereof to the subject. In another aspect is provided a method, including administering the pharmaceutical composition provided herein including embodiments thereof or the virus provided herein including embodiments thereof to a subject.

Described herein, in some embodiments, is a method for treating a disorder in a subject in need thereof. In some embodiments, the subject has the disorder. In some embodiments, the subject is identified as having the disorder. In some embodiments, the subject is at risk of having the disorder. In some embodiments, the subject is identified as at risk of having the disorder. The treatment may have a prophylactic effect. The method may include any aspect of another method described herein, such as modifying expression of MECP2 in a cell of a subject.

The disorder may be or include a genetic disorder. In some embodiments, the subject has or is at risk of having a genetic disorder. The disorder may be or include a neurodevelopmental disorder. In some embodiments, the subject has or is at risk of having a neurodevelopmental disorder. In some embodiments, the neurodevelopmental disorder comprises an intellectual disability or epilepsy. The disorder may include an intellectual disability. The disorder may include epilepsy. The disorder may include Rett syndrome.

The system provided herein has been demonstrated to be effective for regulating expression levels of the MECP2 gene, wherein the expression levels of the MECP2 gene are affected by a genetic disorder (e.g. Rett Syndrome). For example, administering a system provided herein to a subject allows expression of a recombinant version of the MECP2 gene in the subject. Thus, in an aspect is provided a method of treating a genetic disease in a subject, including: administering the system provided herein including embodiments thereof to the subject.

In embodiments, the genetic disease is associated with haploinsufficiency of the endogenous MECP2 gene. In embodiments, the genetic disease is associated with tissue mosaic expression of the endogenous MECP2 gene. In embodiments, the genetic disease includes Rett syndrome.

In some embodiments, the subject has a disorder. The disorder may include a neurodevelopmental disorder. In some embodiments, the subject is at risk of having the neurodevelopmental disorder. The neurodevelopmental disorder may include Rett syndrome.

In some embodiments, the treatment improves a phenotypic severity score. The phenotypic severity score may be for Rett syndrome. The phenotypic severity score may be a product of an assessment of any of the following domains: gait, mobility, hindlimb clasping, breathing abnormalities, tremor, general body condition, or a combination thereof. The phenotypic severity score may be a product of an assessment of the following domains: gait, mobility, hindlimb clasping, breathing abnormalities, tremor, and general body condition. An assessment of each of gait, mobility, hindlimb clasping, breathing abnormalities, tremor, or general body condition may include a score of 0, 1, or 2. An assessment of each of gait, mobility, hindlimb clasping, breathing abnormalities, tremor, and general body condition may include a score of 0, 1, or 2. An example of a phenotypic severity score may include a Bird score. In some embodiments, the treatment improves a subject's gait. In some embodiments, the treatment improves a subject's mobility. In some embodiments, the treatment improves a subject's clasping ability. In some embodiments, the treatment improves a subject's breathing. In some embodiments, the treatment improves (e.g. reduces) a subject's tremor. In some embodiments, the treatment improves a subject's general body condition. An improvement in any of the aforementioned domain may be determined by a score (e.g. a score of 0, 1 or 2. As such, an improvement may be reflected as a change in a score of 1 or 2 for any of they aforementioned domains. In some embodiments, an improvement is determined as an increase or decrease in a measurement. The increase or decrease may be by at least 5%, at least 10%, at least 15%, or more, relative to a baseline measurement obtained before the treatment.

### Methods of Modulating Protein Expression

In an aspect is provided a method including: suppressing protein expression of an endogenous MECP2 RNA in a first cell expressing the endogenous MECP2 RNA; and synthesizing or enhancing protein expression of a recombinant version of the MECP2 RNA in a second cell that otherwise does not express the endogenous MECP2 RNA, or that expresses the endogenous MECP2 RNA at a low level. Some embodiments include synthesizing or enhancing protein expression of the recombinant version of the MECP2 RNA in the first cell. In embodiments, suppressing endogenous MECP2 expression or endogenous MECP2 protein expression is performed upon contacting the first cell with a silencing module provided herein including embodiments thereof.

A construct encoding a synthesis module and a silencing module may be expressed in two cells, where the silencing module suppresses expression of any endogenous MECP2 in either cell that has endogenous MECP2 expression, while the synthesis module synthesizes protein expression of MECP2 in both cells. Some embodiments include suppressing any endogenously expressed MECP2 in a group of target cells, and replacing the endogenous MECP2 expression with a new MECP2 protein in target cells that already express MECP2, while also causing expression of MECP2 in target cells that did not already express MECP2 or that only expressed MECP2 at low levels. In some embodiments, re-expression of MECP2 occurs in all target cells, while knockdown only occurs in target cells that endogenously express MECP2 mRNA.

In embodiments, suppressing endogenous MECP2 protein expression includes suppressing endogenous MECP2 protein expression by at least 10%. In embodiments, endogenous MECP2 protein expression is suppressed by at least 10% compared to the expression level of the endogenous MECP2 protein in the absence of the silencing module. In embodiments, endogenous MECP2 protein expression is suppressed by at least 15% compared to the expression level of the endogenous MECP2 protein in the absence of the silencing module. In embodiments, endogenous MECP2 protein expression is suppressed by at least 20% compared to the expression level of the endogenous MECP2 protein in the absence of the silencing module. In embodiments, endogenous MECP2 protein expression is suppressed by at least 25% compared to the expression level of the endogenous MECP2 protein in the absence of the silencing module. In embodiments, endogenous MECP2 protein expression is suppressed by at least 30% compared to the expression level of the endogenous MECP2 protein in the absence of the silencing module. In embodiments, endogenous MECP2 protein expression is suppressed by at least 35% compared to the expression level of the endogenous MECP2 protein in the absence of the silencing module. In embodiments, endogenous MECP2 protein expression is suppressed by at least 40% compared to the expression level of the endogenous MECP2 protein in the absence of the silencing module. In embodiments, endogenous MECP2 protein expression is suppressed by at least 45% compared to the expression level of the endogenous MECP2 protein in the absence of the silencing module. In embodiments, endogenous MECP2 protein expression is suppressed by at least 50% compared to the expression level of the endogenous MECP2 protein in the absence of the silencing module. In embodiments, endogenous MECP2 protein expression is suppressed by at least 55% compared to the expression level of the endogenous MECP2 protein in the absence of the silencing module. In embodiments, endogenous MECP2 protein expression is suppressed by at least 60% compared to the expression level of the endogenous MECP2 protein in the absence of the silencing module. In embodiments, endogenous MECP2 protein expression is suppressed by at least 65% compared to the expression level of the endogenous MECP2 protein in the absence of the silencing module. In embodiments, endogenous MECP2 protein expression is suppressed by at least 70% compared to the expression level of the endogenous MECP2 protein in the absence of the silencing module. In embodiments, endogenous MECP2 protein expression is suppressed by at least 75% compared to the expression level of the endogenous MECP2 protein in the absence of the silencing module. In embodiments, endogenous MECP2 protein expression is suppressed by at least 80% compared to the expression level of the endogenous MECP2 protein in the absence of the silencing module. In embodiments, endogenous MECP2 protein expression is suppressed by at least 85% compared to the expression level of the endogenous MECP2 protein in the absence of the silencing module. In embodiments, endogenous MECP2 protein expression is suppressed by at least 90% compared to the expression level of the endogenous MECP2 protein in the absence of the silencing module. In embodiments, endogenous MECP2 protein expression is suppressed by at least 95% compared to the expression level of the endogenous MECP2 protein in the absence of the silencing module. In embodiments, endogenous MECP2 protein expression is suppressed by at least 96% compared to the expression level of the endogenous MECP2 protein in the absence of the silencing module. In embodiments, endogenous MECP2 protein expression is suppressed by at least 97% compared to the expression level of the endogenous MECP2 protein in the absence of the silencing module. In embodiments, endogenous MECP2 protein expression is suppressed by at least 98% compared to the expression level of the endogenous MECP2 protein in the absence of the silencing module. In embodiments, endogenous MECP2 protein expression is suppressed by at least 99% compared to the expression level of the endogenous MECP2 protein in the absence of the silencing module. In embodiments, endogenous MECP2 protein expression is suppressed 100% compared to the expression level of the endogenous MECP2 protein in the absence of the silencing module.

In embodiments, the synthesizing or enhancing protein expression is performed upon contacting the second cell with a synthesis module provided herein including embodiments thereof. In embodiments, enhancing protein expression includes enhancing protein expression by at least 10%. In embodiments, enhancing MECP2 expression refers to the expression level of recombinant MECP2 protein. Thus, in embodiments, MECP2 protein (e.g. recombinant MECP2) expression is enhanced by at least 10% compared to the expression level of the MECP2 protein (e.g. recombinant MECP2) in the absence of the synthesis module. In embodiments, MECP2 protein (e.g. recombinant MECP2 protein) expression is enhanced by at least 15% compared to the expression level of the protein in the absence of the synthesis module. In embodiments, MECP2 protein (e.g. recombinant MECP2 protein) expression is enhanced by at least 20% compared to the expression level of the protein in the absence of the synthesis module. In embodiments, MECP2 protein (e.g. recombinant MECP2 protein) expression is enhanced by at least 25% compared to the expression level of the recombinant MECP2 protein in the absence of the synthesis module. In embodiments, MECP2 protein (e.g. recombinant MECP2 protein) expression is enhanced by at least 30% compared to the expression level of the protein in the absence of the synthesis module. In embodiments, MECP2 protein (e.g. recombinant MECP2 protein) expression is enhanced by at least 35% compared to the expression level of the protein in the absence of the synthesis module. In embodiments, MECP2 protein (e.g. recombinant MECP2 protein) expression is enhanced by at least 40% compared to the expression level of the protein in the absence of the synthesis module. In embodiments, MECP2 protein (e.g. recombinant MECP2 protein) expression is enhanced by at least 45% compared to the expression level of the protein in the absence of the synthesis module. In embodiments, MECP2 protein (e.g. recombinant MECP2 protein) expression is enhanced by at least 50% compared to the expression level of the protein in the absence of the synthesis module. In embodiments, protein expression is enhanced by at least 55% compared to the expression level of the protein in the absence of the synthesis module. In embodiments, MECP2 protein (e.g. recombinant MECP2 protein) expression is enhanced by at least 60% compared to the expression level of the protein in the absence of the synthesis module. In embodiments, MECP2 protein (e.g. recombinant MECP2 protein) expression is enhanced by at least 65% compared to the expression level of the protein in the absence of the synthesis module. In embodiments, MECP2 protein (e.g. recombinant MECP2 protein) expression is enhanced by at least 70% compared to the expression level of the protein in the absence of the synthesis module. In embodiments, MECP2 protein (e.g. recombinant MECP2 protein) expression is enhanced by at least 75% compared to the expression level of the protein in the absence of the synthesis module. In embodiments, protein expression is enhanced by at least 80% compared to the expression level of the protein in the absence of the synthesis module. In embodiments, MECP2 protein (e.g. recombinant MECP2 protein) expression is enhanced by at least 85% compared to the expression level of the protein in the absence of the synthesis module. In embodiments, MECP2 protein (e.g. recombinant MECP2 protein) expression is enhanced by at least 90% compared to the expression level of the protein in the absence of the synthesis module. In embodiments, MECP2 protein (e.g. recombinant MECP2 protein) expression is enhanced by at least 91% compared to the expression level of the protein in the absence of the synthesis module. In embodiments, protein expression is enhanced by at least 92% compared to the expression level of the protein in the absence of the synthesis module. In embodiments, MECP2 protein (e.g. recombinant MECP2 protein) expression is enhanced by at least 93% compared to the expression level of the protein in the absence of the synthesis module. In embodiments, MECP2 protein (e.g. recombinant MECP2 protein) expression is enhanced by at least 94% compared to the expression level of the protein in the absence of the synthesis module. In embodiments, MECP2 protein (e.g. recombinant MECP2 protein) expression is enhanced by at least 95% compared to the expression level of the protein in the absence of the synthesis module. In embodiments, MECP2 protein (e.g. recombinant MECP2 protein) expression is enhanced by at least 96% compared to the expression level of the protein in the absence of the synthesis module. In embodiments, MECP2 protein (e.g. recombinant MECP2 protein) expression is enhanced by at least 97% compared to the expression level of the protein in the absence of the synthesis module. In embodiments, MECP2 protein (e.g. recombinant MECP2 protein) expression is enhanced by at least 98% compared to the expression level of the protein in the absence of the synthesis module. In embodiments, MECP2 protein (e.g. recombinant MECP2 protein) expression is enhanced by at least 99% compared to the expression level of the protein in the absence of the synthesis module.

In embodiments, the low level of expression of the endogenous MECP2 RNA in the second cell includes an undetectable level, includes a level below a desired level, includes a level lower than a wild type cell, or includes an expression lower than that of the first cell. In embodiments, the low level of expression of the target RNA in the second cell includes an undetectable level. Expression of the endogenous MECP2 RNA may be measured using any method known by a person skilled in the art, including mass spectrometry, gel electrophoresis, Western Blot, and antibody-based methods (e.g. ELISA). In embodiments, the low level of expression of the endogenous MECP2 RNA in the second cell includes a level below a desired level. For example, a desired level of expression may be the expression level of the endogenous MECP2 RNA in a healthy cell. In embodiments, a healthy cell does not have a genetic disorder. In embodiments, a healthy does not have Rett disease. In embodiments, the low level of expression of the endogenous MECP2 RNA in the second cell includes a level lower than a wild type cell. In embodiments, the wild type cell is a healthy cell. In embodiments, the wild type cell does not have Rett syndrome.

In embodiments, the low level of expression of the endogenous MECP2 RNA in the second cell includes an expression lower than that of the first cell. In embodiments, the low level of expression of the endogenous MECP2 RNA in the second cell includes a level at least 10% lower than that of the first cell. In embodiments, the level of expression of the endogenous MECP2 RNA in the second cell is at least 10% lower than the expression level of the endogenous MECP2 RNA in the first cell. In embodiments, the level of expression of the endogenous MECP2 RNA in the second cell is at least 15% lower than the expression level of the endogenous MECP2 RNA in the first cell. In embodiments, the level of expression of the endogenous MECP2 RNA in the second cell is at least 20% lower than the expression level of the endogenous MECP2 RNA in the first cell. In embodiments, the level of expression of the endogenous MECP2 RNA in the second cell is at least 25% lower than the expression level of the endogenous MECP2 RNA in the first cell. In embodiments, the level of expression of the endogenous MECP2 RNA in the second cell is at least 30% lower than the expression level of the endogenous MECP2 RNA in the first cell. In embodiments, the level of expression of the endogenous MECP2 RNA in the second cell is at least 35% lower than the expression level of the endogenous MECP2 RNA in the first cell. In embodiments, the level of expression of the endogenous MECP2 RNA in the second cell is at least 40% lower than the expression level of the endogenous MECP2 RNA in the first cell. In embodiments, the level of expression of the endogenous MECP2 RNA in the second cell is at least 45% lower than the expression level of the endogenous MECP2 RNA in the first cell. In embodiments, the level of expression of the endogenous MECP2 RNA in the second cell is at least 50% lower than the expression level of the endogenous MECP2 RNA in the first cell. In embodiments, the level of expression of the endogenous MECP2 RNA in the second cell is at least 55% lower than the expression level of the endogenous MECP2 RNA in the first cell. In embodiments, the level of expression of the endogenous MECP2 RNA in the second cell is at least 60% lower than the expression level of the endogenous MECP2 RNA in the first cell. In embodiments, the level of expression of the endogenous MECP2 RNA in the second cell is at least 65% lower than the expression level of the endogenous MECP2 RNA in the first cell. In embodiments, the level of expression of the endogenous MECP2 RNA in the second cell is at least 70% lower than the expression level of the endogenous MECP2 RNA in the first cell. In embodiments, the level of expression of the endogenous MECP2 RNA in the second cell is at least 75% lower than the expression level of the endogenous MECP2 RNA in the first cell. In embodiments, the level of expression of the endogenous MECP2 RNA in the second cell is at least 80% lower than the expression level of the endogenous MECP2 RNA in the first cell. In embodiments, the level of expression of the endogenous MECP2 RNA in the second cell is at least 85% lower than the expression level of the endogenous MECP2 RNA in the first cell. In embodiments, the level of expression of the endogenous MECP2 RNA in the second cell is at least 90% lower than the expression level of the endogenous MECP2 RNA in the first cell. In embodiments, the level of expression of the endogenous MECP2 RNA in the second cell is at least 910% lower than the expression level of the endogenous MECP2 RNA in the first cell. In embodiments, the level of expression of the endogenous MECP2 RNA in the second cell is at least 92% lower than the expression level of the endogenous MECP2 RNA in the first cell. In embodiments, the level of expression of the endogenous MECP2 RNA in the second cell is at least 93% lower than the expression level of the endogenous MECP2 RNA in the first cell. In embodiments, the level of expression of the endogenous MECP2 RNA in the second cell is at least 94% lower than the expression level of the endogenous MECP2 RNA in the first cell. In embodiments, the level of expression of the endogenous MECP2 RNA in the second cell is at least 95% lower than the expression level of the endogenous MECP2 RNA in the first cell. In embodiments, the level of expression of the endogenous MECP2 RNA in the second cell is at least 96% lower than the expression level of the endogenous MECP2 RNA in the first cell. In embodiments, the level of expression of the endogenous MECP2 RNA in the second cell is at least 97% lower than the expression level of the endogenous MECP2 RNA in the first cell. In embodiments, the level of expression of the endogenous MECP2 RNA in the second cell is at least 98% lower than the expression level of the endogenous MECP2 RNA in the first cell. In embodiments, the level of expression of the endogenous MECP2 RNA in the second cell is at least 99% lower than the expression level of the endogenous MECP2 RNA in the first cell.

In embodiments, the silencing module and the synthesis module are encoded together in a nucleic acid construct. For example, the silencing module and the synthesis module may be encoded together in a viral vector or a plasmid.

In embodiments, the silencing module and the synthesis module are encoded in separate nucleic acid constructs. For example, the silencing module and the synthesis module may be encoded in a first viral vector and a second viral vector. In another example, the silencing module and the synthesis module may be encoded in a first plasmid and a second plasmid.

In embodiments, the nucleic acid construct or the separate nucleic acid constructs are delivered to the cells using one or more viral vectors.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Administration

Disclosed herein, in some embodiments, are methods that include administering a composition. The administration may be to a subject. The administration may be to a human subject. The administered composition may include an engineered snRNA. The administered composition may include an expression vector. The administered composition may include an expression vector encoding an engineered snRNA. The administered composition may include a pharmaceutical composition. The administered composition may include a virus. The administered composition may include a virus comprising an expression vector. The administered composition may include a liposome. The administered composition may include a nanoparticle.

The administration may be by a route of administration. The administration be systemic. The administration be intravenous. The administration may include an injection.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 15% of that number. The term “about” a range refers to that range minus 15% of its lowest value and plus 15% of its greatest value.

As may be used herein, the terms “nucleic acid,” “nucleic acid molecule,” “nucleic acid oligomer,” “oligonucleotide,” “nucleic acid sequence,” “nucleic acid fragment” and “polynucleotide” may be used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), small nuclear RNA (snRNA), small interfering RNA (siRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. In embodiments, the nucleic acid provided herein may be part of a lentiviral vector, which may be transduced into a cell. Polynucleotides useful in the methods of the disclosure may comprise natural nucleic acid sequences and variants thereof, artificial nucleic acid sequences, or a combination of such sequences.

Nucleic acids may include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) in place of thymine (T) when the polynucleotide is RNA). Thus, the term “polynucleotide sequence” may be the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, may refer to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Thus, a nucleic acid sequence of the present invention may include a complement of a sequence provided herein.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 75%, 85%, 90%, 91%, 92%, 93%, 94%, 95%, 96%, 97%, 98%, 99%, or higher identity over a specified region).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” may apply to both amino acid and nucleic acid sequences. With respect to particular nucleic acid sequences, “conservatively modified variants” may refer to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations may be “silent variations,” which may be one species of conservatively modified variations. Every nucleic acid sequence herein which encodes a polypeptide may also describe every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The terms “identical” or percent “identity,” in the context of two or more nucleic acids or polypeptide sequences, may refer to two or more sequences or subsequences that are the same or have a specified percentage of amino acid residues or nucleotides that are the same (i.e., about 60% identity, preferably 65%, 70%, 75%, 75%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. The preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” may be determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An amino acid or nucleotide base “position” may be denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

A “comparison window”, as used herein, may include reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that may be suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits may act as seeds for initiating searches to find longer HSPs containing them. The word hits may be extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm may also perform a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

For specific proteins described herein, the named protein may include any of the protein's naturally occurring forms, variants or homologs that maintain activity of the protein (e.g., within at least 50%, 75%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “meCP2 protein”, “meCP2” or “MECP2” as used herein may include any of the recombinant or naturally-occurring forms of methyl CpG binding protein 2 (meCP2), also known as demethylase, DMTase, or variants or homologs thereof that maintain meCP2 activity (e.g. within at least 50%, 80%, 90%, 95%, 96%, 97%, 98%, 99% or 100% activity compared to meCP2). In embodiments, MECP2 refers to a recombinant form of MECP2 protein. In embodiments, MECP2 refers to a naturally occurring form of MECP2 protein. For example, in embodiments, MECP2 refers to a MECP2 protein endogenous to a cell. In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring meCP2 protein. In embodiments, the meCP2 protein is substantially identical to the protein identified by the UniProt reference number P51608 (last updated Oct. 1, 1996) or a variant or homolog having substantial identity thereto.

A “gene” may include a segment of a nucleic acid involved in producing a protein; a gene may include regions preceding and following the coding region (leader and trailer) as well as intervening sequences (introns) between individual coding segments (exons). The leader, the trailer as well as the introns may include regulatory elements that are necessary during the transcription and the translation of a gene. In embodiments, a gene may include a nucleic acid such as a DNA sequence involved in producing a protein.

The terms “plasmid”, “vector” or “expression vector” may refer to a nucleic acid molecule that encodes for genes and/or regulatory elements necessary for the expression of genes. Expression of a gene from a plasmid can occur in cis or in trans. If a gene is expressed in cis, the gene and the regulatory elements may be encoded by the same plasmid. Expression in trans may refer to instances where the gene and the regulatory elements are encoded by separate plasmids.

As used herein, the term “construct” may be intended to mean any recombinant nucleic acid molecule. In embodiments, a construct includes an expression cassette, plasmid, cosmid, virus, autonomously replicating polynucleotide molecule, phage, or linear or circular, single-stranded or double-stranded, DNA or RNA polynucleotide molecule. A construct may be derived from any source, capable of genomic integration or autonomous replication, including a nucleic acid molecule where one or more nucleic acid sequences has been linked in a functionally operative manner, e.g., operably linked.

The terms “operably linked” or “functionally linked”, may be interchangeable and may refer to a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) may be a functional link that allows for expression of the polynucleotide of interest. In this sense, the term “operably linked” refers to the positioning of a regulatory region (e.g. a promoter) and a coding sequence (e.g. polynucleotide encoding a gene editing agent, etc.) to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, operably linked elements may be contiguous or non-contiguous. In addition, in the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, regions, or domains of the engineered antibodies disclosed herein may be operably linked to retain proper folding, processing, targeting, expression, binding, and other functional properties of the engineered antibodies in the cell. Operably linked regions, domains, and segments of the engineered antibodies of the disclosure may be contiguous or non-contiguous (e.g., linked to one another through a linker).

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a lentiviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” may be used according to their plain ordinary meanings and refer to the process by which one or more foreign nucleic acids (i.e. DNA not naturally found in the cell) are introduced into a cell. Typically, transduction occurs by introduction of a virus or viral vector (e.g. a CMV vector, a lentivirus vector, etc.) into the cell.

As used herein, the term “promoter” may refer to a sequence of DNA which proteins bind to initiate gene expression. For example, transcription factors may bind a promoter region of a gene to transcribe RNA from DNA.

“Contacting” may be used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be, for example, a nucleic acid as provided herein and a cell. In embodiments contacting includes, for example, allowing a nucleic acid as described herein to interact with a cell. In embodiments, contacting refers to allowing a system provided herein to interact with a cell. Thus, in embodiments, contacting includes allowing a nucleic acid to interact with a cell, thereby resulting in transduced cell. In embodiments contacting includes, for example, allowing a pharmaceutical composition as described herein to interact with a cell. In embodiments contacting includes, for example, allowing an expression cassette (e.g. expression vector, construct) as described herein to interact with a cell.

A “cell” as used herein, refers to a cell carrying out metabolic function or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “virus” or “virus particle” may be used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “replicate” may be used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule.

In the context of a virus, the term “replicate” may include an ability of a virus to replicate (duplicate the viral genome and packaging said genome into viral particles) in a host cell and subsequently release progeny viruses from the host cell, which results in the lysis of the host cell.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express proteins that are not found within the native (non-recombinant) form of the cell. A recombinant RNA may include an RNA that is encoded unnaturally in a cell, such as being encoded by a vector that is introduced to the cell.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” may refer to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” may refer to a molecule or substance that is native to, or originates within, a given cell or organism.

The term “expression” may include any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

A “control” or “standard control” may refer to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient”, “subject” or “subject in need thereof” may refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. The patient may be male. The patient may be female. For example, in an X-linked haploinsufficiency disease or disorder, the patient may likely be a female.

The terms “disease” or “condition” may refer to a state of being or health status of a patient or subject capable of being treated with the compounds or methods provided herein. The disease may be a genetic disorder. In embodiments, the genetic disorder is Rett Syndrome.

The term “aberrant” as used herein may refer to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

Some embodiments relate to a small nuclear RNA (snRNA). An snRNA may include a class of small RNA molecules found within splicing speckles or Cajal bodies of a eukaryotic cell nucleus. A length of an unmodified snRNA may average about 150 nucleotides. An snRNA may be transcribed by RNA polymerase II or RNA polymerase III. An snRNA may function in the processing of pre-messenger RNA (hnRNA) in the nucleus. Any of these aspects may be modified or missing in a modified or engineered snRNA. An snRNA may associate with a protein or set of proteins to form a complex. The complex may be referred to as a small nuclear ribonucleoprotein (snRNP). Some examples of human snRNA components of such complexes may include: U1 spliceosomal RNA, U2 spliceosomal RNA, U4 spliceosomal RNA, U5 spliceosomal RNA, or U6 spliceosomal RNA. An snRNA may have a high uridine content.

Some embodiments relate to a U7 snRNA. A U7 snRNA may include an RNA molecule and a component of a small nuclear ribonucleoprotein complex (U7 snRNP). The U7 snRNA may affect histone pre-mRNA processing. The U7 snRNA may be modified or engineered. In some embodiments, the modified or engineered U7 snRNA does not affect histone pre-mRNA processing, or has little effect on such. In some embodiments, a U7 snRNA has a 5′ end that binds an HDE (histone downstream element), a conserved purine-rich region, located 15 nucleotides downstream a histone mRNA cleavage site. Any of these aspects may be modified or missing in a modified or engineered U7 snRNA. Binding of an HDE region by a U7 snRNA, through complementary base-pairing, may affect recruitment of cleavage factors during histone pre-mRNA processing.

The terms “treating”, or “treatment” may refer to an indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating includes preventing. In embodiments, treating does not include preventing.

“Treating” or “treatment” as used herein (and as well-understood in the art) may broadly include any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. “Treatment” may include a cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things. Thus in the disclosed method, treatment can refer to a 10%, 20%, 30%, 40%, 50%, 60%, 70%, 75%, 90%, or 100% reduction in the severity of an established disease, condition, or symptom of the disease or condition. For example, a method for treating a disease may be considered to be a treatment when there is a 10% reduction in one or more symptoms of the disease in a subject as compared to a control. Thus the reduction can be a 10%, 20%, 30%, 40%, 50%, 60%, 70%, 75%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 75%, 90% or greater as compared to a control level and such terms can include but do not necessarily include complete elimination.

A sequence identity may include a sequence identity to a reverse complement. In determining a sequence identity, thymine (T) and uracil (U) may be interchangeable. T and U may be interchangeable when describing an oligonucleotide. In some embodiments, Ts and Us are interchangeable depending on whether the oligonucleotide is an RNA or DNA, where RNA includes U and DNA includes T.

A discrepancy between the written description and a sequence listing submitted herein may be resolved in favor of the written description.

“Treating” and “treatment” as used herein may include prophylactic treatment. Treatment methods may include administering to a subject a therapeutically effective amount of an active agent. The administering step may consist of a single administration or may include a series of administrations. The length of the treatment period may depend on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, a composition may be administered to a subject in an amount and for a duration sufficient to treat the subject. In embodiments, the treating or treatment is not prophylactic treatment.

The term “prevent” may refer to a decrease in the occurrence of disease symptoms in a patient. The prevention may be complete (no detectable symptoms) or partial, such that fewer symptoms are observed than would likely occur absent treatment.

As used herein, the term “administering” may be used in accordance with its plain and ordinary meaning. An administration may be systemic. An administration may include an injection.

“Pharmaceutically acceptable excipient” and “pharmaceutically acceptable carrier” may refer to a substance that aids the administration of an active agent to and absorption by a subject and can be included in the compositions of the present disclosure without causing a significant adverse toxicological effect on the patient. Non-limiting examples of pharmaceutically acceptable excipients may include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients may be useful in the present disclosure.

A “therapeutic agent” as used herein may refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Tables

TABLE 1
Sequences of functional elements used in the different gene therapy
vector embodiments described herein.

| Component | Sequence (5′ to 3′) | SEQ ID NO |
|---|---|---|
| UbC Promoter | ggcctccgcgccgggttttggcgcctcccgcgggcgcccc | 1 |
| | cctcctcacggcgagcgctgccacgtcagacgaagggcgc | |
| | agcgagcgtcctgatccttccgcccggacgctcaggacag | |
| | cggcccgctgctcataagactcggccttagaaccccagta | |
| | tcagcagaaggacattttaggacgggacttgggtgactct | |
| | agggcactggttttctttccagagagcggaacaggcgagg | |
| | aaaagtagtcccttctcggcgattctgcggagggatctcc | |
| | gtggggcggtgaacgccgatgattatataaggacgcgccg | |
| | ggtgtggcacagctagttccgtcgcagccgggatttgggt | |
| | cgcggttcttgtttgtggatcgctgtgatcgtcacttggt | |
| PGK Promoter | GGGTAGGGGAGGCGCTTTTCCCAAGGCAGTCTGGAGCATG | 2 |
| | CGCTTTAGCAGCCCCGCTGGGCACTTGGCGCTACACAAGT | |
| | GGCCTCTGGCCTCGCACACATTCCACATCCACCGGTAGGC | |
| | GCCAACCGGCTCCGTTCTTTGGTGGCCCCTTCGCGCCACC | |
| | TTCTACTCCTCCCCTAGTCAGGAAGTTCCCCCCCGCCCCG | |
| | CAGCTCGCGTCGTGCAGGACGTGACAAATGGAAGTAGCAC | |
| | GTCTCACTAGTCTCGTGCAGATGGACAGCACCGCTGAGCA | |
| | ATGGAAGCGGGTAGGCCTTTGGGGCAGCGGCCAATAGCAG | |
| | CTTTGCTCCTTCGCTTTCTGGGCTCAGAGGCTGGGAAGGG | |
| | GTGGGTCCGGGGGCGGGCTCAGGGGGGGGCTCAGGGGCGG | |
| | GGCGGGCGCCCGAAGGTCCTCCGGAGGCCCGGCATTCTGC | |
| | ACGCTTCAAAAGCGCACGTCTGCCGCGCTGTTCTCCTCTT | |
| | CCTCATCTCCGGGCCTTTCG | |
| EF1a-core | gctccggtgcccgtcagtgggcagagcgcacatcgcccac | 3 |
| Promoter | agtccccgagaagttggggggaggggtcggcaattgaacc | |
| | ggtgcctagagaaggtggcgcggggtaaactgggaaagtg | |
| | atgtcgtgtactggctccgcctttttcccgagggtggggg | |
| | agaaccgtatataagtgcagtagtcgccgtgaacgttctt | |
| | tttcgcaacgggtttgccgccagaacacag | |
| UbC-SV40 intron | ggcctccgcgccgggttttggcgcctcccgcgggcgcccc | 4 |
| | cctcctcacggcgagcgctgccacgtcagacgaagggcgc | |
| | agcgagcgtcctgatccttccgcccggacgctcaggacag | |
| | cggcccgctgctcataagactcggccttagaaccccagta | |
| | tcagcagaaggacattttaggacgggacttgggtgactct | |
| | agggcactggttttctttccagagagcggaacaggcgagg | |
| | aaaagtagtcccttctcggcgattctgcggagggatctcc | |
| | gtggggcggtgaacgccgatgattatataaggacgcgccg | |
| | ggtgtggcacagctagttccgtcgcagccgggatttgggt | |
| | cgcggttcttgtttgtggatcgctgtgatcgtcacttggt | |
| | CGGTAAGTTTAGTCTTTTTGTCTTTTATTTCAGGTCCCGG | |
| | ATCCGGTGGTGGTGCAAATCAAAGAACTGCTCCTCAGTGG | |
| | ATGTTGCCTTTACTTCTAG | |
| Hs MECP2 5′ UTR | GCGCGCGCTCCCTCCTCTCGGAGAGAGGGCTGTGGTAAAA | 5 |
| | GCCGTCCGGAAAATG | |
| Mm Mecp2 CDS | ATGGCCGCCGCTGCCGCCACCGCCGCCGCCGCCGCCGCGC | 6 |
| | CGAGCGGAGGAGGAGGAGGAGGCGAGGAGGAGAGACTGGA | |
| | GGAAAAGTCAGAAGACCAGGATCTCCAGGGCCTCAGAGAC | |
| | AAGCCACTGAAGTTTAAGAAGGCGAAGAAAGACAAGAAGG | |
| | AGGACAAAGAAGGCAAGCATGAGCCACTACAACCTTCAGC | |
| | CCACCATTCTGCAGAGCCAGCAGAGGCAGGCAAAGCAGAA | |
| | ACATCAGAAAGCTCAGGCTCTGCCCCAGCAGTGCCAGAAG | |
| | CCTCGGCTTCCCCCAAACAGCGGCGCTCCATTATCCGTGA | |
| | CCGGGGACCTATGTATGATGACCCCACCTTGCCTGAAGGT | |
| | TGGACACGAAAGCTTAAACAAAGGAAGTCTGGCCGATCTG | |
| | CTGGAAAGTATGATGTATATTTGATCAATCCCCAGGGAAA | |
| | AGCTTTTCGCTCTAAAGTAGAATTGATTGCATACTTTGAA | |
| | AAGGTGGGAGACACCTCCTTGGACCCTAATGATTTTGACT | |
| | TCACGGTAACTGGGAGAGGGAGCCCCTCCAGGAGAGAGCA | |
| | GAAACCACCTAAGAAGCCCAAATCTCCCAAAGCTCCAGGA | |
| | ACTGGCAGGGGTCGGGGACGCCCCAAAGGGAGCGGCACTG | |
| | GGAGACCAAAGGCAGCAGCATCAGAAGGTGTTCAGGTGAA | |
| | AAGGGTCCTGGAGAAGAGCCCTGGGAAACTTGTTGTCAAG | |
| | ATGCCTTTCCAAGCATCGCCTGGGGGTAAGGGTGAGGGAG | |
| | GTGGGGCTACCACATCTGCCCAGGTCATGGTGATCAAACG | |
| | CCCTGGCAGAAAGCGAAAAGCTGAAGCTGACCCCCAGGCC | |
| | ATTCCTAAGAAACGGGGTAGAAAGCCTGGGAGTGTGGTGG | |
| | CAGCTGCTGCAGCTGAGGCCAAAAAGAAAGCCGTGAAGGA | |
| | GTCTTCCATACGGTCTGTGCATGAGACTGTGCTCCCCATC | |
| | AAGAAGCGCAAGACCCGGGAGACGGTCAGCATCGAGGTCA | |
| | AGGAAGTGGTGAAGCCCCTGCTGGTGTCCACCCTTGGTGA | |
| | GAAAAGCGGGAAGGGACTGAAGACCTGCAAGAGCCCTGGG | |
| | CGTAAAAGCAAGGAGAGCAGCCCCAAGGGGCGCAGCAGCA | |
| | GTGCCTCCTCCCCACCTAAGAAGGAGCACCATCATCACCA | |
| | CCATCACTCAGAGTCCACAAAGGCCCCCATGCCACTGCTC | |
| | CCATCCCCACCCCCACCTGAGCCTGAGAGCTCTGAGGACC | |
| | CCATCAGCCCCCCTGAGCCTCAGGACTTGAGCAGCAGCAT | |
| | CTGCAAAGAAGAGAAGATGCCCCGAGGAGGCTCACTGGAA | |
| | AGCGATGGCTGCCCCAAGGAGCCAGCTAAGACTCAGCCTA | |
| | TGGTCGCCACCACTACCACAGTTGCAGAAAAGTACAAACA | |
| | CCGAGGGGAGGGAGAGCGCAAAGACATTGTTTCATCTTCC | |
| | ATGCCAAGGCCAAACAGAGAGGAGCCTGTGGACAGCCGGA | |
| | CGCCCGTGACCGAGAGAGTTAGCTGA | |
| Hs MECP2 CDS | ATGGCCGCCGCCGCCGCCGCCGCGCCGAGCGGAGGAGGAG | 7 |
| | GAGGAGGCGAGGAGGAGAGACTGGAAGAAAAGTCAGAAGA | |
| | CCAGGACCTCCAGGGCCTCAAGGACAAACCCCTCAAGTTT | |
| | AAAAAGGTGAAGAAAGATAAGAAAGAAGAGAAAGAGGGCA | |
| | AGCATGAGCCCGTGCAGCCATCAGCCCACCACTCTGCTGA | |
| | GCCCGCAGAGGCAGGCAAAGCAGAGACATCAGAAGGGTCA | |
| | GGCTCCGCCCCGGCTGTGCCGGAAGCTTCTGCCTCCCCCA | |
| | AACAGCGGCGCTCCATCATCCGTGACCGGGGACCCATGTA | |
| | TGATGACCCCACCCTGCCTGAAGGCTGGACACGGAAGCTT | |
| | AAGCAAAGGAAATCTGGCCGCTCTGCTGGGAAGTATGATG | |
| | TGTATTTGATCAATCCCCAGGGAAAAGCCTTTCGCTCTAA | |
| | AGTGGAGTTGATTGCGTACTTCGAAAAGGTAGGCGACACA | |
| | TCCCTGGACCCTAATGATTTTGACTTCACGGTAACTGGGA | |
| | GAGGGAGCCCCTCCCGGCGAGAGCAGAAACCACCTAAGAA | |
| | GCCCAAATCTCCCAAAGCTCCAGGAACTGGCAGAGGCCGG | |
| | GGACGCCCCAAAGGGAGCGGCACCACGAGACCCAAGGCGG | |
| | CCACGTCAGAGGGTGTGCAGGTGAAAAGGGTCCTGGAGAA | |
| | AAGTCCTGGGAAGCTCCTTGTCAAGATGCCTTTTCAAACT | |
| | TCGCCAGGGGGCAAGGCTGAGGGGGGTGGGGCCACCACAT | |
| | CCACCCAGGTCATGGTGATCAAACGCCCCGGCAGGAAGCG | |
| | AAAAGCTGAGGCCGACCCTCAGGCCATTCCCAAGAAACGG | |
| | GGCCGAAAGCCGGGGAGTGTGGTGGCAGCCGCTGCCGCCG | |
| | AGGCCAAAAAGAAAGCCGTGAAGGAGTCTTCTATCCGATC | |
| | TGTGCAGGAGACCGTACTCCCCATCAAGAAGCGCAAGACC | |
| | CGGGAGACGGTCAGCATCGAGGTCAAGGAAGTGGTGAAGC | |
| | CCCTGCTGGTGTCCACCCTCGGTGAGAAGAGCGGGAAAGG | |
| | ACTGAAGACCTGTAAGAGCCCTGGGCGGAAAAGCAAGGAG | |
| | AGCAGCCCCAAGGGGCGCAGCAGCAGCGCCTCCTCACCCC | |
| | CCAAGAAGGAGCACCACCACCATCACCACCACTCAGAGTC | |
| | CCCAAAGGCCCCCGTGCCACTGCTCCCACCCCTGCCCCCA | |
| | CCTCCACCTGAGCCCGAGAGCTCCGAGGACCCCACCAGCC | |
| | CCCCTGAGCCCCAGGACTTGAGCAGCAGCGTCTGCAAAGA | |
| | GGAGAAGATGCCCAGAGGAGGCTCACTGGAGAGCGACGGC | |
| | TGCCCCAAGGAGCCAGCTAAGACTCAGCCCGCGGTTGCCA | |
| | CCGCCGCCACGGCCGCAGAAAAGTACAAACACCGAGGGGA | |
| | GGGAGAGCGCAAAGACATTGTTTCATCCTCCATGCCAAGG | |
| | CCAAACAGAGAGGAGCCTGTGGACAGCCGGACGCCCGTGA | |
| | CCGAGAGAGTTAGCTGA | |
| Mm Mecp2 Intron | gtgagtgggaccgccaaggccgcgggcggggacccttgct | 8 |
| 1 5′ Fragment | gggggggggtaggggcgggacgtggcgcgggaggggcccg | |
| | cggggtcgggcgacacggc | |
| Mm Mecp2 Intron | agccaggggtctcatgtgtggcactcaagctcacctatac | 9 |
| 1 3′ Fragment | tcactactgtgcagacagctgtgttctctgtaatacttac | |
| | atatttgtttaatacttcag | |
| Mm Mecp2 Intron | gtaagtaagagcaagtcttgtgtctatagaacaagaaggc | 10 |
| 3 5′ Fragment | caggtgggtaggaaggctaggatggaaaagctttattgca | |
| | ctgaaatggcttcccaaaaa | |
| Mm Mecp2 Intron | atgtcttagaagaaagcttgtcagcatattttctatctca | 11 |
| 3 3′ Fragment | gtctctatgttgtcaaaaagcttctgactattgtttgtgt | |
| | ctctctgtttgtttctacag | |
| Hs MECP 2 Intron | GTGAGTGGGACCGCCGTGGCCgcgggcggggacccttgcc | 12 |
| 1 5′ Fragment | ggggggcgggggtcaggggcgggacgtggcgcgggagggg | |
| | cccgcggggtCGGACGACAC | |
| Hs MECP 2 Intron | GGGGCCTTGCATGTGGTGGGGGTCCAAGCCTGCCTCTGCT | 13 |
| 1 3′ Fragment | CACTTGTTCTGCAGACTGGCATGTTCTCTGTGATACTTAC | |
| Hs MECP2 Intron | ATACTTGTTTAACACTTCAGGTAAGTAAGAGCAACTCCTA | |
| 3 5′ Fragment | TCTCTACAGGGCAGGGAGGGCAGGGACAAGGATCCCTCAT | 14 |
| | GGAGCAGGAAAATGTATGTGCCCAGGGTGGGGTCGGGGGG | |
| Hs MECP2 Intron | TTTGTCAGAGCGTTGTCACCACCATCCGCTCTGCCCTATC | 15 |
| 3 3′ Fragment | TCTGACATTGCTATGGAGAGCCTCTAATTGTTCCTTGTGT | |
| Hs MECP2 3′ UTR | CTTTCTGTTTGTCCCCACAGCTTTACACGGAGCGGATTGC | |
| Fragments + Distal | AAAGCAAACCAACAAGAATAAAGGCAGCTGTTGTCTCTTC | |
| poly A | TCGGCCAGAAGTAGCTTTGCACTTTTCTAAACTAGAATCC | 16 |
| | AGAAACTTGTTCTTCCAAAGCAGAGAGCATTATGACCAAA | |
| | TATCACCAGGACTGTTACTCAATGTGTGCCGAAAATGACT | |
| | GAAAGCACTGATATATTTAAAAACAAAAGGCAATTTATTA | |
| | AGGAAATTTGTACCATTTCAGTAAACCTGTCTGAATGTAC | |
| | CTGTATACGTTTCAAAAACAccccccccccACTGAATCCC | |
| | TGTAACCTATTTATTATATAAAGAGTTTGCCTTATAAATT | |
| | TACATAAAAATGTCCGTTTGTGTCTTTTGTTGTAAAAATC | |
| | AAGTGATTTTTTCATAAGGTTCTTTTA | |
| bGH poly A | ctgtgccttctagttgccagccatctgttgtttgcccctc | 17 |
| | ccccgtgccttccttgaccctggaaggtgccactcccact | |
| | gtcctttcctaataaaatgaggaaattgcatcgcattgtc | |
| | tgagtaggtgtcattctattctggggggtggggggggcag | |
| | gacagcaagggggaggattgggaagacaatagcaggcatg | |
| | ctgggga | |
| U7 Core Sequence | a(N. . . N)AATTTTTGGAGcaggttttctgacttcgg | 18 |
| (N . . . N) denotes | tcggaaaacccct | |
| insertion site of | | |
| ESS and U7 | | |
| targeting sequences | | |
| set forth in Table 2 | | |
| Mm U7 Promoter | aacaacataggagctgtgattggctgttttcagccaatca | 19 |
| | gcactgactcatttgcatagcctttacaagcggtcacaaa | |
| | ctcaagaaacgagcggttttaatagtcttttagaatattg | |
| | tttatcgaaccgaataaggaactgtgctttgtgattcaca | |
| | tatcagtggaggggtgtggaaatggcaccttgatctcacc | |
| | ctcatcgaaagtggagttgatgtccttccctggctcgcta | |
| | cagacgcacttccgc | |
| Mm U1a1 | TAAAGACTGTGCATCCGACTCCTACATTTATGAAAGTAAA | 20 |
| Promoter | TGCCTATTGTTAGAACAAAAAAGGCTACAGAACAAAAAAC | |
| | AAAGCGAAATACCATCTGCTTTAGGTTCAGTGGTATTTTC | |
| | CCGCTGACAGGGAGGCGGGTTTTTGGGTACAGGAAACGAG | |
| | TCACTATGGAGGCGGTACTATGTAGATGAGAATTCAGGAG | |
| | CAAACTGGGAAAAGCAACTGCTTCCAAATATTTGTGATTT | |
| | TTACAGTGTAGTTTTGGAAAAACTCTTAGCCTACCAATTC | |
| | TTCTAAGTGTTTTAAAATGTGGGAGCCAGTACACATGAAG | |
| | TTATAGAGTGTTTTAATGAGGCTTAAATATTTACCGTAAC | |
| | TATGAAATGCTACGCATATCATGCTGTTCAGGCTCCGTGG | |
| | CCACGCAACTC | |
| Hs U1-1 Promoter | aacaacataggagctgtgattggctgttttcagccaatca | 21 |
| | gcactgactcAGGGCGACTTCTATGTAGATGAGGCAGCGC | |
| | AGAGGCTGCTgcctttacaagcggtcacaaactcaagaaa | |
| | cgagcggttttaatagtcttttagaatattgtttatcgaa | |
| | ccgaataaggaactgtgctttgtgattcacatatcagtgg | |
| | aggggtgtggaaatggcaccttgatGGGAGTGCGCGAGGC | |
| | AAGTGACCGTGTGTGgagttgatgtccttccctggctcgc | |
| | tacagacgcacttccgc | |
| Modified Mm U7 | aacaacataggagctgtgattggctgttttcagccaatca | 22 |
| Promoter | gcactgactcAGGGCGACTTCTATGTAGATGAGGCAGCGC | |
| | AGAGGCTGCTgcctttacaagcggtcacaaactcaagaaa | |
| | cgagcggttttaatagtcttttagaatattgtttatcgaa | |
| | ccgaataaggaactgtgctttgtgattcacatatcagtgg | |
| | aggggtgtggaaatggcaccttgatTTACCGTAACTATGA | |
| | AATGgagttgatgtccttccctggctcgctacagacgcac | |
| | ttccgc | |
| Mm U7 3′ Region | cccaatttcactggtctacaatgaaagcaaaacagttctc | 23 |
| | ttccccgctccccggtgtgtgagaggggctttgatccttc | |
| | tctggtttcctaggaaacgcgtat | |
| Hs U1-1 3′ Region | ACTTTCTGGAGTTTCAAAAGTAGACTGTACGCTAA | 24 |
| AAV2 ITR | cctgcaggcagctgcgcgctcgctcgctcactgaggccgc | 25 |
| | ccgggcaaagcccgggcgtcgggcgacctttggtcgcccg | |
| | gcctcagtgagcgagcgagcgcgcagagagggagtggcca | |
| | actccatcactaggggttcct | |

TABLE 2
Sequences of exonic splicing silencers and U7 targeting sequences tested in the mouse U7
screen and the analogous human targeting sequences based on position relative to intron/exon
junctions.

| | | Mouse U7 Antisense | | | |
|---|---|---|---|---|---|
| | | Sequence (5′ to 3′) | SEQ | Humanized version | SEQ |
| Target | | (Included in Experiments | ID | of U7 Antisense | ID |
| Gene | ID | of Provided Examples) | NO | Sequence (5′ to 3′) | NO |
| N/A | Scramble- | GCCCCTGGCTCGCCTCCTCT | 26 | GCCCCTGGCTCGCCTCCTCT | 122 |
| | 01 | | | | |
| N/A | Scramble- | GGCGGAGGAAACCATCATAT | 27 | GGCGGAGGAAACCATCATAT | 123 |
| | 02 | | | | |
| N/A | Scramble- | GAGACAATAGACCTTAGTAA | 28 | GAGACAATAGACCTTAGTAA | 124 |
| | 03 | | | | |
| N/A | Scramble- | GGCCCACTTCGCGGTGTAAT | 29 | GGCCCACTTCGCGGTGTAAT | 125 |
| | 04 | | | | |
| Mecp2 | e1-3-01 | tcctcctcctccGCtcggcg | 30 | tcctcctcctccGCTcggcg | 126 |
| Mecp2 | e1-3-02 | AGtctctcctcctcgcctcc | 31 | AGtctctcctcctcgcctcc | 127 |
| Mecp2 | e1-3-03 | GCCTTGGCGGTCCCACTCAC | 32 | GCCACGGCGGTCCCACTCAC | 128 |
| Mecp2 | e1-3-04 | agcaagggtccccgcCCGCG | 33 | ggcaagggtccccgcccgcG | 129 |
| Mecp2 | e1-3-05 | tcctcctcctccGCtcggcgcggc | 34 | tcctcctcctccGCTcggcgcggc | 130 |
| Mecp2 | e1-3-06 | AGtctctcctcctcgcctcctcct | 35 | AGtctctcctcctcgcctcctcct | 131 |
| Mecp2 | e1-3-07 | GCCTTGGCGGTCCCACTCACAGtc | 36 | GCCACGGCGGTCCCACTCACAGtc | 132 |
| Mecp2 | e1-3-08 | agcaagggtccccgcCCGCGGCCT | 37 | ggcaagggtccccgcccgcGGCCA | 133 |
| Mecp2 | e1-3-09 | tcctcctcctccGCtcggcgcggcgg | 38 | tcctcctcctccGCTcggcgcggcgg | 134 |
| Mecp2 | e1-3-10 | AGtctctcctcctcgcctcctcctcc | 39 | AGtctctcctcctcgcctcctcctcc | 135 |
| Mecp2 | e1-3-11 | GCCTTGGCGGTCCCACTCACAGtct | 40 | GCCACGGCGGTCCCACTCACAGtct | 136 |
| | | C | | C | |
| Mecp2 | e1-3-12 | agcaagggtccccgcCCGCGGCCTTG | 41 | ggcaagggtccccgcccgcGGCCACG | 137 |
| Mecp2 | e1-3-13 | tcctcctcctccGCtcggcgcggcggcggc | 42 | tcctcctcctccGCTcggcgcggcggcggc | 138 |
| Mecp2 | e1-3-14 | AGtctctcctcctcgcctcctcctcctcct | 43 | AGtctctcctcctcgcctcctcctcctcct | 139 |
| Mecp2 | e1-3-15 | GCCTTGGCGGTCCCACTCACAGtct | 44 | GCCACGGCGGTCCCACTCACAGtct | 140 |
| | | ctcct | | ctcct | |
| Mecp2 | e1-3-16 | agcaagggtccccgcCCGCGGCCTTGGC | 45 | ggcaagggtccccgcccgcGGCCACGGCG | 141 |
| | | GG | | G | |
| Mecp2 | e2-5-01 | AGTTGAAAGTCCCCATTGAG | 46 | AGTTGAAAGCCCCCATTGAG | 142 |
| Mecp2 | e2-5-02 | CTAAAACAAAGAAAATCGTA | 47 | CTAAAACAAAGAAAATTGTA | 143 |
| Mecp2 | e2-5-03 | AGTCTGTATTTTTATGGAGC | 48 | AGTCTGTATTTTTATGGAGC | 144 |
| Mecp2 | e2-5-04 | CATCAAAGCAGGAACTGGTG | 49 | CATCAAAGCAGGAACTGGTG | 145 |
| Mecp2 | e2-5-05 | AGTTGAAAGTCCCCATTGAGCTG | 50 | AGTTGAAAGCCCCCATTGAGCTG | 146 |
| | | C | | C | |
| Mecp2 | e2-5-06 | CTAAAACAAAGAAAATCGTAAGT | 51 | CTAAAACAAAGAAAATTGTAAGT | 147 |
| | | T | | T | |
| Mecp2 | e2-5-07 | AGTCTGTATTTTTATGGAGCCTAA | 52 | AGTCTGTATTTTTATGGAGCCTAA | 148 |
| Mecp2 | e2-5-08 | CATCAAAGCAGGAACTGGTGAGT | 53 | CATCAAAGCAGGAACTGGTGAGT | 149 |
| | | C | | C | |
| Mecp2 | e2-5-09 | AGTTGAAAGTCCCCATTGAGCTG | 54 | AGTTGAAAGCCCCCATTGAGCTG | 150 |
| | | CAC | | CAC | |
| Mecp2 | e2-5-10 | CTAAAACAAAGAAAATCGTAAGT | 55 | CTAAAACAAAGAAAATTGTAAGT | 151 |
| | | TGA | | TGA | |
| Mecp2 | e2-5-11 | AGTCTGTATTTTTATGGAGCCTAA | 56 | AGTCTGTATTTTTATGGAGCCTAA | 152 |
| | | AA | | AA | |
| Mecp2 | e2-5-12 | CATCAAAGCAGGAACTGGTGAGT | 57 | CATCAAAGCAGGAACTGGTGAGT | 153 |
| | | CTG | | CTG | |
| Mecp2 | e2-5-13 | AGTTGAAAGTCCCCATTGAGCTG | 58 | AGTTGAAAGCCCCCATTGAGCTG | 154 |
| | | CACCTTT | | CACGACC | |
| Mecp2 | e2-5-14 | CTAAAACAAAGAAAATCGTAAGT | 59 | CTAAAACAAAGAAAATTGTAAGT | 155 |
| | | TGAAAGT | | TGAAAGC | |
| Mecp2 | e2-5-15 | AGTCTGTATTTTTATGGAGCCTAA | 60 | AGTCTGTATTTTTATGGAGCCTAA | 156 |
| | | AACAAA | | AACAAA | |
| Mecp2 | e2-5-16 | CATCAAAGCAGGAACTGGTGAGT | 61 | CATCAAAGCAGGAACTGGTGAGT | 157 |
| | | CTGTATT | | CTGTATT | |
| Mecp2 | e2-3-01 | TACCATGGAATCCTGTTGGA | 62 | TACCATGGAATCCTGTTGGA | 158 |
| Mecp2 | e2-3-02 | CTGAGCCCTAACATCCCAGC | 63 | CTGAGCCCTAACATCCCAGC | 159 |
| Mecp2 | e2-3-03 | GGAAGGTTACTTACCTGAGC | 64 | GGAAGGTTACTTACCTGAGC | 160 |
| Mecp2 | e2-3-04 | TACCATGGAATCCTGTTGGAGCTG | 65 | TACCATGGAATCCTGTTGGAGCTG | 161 |
| Mecp2 | e2-3-05 | CTGAGCCCTAACATCCCAGCTACC | 66 | CTGAGCCCTAACATCCCAGCTACC | 162 |
| Mecp2 | e2-3-06 | aGGAAGGTTACTTACCTGAGCCCT | 67 | aGGAAGGTTACTTACCTGAGCCCT | 163 |
| Mecp2 | e2-3-07 | TACCATGGAATCCTGTTGGAGCTG | 68 | TACCATGGAATCCTGTTGGAGCTG | 164 |
| | | GT | | GT | |
| Mecp2 | e2-3-08 | CTGAGCCCTAACATCCCAGCTACC | 69 | CTGAGCCCTAACATCCCAGCTACC | 165 |
| | | AT | | AT | |
| Mecp2 | e2-3-09 | aGGAAGGTTACTTACCTGAGCCCT | 70 | aGGAAGGTTACTTACCTGAGCCCT | 166 |
| | | AA | | AA | |
| Mecp2 | e2-3-10 | TACCATGGAATCCTGTTGGAGCTG | 71 | TACCATGGAATCCTGTTGGAGCTG | 167 |
| | | GTCTAC | | GTCTAC | |
| Mecp2 | e2-3-11 | CTGAGCCCTAACATCCCAGCTACC | 72 | CTGAGCCCTAACATCCCAGCTACC | 168 |
| | | ATGGAA | | ATGGAA | |
| Mecp2 | e2-3-12 | aaGGAAGGTTACTTACCTGAGCCC | 73 | aaGGAAGGTTACTTACCTGAGCCC | 169 |
| | | TAACAT | | TAACAT | |
| Mecp2 | e3-5-01 | GTAAGTATTACAGAGAACAC | 74 | GTAAGTATCACAGAGAACAT | 170 |
| Mecp2 | e3-5-02 | CTGAAGTATTAAACAAATAT | 75 | CTGAAGTGTTAAACAAGTAT | 171 |
| Mecp2 | e3-5-03 | GGTCTTCTGACTTTTCCTCC | 76 | GGTCTTCTGACTTTTCTTCC | 172 |
| Mecp2 | e3-5-04 | TCTGAGGCCCTGGAGATCCT | 77 | CTTGAGGCCCTGGAGGTCCT | 173 |
| Mecp2 | e3-5-05 | GTAAGTATTACAGAGAACACAGC | 78 | GTAAGTATCACAGAGAACATGCC | 174 |
| | | T | | A | |
| Mecp2 | e3-5-06 | CTGAAGTATTAAACAAATATGTA | 79 | CTGAAGTGTTAAACAAGTATGTA | 175 |
| | | A | | A | |
| Mecp2 | e3-5-07 | GGTCTTCTGACTTTTCCTCCCTGA | 80 | GGTCTTCTGACTTTTCTTCCCTGA | 176 |
| Mecp2 | e3-5-08 | TCTGAGGCCCTGGAGATCCTGGTC | 81 | CTTGAGGCCCTGGAGGTCCTGGTC | 177 |
| Mecp2 | e3-5-09 | GTAAGTATTACAGAGAACACAGC | 82 | GTAAGTATCACAGAGAACATGCC | 178 |
| | | TGT | | AGT | |
| Mecp2 | e3-5-10 | CTGAAGTATTAAACAAATATGTA | 83 | CTGAAGTGTTAAACAAGTATGTA | 179 |
| | | AGT | | AGT | |
| Mecp2 | e3-5-11 | GGTCTTCTGACTTTTCCTCCCTGA | 84 | GGTCTTCTGACTTTTCTTCCCTGA | 180 |
| | | AG | | AG | |
| Mecp2 | e3-5-12 | TCTGAGGCCCTGGAGATCCTGGTC | 85 | CTTGAGGCCCTGGAGGTCCTGGTC | 181 |
| | | TT | | TT | |
| Mecp2 | e3-5-13 | GTAAGTATTACAGAGAACACAGC | 86 | GTAAGTATCACAGAGAACATGCC | 182 |
| | | TGTCTGC | | AGTCTGC | |
| Mecp2 | e3-5-14 | CTGAAGTATTAAACAAATATGTA | 87 | CTGAAGTGTTAAACAAGTATGTA | 183 |
| | | AGTATTA | | AGTATCA | |
| Mecp2 | e3-5-15 | GGTCTTCTGACTTTTCCTCCCTGA | 88 | GGTCTTCTGACTTTTCTTCCCTGA | 184 |
| | | AGTATT | | AGTGTT | |
| Mecp2 | e3-5-16 | TCTGAGGCCCTGGAGATCCTGGTC | 89 | CTTGAGGCCCTGGAGGTCCTGGTC | 185 |
| | | TTCTGA | | TTCTGA | |
| Mecp2 | e3-3-01 | CTTTCCAGCAGATCGGCCAG | 90 | CTTCCCAGCAGAGCGGCCAG | 186 |
| Mecp2 | e3-3-02 | TTGATCAAATATACATCATA | 91 | TTGATCAAATACACATCATA | 187 |
| Mecp2 | e3-3-03 | CAAGACTTGCTCTTACTTAC | 92 | TAGGAGTTGCTCTTACTTAC | 188 |
| Mecp2 | e3-3-04 | GCCTTCTTGTTCTATAGACA | 93 | CCCTCCCTGCCCTGTAGAGA | 189 |
| Mecp2 | e3-3-05 | CTTTCCAGCAGATCGGCCAGACTT | 94 | CTTCCCAGCAGAGCGGCCAGATTT | 190 |
| Mecp2 | e3-3-06 | TTGATCAAATATACATCATACTTT | 95 | TTGATCAAATACACATCATACTTC | 191 |
| Mecp2 | e3-3-07 | CAAGACTTGCTCTTACTTACTTGA | 96 | TAGGAGTTGCTCTTACTTACTTGA | 192 |
| Mecp2 | e3-3-08 | GCCTTCTTGTTCTATAGACACAAG | 97 | CCCTCCCTGCCCTGTAGAGATAGG | 193 |
| Mecp2 | e3-3-09 | CTTTCCAGCAGATCGGCCAGACTT | 98 | CTTCCCAGCAGAGCGGCCAGATTT | 194 |
| | | CC | | CC | |
| Mecp2 | e3-3-10 | TTGATCAAATATACATCATACTTT | 99 | TTGATCAAATACACATCATACTTC | 195 |
| | | CC | | CC | |
| Mecp2 | e3-3-11 | CAAGACTTGCTCTTACTTACTTGA | 100 | TAGGAGTTGCTCTTACTTACTTGA | 196 |
| | | TC | | TC | |
| Mecp2 | e3-3-12 | GCCTTCTTGTTCTATAGACACAAG | 101 | CCCTCCCTGCCCTGTAGAGATAGG | 197 |
| | | AC | | AG | |
| Mecp2 | e3-3-13 | CTTTCCAGCAGATCGGCCAGACTT | 102 | CTTCCCAGCAGAGCGGCCAGATTT | 198 |
| | | CCTTTG | | CCTTTG | |
| Mecp2 | e3-3-14 | TTGATCAAATATACATCATACTTT | 103 | TTGATCAAATACACATCATACTTC | 199 |
| | | CCAGCA | | CCAGCA | |
| Mecp2 | e3-3-15 | CAAGACTTGCTCTTACTTACTTGA | 104 | TAGGAGTTGCTCTTACTTACTTGA | 200 |
| | | TCAAAT | | TCAAAT | |
| Mecp2 | e3-3-16 | GCCTTCTTGTTCTATAGACACAAG | 105 | CCCTCCCTGCCCTGTAGAGATAGG | 201 |
| | | ACTTGC | | AGTTGC | |
| Mecp2 | e4-5-01 | ACACAAACAATAGTCAGAAG | 106 | ACACAAGGAACAATTAGAGG | 202 |
| Mecp2 | e4-5-02 | CTGTAGAAACAAACAGAGAG | 107 | CTGTGGGGACAAACAGAAAG | 203 |
| Mecp2 | e4-5-03 | GAAAAGCTTTTCCCTGGGGA | 108 | GAAAGGCTTTTCCCTGGGGA | 204 |
| Mecp2 | e4-5-04 | AATCAATTCTACTTTAGAGC | 109 | AATCAACTCCACTTTAGAGC | 205 |
| Mecp2 | e4-5-05 | ACACAAACAATAGTCAGAAGCTT | 110 | ACACAAGGAACAATTAGAGGCTC | 206 |
| | | T | | T | |
| Mecp2 | e4-5-06 | CTGTAGAAACAAACAGAGAGACA | 111 | CTGTGGGGACAAACAGAAAGACA | 207 |
| | | C | | C | |
| Mecp2 | e4-5-07 | GAAAAGCTTTTCCCTGGGGACTGT | 112 | GAAAGGCTTTTCCCTGGGGACTGT | 208 |
| Mecp2 | e4-5-08 | AATCAATTCTACTTTAGAGCGAA | 113 | AATCAACTCCACTTTAGAGCGAA | 209 |
| | | A | | A | |
| Mecp2 | e4-5-09 | ACACAAACAATAGTCAGAAGCTT | 114 | ACACAAGGAACAATTAGAGGCTC | 210 |
| | | TTT | | TCC | |
| Mecp2 | e4-5-10 | CTGTAGAAACAAACAGAGAGACA | 115 | CTGTGGGGACAAACAGAAAGACA | 211 |
| | | CAA | | CAA | |
| Mecp2 | e4-5-11 | GAAAAGCTTTTCCCTGGGGACTGT | 116 | GAAAGGCTTTTCCCTGGGGACTGT | 212 |
| | | AG | | GG | |
| Mecp2 | e4-5-12 | AATCAATTCTACTTTAGAGCGAA | 117 | AATCAACTCCACTTTAGAGCGAA | 213 |
| | | AAG | | AGG | |
| Mecp2 | e4-5-13 | ACACAAACAATAGTCAGAAGCTT | 118 | ACACAAGGAACAATTAGAGGCTC | 214 |
| | | TTTGACA | | TCCATAG | |
| Mecp2 | e4-5-14 | CTGTAGAAACAAACAGAGAGACA | 119 | CTGTGGGGACAAACAGAAAGACA | 215 |
| | | CAAACAA | | CAAGGAA | |
| Mecp2 | e4-5-15 | GAAAAGCTTTTCCCTGGGGACTGT | 120 | GAAAGGCTTTTCCCTGGGGACTGT | 216 |
| | | AGAAAC | | GGGGAC | |
| Mecp2 | e4-5-16 | AATCAATTCTACTTTAGAGCGAA | 121 | AATCAACTCCACTTTAGAGCGAA | 217 |
| | | AAGCTTT | | AGGCTTT | |

TABLE 3
Sequences of intron/exon junction regions targeted with U7 in mouse and the homologous
regions targeted in human. Expanded regions including 100 nt upstream and downstream of
each intron/exon junction are shown, except in the case of exon 2, where the region
comprises 100 nt upstream, 100 nt downstream, and the entirety of exon 2.

| | | | SEQ |
|---|---|---|---|
| Sequence Name | Region | Sequence (5′ to 3′) | ID NO |
| MmMecp2_Exon1-3 | Exon 1/Intron 1 | gccgccgccgcgccgaGCggaggaggaggaggaggcgaggagg | 218 |
| | Junction | agagaCTGTGAGTGGGACCGCCAAGGCCGCGG | |
| | | gcggggacccttgct | |
| MmMecp2_Exon2-5 | Intron 1/Exon 2 | AAAGGTGCAGCTCAATGGGGACTTTCAACTT | 219 |
| | Junction | ACGATTTTCTTTGTTTTAGGCTCCATAAAAAT | |
| | | ACAGACTCACCAGTTCCTGCTTTGATG | |
| MmMecp2_Exon2-3 | Exon 2/Intron 2 | GTAGACCAGCTCCAACAGGATTCCATGGTAG | 220 |
| | Junction | CTGGGATGTTAGGGCTCAGGTAAGTAACCTT | |
| | | CCtt | |
| MmMecp2_Exon3-5 | Intron 2/Exon 3 | GCAGACAGCTGTGTTCTCTGTAATACTTACA | 221 |
| | Junction | TATTTGTTTAATACTTCAGGGAGGAAAAGTC | |
| | | AGAAGACCAGGATCTCCAGGGCCTCAGA | |
| MmMecp2_Exon3-3 | Exon 3/Intron 3 | CAAAGGAAGTCTGGCCGATCTGCTGGAAAG | 222 |
| | Junction | TATGATGTATATTTGATCAAGTAAGTAAGAG | |
| | | CAAGTCTTGTGTCTATAGAACAAGAAGGC | |
| MmMecp2_Exon4-5 | Intron 3/Exon 4 | TGTCAAAAAGCTTCTGACTATTGTTTGTGTCT | 223 |
| | Junction | CTCTGTTTGTTTCTACAGTCCCCAGGGAAAA | |
| | | GCTTTTCGCTCTAAAGTAGAATTGATT | |
| HsMecp2_Exon1-3 | Exon 1/Intron 1 | gccgccgccgcgccgAGCggaggaggaggaggaggcgaggagg | 224 |
| | Junction | agagaCTGTGAGTGGGACCGCCGTGGCCggggc | |
| | | ggggacccttgcc | |
| HsMecp2_Exon2-5 | Intron 1/Exon 2 | GGTCGTGCAGCTCAATGGGGGCTTTCAACTT | 225 |
| | Junction | ACAATTTTCTTTGTTTTAGGCTCCATAAAAAT | |
| | | ACAGACTCACCAGTTCCTGCTTTGATG | |
| HsMecp2_Exon2-3 | Exon 2/Intron 2 | GTAGACCAGCTCCAACAGGATTCCATGGTAG | 226 |
| | Junction | CTGGGATGTTAGGGCTCAGGTAAGTAACCTT | |
| | | CCtt | |
| HsMecp2_Exon3-5 | Intron 2/Exon 3 | GCAGACTGGCATGTTCTCTGTGATACTTACA | 227 |
| | Junction | TACTTGTTTAACACTTCAGGGAAGAAAAGTC | |
| | | AGAAGACCAGGACCTCCAGGGCCTCAAG | |
| HsMecp2_Exon3-3 | Exon 3/Intron 3 | CAAAGGAAATCTGGCCGCTCTGCTGGGAAG | 228 |
| | Junction | TATGATGTGTATTTGATCAAGTAAGTAAGAG | |
| | | CAACTCCTATCTCTACAGGGCAGGGAGGG | |
| HsMecp2_Exon4-5 | Intron 3/Exon 4 | CTATGGAGAGCCTCTAATTGTTCCTTGTGTCT | 229 |
| | Junction | TTCTGTTTGTCCCCACAGTCCCCAGGGAAAA | |
| | | GCCTTTCGCTCTAAAGTGGAGTTGATT | |
| MmMecp2_Exon1- | Exon 1/Intron 1 | CTGTGGTAAAACCCGTCCGGAAAATGgccgccg | 230 |
| 3_Extended | Junction | ctgccgccaccgccgccgccgccgccgcgccgaGCggaggaggag | |
| | | gaggaggcgaggaggagagaCTGTGAGTGGGACCGCC | |
| | | AAGGCCGCGGgcggggacccttgctggggggcgggtagggg | |
| | | cgggacgtggcgcgggaggggcccgcggggTCGGGCGACA | |
| | | CGGC | |
| MmMecp2_Exon2 | Intron 1/Exon | ACATAGTATGTTGTCTGTTTATCTTCAGAAT | 231 |
| | 2/Intron 2 | GTTCCAAAAAGCCTTGGGAAAAGGTGCAGC | |
| | | TCAATGGGGACTTTCAACTTACGATTTTCTTT | |
| | | GTTTTAGGCTCCATAAAAATACAGACTCACC | |
| | | AGTTCCTGCTTTGATGTGACATGTGACTCCC | |
| | | CAGAATACACCTTGCTTCTGTAGACCAGCTC | |
| | | CAACAGGATTCCATGGTAGCTGGGATGTTAG | |
| | | GGCTCAGGTAAGTAACCTTCCtttttttttttttAGTAT | |
| | | ATGTCCTGGTTTGGCCATCTGTTTTTaaaaaaaaa | |
| | | aaaaaggaaaaaaaGATATACTATTCTTGGACA | |
| MmMecp2_Exon3- | Intron 2/Exon 3 | AGCCAGGGGTCTCATGTGTGGCACTCAAGCT | 232 |
| 5_Extended | Junction | CACCTATACTCACTACTGTGCAGACAGCTGT | |
| | | GTTCTCTGTAATACTTACATATTTGTTTAATA | |
| | | CTTCAGGGAGGAAAAGTCAGAAGACCAGGA | |
| | | TCTCCAGGGCCTCAGAGACAAGCCACTGAA | |
| | | GTTTAAGAAGGCGAAGAAAGACAAGAAGGA | |
| | | GGACAAAGAAGGCAAG | |
| MmMecp2_Exon3- | Exon 3/Intron 3 | CTATGTATGATGACCCCACCTTGCCTGAAGG | 233 |
| 3_Extended | Junction | TTGGACACGAAAGCTTAAACAAAGGAAGTC | |
| | | TGGCCGATCTGCTGGAAAGTATGATGTATAT | |
| | | TTGATCAAGTAAGTAAGAGCAAGTCTTGTGT | |
| | | CTATAGAACAAGAAGGCCAGGTGGGTAGGA | |
| | | AGGCTAGGATGGAAAAGCTTTATTGCACTGA | |
| | | AATGGCTTCCCAAAAA | |
| MmMecp2_Exon4- | Intron 3/Exon 4 | ATGTCTTAGAAGAAAGCTTGTCAGCATATTT | 234 |
| 5_Extended | Junction | TCTATCTCAGTCTCTATGTTGTCAAAAAGCTT | |
| | | CTGACTATTGTTTGTGTCTCTCTGTTTGTTTC | |
| | | TACAGTCCCCAGGGAAAAGCTTTTCGCTCTA | |
| | | AAGTAGAATTGATTGCATACTTTGAAAAGGT | |
| | | GGGAGACACCTCCTTGGACCCTAATGATTTT | |
| | | GACTTCACGGTA | |
| HsMecp2_Exon1- | Exon 1/Intron 1 | CTCTCGGAGAGAGGGCTGTGGTAAAAGCCG | 235 |
| 3_Extended | Junction | TCCGGAAAATGgccgccgccgccgccgccgcgccgAGC | |
| | | ggaggaggaggaggaggcgaggaggagagaCTGTGAGTGG | |
| | | GACCGCCGTGGCCgcgggcggggacccttgccggggggc | |
| | | gggggtcaggggcgggacgtggcgcgggaggggcccgcggggtC | |
| | | GGACGACAC | |
| HsMecp2_Exon2 | Intron 1/Exon | CTTTGTCTATGTGTTTATCTTCAAAATGTCCC | 236 |
| | 2/Intron 2 | AAATAGCCCTGGGAAAAAGGTCGTGCAGCT | |
| | | CAATGGGGGCTTTCAACTTACAATTTTCTTT | |
| | | GTTTTAGGCTCCATAAAAATACAGACTCACC | |
| | | AGTTCCTGCTTTGATGTGACATGTGACTCCC | |
| | | CAGAATACACCTTGCTTCTGTAGACCAGCTC | |
| | | CAACAGGATTCCATGGTAGCTGGGATGTTAG | |
| | | GGCTCAGGTAAGTAACCTTCCtttttttttttttAGTAT | |
| | | ATGTCCTGGTTTGGCCATCTGTTTTTTTTTTT | |
| | | TTTaaaaaaaaaaaaaaaaggaaaagaggaaaaaaa | |
| HsMecp2_Exon3- | Intron 2/Exon 3 | GGGGCCTTGCATGTGGTGGGGGTCCAAGCCT | 237 |
| 5_Extended | Junction | GCCTCTGCTCACTTGTTCTGCAGACTGGCAT | |
| | | GTTCTCTGTGATACTTACATACTTGTTTAACA | |
| | | CTTCAGGGAAGAAAAGTCAGAAGACCAGGA | |
| | | CCTCCAGGGCCTCAAGGACAAACCCCTCAA | |
| | | GTTTAAAAAGGTGAAGAAAGATAAGAAAGA | |
| | | AGAGAAAGAGGGCAAG | |
| HsMecp2_Exon3- | Exon 3/Intron 3 | CCATGTATGATGACCCCACCCTGCCTGAAGG | 238 |
| 3_Extended | Junction | CTGGACACGGAAGCTTAAGCAAAGGAAATC | |
| | | TGGCCGCTCTGCTGGGAAGTATGATGTGTAT | |
| | | TTGATCAAGTAAGTAAGAGCAACTCCTATCT | |
| | | CTACAGGGCAGGGAGGGCAGGGACAAGGAT | |
| | | CCCTCATGGAGCAGGAAAATGTATGTGCCCA | |
| | | GGGTGGGGTCGGGGGG | |
| HsMecp2_Exon4- | Intron 3/Exon 4 | TTTGTCAGAGCGTTGTCACCACCATCCGCTC | 239 |
| 5_Extended | Junction | TGCCCTATCTCTGACATTGCTATGGAGAGCC | |
| | | TCTAATTGTTCCTTGTGTCTTTCTGTTTGTCC | |
| | | CCACAGTCCCCAGGGAAAAGCCTTTCGCTCT | |
| | | AAAGTGGAGTTGATTGCGTACTTCGAAAAG | |
| | | GTAGGCGACACATCCCTGGACCCTAATGATT | |
| | | TTGACTTCACGGTA | |

TABLE 4
Exemplary components of the expressing system provided herein.

| Sequence Name | Sequence (5′ to 3′) | SEQ ID NO |
|---|---|---|
| ESS | ATGATAGGGACTTAGGGTGA | 240 |
| ESS | TTTGTTCCGTGGGTGGTTTA | 241 |
| ESS | TGGGGGGAGGTAGGTAGGTA | 242 |
| Sm binding site | AAUUUGUCUAG | 243 |
| Sm binding site (smOPT) | AAUUUUUGGAG | 244 |

TABLE 5
Sequences of example components of the expressing system described herein.

| Sequence | | SEQ ID |
|---|---|---|
| Name | Sequence | NO. |
| Mm | ggcctccgcgccgggttttggcgcctcccgcgggcgcccccctcctcacggcgagcgctgccacgtcagacg | 245 |
| Candidate- | aagggcgcagcgagcgtcctgatccttccgcccggacgctcaggacagcggcccgctgctcataagactcgg | |
| 02 | ccttagaaccccagtatcagcagaaggacattttaggacgggacttgggtgactctagggcactggttttct | |
| | ttccagagagcggaacaggcgaggaaaagtagtcccttctcggcgattctgcggagggatctccgtggggcg | |
| | gtgaacgccgatgattatataaggacgcgccgggtgtggcacagctagttccgtcgcagccgggatttgggt | |
| | cgcggttcttgtttgtggatcgctgtgatcgtcacttggtCGGTAAGTTTAGTCTTTTTGTCTTTTATTTCA | |
| | GGTCCCGGATCCGGTGGTGGTGCAAATCAAAGAACTGCTCCTCAGTGGATGTTGCCTTTACTTCTAGGCGCG | |
| | CGCGCTCCCTCCTCTCGGAGAGAGGGCTGTGGTAAAAGCCGTCCGGAAAATGTACCCATACGATGTTCCAGA | |
| | TTACGCTcctaggggaggcggaggcATGGCCGCCGCTGCCGCCACCGCCGCCGCCGCCGCCGCGCCGA | |
| | GCGGAGGAGGAGGAGGAGGCGAGGAGGAGAGACTGGAGGAAAAGTCAGAAGA | |
| | CCAGGATCTCCAGGGCCTCAGAGACAAGCCACTGAAGTTTAAGAAGGCGAAGA | |
| | AAGACAAGAAGGAGGACAAAGAAGGCAAGCATGAGCCACTACAACCTTCAGCC | |
| | CACCATTCTGCAGAGCCAGCAGAGGCAGGCAAAGCAGAAACATCAGAAAGCTC | |
| | AGGCTCTGCCCCAGCAGTGCCAGAAGCCTCGGCTTCCCCCAAACAGCGGCGCTC | |
| | CATTATCCGTGACCGGGGACCTATGTATGATGACCCCACCTTGCCTGAAGGTTG | |
| | GACACGAAAGCTTAAACAAAGGAAGTCTGGCCGATCTGCTGGAAAGTATGATG | |
| | TATATTTGATCAATCCCCAGGGAAAAGCTTTTCGCTCTAAAGTAGAATTGATTGC | |
| | ATACTTTGAAAAGGTGGGAGACACCTCCTTGGACCCTAATGATTTTGACTTCAC | |
| | GGTAACTGGGAGAGGGAGCCCCTCCAGGAGAGAGCAGAAACCACCTAAGAAGC | |
| | CCAAATCTCCCAAAGCTCCAGGAACTGGCAGGGGTCGGGGACGCCCCAAAGGG | |
| | AGCGGCACTGGGAGACCAAAGGCAGCAGCATCAGAAGGTGTTCAGGTGAAAAG | |
| | GGTCCTGGAGAAGAGCCCTGGGAAACTTGTTGTCAAGATGCCTTTCCAAGCATC | |
| | GCCTGGGGGTAAGGGTGAGGGAGGTGGGGCTACCACATCTGCCCAGGTCATGG | |
| | TGATCAAACGCCCTGGCAGAAAGCGAAAAGCTGAAGCTGACCCCCAGGCCATT | |
| | CCTAAGAAACGGGGTAGAAAGCCTGGGAGTGTGGTGGCAGCTGCTGCAGCTGA | |
| | GGCCAAAAAGAAAGCCGTGAAGGAGTCTTCCATACGGTCTGTGCATGAGACTGT | |
| | GCTCCCCATCAAGAAGCGCAAGACCCGGGAGACGGTCAGCATCGAGGTCAAGG | |
| | AAGTGGTGAAGCCCCTGCTGGTGTCCACCCTTGGTGAGAAAAGCGGGAAGGGA | |
| | CTGAAGACCTGCAAGAGCCCTGGGCGTAAAAGCAAGGAGAGCAGCCCCAAGGG | |
| | GCGCAGCAGCAGTGCCTCCTCCCCACCTAAGAAGGAGCACCATCATCACCACCA | |
| | TCACTCAGAGTCCACAAAGGCCCCCATGCCACTGCTCCCATCCCCACCCCCACC | |
| | TGAGCCTGAGAGCTCTGAGGACCCCATCAGCCCCCCTGAGCCTCAGGACTTGAG | |
| | CAGCAGCATCTGCAAAGAAGAGAAGATGCCCCGAGGAGGCTCACTGGAAAGCG | |
| | ATGGCTGCCCCAAGGAGCCAGCTAAGACTCAGCCTATGGTCGCCACCACTACCA | |
| | CAGTTGCAGAAAAGTACAAACACCGAGGGGAGGGAGAGCGCAAAGACATTGTT | |
| | TCATCTTCCATGCCAAGGCCAAACAGAGAGGAGCCTGTGGACAGCCGGACGCC | |
| | CGTGACCGAGAGAGTTAGCTGACTTTACACGGAGCGGATTGCAAAGCAAACCA | |
| | ACAAGAATAAAGGCAGCTGTTGTCTCTTCTCGGCCAGAAGTAGCTTTGCACTTTT | |
| | CTAAACTAGAATCCAGAAACTTGTTCTTCCAAAGCAGAGAGCATTATGACCAAA | |
| | TATCACCAGGACTGTTACTCAATGTGTGCCGAAAATGACTGAAAGCACTGATAT | |
| | ATTTAAAAACAAAAGGCAATTTATTAAGGAAATTTGTACCATTTCAGTAAACCT | |
| | GTCTGAATGTACCTGTATACGTTTCAAAAACAccccccccccACTGAATCCCTGTAAC | |
| | CTATTTATTATATAAAGAGTTTGCCTTATAAATTTACATAAAAATGTCCGTTTGT | |
| | GTCTTTTGTTGTAAAAATCAAGTGATTTTTTCATAAGGTTCTTTTAgcatgcTAAAG | |
| | ACTGTGCATCCGACTCCTACATTTATGAAAGTAAATGCCTATTGTTAGAACAAA | |
| | AAAGGCTACAGAACAAAAAACAAAGCGAAATACCATCTGCTTTAGGTTCAGTG | |
| | GTATTTTCCCGCTGACAGGGAGGCGGGTTTTTGGGTACAGGAAACGAGTCACTA | |
| | TGGAGGCGGTACTATGTAGATGAGAATTCAGGAGCAAACTGGGAAAAGCAACT | |
| | GCTTCCAAATATTTGTGATTTTTACAGTGTAGTTTTGGAAAAACTCTTAGCCTAC | |
| | CAATTCTTCTAAGTGTTTTAAAATGTGGGAGCCAGTACACATGAAGTTATAGAG | |
| | TGTTTTAATGAGGCTTAAATATTTACCGTAACTATGAAATGCTACGCATATCATG | |
| | CTGTTCAGGCTCCGTGGCCACGCAACTCaATGATAGGGACTTAGGGTGAagcaaggg | |
| | tccccgcCCGCGGCCTTGGCGGAATTTTTGGAGcaggttttctgacttcggtcggaaaacccctcccaatt | |
| | tcactgGTCTACAATGAAAgcaaaacagttctcttccccgctccccggtgtgctagggaaacgcgtatctgT | |
| | CTGAGTCGACCAAGGACACAATCGAGCTCCGATCCGTACTGTCGAGAAACTTGTATCCGT | |
| | AAAGACTGTGCATCCGACTCCTACATTTATGAAAGTAAATGCCTATTGTTAGAA | |
| | CAAAAAAGGCTACAGAACAAAAAACAAAGCGAAATACCATCTGCTTTAGGTTC | |
| | AGTGGTATTTTCCCGCTGACAGGGAGGCGGGTTTTTGGGTACAGGAAACGAGTC | |
| | ACTATGGAGGCGGTACTATGTAGATGAGAATTCAGGAGCAAACTGGGAAAAGC | |
| | AACTGCTTCCAAATATTTGTGATTTTTACAGTGTAGTTTTGGAAAAACTCTTAGC | |
| | CTACCAATTCTTCTAAGTGTTTTAAAATGTGGGAGCCAGTACACATGAAGTTAT | |
| | AGAGTGTTTTAATGAGGCTTAAATATTTACCGTAACTATGAAATGCTACGCATA | |
| | TCATGCTGTTCAGGCTCCGTGGCCACGCAACTCaATGATAGGGACTTAGGGTGAa | |
| | gcaagggtccccgcCCGCGGCCTTGGCGGAATTTTTGGAGcaggttttctgacttcggtcggaaaacccct | |
| | cccaatttcactgGTCTACAATGAAAgcaaaacagttctcttccccgctccccggtgtgctagggaaacgcg | |
| | tatctgTGAGCGTTCCCAGCACTTCAGCCAAGGAAGCTACCAATTTTTAGTTTCCGAGTGTC | |
| | ACGTTAAAGACTGTGCATCCGACTCCTACATTTATGAAAGTAAATGCCTATTGTT | |
| | AGAACAAAAAAGGCTACAGAACAAAAAACAAAGCGAAATACCATCTGCTTTAG | |
| | GTTCAGTGGTATTTTCCCGCTGACAGGGAGGCGGGTTTTTGGGTACAGGAAACG | |
| | AGTCACTATGGAGGCGGTACTATGTAGATGAGAATTCAGGAGCAAACTGGGAA | |
| | AAGCAACTGCTTCCAAATATTTGTGATTTTTACAGTGTAGTTTTGGAAAAACTCT | |
| | TAGCCTACCAATTCTTCTAAGTGTTTTAAAATGTGGGAGCCAGTACACATGAAG | |
| | TTATAGAGTGTTTTAATGAGGCTTAAATATTTACCGTAACTATGAAATGCTACGC | |
| | ATATCATGCTGTTCAGGCTCCGTGGCCACGCAACTCaATGATAGGGACTTAGGGT | |
| | GAagcaagggtccccgcCCGCGGCCTTGGCGGAATTTTTGGAGcaggttttctgacttcggtcggaaaac | |
| | ccctcccaatttcactgGTCTACAATGAAAgc | |
| Hs | ggcctccgcgccgggttttggcgcctcccgcgggcgcccccctcctcacggcgagcgctgccacgtcagacg | 246 |
| Candidate- | aagggcgcagcgagcgtcctgatccttccgcccggacgctcaggacagcggcccgctgctcataagactcgg | |
| 02-1 | ccttagaaccccagtatcagcagaaggacattttaggacgggacttgggtgactctagggcactggttttct | |
| | ttccagagagcggaacaggcgaggaaaagtagtcccttctcggcgattctgcggagggatctccgtggggcg | |
| | gtgaacgccgatgattatataaggacgcgccgggtgtggcacagctagttccgtcgcagccgggatttgggt | |
| | cgcggttcttgtttgtggatcgctgtgatcgtcacttggtCGGTAAGTTTAGTCTTTTTGTCTTTTATTTCA | |
| | GGTCCCGGATCCGGTGGTGGTGCAAATCAAAGAACTGCTCCTCAGTGGATGTTGCCTTTACTTCTAGGCGCG | |
| | CGCGCTCCCTCCTCTCGGAGAGAGGGCTGTGGTAAAAGCCGTCCGGAAAATGATGGCCGCCGCCGCCGCCGC | |
| | CGCGCCGAGCGGAGGAGGAGGAGGAGGCGAGGAGGAGAGACTGGAAGAAAAGTCAGAAG | |
| | ACCAGGACCTCCAGGGCCTCAAGGACAAACCCCTCAAGTTTAAAAAGGTGAAG | |
| | AAAGATAAGAAAGAAGAGAAAGAGGGCAAGCATGAGCCCGTGCAGCCATCAG | |
| | CCCACCACTCTGCTGAGCCCGCAGAGGCAGGCAAAGCAGAGACATCAGAAGGG | |
| | TCAGGCTCCGCCCCGGCTGTGCCGGAAGCTTCTGCCTCCCCCAAACAGCGGCGC | |
| | TCCATCATCCGTGACCGGGGACCCATGTATGATGACCCCACCCTGCCTGAAGGC | |
| | TGGACACGGAAGCTTAAGCAAAGGAAATCTGGCCGCTCTGCTGGGAAGTATGA | |
| | TGTGTATTTGATCAATCCCCAGGGAAAAGCCTTTCGCTCTAAAGTGGAGTTGATT | |
| | GCGTACTTCGAAAAGGTAGGCGACACATCCCTGGACCCTAATGATTTTGACTTC | |
| | ACGGTAACTGGGAGAGGGAGCCCCTCCCGGCGAGAGCAGAAACCACCTAAGAA | |
| | GCCCAAATCTCCCAAAGCTCCAGGAACTGGCAGAGGCCGGGGACGCCCCAAAG | |
| | GGAGCGGCACCACGAGACCCAAGGCGGCCACGTCAGAGGGTGTGCAGGTGAAA | |
| | AGGGTCCTGGAGAAAAGTCCTGGGAAGCTCCTTGTCAAGATGCCTTTTCAAACT | |
| | TCGCCAGGGGGCAAGGCTGAGGGGGGTGGGGCCACCACATCCACCCAGGTCAT | |
| | GGTGATCAAACGCCCCGGCAGGAAGCGAAAAGCTGAGGCCGACCCTCAGGCCA | |
| | TTCCCAAGAAACGGGGCCGAAAGCCGGGGAGTGTGGTGGCAGCCGCTGCCGCC | |
| | GAGGCCAAAAAGAAAGCCGTGAAGGAGTCTTCTATCCGATCTGTGCAGGAGAC | |
| | CGTACTCCCCATCAAGAAGCGCAAGACCCGGGAGACGGTCAGCATCGAGGTCA | |
| | AGGAAGTGGTGAAGCCCCTGCTGGTGTCCACCCTCGGTGAGAAGAGCGGGAAA | |
| | GGACTGAAGACCTGTAAGAGCCCTGGGCGGAAAAGCAAGGAGAGCAGCCCCAA | |
| | GGGGCGCAGCAGCAGCGCCTCCTCACCCCCCAAGAAGGAGCACCACCACCATC | |
| | ACCACCACTCAGAGTCCCCAAAGGCCCCCGTGCCACTGCTCCCACCCCTGCCCC | |
| | CACCTCCACCTGAGCCCGAGAGCTCCGAGGACCCCACCAGCCCCCCTGAGCCCC | |
| | AGGACTTGAGCAGCAGCGTCTGCAAAGAGGAGAAGATGCCCAGAGGAGGCTCA | |
| | CTGGAGAGCGACGGCTGCCCCAAGGAGCCAGCTAAGACTCAGCCCGCGGTTGC | |
| | CACCGCCGCCACGGCCGCAGAAAAGTACAAACACCGAGGGGAGGGAGAGCGC | |
| | AAAGACATTGTTTCATCCTCCATGCCAAGGCCAAACAGAGAGGAGCCTGTGGAC | |
| | AGCCGGACGCCCGTGACCGAGAGAGTTAGCTGACTTTACACGGAGCGGATTGC | |
| | AAAGCAAACCAACAAGAATAAAGGCAGCTGTTGTCTCTTCTCGGCCAGAAGTA | |
| | GCTTTGCACTTTTCTAAACTAGAATCCAGAAACTTGTTCTTCCAAAGCAGAGAG | |
| | CATTATGACCAAATATCACCAGGACTGTTACTCAATGTGTGCCGAAAATGACTG | |
| | AAAGCACTGATATATTTAAAAACAAAAGGCAATTTATTAAGGAAATTTGTACCA | |
| | TTTCAGTAAACCTGTCTGAATGTACCTGTATACGTTTCAAAAACAccccccccccACT | |
| | GAATCCCTGTAACCTATTTATTATATAAAGAGTTTGCCTTATAAATTTACATAAA | |
| | AATGTCCGTTTGTGTCTTTTGTTGTAAAAATCAAGTGATTTTTTCATAAGGTTCTT | |
| | TTAgcatgctggggaGCACGCACGCGGGATATCCTTAAAGACTGTGCATCCGACTCCT | |
| | ACATTTATGAAAGTAAATGCCTATTGTTAGAACAAAAAAGGCTACAGAACAAA | |
| | AAACAAAGCGAAATACCATCTGCTTTAGGTTCAGTGGTATTTTCCCGCTGACAG | |
| | GGAGGCGGGTTTTTGGGTACAGGAAACGAGTCACTATGGAGGCGGTACTATGTA | |
| | GATGAGAATTCAGGAGCAAACTGGGAAAAGCAACTGCTTCCAAATATTTGTGAT | |
| | TTTTACAGTGTAGTTTTGGAAAAACTCTTAGCCTACCAATTCTTCTAAGTGTTTT | |
| | AAAATGTGGGAGCCAGTACACATGAAGTTATAGAGTGTTTTAATGAGGCTTAAA | |
| | TATTTACCGTAACTATGAAATGCTACGCATATCATGCTGTTCAGGCTCCGTGGCC | |
| | ACGCAACTCAATGATAGGGACTTAGGGTGAAAACAAGTATGTAAGTATCACAG | |
| | AGAACATAATTTTTGGAGcaggttttctgacttcggtcggaaaacccctACTTTCTGGAGTTTCAAA | |
| | AGTAGACTGTACGCTAAGGGTCATATCTTTTTTTGTTTTGGTTTGTTCTGAGTCG | |
| | ACCAAGGACACAATCGAGCTCCGATCCGTACTGTCGAGAAACTTGTATCCGTAA | |
| | AGACTGTGCATCCGACTCCTACATTTATGAAAGTAAATGCCTATTGTTAGAACA | |
| | AAAAAGGCTACAGAACAAAAAACAAAGCGAAATACCATCTGCTTTAGGTTCAG | |
| | TGGTATTTTCCCGCTGACAGGGAGGCGGGTTTTTGGGTACAGGAAACGAGTCAC | |
| | TATGGAGGCGGTACTATGTAGATGAGAATTCAGGAGCAAACTGGGAAAAGCAA | |
| | CTGCTTCCAAATATTTGTGATTTTTACAGTGTAGTTTTGGAAAAACTCTTAGCCT | |
| | ACCAATTCTTCTAAGTGTTTTAAAATGTGGGAGCCAGTACACATGAAGTTATAG | |
| | AGTGTTTTAATGAGGCTTAAATATTTACCGTAACTATGAAATGCTACGCATATCA | |
| | TGCTGTTCAGGCTCCGTGGCCACGCAACTCAATGATAGGGACTTAGGGTGAAAA | |
| | CAAGTATGTAAGTATCACAGAGAACATAATTTTTGGAGcaggttttctgacttcggtcggaaaac | |
| | ccctACTTTCTGGAGTTTCAAAAGTAGACTGTACGCTAAGGGTCATATCTTTTTTTG | |
| | TTTTGGTTTGTTGAGCGTTCCCAGCACTTCAGCCAAGGAAGCTACCAATTTTTAG | |
| | TTTCCGAGTGTCACGTTAAAGACTGTGCATCCGACTCCTACATTTATGAAAGTAA | |
| | ATGCCTATTGTTAGAACAAAAAAGGCTACAGAACAAAAAACAAAGCGAAATAC | |
| | CATCTGCTTTAGGTTCAGTGGTATTTTCCCGCTGACAGGGAGGCGGGTTTTTGGG | |
| | TACAGGAAACGAGTCACTATGGAGGCGGTACTATGTAGATGAGAATTCAGGAG | |
| | CAAACTGGGAAAAGCAACTGCTTCCAAATATTTGTGATTTTTACAGTGTAGTTTT | |
| | GGAAAAACTCTTAGCCTACCAATTCTTCTAAGTGTTTTAAAATGTGGGAGCCAG | |
| | TACACATGAAGTTATAGAGTGTTTTAATGAGGCTTAAATATTTACCGTAACTATG | |
| | AAATGCTACGCATATCATGCTGTTCAGGCTCCGTGGCCACGCAACTCAATGATA | |
| | GGGACTTAGGGTGAAAACAAGTATGTAAGTATCACAGAGAACATAATTTTTGGA | |
| | GcaggttttctgacttcggtcggaaaacccctACTTTCTGGAGTTTCAAAAGTAGACTGTACGCTAA | |
| | GGGTCATATCTTTTTTTGTTTTGGTTTGTACATGGA | |
| Hs | ggcctccgcgccgggttttggcgcctcccgcgggcgcccccctcctcacggcgagcgctgccacgtcagacg | 247 |
| Candidate- | aagggcgcagcgagcgtcctgatccttccgcccggacgctcaggacagcggcccgctgctcataagactcgg | |
| 02-2 | ccttagaaccccagtatcagcagaaggacattttaggacgggacttgggtgactctagggcactggttttct | |
| | ttccagagagcggaacaggcgaggaaaagtagtcccttctcggcgattctgcggagggatctccgtggggcg | |
| | gtgaacgccgatgattatataaggacgcgccgggtgtggcacagctagttccgtcgcagccgggatttgggt | |
| | cgcggttcttgtttgtggatcgctgtgatcgtcacttggtCGGTAAGTTTAGTCTTTTTGTCTTTTATTTCA | |
| | GGTCCCGGATCCGGTGGTGGTGCAAATCAAAGAACTGCTCCTCAGTGGATGTTGCCTTTACTTCTAGGCGCG | |
| | CGCGCTCCCTCCTCTCGGAGAGAGGGCTGTGGTAAAAGCCGTCCGGAAAATGGCCGCCGCCGCCGCCGCCGC | |
| | GCCGAGCGGAGGAGGAGGAGGAGGCGAGGAGGAGAGACTGGAAGAAAAGTCAGAAGACC | |
| | AGGACCTCCAGGGCCTCAAGGACAAACCCCTCAAGTTTAAAAAGGTGAAGAAA | |
| | GATAAGAAAGAAGAGAAAGAGGGCAAGCATGAGCCCGTGCAGCCATCAGCCCA | |
| | CCACTCTGCTGAGCCCGCAGAGGCAGGCAAAGCAGAGACATCAGAAGGGTCAG | |
| | GCTCCGCCCCGGCTGTGCCGGAAGCTTCTGCCTCCCCCAAACAGCGGCGCTCCA | |
| | TCATCCGTGACCGGGGACCCATGTATGATGACCCCACCCTGCCTGAAGGCTGGA | |
| | CACGGAAGCTTAAGCAAAGGAAATCTGGCCGCTCTGCTGGGAAGTATGATGTGT | |
| | ATTTGATCAATCCCCAGGGAAAAGCCTTTCGCTCTAAAGTGGAGTTGATTGCGT | |
| | ACTTCGAAAAGGTAGGCGACACATCCCTGGACCCTAATGATTTTGACTTCACGG | |
| | TAACTGGGAGAGGGAGCCCCTCCCGGCGAGAGCAGAAACCACCTAAGAAGCCC | |
| | AAATCTCCCAAAGCTCCAGGAACTGGCAGAGGCCGGGGACGCCCCAAAGGGAG | |
| | CGGCACCACGAGACCCAAGGCGGCCACGTCAGAGGGTGTGCAGGTGAAAAGGG | |
| | TCCTGGAGAAAAGTCCTGGGAAGCTCCTTGTCAAGATGCCTTTTCAAACTTCGC | |
| | CAGGGGGCAAGGCTGAGGGGGGTGGGGCCACCACATCCACCCAGGTCATGGTG | |
| | ATCAAACGCCCCGGCAGGAAGCGAAAAGCTGAGGCCGACCCTCAGGCCATTCC | |
| | CAAGAAACGGGGCCGAAAGCCGGGGAGTGTGGTGGCAGCCGCTGCCGCCGAGG | |
| | CCAAAAAGAAAGCCGTGAAGGAGTCTTCTATCCGATCTGTGCAGGAGACCGTAC | |
| | TCCCCATCAAGAAGCGCAAGACCCGGGAGACGGTCAGCATCGAGGTCAAGGAA | |
| | GTGGTGAAGCCCCTGCTGGTGTCCACCCTCGGTGAGAAGAGCGGGAAAGGACT | |
| | GAAGACCTGTAAGAGCCCTGGGCGGAAAAGCAAGGAGAGCAGCCCCAAGGGG | |
| | CGCAGCAGCAGCGCCTCCTCACCCCCCAAGAAGGAGCACCACCACCATCACCA | |
| | CCACTCAGAGTCCCCAAAGGCCCCCGTGCCACTGCTCCCACCCCTGCCCCCACC | |
| | TCCACCTGAGCCCGAGAGCTCCGAGGACCCCACCAGCCCCCCTGAGCCCCAGGA | |
| | CTTGAGCAGCAGCGTCTGCAAAGAGGAGAAGATGCCCAGAGGAGGCTCACTGG | |
| | AGAGCGACGGCTGCCCCAAGGAGCCAGCTAAGACTCAGCCCGCGGTTGCCACC | |
| | GCCGCCACGGCCGCAGAAAAGTACAAACACCGAGGGGAGGGAGAGCGCAAAG | |
| | ACATTGTTTCATCCTCCATGCCAAGGCCAAACAGAGAGGAGCCTGTGGACAGCC | |
| | GGACGCCCGTGACCGAGAGAGTTAGCTGACTTTACACGGAGCGGATTGCAAAG | |
| | CAAACCAACAAGAATAAAGGCAGCTGTTGTCTCTTCTCGGCCAGAAGTAGCTTT | |
| | GCACTTTTCTAAACTAGAATCCAGAAACTTGTTCTTCCAAAGCAGAGAGCATTA | |
| | TGACCAAATATCACCAGGACTGTTACTCAATGTGTGCCGAAAATGACTGAAAGC | |
| | ACTGATATATTTAAAAACAAAAGGCAATTTATTAAGGAAATTTGTACCATTTCA | |
| | GTAAACCTGTCTGAATGTACCTGTATACGTTTCAAAAACAccccccccccACTGAATC | |
| | CCTGTAACCTATTTATTATATAAAGAGTTTGCCTTATAAATTTACATAAAAATGT | |
| | CCGTTTGTGTCTTTTGTTGTAAAAATCAAGTGATTTTTTCATAAGGTTCTTTTAgca | |
| | tgctggggaGCACGCACGCGGGATATCCTTAAAGACTGTGCATCCGACTCCTACATTT | |
| | ATGAAAGTAAATGCCTATTGTTAGAACAAAAAAGGCTACAGAACAAAAAACAA | |
| | AGCGAAATACCATCTGCTTTAGGTTCAGTGGTATTTTCCCGCTGACAGGGAGGC | |
| | GGGTTTTTGGGTACAGGAAACGAGTCACTATGGAGGCGGTACTATGTAGATGAG | |
| | AATTCAGGAGCAAACTGGGAAAAGCAACTGCTTCCAAATATTTGTGATTTTTAC | |
| | AGTGTAGTTTTGGAAAAACTCTTAGCCTACCAATTCTTCTAAGTGTTTTAAAATG | |
| | TGGGAGCCAGTACACATGAAGTTATAGAGTGTTTTAATGAGGCTTAAATATTTA | |
| | CCGTAACTATGAAATGCTACGCATATCATGCTGTTCAGGCTCCGTGGCCACGCA | |
| | ACTCATGGGGGGAGGTAGGTAGGTATTTCTTCCCTGAAGTGTTAAACAAAATTT | |
| | TTGGAGcaggttttctgacttcggtcggaaaacccctACTTTCTGGAGTTTCAAAAGTAGACTGTAC | |
| | GCTAAGGGTCATATCTTTTTTTGTTTTGGTTTGTTCTGAGTCGACCAAGGACACA | |
| | ATCGAGCTCCGATCCGTACTGTCGAGAAACTTGTATCCGTAAAGACTGTGCATC | |
| | CGACTCCTACATTTATGAAAGTAAATGCCTATTGTTAGAACAAAAAAGGCTACA | |
| | GAACAAAAAACAAAGCGAAATACCATCTGCTTTAGGTTCAGTGGTATTTTCCCG | |
| | CTGACAGGGAGGCGGGTTTTTGGGTACAGGAAACGAGTCACTATGGAGGCGGT | |
| | ACTATGTAGATGAGAATTCAGGAGCAAACTGGGAAAAGCAACTGCTTCCAAAT | |
| | ATTTGTGATTTTTACAGTGTAGTTTTGGAAAAACTCTTAGCCTACCAATTCTTCT | |
| | AAGTGTTTTAAAATGTGGGAGCCAGTACACATGAAGTTATAGAGTGTTTTAATG | |
| | AGGCTTAAATATTTACCGTAACTATGAAATGCTACGCATATCATGCTGTTCAGG | |
| | CTCCGTGGCCACGCAACTCATGGGGGGAGGTAGGTAGGTATTTCTTCCCTGAAG | |
| | TGTTAAACAAAATTTTTGGAGcaggttttctgacttcggtcggaaaacccctACTTTCTGGAGTTTC | |
| | AAAAGTAGACTGTACGCTAAGGGTCATATCTTTTTTTGTTTTGGTTTGTTGAGCG | |
| | TTCCCAGCACTTCAGCCAAGGAAGCTACCAATTTTTAGTTTCCGAGTGTCACGTT | |
| | AAAGACTGTGCATCCGACTCCTACATTTATGAAAGTAAATGCCTATTGTTAGAA | |
| | CAAAAAAGGCTACAGAACAAAAAACAAAGCGAAATACCATCTGCTTTAGGTTC | |
| | AGTGGTATTTTCCCGCTGACAGGGAGGCGGGTTTTTGGGTACAGGAAACGAGTC | |
| | ACTATGGAGGCGGTACTATGTAGATGAGAATTCAGGAGCAAACTGGGAAAAGC | |
| | AACTGCTTCCAAATATTTGTGATTTTTACAGTGTAGTTTTGGAAAAACTCTTAGC | |
| | CTACCAATTCTTCTAAGTGTTTTAAAATGTGGGAGCCAGTACACATGAAGTTAT | |
| | AGAGTGTTTTAATGAGGCTTAAATATTTACCGTAACTATGAAATGCTACGCATA | |
| | TCATGCTGTTCAGGCTCCGTGGCCACGCAACTCATGGGGGGAGGTAGGTAGGTA | |
| | TTTCTTCCCTGAAGTGTTAAACAAAATTTTTGGAGcaggttttctgacttcggtcggaaaacccctA | |
| | CTTTCTGGAGTTTCAAAAGTAGACTGTACGCTAAGGGTCATATCTTTTTTTGTTT | |
| | TGGTTTGTACATGGA | |
| HsMECP2 | ATGGCCGCCGCCGCCGCCGCCGCGCCGAGCGGAGGAGGAGGAGGAGGCGAGG | 248 |
| CDS | AGGAGAGACTGGAAGAAAAGTCAGAAGACCAGGACCTCCAGGGCCTCAAGGAC | |
| | AAACCCCTCAAGTTTAAAAAGGTGAAGAAAGATAAGAAAGAAGAGAAAGAGG | |
| | GCAAGCATGAGCCCGTGCAGCCATCAGCCCACCACTCTGCTGAGCCCGCAGAG | |
| | GCAGGCAAAGCAGAGACATCAGAAGGGTCAGGCTCCGCCCCGGCTGTGCCGGA | |
| | AGCTTCTGCCTCCCCCAAACAGCGGCGCTCCATCATCCGTGACCGGGGACCCAT | |
| | GTATGATGACCCCACCCTGCCTGAAGGCTGGACACGGAAGCTTAAGCAAAGGA | |
| | AATCTGGCCGCTCTGCTGGGAAGTATGATGTGTATTTGATCAATCCCCAGGGAA | |
| | AAGCCTTTCGCTCTAAAGTGGAGTTGATTGCGTACTTCGAAAAGGTAGGCGACA | |
| | CATCCCTGGACCCTAATGATTTTGACTTCACGGTAACTGGGAGAGGGAGCCCCT | |
| | CCCGGCGAGAGCAGAAACCACCTAAGAAGCCCAAATCTCCCAAAGCTCCAGGA | |
| | ACTGGCAGAGGCCGGGGACGCCCCAAAGGGAGCGGCACCACGAGACCCAAGG | |
| | CGGCCACGTCAGAGGGTGTGCAGGTGAAAAGGGTCCTGGAGAAAAGTCCTGGG | |
| | AAGCTCCTTGTCAAGATGCCTTTTCAAACTTCGCCAGGGGGCAAGGCTGAGGGG | |
| | GGTGGGGCCACCACATCCACCCAGGTCATGGTGATCAAACGCCCCGGCAGGAA | |
| | GCGAAAAGCTGAGGCCGACCCTCAGGCCATTCCCAAGAAACGGGGCCGAAAGC | |
| | CGGGGAGTGTGGTGGCAGCCGCTGCCGCCGAGGCCAAAAAGAAAGCCGTGAAG | |
| | GAGTCTTCTATCCGATCTGTGCAGGAGACCGTACTCCCCATCAAGAAGCGCAAG | |
| | ACCCGGGAGACGGTCAGCATCGAGGTCAAGGAAGTGGTGAAGCCCCTGCTGGT | |
| | GTCCACCCTCGGTGAGAAGAGCGGGAAAGGACTGAAGACCTGTAAGAGCCCTG | |
| | GGCGGAAAAGCAAGGAGAGCAGCCCCAAGGGGCGCAGCAGCAGCGCCTCCTCA | |
| | CCCCCCAAGAAGGAGCACCACCACCATCACCACCACTCAGAGTCCCCAAAGGC | |
| | CCCCGTGCCACTGCTCCCACCCCTGCCCCCACCTCCACCTGAGCCCGAGAGCTC | |
| | CGAGGACCCCACCAGCCCCCCTGAGCCCCAGGACTTGAGCAGCAGCGTCTGCA | |
| | AAGAGGAGAAGATGCCCAGAGGAGGCTCACTGGAGAGCGACGGCTGCCCCAAG | |
| | GAGCCAGCTAAGACTCAGCCCGCGGTTGCCACCGCCGCCACGGCCGCAGAAAA | |
| | GTACAAACACCGAGGGGAGGGAGAGCGCAAAGACATTGTTTCATCCTCCATGC | |
| | CAAGGCCAAACAGAGAGGAGCCTGTGGACAGCCGGACGCCCGTGACCGAGAGA | |
| | GTTAGCTGA | |
| HsU1-1 | ACTTTCTGGAGTTTCAAAAGTAGACTGTACGCTAAGGGTCATATCTTTTTTTGTT | 249 |
| Terminator | TTGGTTTGT | |

TABLE 6
Sequences of exonic splicing silencers and antisense sequences components and quantitative
assessment of Mecp2 splicing of the expressing system provided herein.

| | | SEQ | | Relative | SEQ |
|---|---|---|---|---|---|
| | | ID | | Normalized | ID |
| ID | ESS | NO. | Antisense | Luciferase | NO. |
| Scram-01 | ATGATAGGGACTTAGGG | 240 | GCACCCCATCCCCCACT | 1.029023586 | 250 |
| | TGA | | ACA | | |
| Scram-02 | TGGGGGGAGGTAGGTA | 242 | GGGATCGAATCAGAGTC | 0.973409073 | 251 |
| | GGTA | | ATGCGGAGGTGTA | | |
| Scram-03 | ATGATAGGGACTTAGGG | 240 | GACAGTGCTCTAGGATT | 0.997567341 | 252 |
| | TGA | | ATTCTGGTAGTTA | | |
| HsM2-e13- | ATGATAGGGACTTAGGG | 240 | CGGCGGTCCCACTCACA | 0.427931941 | 253 |
| 24-01 | TGA | | Gtctctc | | |
| HsM2-e13- | ATGATAGGGACTTAGGG | 240 | ccgcccgcGGCCACGGCGG | 0.429327383 | 254 |
| 24-02 | TGA | | TCCCA | | |
| HsM2-e13- | ATGATAGGGACTTAGGG | 240 | ccggcaagggtccccgcccgcGGC | 0.377507942 | 255 |
| 24-03 | TGA | | | | |
| HsM2-e13- | ATGATAGGGACTTAGGG | 240 | cGGCCACGGCGGTCCCA | 0.456879411 | 256 |
| 30-01 | TGA | | CTCACAGtctctc | | |
| HsM2-e13- | ATGATAGGGACTTAGGG | 240 | tccccgcccgcGGCCACGGCG | 0.259898929 | 257 |
| 30-02 | TGA | | GTCCCACTC | | |
| HsM2-e13- | ATGATAGGGACTTAGGG | 240 | ccggcaagggtccccgcccgcGGC | 0.732517806 | 258 |
| 30-03 | TGA | | CACGGC | | |
| HsM2-e13- | TGGGGGGAGGTAGGTA | 242 | CGGCGGTCCCACTCACA | 0.359760782 | 259 |
| E2-24-01 | GGTA | | Gtctctc | | |
| HsM2-e13- | TGGGGGGAGGTAGGTA | 242 | ccgcccgcGGCCACGGCGG | 0.196511306 | 260 |
| E2-24-02 | GGTA | | TCCCA | | |
| HsM2-e13- | TGGGGGGAGGTAGGTA | 242 | ccggcaagggtccccgcccgcGGC | 0.294398179 | 261 |
| E2-24-03 | GGTA | | | | |
| HsM2-e13- | TGGGGGGAGGTAGGTA | 242 | cGGCCACGGCGGTCCCA | 0.23882839 | 262 |
| E2-30-01 | GGTA | | CTCACAGtctctc | | |
| HsM2-e13- | TGGGGGGAGGTAGGTA | 242 | tccccgcccgcGGCCACGGCG | 0.581988791 | 263 |
| E2-30-02 | GGTA | | GTCCCACTC | | |
| HsM2-e13- | TGGGGGGAGGTAGGTA | 242 | ccggcaagggtccccgcccgcGGC | 0.887371433 | 264 |
| E2-30-03 | GGTA | | CACGGC | | |
| HsM2-e35- | ATGATAGGGACTTAGGG | 240 | AAGTGAGCAGAGGCAG | 0.741326829 | 265 |
| 24-01 | TGA | | GCTTGGAC | | |
| HsM2-e35- | ATGATAGGGACTTAGGG | 240 | GAACATGCCAGTCTGCA | 0.831586669 | 266 |
| 24-02 | TGA | | GAACAAG | | |
| HsM2-e35- | ATGATAGGGACTTAGGG | 240 | CAAGTATGTAAGTATCA | 0.595553327 | 267 |
| 24-03 | TGA | | CAGAGAA | | |
| HsM2-e35- | ATGATAGGGACTTAGGG | 240 | TTTCTTCCCTGAAGTGTT | 0.772106398 | 268 |
| 24-04 | TGA | | AAACAA | | |
| HsM2-e35- | ATGATAGGGACTTAGGG | 240 | AGAACAAGTGAGCAGA | 0.721370045 | 269 |
| 30-01 | TGA | | GGCAGGCTTGGACC | | |
| HsM2-e35- | ATGATAGGGACTTAGGG | 240 | CAGAGAACATGCCAGTC | 0.436592681 | 270 |
| 30-02 | TGA | | TGCAGAACAAGTG | | |
| HsM2-e35- | ATGATAGGGACTTAGGG | 240 | AAACAAGTATGTAAGTA | 0.2188879 | 271 |
| 30-03 | TGA | | TCACAGAGAACAT | | |
| HsM2-e35- | ATGATAGGGACTTAGGG | 240 | CTTTTCTTCCCTGAAGTG | 0.434246075 | 272 |
| 30-04 | TGA | | TTAAACAAGTAT | | |
| HsM2-e35- | TGGGGGGAGGTAGGTA | 242 | AAGTGAGCAGAGGCAG | 0.643692322 | 273 |
| E2-24-01 | GGTA | | GCTTGGAC | | |
| HsM2-e35- | TGGGGGGAGGTAGGTA | 242 | GAACATGCCAGTCTGCA | 0.607110712 | 274 |
| E2-24-02 | GGTA | | GAACAAG | | |
| HsM2-e35- | TGGGGGGAGGTAGGTA | 242 | CAAGTATGTAAGTATCA | 0.562209476 | 275 |
| E2-24-03 | GGTA | | CAGAGAA | | |
| HsM2-e35- | TGGGGGGAGGTAGGTA | 242 | TTTCTTCCCTGAAGTGTT | 0.317275799 | 276 |
| E2-24-04 | GGTA | | AAACAA | | |
| HsM2-e35- | TGGGGGGAGGTAGGTA | 242 | AGAACAAGTGAGCAGA | 1.315210216 | 277 |
| E2-30-01 | GGTA | | GGCAGGCTTGGACC | | |
| HsM2-e35- | TGGGGGGAGGTAGGTA | 242 | CAGAGAACATGCCAGTC | 1.333693861 | 278 |
| E2-30-02 | GGTA | | TGCAGAACAAGTG | | |
| HsM2-e35- | TGGGGGGAGGTAGGTA | 242 | AAACAAGTATGTAAGTA | 0.423330542 | 279 |
| E2-30-03 | GGTA | | TCACAGAGAACAT | | |
| HsM2-e35- | TGGGGGGAGGTAGGTA | 242 | CTTTTCTTCCCTGAAGTG | 0.285016635 | 280 |
| E2-30-04 | GGTA | | TTAAACAAGTAT | | |

TABLE 7
Sequences of exonic splicing silencers and antisense sequences components and
quantitative assessment of Mecp2 splicing of the expressing system provided
herein.

| | | SEQ | | Relative | SEQ |
|---|---|---|---|---|---|
| | | ID | | Normalized | ID |
| ID | ESS | NO: | Antisense | Luciferase | NO: |
| Scram-01 | ATGATAGGGACTTAGGG | 240 | GCACCCCATCCCCCACT | 0.892754966 | 281 |
| | TGA | | ACA | | |
| Scram-02 | TGGGGGGAGGTAGGTA | 242 | GGGATCGAATCAGAGTC | 0.996521948 | 282 |
| | GGTA | | ATGCGGAGGTGTA | | |
| Scram-03 | ATGATAGGGACTTAGGG | 240 | GACAGTGCTCTAGGATT | 1.110723085 | 283 |
| | TGA | | ATTCTGGTAGTTA | | |
| HsM2-e33- | ATGATAGGGACTTAGGG | 240 | AGTTGCTCTTACTTACTT | 0.675321951 | 284 |
| 24-01 | TGA | | GATCAA | | |
| HsM2-e33- | ATGATAGGGACTTAGGG | 240 | TCCCTGCCCTGTAGAGA | 0.74024895 | 285 |
| 24-02 | TGA | | TAGGAGT | | |
| HsM2-e33- | ATGATAGGGACTTAGGG | 240 | GAGGGATCCTTGTCCCT | 0.708705245 | 286 |
| 24-03 | TGA | | GCCCTCC | | |
| HsM2-e33- | ATGATAGGGACTTAGGG | 240 | ACATACATTTTCCTGCT | 0.762571758 | 287 |
| 24-04 | TGA | | CCATGAG | | |
| HsM2-e33- | ATGATAGGGACTTAGGG | 240 | TAGGAGTTGCTCTTACT | 0.556829995 | 288 |
| 30-01 | TGA | | TACTTGATCAAAT | | |
| HsM2-e33- | ATGATAGGGACTTAGGG | 240 | CCCTCCCTGCCCTGTAG | 0.74488039 | 289 |
| 30-02 | TGA | | AGATAGGAGTTGC | | |
| HsM2-e33- | ATGATAGGGACTTAGGG | 240 | ATGAGGGATCCTTGTCC | 1.243665222 | 290 |
| 30-03 | TGA | | CTGCCCTCCCTGC | | |
| HsM2-e33- | ATGATAGGGACTTAGGG | 240 | CACATACATTTTCCTGC | 0.765416272 | 291 |
| 30-04 | TGA | | TCCATGAGGGATC | | |
| HsM2-e45- | ATGATAGGGACTTAGGG | 240 | TGGTGGTGACAACGCTC | 0.504345012 | 292 |
| 24-01 | TGA | | TGACAAA | | |
| HsM2-e45- | ATGATAGGGACTTAGGG | 240 | TCAGAGATAGGGCAGA | 0.75525366 | 293 |
| 24-02 | TGA | | GCGGATGG | | |
| HsM2-e45- | ATGATAGGGACTTAGGG | 240 | TAGAGGCTCTCCATAGC | 0.732920737 | 294 |
| 24-03 | TGA | | AATGTCA | | |
| HsM2-e45- | ATGATAGGGACTTAGGG | 240 | CAGAAAGACACAAGGA | 0.455379762 | 295 |
| 24-04 | TGA | | ACAATTAG | | |
| HsM2-e45- | ATGATAGGGACTTAGGG | 240 | CCTGGGGACTGTGGGGA | 0.728080583 | 296 |
| 24-05 | TGA | | CAAACAG | | |
| HsM2-e45- | ATGATAGGGACTTAGGG | 240 | CAATGTCAGAGATAGGG | 0.849347861 | 297 |
| 30-01 | TGA | | CAGAGCGGATGGT | | |
| HsM2-e45- | ATGATAGGGACTTAGGG | 240 | CAATTAGAGGCTCTCCA | 0.869424079 | 298 |
| 30-02 | TGA | | TAGCAATGTCAGA | | |
| HsM2-e45- | ATGATAGGGACTTAGGG | 240 | AAACAGAAAGACACAA | 0.590523269 | 299 |
| 30-03 | TGA | | GGAACAATTAGAGG | | |
| HsM2-e45- | ATGATAGGGACTTAGGG | 240 | TCCCTGGGGACTGTGGG | 0.613800776 | 300 |
| 30-04 | TGA | | GACAAACAGAAAG | | |
| HsM2-e33- | TGGGGGGAGGTAGGTA | 242 | AGTTGCTCTTACTTACTT | 0.938237731 | 301 |
| E2-24-01 | GGTA | | GATCAA | | |
| HsM2-e33- | TGGGGGGAGGTAGGTA | 242 | TCCCTGCCCTGTAGAGA | 0.578434006 | 302 |
| E2-24-02 | GGTA | | TAGGAGT | | |
| HsM2-e33- | TGGGGGGAGGTAGGTA | 242 | GAGGGATCCTTGTCCCT | 0.939000079 | 303 |
| E2-24-03 | GGTA | | GCCCTCC | | |
| HsM2-e33- | TGGGGGGAGGTAGGTA | 242 | ACATACATTTTCCTGCT | 0.861738519 | 304 |
| E2-24-04 | GGTA | | CCATGAG | | |
| HsM2-e33- | TGGGGGGAGGTAGGTA | 242 | TAGGAGTTGCTCTTACT | 0.800587966 | 305 |
| E2-30-01 | GGTA | | TACTTGATCAAAT | | |
| HsM2-e33- | TGGGGGGAGGTAGGTA | 242 | CCCTCCCTGCCCTGTAG | 0.60121923 | 306 |
| E2-30-02 | GGTA | | AGATAGGAGTTGC | | |
| HsM2-e33- | TGGGGGGAGGTAGGTA | 242 | ATGAGGGATCCTTGTCC | 0.836164721 | 307 |
| E2-30-03 | GGTA | | CTGCCCTCCCTGC | | |
| HsM2-e33- | TGGGGGGAGGTAGGTA | 242 | CACATACATTTTCCTGC | 0.988427358 | 308 |
| E2-30-04 | GGTA | | TCCATGAGGGATC | | |
| HsM2-e45- | TGGGGGGAGGTAGGTA | 242 | TGGTGGTGACAACGCTC | 0.868080282 | 309 |
| E2-24-01 | GGTA | | TGACAAA | | |
| HsM2-e45- | TGGGGGGAGGTAGGTA | 242 | TCAGAGATAGGGCAGA | 0.839606614 | 310 |
| E2-24-02 | GGTA | | GCGGATGG | | |
| HsM2-e45- | TGGGGGGAGGTAGGTA | 242 | TAGAGGCTCTCCATAGC | 0.800816658 | 311 |
| E2-24-03 | GGTA | | AATGTCA | | |
| HsM2-e45- | TGGGGGGAGGTAGGTA | 242 | CAGAAAGACACAAGGA | 0.783169354 | 312 |
| E2-24-04 | GGTA | | ACAATTAG | | |
| HsM2-e45- | TGGGGGGAGGTAGGTA | 242 | CCTGGGGACTGTGGGGA | 0.951355203 | 313 |
| E2-24-05 | GGTA | | CAAACAG | | |
| HsM2-e45- | TGGGGGGAGGTAGGTA | 242 | CAATGTCAGAGATAGGG | 0.784817915 | 314 |
| E2-30-01 | GGTA | | CAGAGCGGATGGT | | |
| HsM2-e45- | TGGGGGGAGGTAGGTA | 242 | CAATTAGAGGCTCTCCA | 0.951770201 | 315 |
| E2-30-02 | GGTA | | TAGCAATGTCAGA | | |
| HsM2-e45- | TGGGGGGAGGTAGGTA | 242 | AAACAGAAAGACACAA | 1.150321876 | 316 |
| E2-30-03 | GGTA | | GGAACAATTAGAGG | | |
| HsM2-e45- | TGGGGGGAGGTAGGTA | 242 | TCCCTGGGGACTGTGGG | 1.06876008 | 317 |
| E2-30-04 | GGTA | | GACAAACAGAAAG | | |

TABLE 8
Example promoter sequences

| Sequence | | SEQ ID |
|---|---|---|
| Name | Sequence | NO: |
| hRNU1-1- | TTTCTTTTGTAATCCGAAACATTCTAGTCTGCGAATTAAAAGCCATTATTTGAA | 318 |
| Promoter | GAAGGATGCCCCGGCTCCATCTGGCCACCGAAAGATTGCTCCTTAACACAGG | |
| | CTAAGGACCAGCTTCTTTGGGAGAGAACAGACGCAGGGGGGGGAGGGAAAA | |
| | AGGGAGAGGCAGACGTCACTTCCTCTTGGCGACTCTGGCAGCAGATTGGTCG | |
| | GTTGAGTGGCAGAAAGGCAGACGGGGACTGGGCAAGGCACTGTCGGTGACAT | |
| | CACGGACAGGGCGACTTCTATGTAGATGAGGCAGCGCAGAGGCTGCTGCTTC | |
| | GCCACTTGCTGCTTCGCCACGAAGGGAGTTCCCGTGCCCTGGGAGCGGGTTCA | |
| | GGACCGCTGATCGGAAGTGAGAATCCCAGCTGTGTGTCAGGGCTGGAAAGGG | |
| | CTCGGGAGTGCGCGGGGCAAGTGACCGTGTGTGTAAAGAGTGAGGCGTATGA | |
| | GGCTGTGTCGGGGCAGAGGCACAACGTTTC | |
| hRNU1-2- | GTTTCTTCTGTAATCCGAAACATTCTAGTCTGCGAATTAAAAGCCATTATTTGA | 319 |
| Promoter | AGAAGGATGCCCCGGCTCCATCTGGCCACCGAAAGGTTGCTCCTTAACACAG | |
| | GCTAAGGACCAGCTTTTTTGGGAGAGAACAGACGCAGGGGGGGGAGGGAAA | |
| | AAGGGAGAGGCAGACGTCACTTCCTCTTGGCGGCTCTGGCAGCAGATTGGTC | |
| | AGTTGAGTGGCAGAAAAGCAGACGGGGACTGGGCAAGGCACTGTCGGTGAC | |
| | ATCACGGACAGGGCGACTTCTATGTAGATGAGGCAGCGCAGAGGCTGCTGCT | |
| | TCGCCACTTGCTGCTTCGCCACGAAGGAGTTCCCCTGCCCTGGGAGCGGGTTC | |
| | AGGACCGCGGATCGGAAGAGAGAATCCCAGCTGTGTGTCAGGGCTGGAAAGG | |
| | GCTCGGGAGTGCGCGGGGCAAGTGACCGTGTGTGTAAAGAGTGAGGCGTATG | |
| | AGGCTGTGTCGGGGCAGAGCCCGAAGATCTC | |
| hRNU1-3- | GTTTCTTTTGTAATCCGAAACATTCTAGTCTGCGAATTAAAAGCCATTATTTGA | 320 |
| Promoter | AGAAGGATGCCCCGGCTCCATCTGGCCACCGAAAGGTTGCTCCTTAACACAG | |
| | GCTAAGGACCAGCTTCTTTGGGAGAGAACAGACGCAGGGGGGGGAGGGAAA | |
| | AAGGGAGAGGCAGACGTCACTTCCCCTTGGCGGCTCTGGCAGCAGATTGGTC | |
| | GGTTGAGTGGCAGAAAGGCAGACGGGGACTGGGCAAGGCACTGTCGGTGAC | |
| | ATCACGGACAGGGCGACTTCTATGTAGATGAGGCAGCGCAGAGGCTGCTGCT | |
| | TCGCCACTTGCTGCTTCGCCACGAAGGAGTTCCCGTGCCCTGGGAGCGGGTTC | |
| | AGGACCGCGGATCGGAAGTGAGAATCCCAGCTGTGTGTCAGGGCTGGAAAGG | |
| | GCTCGGGAGTGCGCGGGGCAAGTGACCGTGTGTGTAAAGAGTGAGGCGTATG | |
| | AGGCTGTGTCGGGGCAGAGCCCGAAGATCTC | |
| hRNU2-1- | TCTCCCCGCCCGCGGGCGGCGAAGTAAAGGCCCAGCGCAGCCCGCGCTCCTG | 321 |
| Promoter | CCCTGGAGCCTCGTCTTTCTCCAGGAAAACGTGGACCGCTCTCCGCCGACAGG | |
| | TCTCTTCCACAGACCCCTGTCGCCTTCGCCCCCGGTCTCTTCCGGTTCTGTCTT | |
| | TTCGCTGGCTCGATACGAACAAGGAAGTCGCCCCCAGCGGAGCCCCGGCTCC | |
| | CCCAGGCAGAGGCGGCCCCGGGGGCGGAGTCAACGGCGGAGGCCACGCCCTC | |
| | TGTGAAAGGGCGGGGCATGCAAATTCGAAATGAAAGCCCGGGAACGCCGGA | |
| | AGAAGCACGGGTGTAAGATTTCCCTTTTCAAAGGCAGAGAATAAGAAATCAG | |
| | CCCGAGAGTGTAAGGGCGTCAATAGCGCTGTGGACGAGACAGAGGGAATGG | |
| | GGCAAGGAGCGAGGCTGGGGCTCTCACCGCGACTTGAATGTGGATGAGAGTG | |
| | GGACGGTGACGGCGGGCGCGAAGGCGAGCGC | |
| hU3-Promoter | CCAACAAGAGAGACCCTCATCTCTACAAAATATTTTTTAAAATAACTGGGCCT | 322 |
| | CATGGTGCATTCCTGTAATCTGAGCTAGTTGGGAGGATAGCTAGAGCCCAGG | |
| | AGATCGAGGCTGCAGAGAACCGTGATCACACGACTGCACTCCAGCCTGGGTG | |
| | ACAGAGCAAGACCCTGTCTCAATAGATAGATAGATAGATAGATAGATAGATA | |
| | GATAGATAGATAGATAGATAAAAACAGAGAGGTGAAAAATATGGTTTTAAGG | |
| | GTTCTGTTTAAGAATACTCTGTTCCAACATGGAGTTTGTCAACTCTAAAGTGG | |
| | TAAAAAACAAGACAGATTGTAGACATTTTCATAAAAGGCAGGATTATTTGGT | |
| | GTCACCAAATTAAATATGTTAATGGTCACAGCGGAGGCCGACTCAATCAGCCT | |
| | TTTATTCTGCCAGGCTATTTGATACTGCTGAGGGCTTACCTATTTCCTGAGGAG | |
| | AAAAATAGTCATTAAAATGACCTTATA | |
| hRNU4-1- | GGGCATGAAAGATAAAGTTCTGGAGGTAAGTAGTGATGATGGTTACACAACA | 323 |
| Promoter | ATGTGAACATATTCTATGCCAGTGAATTTACACTTAAGGTTAAAATGGTAAAT | |
| | TTTATGTATACTTTGGCATAATTTAAAAAAAAGTTTGAAAAAGAAAAAAGAC | |
| | AAAACAATAATCGTCTTTGGAGAAGTCTGTTACACTGAATCTGGAGCCAAGTG | |
| | AACAGAACCCTGATTTTGATCCCTTTTCTCTCAAAAGCCCTTCGCAGTCTCTGA | |
| | ATTAAGTCTATTAGCATGTTCCTCCCATAGTGCTTTGCTTCATATCAACAAAAA | |
| | CCTAGCTAAGTGAAATCAGCAACGATATGCAGAAACCACCTACGCAGGTCAC | |
| | AAACATCTTTCTATGATTGTATAATTTTCAAGCAAGCAATAAGTGAAGATTTT | |
| | TCCATAGGCCCTAAACTCACCTTTGCGAAATAGGAAGCTGGTTTATTGGGAGT | |
| | GATGAGCAGGGGGCGTAACAAATT | |
| hRNU5A-1- | GAGACAGGGCTTAAAAGTCCTGGGCAATTAGCTGGGCACAGTGTCGTGCGCC | 324 |
| Promoter | TGTAATCCCACCTAGTTGGGAGGCTGAGGCAGAAGAATCTCTTGAACCCGAG | |
| | GTAGAGGTTGCAGTGAGCCGAGATCGCGCCACTGCACTCCAGCCTGGATGAC | |
| | AGAGTGAGACCCTGTCTCAAAAAAAAAAAAAGTCCTGGGCAGCAAGGCCTCC | |
| | ACTTCACCCCCTAAAGGTTGCCCCAAGAGCACCGTGTGACTGCTAAGGTATTT | |
| | CCGGAGTCTAAAGACGATTATTCAGGTCTCATTTGCATACCCATAATACACTG | |
| | CAAACAGTATTTTTTTCGGAAAAACATTTATATATTGCTTGACATTTTTAAGTA | |
| | TGAGAATTTTGCATGCAGAATTTTTTTGTATAAACTTTCTCAGGTAGTAACCCT | |
| | TGGGATTAGTAGACACCATCAGTGTACTAGGAATTGCAGTTACCCGAAAATTG | |
| | AGTTACAGAAGTAACTGGT | |
| hRNU6-1- | CGACTCGCAACCTTTTCGGGGTCCCGAGTCCAACACCCGTGGGAATCCCATGG | 325 |
| Promoter | GCACCATGGCCCCTCGCTCCAAAAATGCTTTCGCGTCGCGCAGACACTGCTCG | |
| | GTAGTTTCGGGGATCAGCGTTTGAGTAAGAGCCCGCGTCTGAACCCTCCGCGC | |
| | CGCCCCGGCCCCAGTGGAAAGACGCGCAGGCAAAACGCACCACGTGACGGA | |
| | GCGTGACCGCGCGCCGAGCGCGCGCCAAGGTCGGGCAGGAAGAGGGCCTATT | |
| | TCCCATGATTCCTTCATATTTGCATATACGATACAAGGCTGTTAGAGAGATAA | |
| | TTAGAATTAATTTGACTGTAAACACAAAGATATTAGTACAAAATACGTGACGT | |
| | AGAAAGTAATAATTTCTTGGGTAGTTTGCAGTTTTAAAATTATGTTTTAAAAT | |
| | GGACTATCATATGCTTACCGTAACTTGAAAGTATTTCGATTTCTTGGCTTTATA | |
| | TATCTTGTGGAAAGGACGAAACACC | |
| RNU7-1- | CAAAGCCGGTTATGAAGCGGGGGTGGGGTGGGCTAGTTTTAATAGGTCCAGG | 326 |
| Promoter | CGATTAGTACCTGGCATCCTTAACCACCTACAGTTTGAGAAGGGAGTGGGTGA | |
| | TAAAAGCCTGGAAGGGAAGGGAAATCGGGCCGGGCATTAGGCTCCATCGCTC | |
| | ATCAATAGACAAGGCCTTTAGGAAACTGCGACAACGGCTTTTGCTCTGGGCCT | |
| | TTACTGCCGAATCCAGGTCTCCGGGCTTAACAACAACGAAGGGGCTGTGACT | |
| | GGCTGCTTTCTCAACCAATCAGCACCGAACTCATTTGCATGGGCTGAGAACAA | |
| | ATGTTCGCGAACTCTAGAAATGAATGACTTAAGTAAGTTCCTTAGAATATTAT | |
| | TTTTCCTACTGAAAGTTACCACATGCGTCGTTGTTTATACAGTAATAGGAACA | |
| | AGAAAAAAGTCACCTAAGCTCACCCTCATCAATTGTGGAGTTCCTTTATATCC | |
| | CATCTTCTCTCCAAACACATACGCAG | |
| hRNU11- | CACGCCTGCCTGGGCTGCTCCAAGGTGGTGGCTAGGGGTGGCACAAGTTACA | 327 |
| Promoter | CATTTTCAATTTGGATAGGTTGTGATTCTTGTCTTCCCCAGTCAGATACCTACA | |
| | GCCGCTGAGAGCCGGTATTCCAACGCCATGAGACGCCTTCTGTCGTGAGCAGT | |
| | TGACAAGAAAGACCCAGCACTTACTGCAACCGGAAGGAGCGGTTCCTCCCTC | |
| | GCTCGCGCAGCCGCTCTTCCCCGCCACTCCCTCGGTGCCCGCCAGCACATTCC | |
| | CAGCAAGCCCTGAGTATATTTGCATATCAACTCACTACATTTTTTTCTTCTAAC | |
| | TAAAAAATCGAAAGGACAAATTCCAGATTCTCCTTGTGAAGTCTTCCTTTCAG | |
| | TTCAGAAGAAATGGAATTCGCTCTTCAACTTCAGGAAGTTGAAATAAAGAGTT | |
| | GCTTGGATTTTGTGTTCACCTTTACCAAAAAATGGATTTGGTAACACTGCCAC | |
| | CCTGCTTTGGTGACAGAGAAAGC | |
| hRNU12- | TCGAGCTCCACTAGGCCGAGGACCCTAAACCCCGAAGAAAGGAAAGGCCTCT | 328 |
| Promoter | CACCGTCCTTTCGCCGCCGCCCCGCGCTTCCTGATGAGTTCGTCCAGGGAGAT | |
| | GTCCGCCATCTTGCTCCGCCGAGCAAGCCGAAAGCAGTCGAGACCCCGCGAG | |
| | CCCGCCCCTTCGGCCGCTAGGGAGCGACGAACTACTTCCGGCGTCCGCGGACC | |
| | AACTCTCGCGACAGCCAGCTCAAAGCAGGCAAGAACCGGAAGGGGGGGGA | |
| | CGTTCCCCGTGAGCCTTCGCGGTGCTGGCTGCTCATCTGCATACGGAAGTTCG | |
| | GCACATTATGAATTATTTATTTTCCTCGAGGGAAAAAATTAAATGAAAAGCAA | |
| | CAAAATACATTATTAACAAGTGAGACAAACTTCAATGGAACTGGATCATGAC | |
| | CTCAACAGTCAACTACGATAGTCATCATACGCCTAATGAGAATAGAATTCATT | |
| | ACCTAGGAAATAAACTAAAAACGTCCTT | |
| hRN7SK- | GACAAGAGAAGGACTGAACTGGGGGAGTGAGGGAAGAATGCGAGAGTGAGA | 329 |
| Promoter | CTCAGAGAGTAAAGGGGAGGGAAAAAAAAGAAGAGAGAAAGGGCGAGGAA | |
| | GGGGGCGACCGCAGGAGGGAGACAGACCTGGAGTCCACTCGGAGGCCTGGA | |
| | GCCGCACAAAGCGCCAGGTCAGCGGTCCCGGCTGGGTGAGACCAGCAGGCGG | |
| | CTCTAGCGCGCGGGAGCTGGGCGAGGCTCCGGGACGACCTCACCAATGGAGA | |
| | CTGCAGTATTTAGCATGCCCCACCCATCTGCAAGGCATTCTGGATAGTGTCAA | |
| | AACAGCCGGAAATCAAGTCCGTTTATCTCAAACTTTAGCATTTTGGGAATAAA | |
| | TGATATTTGCTATGCTGGTTAAATTAGATTTTAGTTAAATTTCCTGCTGAAGCT | |
| | CTAGTACGATAAGCAACTTGACCTAAGTGTAAAGTTGAGACTTCCTTCAGGTT | |
| | TATATAGCTTGTGCGCCGCTTGGGTACCTCG | |
| mRnu1a1- | CACAAGTAATGTACTTTTAAAATTAACCTTTCTATACCGGTTTTCACAGTGCG | 330 |
| Promoter | GCTGTTTTTGTTGTTCCTCTTAGTGTTAATTCACACTAAAGACTGTGCATCCGA | |
| | CTCCTACATTTATGAAAGTAAATGCCTATTGTTAGAACAAAAAAGGCTACAGA | |
| | ACAAAAAACAAAGCGAAATACCATCTGCTTTAGGTTCAGTGGTATTTTCCCGC | |
| | TGACAGGGAGGCGGGTTTTTGGGTACAGGAAACGAGTCACTATGGAGGCGGT | |
| | ACTATGTAGATGAGAATTCAGGAGCAAACTGGGAAAAGCAACTGCTTCCAAA | |
| | TATTTGTGATTTTTACAGTGTAGTTTTGGAAAAACTCTTAGCCTACCAATTCTT | |
| | CTAAGTGTTTTAAAATGTGGGAGCCAGTACACATGAAGTTATAGAGTGTTTTA | |
| | ATGAGGCTTAAATATTTACCGTAACTATGAAATGCTACGCATATCATGCTGTT | |
| | CAGGCTCCGTGGCCACGCAACTC | |
| mRnu1b1- | AGTCCTTGCTCTTAACTACTGAACCAACTCTCCCGTCCTATAAGTAAATTTTCA | 331 |
| Promoter | GAGCACTAATCTATTCTACTTCAAGTTTCTCCTGCATCAGATCCAGGCCAGCT | |
| | GGAGCGAGCTACACGGTTACCCCCCCTCCTCCCAAAGGGGAAAATAAGGATC | |
| | AGGTGAAAATGTTTGGAAATTACAATAGTAGAATATAAAGAAAGAATATGAA | |
| | GGCGGCCATGAAGGGGTAGAAAGGTAGATGACCAGGCGTCATCCCTGATGTC | |
| | ACGGGTATGGGCGTGGTGTATGCAGATAAGCGCGCAGGGTCTGCCGGGTCCG | |
| | CGCTCCGTGCAGAAATGTGAGGGGTTTCTCCCGCGGCGAGGGAGGGAGCGCG | |
| | GATACGCGGGTGTCAGCAGAGCGCAAGAATGTCGTGTGCGACGAGGGAGACC | |
| | CGAGATCGGCCAGGGCTAAGTGACCGTGTGTTAAGAGTGTAGAGGCGACGGG | |
| | TGTGAGCCGGGGCCGGGCAGTGGGAAAGC | |
| mRnu1b6- | CGGTGGGGCTGGCTCCTGTGCTGCCCGACTCCTGTGCTCCGACTTGGAGAAGA | 332 |
| Promoter | CGGGCGTCCGTTCGTCCATACCCAAAAGAGTCGGCCACAAGGATGTTGGCAC | |
| | CTGGGGGTGGGCCACGAGCGGCCAGTTAGGGGTAAGAGACGGTGCAAGGTG | |
| | ACTTGTCCCTGGTCCTTCACCGCTCCAGCACTGCGCCAGTGAGAAAGTCCAAC | |
| | AGGCCCCGGGATCGGGCGGCGAAAGGAAGGGGCGTGATGTCACTCCTGGGTG | |
| | GCTGACGGGGGGGGGTGTATGCAGATGAGGGTCGCGCAGGGGCTGCGGCGTC | |
| | CGCAGCGGCCGGGGAAACGCGAGAGATTTCTCGCGCGGCGTGGGAGAGAAC | |
| | GCGGGTCGCCAGATGTCAGCGGAGCGCCGAGACGGCGTGTGCGACGAGGGA | |
| | GACCCGAGATCGGGGGGGGCTAAGTGACCGTGTGTTAAGAGTGCAGGGGCGG | |
| | CGGGTGTGAGCCGGGGCCGGGCGCTGGGAGAGC | |
| mRnu2-10- | GCTCCTCCTAGGAGCTAACACTGGAGCCAGGGGGATCAAAGCTGTGTAGTGA | 333 |
| Promoter | GTGTCACAAAACATCCTAGCACTAAGGAGACTGAGACGGGAACATTGTAGTA | |
| | AGTTCTAGCCTGGGCTACAGTAAGAGACGTAGTCTCAAACAAGGAAATGCGC | |
| | TATAAATAAATAAGATGGAGCTTCAATGGTCTGTGCATAACTGGGGGGTTGA | |
| | AGGAGCCTTCCAAAGGCTGGGTTCCGGCTCCGTTCAGTCGTTGACTTGAGCTG | |
| | GGCGGGGCATGCAAATAAGGACGCCGCCTGGTCTCCGCGGAACTCTGGGAAC | |
| | GAAAAGCGCAACAAAATCCTCTCGGCCCCCTTGGATTCCTCGGGCTTTCCCTG | |
| | GCGTAAAGGTGGTGGACAGGATAGAAGAGGAGTTTCGGCGTCGGGCGCGTGT | |
| | TTGCGGTCGCGCGAGTGTTGGCGACCGTGAGTTGAAGGTGTGTGTGGTGTTGA | |
| | GTGTATGGGGCGTGTGGGCGCAGTTCGGT | |
| mRnu3b1- | ACAGAATTTACTAAATAGAGAGTGCAGTCATGGAATTGCCATTTTCCCCACGT | 334 |
| Promoter | GGTTTTTCGTTTGTTCCTCACCGGAGGCCTCCGGTTTGAGCCCGAAGTCTAAA | |
| | CCGGTGCGCGTCTGCGAGCTCGCGTGCGCCTCCGCGGCCGGAGCACAGTCGC | |
| | GGGCGGGCATCAGGGCGGTGCGTGGCCGTCAGTGACGCATGACCCTTCGCAG | |
| | GCAGAAGTGGGACGGGTCAAGAGAGGACTGTGATCCGATTGGCTGTTCGGCG | |
| | ACCGTATGCTAATCGCGGTGACAGTCTGCGTGGTGTAGGGACACGGTAGCGA | |
| | CTCAAAATGCCCCGTGGACGGGGTCTGGAGAGGTGTCGGTCTGTCAGAGAAG | |
| | GTGTCCCCGCGAGGGCAGACGCGGTGTGAGATAGAGGCGCGGACAGGGATGT | |
| | CACGGGTCGTCTGTGCGTCAGTCGCCGTGACTAAGATGTGGGGGCGGCCGGT | |
| | GCAGTGCGGCGCGGTGTGGAGCGTGAGGTTT | |
| mRnu5g- | TTATCTACGTTGGTTTTACTACAGAAACTACTGTTCTCAGTAAAAGCAGAGAG | 335 |
| Promoter | AGTACAATGCCAACAGCTTTGAAATCCTCTGTGCTCTTGTGCCAATCCCACCC | |
| | TCAGAACAGGGAGTCTGTCGGTTATGTTTTCGTCCTTTACAGTTCTTCGCCGTC | |
| | TAATCCATTGGAAACCTCCTTTATTTAAAGGAAACCGTGTCCAAATTCAGTGT | |
| | ACAGAGGTGCAAACCTCCGCCCCGCTCCTTGTAGGAAGCCCACGGGAACTGG | |
| | GCCAATCGGACGGCGCTTACTCGGCTCATTTACATACCCATAACACACCGCGG | |
| | CTAATGCAAATATTTTCGTGAAAAGATCTTCCATTACTCGAAGTTTTAAAACT | |
| | AAAAACAAAATATTATTTCAAAAGATCATGAAGAAATTTCTGTCGGTAACAG | |
| | CAGTTTCAATTGATAAATCACCATCACTTATCTAGGAGGTTCTGTTACTCTAG | |
| | AAGTGAATTAAGCAGGACAGCTGT | |
| mRnu6- | GGCATGTGATTTTGTTTTGTTTAGGTTTTTTTTTAACTTTATTTGATCAACATGA | 336 |
| Promoter | AATAATACAATAAGCTATGTCTTCTAACCAGCAGGCAGCCAGCCAGGAGCCA | |
| | TGTGTAGCAGTGTTAGAAGCTGCAGCAGGCATGTGGGTAATTGTGGGGAGCT | |
| | TTTAGGAACAGCTAGGGAGCTGTGTGGAGCTGCAGAGATGCAGAGGAGCCTG | |
| | GAGGAACAGACCAGTGCTGTGGGCTGGGAGGAGCCCCCCTCACAGGTACTTG | |
| | CTCAACGGCTCCACGACCATAAGCCTTGAATAAGTTACATGGACATTTTACAA | |
| | ATGGCACAGGATTGATTATGTCTGCTGCCAGAAGCCTCATGTCGATAAGGAG | |
| | AGAGCTGGCCAAAAGGTAAAGTATGCTAGCTCCATACAGTGCAATAGCCACG | |
| | TAGTCTCACAGAGAAAGAGGCAGGCCTGCATCATATCCAAATATGGCCAAGA | |
| | ATTTTATTAAATTAAAATGCAAACAGCA | |
| mRnu7- | TATTTTTAATGAAGCAGGGCCATGGAGCCCAAGCTGGCCTTGAACTTCTCAGC | 337 |
| Promoter | TTTTGTGCTGGTATCACAGAAATGTGGCGCTATGCCCGGTCGGGCCTGATGTT | |
| | CCAGAGTCTTTGCAGTGTGGGGAACTTGTAAGGATGAGACTACAAGACATCG | |
| | GGCCACATCGCCTGCCACTACTTAAGTCCGATTCACTTCGGCTTTAGCTCCAA | |
| | GCCTTTAATCTCGCGAAGCTCTTTTTTTTTTTTTAACAACATAGGAGCTGTGAT | |
| | TGGCTGTTTTCAGCCAATCAGCACTGACTCATTTGCATAGCCTTTACAAGCGG | |
| | TCACAAACTCAAGAAACGAGCGGTTTTAATAGTCTTTTAGAATATTGTTTATC | |
| | GAACCGAATAAGGAACTGTGCTTTGTGATTCACATATCAGTGGAGGGGTGTG | |
| | GAAATGGCACCTTGATCTCACCCTCATCGAAAGTGGAGTTGATGTCCTTCCCT | |
| | GGCTCGCTACAGACGCACTTCCGC | |
| mRnu11- | GTTCTTTGCTTTTACAGACCGTCTCCTGTGTGTCCCTGTCAGAGACCTGAATTC | 338 |
| Promoter | GGTTCCTAGCACCCATATGTTAGAACATAACGTCCAAGTTGGAGGCTACAAGT | |
| | GGCACTAAATTACACATTATCAGTTTTGACAGGTTGGGGCACCGTGTAACCCT | |
| | AGTAACTTAGCGAAAGGCTCCTCGAACCCAACATCCCACCTCTAGCTGGGAG | |
| | CAGCTGTTTTGCACCAGCGCCGCCTACCTCTCCCTCCGCGTCGGCCAGAGCAT | |
| | TCCCAGCAAGCACTGCTCTGATTTGCATATTGACTCACGGACTTTCCTTTACTG | |
| | CTATACAAAAAACGACACAACAGTTCTCTTTGTGAAGAGCTTTCCCTCAGCCC | |
| | GAGAAGAATCGATAGTTTGCCTTTTAGCTTCGACAATATACAGTTGAAAGTTG | |
| | GTTAGACTTTATTTTCACCTTTACCGTAAGATGGGTGGACCGACAATGCCACT | |
| | GTGCTTCCGAACGCGCTCGAGC | |
| mRnu12- | GAAGCAGCGAGGCCCCTACGAGCCTCGAGGAAAAGGGAAAGGGCCGCTCAC | 339 |
| Promoter | CGTCCCTTGGCCGCCGTCCCGCGCTTCCGGATGAGCTCGTCCAGCGAGAGATC | |
| | GGCCATCTTGCGGCGCGCAGCTAGCGGAGAACGGAGGAGAAGCCGACTGCGG | |
| | CGAGCCCGCCCCGCGGCCGGAGTCAAGAGCGGCTTCCGGCAACCCGGACGGC | |
| | CAACTCTCGCGACAGCTCAAACGAAAAGCTCGAGACGGAAGGGGTGGAGCCT | |
| | CTCTCCCCGTGAGCCTTTGCGGTGCTCGCTTCTAATTTGCATGGAGGAGTCCG | |
| | GAGCCTTATGGGCTCTCTCTCCCCCCTCCTCCTTTTGCTAATGAGGAATTAGAA | |
| | TTATATCGATCATAAACCATTAAAAAGCATTATCAAGAGGACAAGTCTTTTAG | |
| | TCATACAGCCGATCGGACACTCACCATACTTGTAATGAGAATATAATTTCTTA | |
| | CCCCTGAGGAACGTAGACACGTTCCTC | |
| mRn7sk- | CGCTTCGGTGTGTCACTTCGGCCACAGAGGGAGACAGACGATTACGGCCCAC | 340 |
| Promoter | GGAAGGCCGCGTGAGTCTCGTGAAGCTGGAGATGCGCTGGGTCTACCTTTCGC | |
| | CGTCCGCTCCGCCTTGGCAGAGGCGCCGACACGCACCGAAGGCAGAGAACCC | |
| | ATCTCGTATGGGACCGCACCGGACACGGGCGCCTGGGCCAGGAGCAGAGCCG | |
| | GGCCGTAGAATAGACATGGCCGTCGGGGGCGGGGCTTCGGAAGGTTTAACCA | |
| | ATCCAAACTGTTGTATTTTGCATAGCCCCAAAGCATTTTGGTTAACAGTAAAA | |
| | ACATCCTAAATTTAAGTATTTTAATTTAAACTTAGAACGAAGCGAGTATAAAA | |
| | AGGATTATTTAACCCTAAAACGGATTCAGGATTTGTTATAATATCAAGTACAG | |
| | TCGGCTACATAAGGTCACCACATGTGTAAAGTTACAAAATTCTATGGCCTTAT | |
| | ATACCTACCAAGAGCCTGCTTACTCTC | |

TABLE 9
Example terminator sequences

| Sequence | | SEQ ID |
|---|---|---|
| Name | Sequence | NO: |
| hRNU1-1- | ACTTTCTGGAGTTTCAAAAACAGACTGTACGCCAAGGGTCATATCTTTTTTTGT | 341 |
| Terminator | ATTGGTTTGTGTCTTGGTTGGTGTCTTAGGTGTTAATCCTACAGTGGAGGGTTT | |
| | ATGGGAATAGGAAGTAACATGTCGCCTGCATGTCATAGGAGAAAAAGCGAGC | |
| | ATCAGCCGTATCGGCTTTGTAACACAAATTAGCTATCGTGAAGTCCGCTCAGC | |
| | TCTTCCCTTTCTACCCTGGCTGCTTTTTGCAGGGATTGGTCCGTGGTCTCCAGT | |
| | CTCTTGGGTTCTCACCCTGTGTGAAAATCTTCGTGTTTTTCCCTACCCCCCAGT | |
| | CACCTCTTACACAGCCTCTGCTTCCAAGCGCAGCCCCCACAGGAGTTTGTAGG | |
| | ATTTCTGTGCTAGCGGGGAATGTGTTCTCACCTCATAGAGCCAGGTAGAAATT | |
| | ATGCAGATGGGCAGTTCTCTGGGAAGAAAGCAGGGCCTTTGGGGCTCTCAGT | |
| | GTCCCCGTTGGGTTGTAGACA | |
| hRNU1-2- | ACTTTCTGGAGTTTCTAAAAGTAGACTGTACGCTAAGGGTCATATCTTTTTTTG | 342 |
| Terminator | TTTTGGTTTGTGTCTTGGTTGGCGTCTTAAATGTTAATCCTACAGTGGAGGGCT | |
| | GGCGAATAGGAAGTAACATGTCGCCTGCACGCCATAGGAGAAAAAGCGAGC | |
| | ATCAGCCGTATCGGCTTTGTAACACAAATTAGCTATCGTGAAGTCCGCTCAGC | |
| | TCTTCCCTTTCTACCCTGGCTGCTTTTTGCAGGGATTGGTCCGTGGTCTCCAGT | |
| | CTCTTGGGTTCTCACCCTGTGTGAAAATCTTCCTGTTTTTCCCTACCCCCCAAG | |
| | TCACCTCTTACACAGCCTCTGCTTCCAAGCGCAGCCCCCACAGGAGTTTGTAG | |
| | GATTTCTGTGCTAGCGGGGAGTGTGTTCTCACCTCATAGAGCCAGGTAGAAAC | |
| | TACGCAGATGGGTGCTGTTCTCCAGGAAGAAAGCAGGGCCTTTGGGGCTCTC | |
| | AGTGTCCCCGTTGGGTTGTAGA | |
| hRNU1-3- | ACTTTCTGGAGTTTCAAAAACAGACCGTACGCCAAGGGTCATGTCTTTTTTCG | 343 |
| Terminator | TATTGGTTTGTGTCTTAGTTGTTAATCCTACAGTGGAGGCCTGGGGAATAAGA | |
| | AGTAACATGTGGCCTGCACGCCATAGGAGAAAAAGCGAGCATCAGCCGTATC | |
| | GGCTTTGTAACACAAATTAGCTATCGTGAAGTCCGCTCAGCTCTTCCATTTCTA | |
| | CCCTGGCTGCTTTTTGCAGGGATTGGTCCGTGGTCTCCAGTCTCTTGGGTTCTC | |
| | ACCCTGTGTGAAAATCTTCGTGTTTTTCCCTACCCCCCAAGTCACCTCTTACAC | |
| | AGCCTCTGCTTCCAAGCGCAGCCCCCACAGGAGTTTGTAGGATTTCTGTGCTA | |
| | GCGGGGAGTGTGTTCTCACCTCATAGAGCCAGGTAGAAATTATGCAGATGGG | |
| | CGCTGTTCTCTGGGAAGAAAGCAGGGCCTTTGGGGCTCTCAGTGTCCCCGTTG | |
| | GGTTGTAGACATAACACTCTTA | |
| hRNU2-1- | CCTCCGGGGATACAACGTGTTTCCTAAAAGTAGAGGGAGGTAAGAGACGGTA | 344 |
| Terminator | GCACCTGCGGGGCGGCTTGCACGCCGAGTGCCTGTGACGCGCCGGCTTAACTT | |
| | AACTGCTTCCCTGAAGTACCTTGAGGTTCCTGATGTGCGGGCGGTAGACGGTA | |
| | GGCTTATGCGGCACGCTGTCGTTTCCACCGTGGCTACTGCGCTTTGGGAAGGC | |
| | CACGACCTCCTCCTTTGGGGAGGTCCTTAGGATCTCAGCTTGGCAGTCGAGTG | |
| | GGTGGCGACCTTTTAAAGGAATGGGACCCACCCGGAGTTC | |
| hU3- | TTCCCTTCTTGTAATACAAAATAATAACAACCACAGAGGACATCGCAAGTAA | 345 |
| Terminator | AATGTGTATAACAACCCCATCTAAAGAACCCTATCATCTTTCTACTATCTGTCT | |
| | ACCTAAATATGTATAGTATAAATAACCCTATATAGCACTCTAAGCAACTTAAG | |
| | TTAGGTAACTTAGAAAACTCTTTTTTTTTTTTGAGACTGAGTCTCACTCTGTCG | |
| | CACCCAGGCTGGAGTGCAGTGGCGTGATCTCGGCTCACTGCAACCTCCACCTT | |
| | CTGGGTTCAAGTGATTCTCCCATCTCAGTCTCCCAAGTAGCTGGGATTACAGG | |
| | CACGCCACCATGCCCTGCTACATTTTTTTTTTTTTAGTAGAGATGAGGTTTCAC | |
| | CACATTGGCCAGGCTGTTCTCGAACTCCTGACCTCAGGTGATCCGCCCACCTT | |
| | GGCCTCATGAAGTGCTGGGATTACAGGTGTGAGCCACTGTACCCAGCCAGAA | |
| | AACTCTTAAAAAAC | |
| hRNU4-1- | CTGAATTTTCTTGCAGTTGAACAACAGAGGCTTTTTTTGTGTGTGTGGGGGTG | 346 |
| Terminator | CTTGGTTTTGGGAGGTTGAAGAGTACTTGTTCGCAAACTCTCTAAATGAGAGA | |
| | TGGGGAAGTGTCTTACCAGCTGATTCGCGAGAGCACCTGCAAGTTTACCTGTT | |
| | TTAGTGATTTGCGTTTATGAAAGTTGGAGTTGCTGATTTTGTGATCTCTACTTT | |
| | TAAGGTAAAAGCAGGGCGGTGTTGACTTTTCTTGCCAGTTGGCTGTCCTGTTT | |
| | TCTTTCTCGCACTGTTTTCTTTTTGAGACGAAGTCTGTCGCTCAGGCTGGAGTG | |
| | CACTGGCGTGATCTCGGCTCGCTGCAAACTTTGCCGAACCCCTGTTTAGCGAT | |
| | TTCCGTTTG | |
| hRNU5A-1- | ATATGTGGTAATCCAACAATAGAAATTATTTTTAAGTTTGTGTGTTCCTTTTTC | 347 |
| Terminator | TGTTCAATGGTGCTTTTGATATTGTTGTAAAGCAGTGACTAGCAGATTCTGTGT | |
| | GGTAAAAGCACTGAACCCTGTTTAGTTCTTTTCTCCCTACCTTATTGGTAATTA | |
| | TTTCTGTGTATATATGTTCCAAAGAAATGCGGCTTGTATATTCATGCTTTTCAA | |
| | CTTTTTGTGATAGACAGTTGATACACAAGTAGTACCTCCTCCGGGATCTTCTTA | |
| | GTCAAGACTGTTTGGTAGATGCATGTTTTTTGTTACTTGTAATGTACAGTATTC | |
| | AGTTCATTTTCTTTTTGCAGCACTGTCCGGTTGAAACACAACGCGAGGATCGG | |
| | GCGTGGTGGCTCACGCCTGTAATCCCAGCACTTTGGGAGGCCGAGGCAGGAC | |
| | AATCAAGACCAATCCTGGTCATCAAGGCAGGAGAATCAAGACCATCGAGGTC | |
| | AGGAGATCAAGACCATCCT | |
| hRNU6-1- | ACATCAGGTTGTTTTTCTGTTTTTACATCAGGTTGTTTTTCTGTTTGGTTTTTTT | 348 |
| Terminator | TTTACACCACGTTTATACGCCGGTGCACGGTTTACCACTGAAAACACCTTTCA | |
| | TCTACAGGTGATATCTTTTAACACAAATAAAATGTAGTAGTCCTAGGAGACGG | |
| | AATAGAAGGAGGTGGGGCCTAGGCAGATTCATCTCTGCGGTGCATTTTGCCTC | |
| | TGGCCCTCGGGCCCTAGTTTGCGATCTTTCCCATCTTGCCTCTGCTTCCCTCTG | |
| | CACAATCCTGAGTATTGCAGTACCTTACACTTAAGGAAATTTCGTCTTGCCAA | |
| | AGTTGATCCATTTCAGCTCCGAAACATAACAGGAACTAGTGCAACGATAAAC | |
| | GTATGAGGAACTTGCAATAAGAGTGACAGAATCCTTTGAATCAAAAAAAAAA | |
| | AAAGCCACCACTTAATGAGTATCTACCATATGTCAAGGGCTTCACCAATATTT | |
| | CATTGAAATATTCCAGACCA | |
| RNU7-1- | CTTATGATGTTTGTTGCCAATGATAGATTGTTTTCACTGTGCAAAAATTATGGG | 349 |
| Terminator | TAGTTTTGGTGGTCTTGATGCAGTTGTAAGCTTGGGGTATGAAGGTTTGGGCC | |
| | ACGCCTGGGCGCTTCCGGCTGCGCCGGATGCTGTTTCCTTTCCGCTCCCAGGG | |
| | GCGTTGGGAACGGTTGTAGGACGTGGCTCTTTATTCGTGAGTTTTCCATTTACC | |
| | TCCGCTGAACCTAGAGCTTCAGACGCCCTATGGCGTCCGCCTCGACCCAACCG | |
| | GCGGCCTTGAGCGCTGAGCAAGCAAAGGGTGAGAATCGTCCTAGTCAAGGCA | |
| | TAGGCTGCTGGCCTGGGGTAGTCAAGGCATGGGCTGCTGGTCTGGGGAGGAT | |
| | GCGGGCGGAGGATGTGGGGCGACAAACCTGGCTACGTCCGCCGGGAAAATGG | |
| | GGTAGGGGACGCCGGGTACCGTCCTTCTAAGTGGGGCGCTTGCCCCCAAGAC | |
| | TTGGGATCTTATTGGGTTACCTACA | |
| hRNU11- | TCTTTACTGTTATATGTTAGGCGAAATATTACGCGTTTGGAGTAAGTGGTGCTT | 350 |
| Terminator | TTTGTAACTGAAAAGAGATTCTGTGTGTGTTTTTTTTTTTTTAGAGGCTGCATA | |
| | GTTATTTGTATGAACCGCAGGTGACCTAATTAGCAAGGTAATTCAGACCTGTA | |
| | TCCTAGCACTTAAGAAGTGGAAGTGGGATAACCCTGCGTGTTCTCGTGTGGGA | |
| | GGCTAGCTTAAGCAACAAAGCAGGAAGGCCCCACCAACAAAAGAATTTAAAA | |
| | ATTATACGTGCGTGGTGGCGCGCGTATGAGGAAATTATTTGGCAACTGTGTTA | |
| | CAGTATAGCAGCAAATGGAATAGACCTAGTCCCTGACTTTAGGAGACCTGCCT | |
| | CAGCCCCCCGAATGGCTGGGACTACAGGCGTGCGCCTCCGCGCCCAGCTAATT | |
| | TTTGCATTTTTGGTAGAGACGGGTTCACCATGTTGGCCAGGATGGTCTCCATC | |
| | TCTTCATCTGGTGATCCGCC | |
| hRNU12- | ACCTTATTCACGCCTAAAAAGTAGACTGACTGTGGGGTGGTCGTGTTTTTTGT | 351 |
| Terminator | TTCTTGTTGGTAGGTGGTGAATGCGTTTTTTTCGTTGTTTTCTCCGTTACTCAG | |
| | GCTGCCAGTTGCTTGGCAGTCTTGTCGCTGGCTGTGGACGCTCTGCACTTCATC | |
| | ACCGTCCCCAGCTGCGCTTTGAGCCGGTCTAAGGTTGCCCACAGTATTGACGT | |
| | TGGGACCCACGAAACTGGCAATTTCCATGTGTGCGGCCTTACCCAGTTCTCCC | |
| | AAACTTCTGCGTATAACCGCTTCTCTGTGCTTCAGAAAGATAAGTCCGGCCCC | |
| | GGCCTCACACAGCAGCTCAGTTCGTGACAGCGTTTAATATGTAAAATGGAGC | |
| | AAATAATTTATAGGGCGTTGGGAGAGTTAAAGAGACTAATTCATTTACGTGTT | |
| | AAATACAGGGAATGGCTTTTGCATTGATATAACCCCAGCCTCCAGCATGCGCC | |
| | AGACCTGACGTAGGTGTTCGGC | |
| hRN7SK- | TTTGACCCATTACCCATCTAAGTTAGATGCTTTTTTAAATGTTTTTTAATTTTTA | 352 |
| Terminator | AATTTTTAATTTTTTTCATTATTTATTTTTTATTTTTGAGACGGATCTCGGCTCA | |
| | CTGTAACCTCCACCTCCCGGGTTCAAGCGATTCTCCTGACTCAGCCTCCCGAG | |
| | TAGCTGGGATTACAGGCGCGCGCCACCATGCCCGGCTAATTTTTCTATTTTTA | |
| | GTAAAGACGGGGTTTCGCCATGTTGACCAGGGTGGTCTTGAACTCCTGACGTC | |
| | AGGTGATCCTCCTGCCTCGTGCTGGGATTGCAGGCGTGAGCCACCGCTCCCGG | |
| | CCCTTAGATGCTTTTTAATCAGCAAGTACGCAATGCTTTCCTGACAGGAATGT | |
| | CAAGTCCGTCAGGAAATCCACATAAGGGTGTTAAGCAGGCAAAGTACCACAT | |
| | ACAGATTAAAATCAAAAGCACTGTCAACGCTTATTTAATTTATACAGTAGACC | |
| | CTAACTTTTCTAATTTTGTG | |
| mRnu1a1- | GTTTACTTGGTTTTAAAAATAGCTTGCACTAGCGATACCGCGAATATGGTTAT | 353 |
| Terminator | TAGGTTTGTTAGGCACAGTCGTGTCTTACTATAGAAAAATAACGTAGTGTTCA | |
| | TTTTGCTGCCTGTATGTGTTAATTTGTCCTTATTGCGCCATTGTTCTTGTTAAGT | |
| | CTTCTGTAAGGAGTTGCGGGTTTCAAACTGTCAGTCTGAGAGCATAAGCTCAC | |
| | TTTTCAGTCAGTTGAATTGAATGTTACATTCGATTGGTTCTGGATATTGATATT | |
| | ACCCAAGGCTTGTCCTGTTTGTAAGTCTTTTCCTACACCTGACAATATGATTAT | |
| | GTTTACAATGTCCCTAGAACTCTGGGTGTATCGTTCCCACTGAATAAAATTTG | |
| | ATCTCTAAGTTGACCTATAAATTTAAGTACCAAAAAACTGGTTTGTAAGATTT | |
| | TGGAAGGCAGAGGCAGGGAAGAGACCAACGTGGTCTACATATAAAGTACCA | |
| | GGTACATATAAAGTACCAGGT | |
| mRnu1b1- | ATTTTTGTGGTGCTAAAAGTTAGATTGCATTCCGCCCTTCTCACGTCTCTTTAC | 354 |
| Terminator | GTGTTGTTTGCGAGGCGTGGCCTCGGAGGTAAATGGTCTCCTTTTACCTCAGA | |
| | TAAGCTTACCTTTGCTCTGTGAGTAAGAATGTAGCTACACTTCCAAATTTAGC | |
| | TATTGTCCTTTGCGGTCCAAATGAAAATATTACCTTCCGATCTAACAATGTTA | |
| | AACTAGTCCCAGGCCCTTCTATCGATTGCCAGAATAGACTAGACTACTACACA | |
| | GACACACCCTCTCAAATCAGGCCAGCTCCTGCAACCTGCACCTACAGTTTGTC | |
| | CCCTGGGACAAATGAAAGGACAGGCCCTCTTTCCCCCCCCCCCCCAAATCAGA | |
| | GCACACACAGATCTTGGGGTCTGGGGCAGAAATTTCGTTCATGGAAAAAACA | |
| | AACAAACAAACAAACACGATCAAAGGAAGCTCGTTCCCTTTGGCCTCAGGGT | |
| | TTCTTAGACGCCGTGTTCCC | |
| mRnu1b6- | ATAACTGGTAACCAAAAGCAGATTACTTTCCCCCTTGTGGTCTTCGCGTTTGC | 355 |
| Terminator | GGACGGGAGAGAACTTTCGGCCATTTTCCTTTTAGAAATTTAACTTCTTGAAG | |
| | GCCACCAGGTGACCCTTACTTCCCTCTCACCTTTCCTTCTACCTGTACTTTCTCT | |
| | ATTAAAAAGCCTGCAGAAGTACCCTTGACAAATTGCCAAAAATGACACCTAA | |
| | AAAAAAAAAAAGGAAAAACGACACCTTAGGTAATTATAGGTTGTAAGAAAA | |
| | AGATTTGACCTCTTGGAGTTTTTATTTCTTAATACCAAAGGACACCCAAGGAA | |
| | CGTATCTCTCAAAATTGGGTTTCAAACCTCAGCCGTTTCCTTCTTAGTTCCAGT | |
| | GCTTAACACGCAACAGACCTGCACACCACTACACTGCATACGAGTTTTAAAGT | |
| | TAATAAATCAGTCACCCCTCCATAGTCTTCTACCTACTTGAAGACAGAAACGG | |
| | CTGCTAGGAAGCAATTCTTC | |
| mRnu2-10- | CTGGGGAATAAGAGTGGTTTAAAGAAAGAACAATTTGAGAGATCTGCTTAGA | 356 |
| Terminator | TCTAATAAAACCCAAGTATAGTTTCTTTAGACGTCTTATATCTCACGTAGGAC | |
| | CTCTTATTTCAGACTCACTGCAAGAACAAGCTAAAGAACTATATACAGTCAGG | |
| | AACAAGGTGAGCCATCGCGGTTCTCAGTCCCATGATCTTAAGAGTTTTTCCTC | |
| | TTCCCCTCTTCCCTGGCTGTCCTGGAGCTCACTCTGTAGACCAGGCTGGTCTCA | |
| | AACTCAGAAATCCACTTACCTCTGCCTCCCAAGGGCTGGGATTAAAGGCATGC | |
| | ACCACCATTGCCCGGCAGGGTATGTGGACTATGCTGTCCTTCAACTCAGAGAT | |
| | CGTGGTACTTTAAATCTCATTTTCCGAAAGAAAAGAAAAGAAAGGAAGGGGG | |
| | GAAATAAAGCCAGTATATTTTGCACCCACAATCTGAAGTTCTACAAATATTGT | |
| | TAGAAAATGCAAAGGACCCAAGTC | |
| mRnu3b1- | CTCGGTTTATCTTAAAGGGTTTTAAATATAGAAAGCTTCCTTAGTCTTATGGGT | 357 |
| Terminator | GAGAGTGCTGTTTAAGCCTTACCTAGGTATGTCTTTGAACATTTATTACAGATT | |
| | GTTATTTGTGACTATGACCACTCAGGTGTTCTGTTCTTGTGAATTTAGACACAC | |
| | TCAGAAAGGCTACACACTCAAAGACCACACCAAAGTGAAGTACTATCTGGGC | |
| | CGTGAAACTGGTCACATATCAGTTATTTTGCAGTAAAACAAGGAAGTCAATCA | |
| | TACTGCTTCAGTGGGTCAGGGAGTCAAGAATGCACAGTACAAGAGCTGGTGA | |
| | GATTCCTCAGCAGGTAAGAGCACTGAATGCTCTTCCAAAGATTCTGAGTTCAA | |
| | ATCCCAGCAACCACGTGGTGGATTTGAACAAAGGATGAAAACAACCATCCAT | |
| | AACCAGATCTGATGCCCTCTTCTGGAGTGTCAAAAGACAGCTATGGTGTACTT | |
| | ACATAAATAAATAAGTAAAAACA | |
| mRnu5g- | CCACATTTTGTGTTTAAAAATAGAAATATTTAAGTGAGATCAGTTTAAATCTG | 358 |
| Terminator | CTTTATCTAGGGTGTCTAACTGCTTGCATCTTTTTAACTTTTCCTTATTTGTGAG | |
| | AAGGTCTGTAAAACTTATTATATGTTAGTACACTGTAGCTGTCTTCAGACACT | |
| | CCAGAAGAGGGATTCAGATCACCTTACAGATGGTTTTGAGCCACCATGTGGTT | |
| | GCTGGGATTTGAACTCAGGACCTTTAGAAAAGTAGTTGGGTGCTCTTACCCGC | |
| | TGAGCCATCTCACCAGCCCTTTTTTTTTCTTTTTTCCAGTTCGGGTTTCTCTGTG | |
| | TGTAGCCCTGGCTGTCCTGGAACTCATTCTGTAGACCGGGCTGGCCTCGAACT | |
| | CAGAAATCTGCCTGCCTCTGCCTCCCAAGTGCTGGGATTAAAGGAATGTGCCT | |
| | TCATTGCCCGGCTCTAGGGCTTTTTGTTGTTGTTTGTTTGTTTGTTTGTTTGTTT | |
| | GCTTTTGTGGATGGCCT | |
| mRnu6- | AAAACCAGTAAGGGAGAAAGGTCAAGTAACATATAAAGGAAGGCCTATCAG | 359 |
| Terminator | AATTACACCAGACTTTTCACCAGAGACTATGAAAGCCAGAAGAGCCTGGACA | |
| | GATGTTATACAGACACTAAGAGAACACAAATGCCAGCCCAGGCTACTATACC | |
| | TGGCCAAACTCTCAATTACCATAGATGGAGAAACCAAAGTATTCCACGACAA | |
| | AAACAAATTCACACAATATCTTTCCACGAATCCAGCCCTTCAAAGGATAATGA | |
| | CAGAAAAGAAGCAATACAAGGACGGAAATCACGCCCTAGAACAACCAAGAA | |
| | ACTAATCATTCAACAAACCAAAAAGAAGACAGCCACAAGAACAGAATGCCA | |
| | ACTCTAACAACAAAAATAAAAGGAAGCAACAATTACTTTTCCTTAATATCTCT | |
| | TAATATCAATGGACTCAATTCCCCAATAAAAAGACATAGACTAACAGACTGG | |
| | CTACACAAACAGGACCCAACATTCTGCTGCTTA | |
| mRnu7- | CCCAATTTCACTGGTCTACAATGAAAGCAAAACAGTTCTCTTCCCCGCTCCCC | 360 |
| Terminator | GGTGTGTGAGAGGGGCTTTGATCCTTCTCTGGTTTCCTAGGAAACGCGTATGT | |
| | GCTAGAGCCACGCTCTGAGACTTCCGCCTCGTGCGGTCCCGCTTCCTTTCTGCC | |
| | TCCTCTGGCCTGCATCCGTGGGGGAGGTGGCTGGCTGCAGGGTTCTCCGGATC | |
| | TCTTCTGTGTTCCCACTACTCAAGCACCGAGTGGCGTTCTATGGCGTCCGCCTC | |
| | GGCTCAGCCCGCGGCCCTGAGCGCGGAGCAGGCCAAGGGTGAGGAAAGAGC | |
| | CTAGTCTGGGTGTGGGCCGTCTTCTGGGGGGAGGGCGCGATACTATTCTTTGC | |
| | CTCTCCAAGTGGGGCCCTTGCCCATTAGGGCGAAGGGTTGGGAGGACAAGCT | |
| | TATCGGGTTGCTTGCAGGCCTCGGCCCACTAATTCCCTGCCTCTTCCCTCCTCT | |
| | GGGCTCGCCCGCCGTCCTCCGCG | |
| mRnu11- | GAATTTGTTAAATTACAGGAGGAAGCTTTTTTAGTTCATTTTACAAATACTTTC | 361 |
| Terminator | TTGTTAGGTAGATAATACCTTCTAAGGTTATTATCTGGATCTTCTAAGTCTTTT | |
| | TATCGCTAGCAAGTCTAAGTTTCTGGACTTTGGAATTAGAAGTTAATGTCGTA | |
| | GGCGGGCATGGTGGTCCACGCCTTTAATCCCAGAGTTTGGGAGGCAGACGCA | |
| | GGTGGTTTTCTGAGTTCGAGGCCATCCTGCTCTTCAATGTTAGTTCCAGGACA | |
| | GCCAGGGTTATACAAAGAAACCCTGTCTCGAGAAACAAAACAAACAAAAAA | |
| | AGAGTAGCTGAGTGCTCTGAGTTGTCTCTCCAGCCCCTTAGCTATTTATTTTCA | |
| | TTTAAATTATTTTTGTTTTATTCGCTTGTGTGCCTGAGTTTGAATTTCCCGGGAT | |
| | TAACAGTTGAAGACTAGGGCTGGTGAGATGGCTCAGCGGTTAAGATTGCCGA | |
| | CTGCTCTTCCAAAGGTCCTGAG | |
| mRnu12- | ACCCCCTAATTCTACAAGAATAGAAAGCTTAGCAGCTCCTCTGAATGGTTGCT | 362 |
| Terminator | CTCAGGTGCTAGTGGTTGCAAGTCGTATGTTGATGCCTGCCTTTGCCCTACCTG | |
| | GTGTGTGAGCGTGAGCGCGCCCCACCTCTCCAGCCTCTCTCTTACTGTTTTTCA | |
| | GACAAGATGGGACTGTTGTTCTCCTACTGCTATGTACCTCAATTTCAATTGATC | |
| | ATGCCTGGACATAACTGCCCATGTTGTAAGGTCGTCTACGTTGTAAGGTCGTG | |
| | CTCAGTTTCTGATGCCTAGACTAGTCACCTTTCCGTTTGCCTCCTGACTGTGTA | |
| | AATGAGGCAAATGAAGGTGGTCTCTTAACTGACATGTTAGTGTTTGACCTATG | |
| | GGATAGTTTCTCATTGTTGAGACAGAATTTCTAGAAGCTGGTCCGCTGAGCGG | |
| | CTAAGAGCACTTGCTGATATTGCAAAGGATCTACCTGGGTTTGGTTCTCAGCA | |
| | CTCACATGGAAGTTCACAT | |
| mRn7sk- | TGTAGTTCCTGCAATTTAATTTTCGTTTAAAACTTTATTTTATTCTTTAACTTAA | 363 |
| Terminator | AAAAAATATATA | |

### EXAMPLES

### Example 1: Exemplary Vectors for Modulating MECP2 Expression

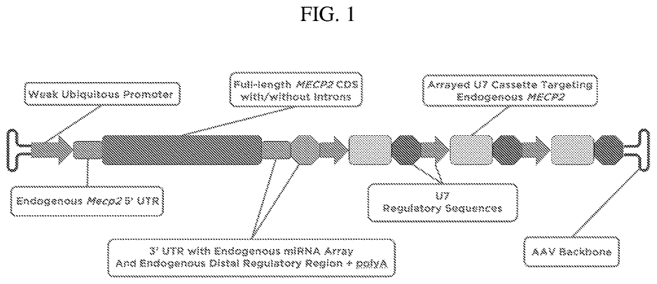

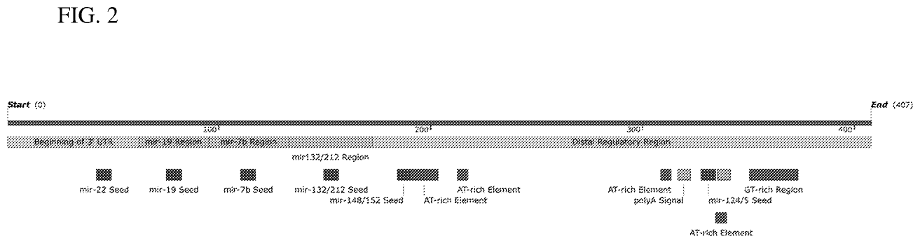

An AAV-based gene therapy approach was designed for treating Rett Syndrome without toxic transgene overexpression including multiple functional units (FIG. 1; Table 1):
1. A weak, ubiquitous promoter (e.g. −Ubc, PGK, EF1a-core). In instances, the promoter can be strengthened via the SV40 intron.
2. The endogenous MECP2 5′ UTR and Kozak sequence. In instances, the vector does not include the MECP2 5′ UTR and/or uses a standard Kozak sequence (GCC(RCC)ATG, where R is any nucleotide and ATG is the start codon).
3. The full-length MECP2 coding sequence including exons 1, 3, and 4. In instances, endogenous intron fragments are included between exons 1 and 3 or 3 and 4.
4. An engineered 3′ UTR including multiple fragments from the endogenous MECP2 3′ UTR and the distal MECP2 polyA signal. In instances, the vector does not include this element, and only includes a standard polyA signal (e.g. −bGH, SV40, hGH).
5. A U7 module to suppress splicing of endogenous MECP2 transcripts. The U7 module can include a single U7 expression cassette or an array. The U7 module can include standard U7 regulatory sequences (promoter, 3′ element) or altered sequences from other small RNAs or engineered sequences.

With this approach, an aim is to avoid toxic overexpression of MECP2 by depleting endogenous MECP2 in cells expressing from the wildtype X chromosome, while simultaneously expressing wildtype MECP2 under the control of a constitutively expressed promoter and endogenous regulatory elements.

### Example 2: Identification and Validation of U7 Cassettes for Depletion of Endogenous MECP2

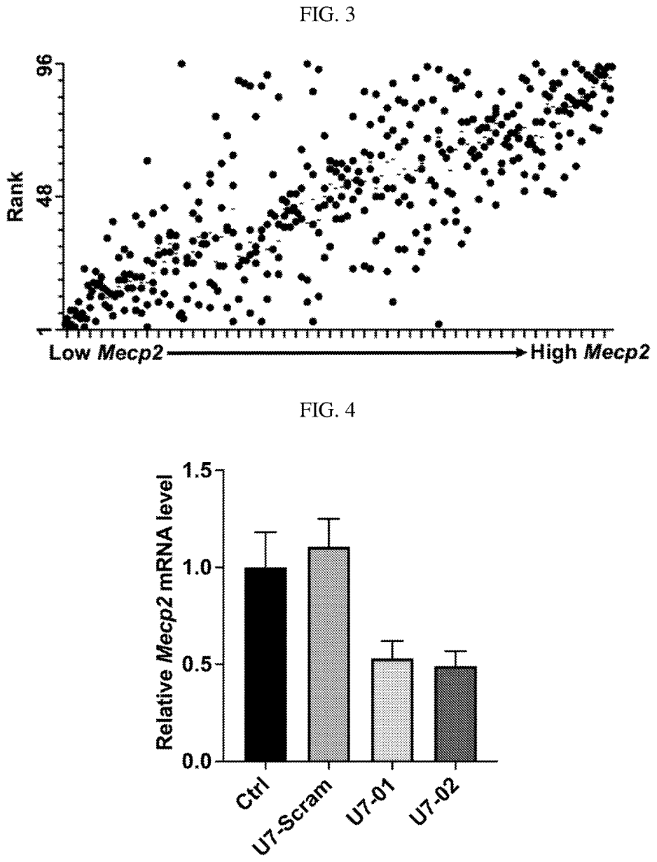

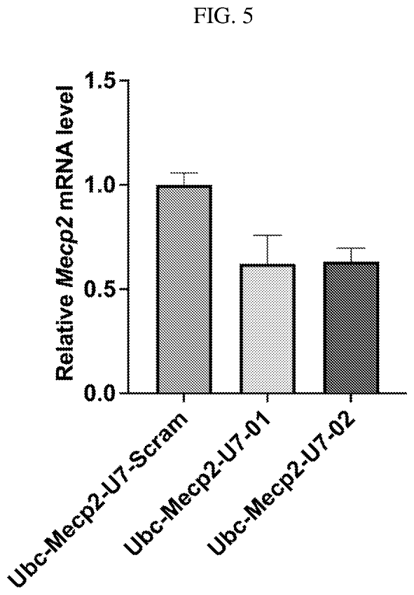

We began by performing a screen in mouse Neuro-2a cells (ATCC CCL-131) for U7 targeting sequences that can efficiently reduce total Mecp2 transcript levels. We screened 92 different U7 antisense sequences targeted to regions surrounding the intron/exon junctions of Mecp2 and paired them with a canonical exonic splicing silencer sequence (Tables 2 and 3) and ranked them via qRT-PCR based on their ability to suppress Mecp2 expression (FIG. 3). Based on our screening we identified several high-confidence hits, including two top candidates targeting the 5′ end of intron 1 and then 3′ end of exon 3, and further validated their activity in N2a cells (FIG. 4). Next, we generated expression constructs carrying a Ubc-Mecp2-2A-EGFP cassette with and without fragments of introns 1 and 3. These transcripts are efficiently spliced and allowed us to test whether the identified U7 targeting sequences could auto-regulate transgene splicing and overall transgene-derivedMecp2 levels. We transfected these constructs into Neuro-2a cells with plasmids expressing U7 candidates and observed significant reduction of Mecp2 levels in constructs containing introns compared to those lacking introns (FIG. 5).

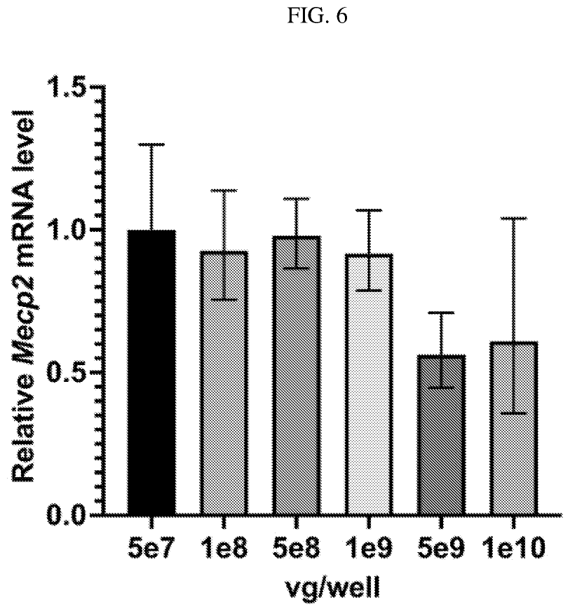

Although we observed significant reduction of Mecp2 levels in Neuro-2a cells, we sought to improve the strength of our U7 cassettes to block Mecp2 splicing more efficiently by generating a second generation of U7 cassettes expressing an array of 4 identical U7 modules. We packaged the top candidate construct, e1-3, in AAV and then performed a dose-response assay in cultured primary mouse hippocampal neurons to assess U7-dependent reduction of Mecp2. FIG. 6 shows that increasing the dose of U7 AAV progressively decreases Mecp2 down to ~50% compared to wildtype. Of note, AAV9 is not capable of transducing many glial cell types found in these mixed cultures (~50% neurons), suggesting that our constructs can achieve near-complete silencing of Mecp2 expression at high doses. To validate our U7 targeting sequences in a more therapeutically relevant tissue, we injected wildtype C57Bl/6J mice with these 4× constructs expressing either a scramble U7 targeting sequence or the top candidates (e1-3 or e3-3), waited 3 weeks, and then collected tissue samples and performed immunohistochemistry for MECP2 protein. Because these AAVs co-expressed GFP, we binned GFP+ cells and analyzed their distribution of MECP2 protein levels. As seen in FIG. 7, cells from animals injected with e1-3 or e3-3 constructs show significantly reduced levels of MECP2 protein across analyzed cells compared to those from animals injected with a Scramble construct. Together, these data show that our U7 constructs reduce MECP2 protein levels in vivo.

### Example 3: Therapeutic Vectors Combining MECP2 Gene Replacement and U7-Based Depletion of Endogenous MECP2

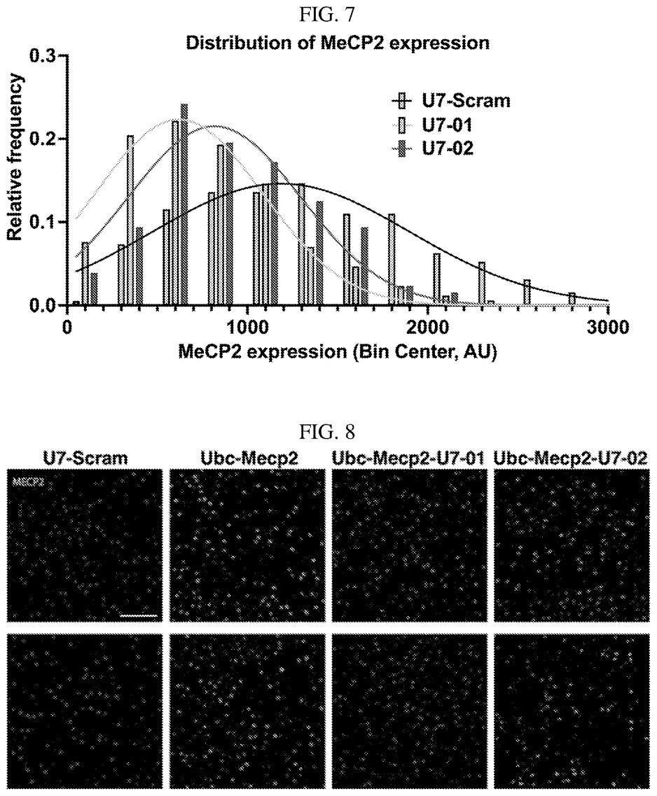

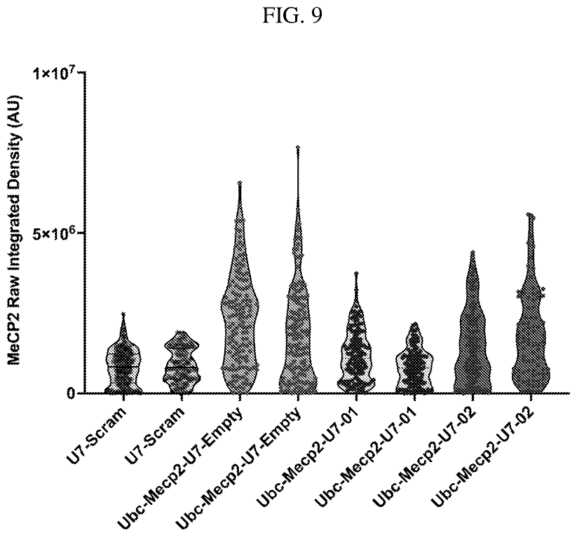

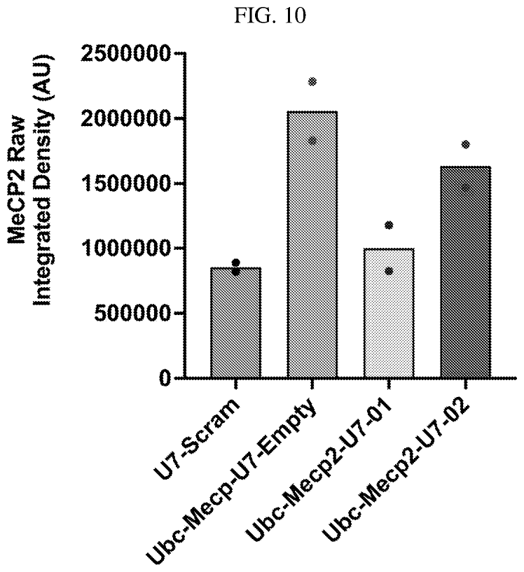

We next sought to combine our U7-based approach for depletion of endogenous Mecp2 with replacement of Mecp2 under the control of a weak, ubiquitous promoter. In our first embodiment, we chose the UbC promoter, which drives expression across all cell types at a low level. We systemically injected wild-type C57B16/J mice with AAVs expressing 1) a scrambled U7 cassette, 2) Ubc-Mecp2, 3), Ubc-Mecp2-U7-e1-3, or 4) Ubc-Mecp2-U7-e3-3 and then performed immunohistochemistry for MECP2 (FIG. 8). Analysis of MECP2 expression levels across cells showed that expression of Ubc-Mecp2 without a U7 cassette targeting endogenous Mecp2 resulted in a significant number of cells overexpressing MECP2, but that this was either reduced or ablated in animals injected with AAVs co-expressing such U7 cassettes (FIGS. 9-10).

As a proof-of-concept that these gene therapy vectors can successfully rescue MECP2 to wild-type levels without widespread overexpression in a therapeutically relevant animal, we injected heterozygous Mecp2 mutant female mice with control or therapeutic AAVs and performed the same immunohistochemistry-based analyses described in FIGS. 8-10. Once again, we found that co-expression of Mecp2 from the Ubc promoter alongside a 4×U7 cassette was capable of restoring MECP2 levels to those similar to control animals and eliminated the presence of cells expressing higher-than-wildtype levels of MECP2 that are seen in mice injected with Ubc-Mecp2 alone (FIG. 11). Together, these data demonstrate the ability of our vectors to reduce endogenous Mecp2 expression and restore normal MECP2 levels without overexpression.

### Example 4: Vector Modifications

We have also developed additional versions of this gene therapy vector to improve different aspects. In the data shown in FIG. 11, we noticed that the distribution of MECP2 levels in Ubc-Mecp2-U7-injected animals was shifted to a lower level compared to wild-type animals, suggesting that most transduced cells did not have their MECP2 levels fully rescued. Thus, we developed several vectors with different modifications to strengthen expression:
Ubc promoter replaced with PGK promoter
Ubc promoter replaced with EF1a core promoter
SV40 intron inserted immediately downstream of Ubc promoter
All promoter backbones+a shortened version of intron 1 inserted in the Mecp2 CDS
All promoter backbones+a shortened version of intron 3 inserted in the Mecp2 CDS
All promoter backbones+shortened versions of introns 1 and 3 inserted in the Mecp2 CDS

Second, in an effort to further strengthen the expression and function of the U7 cassettes included in these gene therapy vectors, we modified the U7 promoter and 3′ terminator elements in several ways:
Replaced mouse U7 promoter with mouse U1a1 promoter
Replaced mouse U7 promoter with mouse U1a1 promoter and replaced mouse U7 3′ element with human U1-1 3′ element
Replaced mouse U7 promoter with human U1-1 promoter
Replaced mouse U7 promoter and 3′ element with human U1-1 promoter and 3′ element
Replaced DSE of mouse U7 promoter with DSE of human U1-1 and PSE of mouse U1a1

### Example 5: Validation of Therapeutic Approach in Mouse

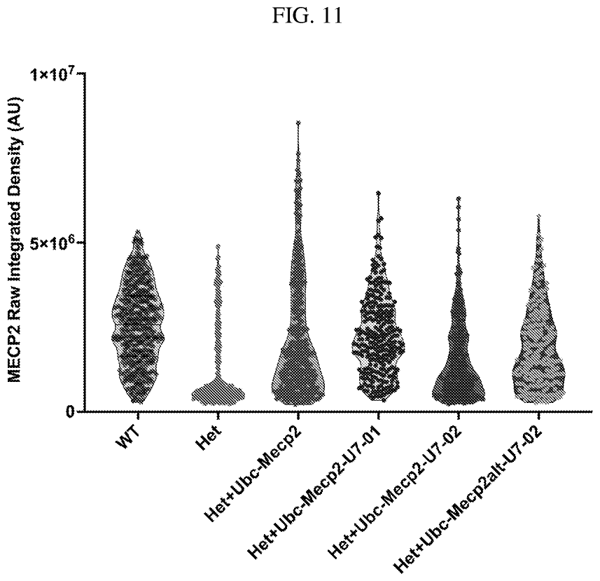

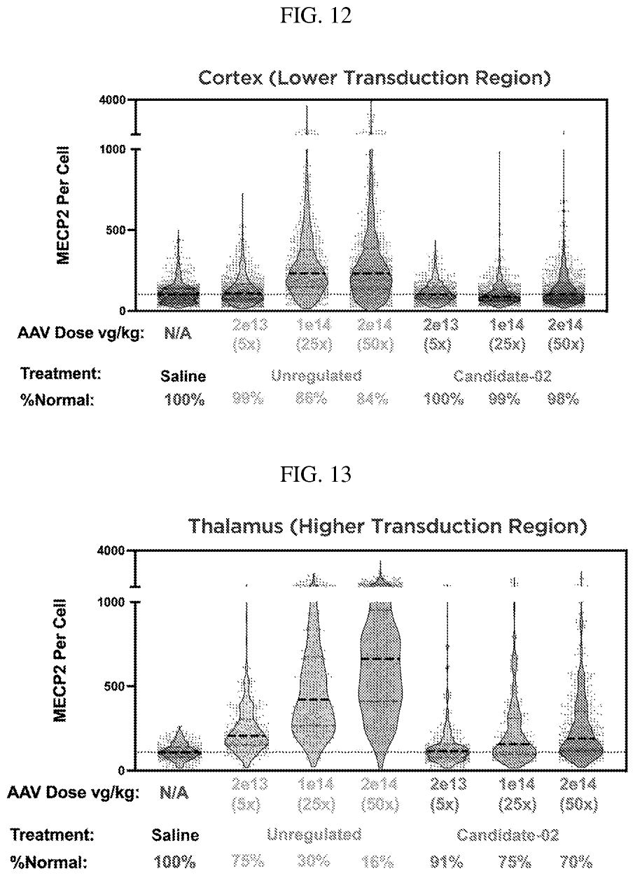

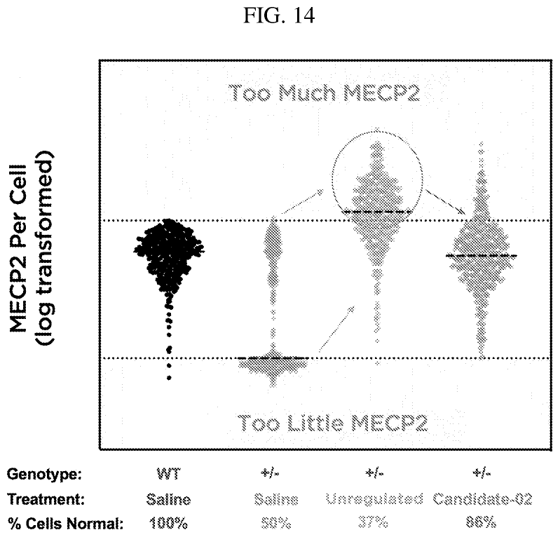

Candidates 1 and 2 are e1-3-16 and e3-3-06. U7 candidate 1 was selected for further validation of the therapeutic approach. First, the ability of this vector to control MECP2 protein levels across a 50× dose range was tested in wild-type mice injected systemically with AAV encoding either the therapeutic candidate or a control unregulated vector consisting of the Ubc-SV40int promoter driving MECP2 with an SV40 polyA terminator and no additional regulatory elements. Per cell MECP2 levels were measured via immunohistochemistry in the cortex and thalamus using an in-house machine learning pipeline. Animals injected with the control unregulated vector showed significant dose-mediated overexpression of MECP2 in both brain regions (FIGS. 12-13), but animals injected with the erase-and-replace vector, which we will refer to as U7 candidate 2 from hereon, were resistant to widespread overexpression even at the 50× dose and even in the thalamus, which is transduced especially well by PHP.eB (FIG. 13). We further confirmed the molecular therapeutic potential of U7 candidate 2 in heterozygous Mecp2^{+/-} female mice, where treatment with U7 candidate 2 resulted in 86% of thalamic cells expressing normal levels of MECP2 at a clinically-relevant dose, as compared to only 37% of cells in animals treated with the unregulated vector (FIG. 14).

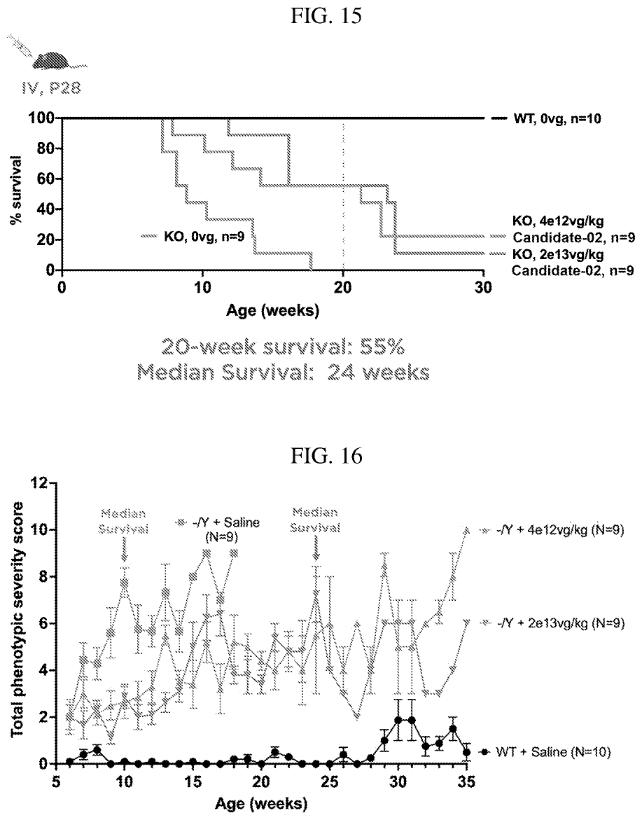

To assess the phenotypic therapeutic potential of U7 candidate 2, hemizygous Mecp2 male mice and wild-type control animals were injected with either saline or U7 candidate 2 via IV injection at P28 at 2 doses: 4e12 vg/kg (1×) and 2e13 vg/kg (5×). While hemizygous males are not a genetically accurate model of Rett Syndrome, which is observed almost exclusively in females, they serve as an important preclinical tool for evaluating therapeutic potential due to their severe phenotypic manifestations (in contrast to heterozygous female mice that exhibit extremely mild phenotypes). Hemizygous males injected with saline survived an average of 9 weeks, while those injected with either 1× or 5× doses of U7 candidate 2 survived for an average of 24 weeks (FIG. 15). Furthermore, animals injected with U7 candidate 2 showed a significantly reduced progression of phenotypic severity (FIG. 16), as measured using the “Bird Score,” which assays gait, mobility, hindlimb clasping, breathing abnormalities, tremor, and general body condition across a 12-point scale (each domain scored 0-2).

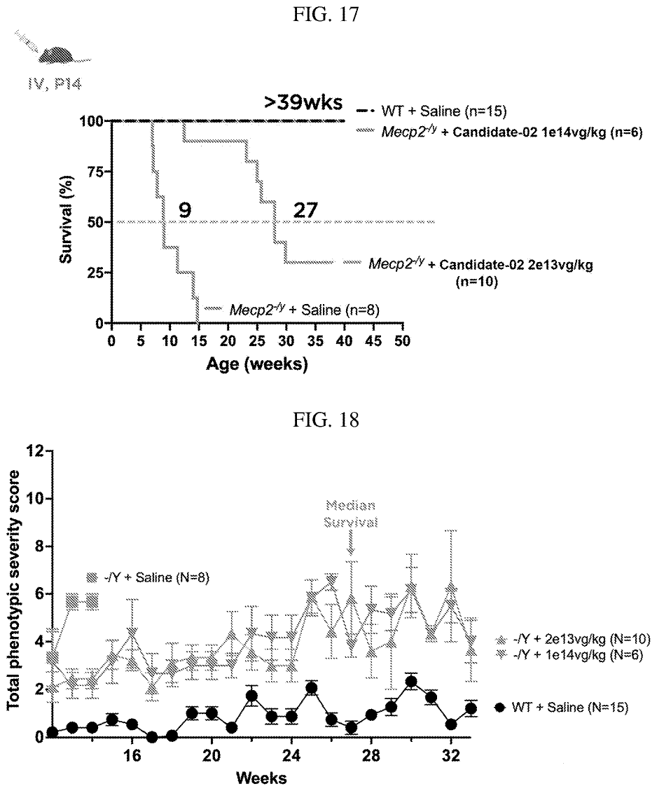

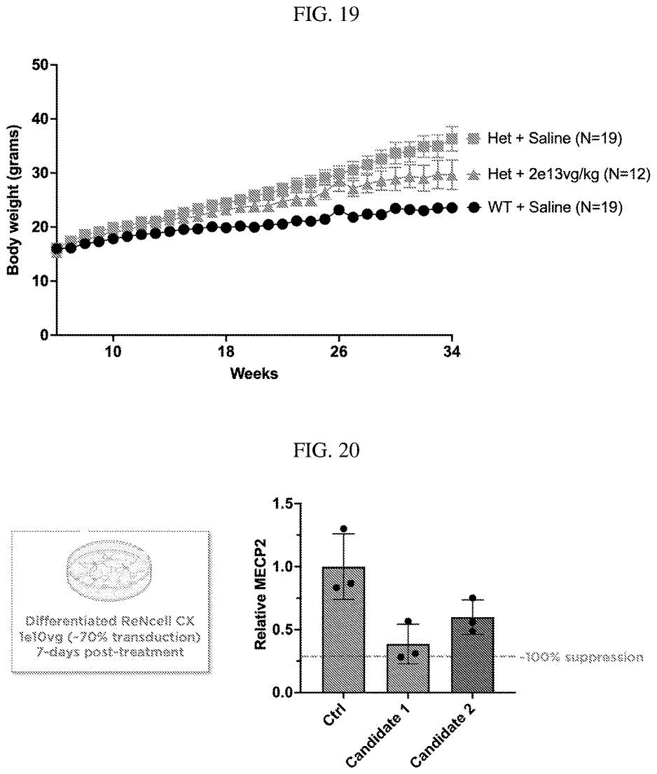

Efficacy was also assessed in two additional settings. First, hemizygous males were injected systemically with saline, 2e13 vg/kg AAV, or 1e14 vg/kg AAV at P14. This models a more clinically-relevant delivery time and allows delivery of therapeutic vector at a time when phenotypic progression is less severe. In this cohort a dose-dependent rescue of lethality was observed when administering U7 candidate 2 as compared to saline, which showed a median survival of 9 weeks. Mice injected with 2e13 vg/kg AAV showed a median survival of 27 weeks and as of 39 weeks-of-age no death was observed in the 1e14 vg/kg group (FIG. 17). Once again, suppression of severe phenotypic progression was also observed in AAV-treated animals (FIG. 18). Heterozygous female Mecp2 mutants were also injected with saline or AAV at P28, though females exhibit an extremely mild phenotype with the most overt abnormality being obesity. Female mutants injected with 2e13 vg/kg AAV showed a clear suppression of obesity at 34 weeks-of-age (FIG. 19), further supporting the therapeutic efficacy of the presented gene therapy approach across Mecp2 phenotypes and ages.

### Example 6: Screening for Human-Optimized U7 Candidates

Proof-of-concept studies demonstrated effective control of MECP2 protein levels across cells and AAV doses using a mouse-optimized vector. For enhanced therapeutic efficacy in humans, human-specific U7 constructs were designed and tested to identify cassettes capable of efficient suppression of endogenous MECP2. Previous work found that qPCR-based screening of MECP2 expression levels is difficult due to MECP2-dependent changes across the transcriptome, which hinder normalization required for accurate quantification. Therefore, luciferase-based reporters were designed to enable quantitative assessment of MECP2 splicing from either exon 1-3 or exon 3-4. Briefly, each reporter was driven by a Ubc promoter placed upstream of a Kozak sequence, exon 1/3, a 5′ fragment of intron 1/3, a 3′ fragment of intron 1/3, and a fragment of exon 3/4 in-frame with GSG-T2A-fLuc. Proper splicing of each reporter leads to expression of function firefly luciferase, while splicing suppression decreases luciferase levels and subsequent luciferase activity, which can be assayed via dual-luciferase assay. Multiple U7 libraries were designed targeting the intron/exon junctions of exons 1, 3, and 4 and libraries were co-transfected into HEK293T cells along with either the exon 1-3 splice reporter or the exon 3-4 splice reporter along with a control Renilla plasmid (Tables 6 and 7). Firefly luciferase was normalized to Renilla and relative luciferase levels were calculated compared to control U7 vectors to identify candidates that could significantly suppress reporter activity (Tables 6 and 7).

To assess the validity of the screening platform, two candidates from the screen that showed high levels of MECP2 suppression (via luciferase reporter) were cloned into 3×U7 AAV vectors and differentiated ReNcell CX cultures (human cortical neurons) were treated with 1e10 vg AAV or formulation buffer. 1e10 vg transduces ~70% of neurons in culture, so a reduction of MECP2 of ~70% would represent near-complete suppression of MECP2 by a tested U7 candidate. Compared to cells treated with formulation buffer, candidates 1 and 2 were both capable of significant reduction of MECP2 protein levels, with candidate 1 showing near-complete suppression (FIG. 20)

### Example 7: Design and Molecular Validation of Humanized Vector

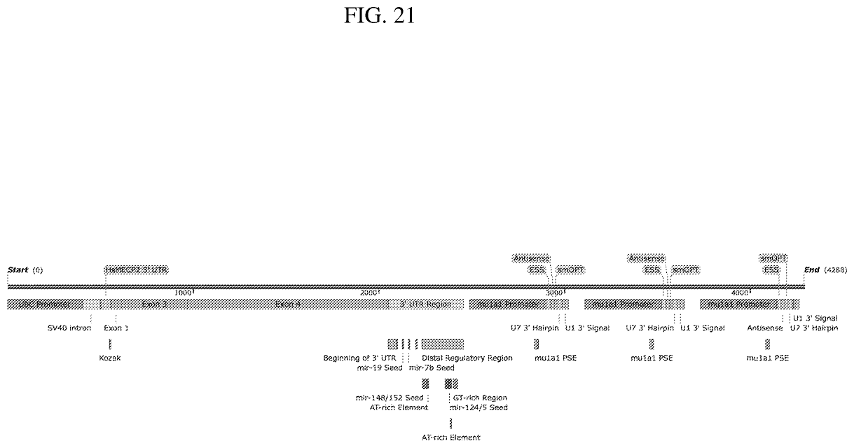

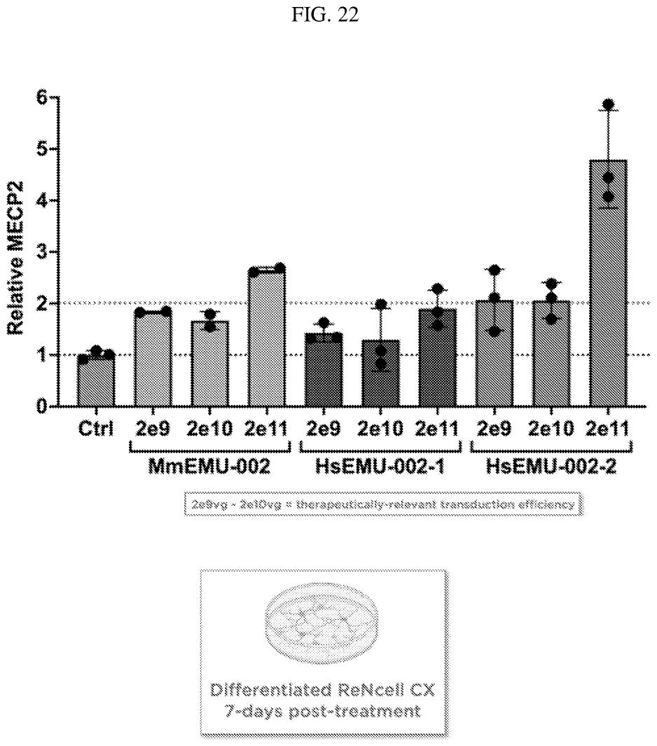

Previous studies of molecular efficacy of the therapeutic construct were performed using a vector encoding an HA-tagged version of the mouse Mecp2 CDS and mouse U7 cassettes. To test the ability of a fully humanized vector to control MECP2 protein levels across a range of AAV doses in a therapeutically relevant setting, we designed new vectors lacking the HA tag, replaced the mouse CDS with the human CDS, and replaced the 3× mouse U7 array with a human 3× U7 array encoding one of two candidate human U7 sequences identified in the human U7 screen (FIG. 21). The U7 terminator sequence was also replaced with the human U1-1 terminator (HU1-1). AAV was produced and differentiated ReNcell CX cultures (human cortical neurons) were transduced with 3 escalating doses of AAV or AAV formulation buffer before assaying MECP2 protein levels. For comparison, the validated mouse vector used in in vivo validation studies was also transduced. All three vectors showed strong homeostatic regulation of MECP2 protein levels at clinically-relevant doses (2e9 vg & 2e10 vg) and the human vector encoding U7 Candidate #1 showed significant homeostatic control even at the very high dose of 2e11 vg, indicating that the erase-and-replace approach has strong therapeutic potential in human neurons (FIG. 22).

1. An expression system for altering gene expression, comprising:
a silencing module deoxyribonucleic acid (DNA) sequence comprising: a first promoter sequence, an optional exonic splicing silencer (ESS) nucleic acid sequence, an antisense nucleic acid sequence that targets an MECP2 ribonucleic acid (RNA), an Sm binding site sequence, a 3′ hairpin sequence, and a 3′ terminator sequence, wherein the silencing module encodes a modified U7 small nuclear RNA (snRNA) that silences or reduces endogenous MECP2 protein expression; and
an MECP2 synthesis module DNA sequence comprising: a second promoter sequence, a 5′ untranslated region (UTR) sequence, a nucleic acid coding sequence (CDS) encoding MECP2, and a 3′ UTR sequence, wherein the MECP2 synthesis module encodes a recombinant messenger RNA (mRNA) that generates MECP2 protein.

2. The system of claim 1, wherein the DNA molecule of the silencing module comprises an arrayed series of silencing modules.

3. The system of claim 1, wherein the first promoter comprises a mouse U1 snRNA promoter sequence, a human U1 snRNA promoter sequence, a mouse U7 snRNA promoter sequence, a human U7 snRNA promoter sequence, or a combination thereof.

4. The system of claim 1, wherein the silencing module comprises the ESS.

5. The system of claim 4, wherein the ESS recruits a protein factor or group of factors that reduce or silence splicing of the endogenous MECP2 RNA.

6. The system of claim 1, wherein the antisense nucleic acid sequence is fully reverse complementary or partially reverse complementary to the targeted region.

7. The system of claim 1, wherein the targeted region is within an intron of the endogenous MECP2 RNA.

8. The system of claim 7, wherein the intron comprises an intron between exons 1 and 3 of MECP2, or an intron between exons 3 and 4 of MECP2.

9. The system of claim 1, wherein the targeted region is within an exon of the endogenous MECP2 RNA.

10. The system of claim 1, wherein the antisense nucleic acid sequence targets an alternatively spliced exon of the endogenous MECP2 RNA.

11. The system of claim 1, wherein the targeted region is within 100 nucleotides of an intron/exon junction.

12. The system of claim 1, wherein the antisense nucleic acid sequence is 10-60 nucleotides in length.

13. The system of claim 1, wherein the silencing module further comprises an Sm binding site sequence.

14. The system of claim 1, wherein the hairpin sequence comprises a U7 small nuclear RNA (snRNA) 3′ hairpin sequence

15. The system of claim 1, wherein the 3′ terminator sequence comprises a mouse U1 snRNA terminator sequence, a human U1 snRNA terminator sequence, a mouse U7 snRNA terminator sequence, a human U7 snRNA terminator sequence, or a combination thereof.

16. The system of claim 1, wherein the second promoter sequence comprises a weak promoter that drives expression of mRNA molecules at a rate no greater than an endogenous MECP2 promoter.

17. The system of claim 1, wherein the second promoter sequence comprises a promoter sequence of a −Ubc promoter, a PGK promoter, or an EF1a-core promoter.

18. The system of claim 1, wherein the synthesis module further comprises an SV40 intron sequence.

19. The system of claim 1, wherein the synthesis module further comprises a 5′ untranslated region (UTR) sequence of the MECP2 RNA.

20. The system of claim 1, wherein the synthesis module further comprises a Kozak sequence.

21. The system of claim 1, wherein the CDS comprises an intron.

22. The system of claim 1, wherein the CDS does not comprise an intron.

23. The system of claim 1, wherein the CDS comprises MECP2 exons 1, 3 and 4.

24. The system of claim 1, wherein the CDS excludes MECP2 exon 2.

25. The system of claim 1, wherein the synthesis module comprises a polyA signal sequence.

26. The system of claim 25, wherein the polyA signal sequence comprises a −bGH signal sequence, a SV40 signal sequence, or a hGH polyA signal sequence.

27. The system of claim 1, wherein the silencing module reduces an MECP2 measurement in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%, relative to a baseline MECP2 measurement.

28. The system of claim 1, wherein the synthesis module increases an MECP2 measurement in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, or at least 250%, relative to a baseline MECP2 measurement.

29. The system of claim 1, wherein contact or expression of the system with a cell or cell population results in an MECP2 measurement between 1× and 2× relative to a control.

30. A dual RNA system for altering gene expression, comprising:
a U7 small nuclear RNA (snRNA) silencing module comprising: an exonic splicing silencer (ESS) nucleic acid sequence, an antisense nucleic acid sequence that binds an MECP2 ribonucleic acid (RNA), an Sm binding site sequence, and a 3′ hairpin sequence; and
an MECP2 messenger RNA (mRNA) synthesis module comprising: a 5′ untranslated region (UTR) sequence, a nucleic acid coding sequence (CDS) encoding MECP2, and a 3′ UTR sequence;
wherein the U7 snRNA silencing module silences or reduces endogenous MECP2 protein expression, and the MECP2 mRNA synthesis module generates MECP2 protein.

31. A system for altering gene expression, comprising:
a silencing module comprising an exonic splicing silencer (ESS) nucleic acid sequence coupled with an antisense nucleic acid sequence that targets an endogenous MECP2 ribonucleic acid (RNA); and
a synthesis module comprising a nucleic acid coding sequence (CDS) that encodes a recombinant version of the MECP2 RNA.

32. A pharmaceutical composition comprising the system of any one of claims 1-31, and a pharmaceutically acceptable carrier.

33. A method, comprising administering the pharmaceutical composition of claim 32 to a subject.

34. The method of claim 33, wherein the subject has been identified as having a genetic disease prior to the treatment.

35. The method of claim 34, wherein the genetic disease is associated with haploinsufficiency of the endogenous MECP2 RNA.

36. The method of claim 34, wherein the genetic disease is associated with tissue mosaic expression of the endogenous MECP2 RNA.

37. The method of claim 34, wherein the genetic disease comprises Rett syndrome.

38. A method, comprising:
suppressing protein expression of an endogenous MECP2 RNA in a first cell expressing the endogenous MECP2 RNA; and
synthesizing or enhancing protein expression of a recombinant version of the MECP2 RNA in a second cell that otherwise does not express the endogenous MECP2 RNA, or that expresses the endogenous MECP2 RNA at a low level.

39. The method of claim 38, further comprising synthesizing or enhancing protein expression of the recombinant version of the MECP2 RNA in the first cell.

40. The method of claim 38, wherein said suppressing is performed upon contacting the first cell with a silencing module or with a vector encoding the silencing module.

41. The method of claim 38, wherein suppressing protein expression comprises suppressing endogenous MECP2 protein expression by at least 10%.

42. The method of claim 38, wherein said synthesizing or enhancing recombinant MECP2 protein expression is performed upon contacting the second cell with a synthesis module or with a vector encoding the synthesis module.

43. The method of claim 38, wherein enhancing recombinant MECP2 protein expression comprises enhancing protein expression by at least 10%.

44. The method of claim 38, wherein the low level of expression of the endogenous MECP2 RNA in the second cell comprises an undetectable level, comprises a level below a desired level, comprises a level lower than a wild type cell, or comprises an expression lower than that of the first cell.

45. The method of claim 38, wherein the low level of expression of the endogenous MECP2 RNA in the second cell comprises a level at least 10% lower than that of the first cell.

46. The method of claim 38, wherein the silencing module and the synthesis module are encoded together in a nucleic acid construct.

47. The method of claim 46, wherein the nucleic acid construct is delivered to the first and second cell using one or more viral vectors.

48. The method of claim 38, wherein the silencing module and the synthesis module are encoded in separate nucleic acid constructs.

49. The method of claim 48, wherein the separate nucleic acid constructs are delivered to the first and second cell using one or more viral vectors.


SEQUENCE LISTING (370 sequences)

SEQ ID NO: 1 - DNA, 400 bp, Homo sapiens
  source 1..400: mol_type = genomic DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggt

SEQ ID NO: 2 - DNA, 500 bp, Homo sapiens
  source 1..500: mol_type = genomic DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 3 - DNA, 230 bp, Homo sapiens
  source 1..230: mol_type = genomic DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 4 - DNA, 499 bp, unidentified
  source 1..499: mol_type = unassigned DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtcggtaagtttagtctttttgtcttttatttcaggtcccggatccggtggtggtgcaaatcaaagaactgctcctcagtggatgttgcctttacttctag

SEQ ID NO: 5 - RNA, 55 nt, Homo sapiens
  source 1..55: mol_type = other RNA
gcgcgcgctccctcctctcggagagagggctgtggtaaaagccgtccggaaaatg

SEQ ID NO: 6 - DNA, 1506 bp, Mus musculus
  source 1..1506: mol_type = genomic DNA
atggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctga

SEQ ID NO: 7 - DNA, 1497 bp, Homo sapiens
  source 1..1497: mol_type = genomic DNA
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 8 - DNA, 100 bp, Mus musculus
  source 1..100: mol_type = genomic DNA
gtgagtgggaccgccaaggccgcgggcggggacccttgctggggggcgggtaggggcgggacgtggcgcgggaggggcccgcggggtcgggcgacacggc

SEQ ID NO: 9 - DNA, 100 bp, Mus musculus
  source 1..100: mol_type = genomic DNA
agccaggggtctcatgtgtggcactcaagctcacctatactcactactgtgcagacagctgtgttctctgtaatacttacatatttgtttaatacttcag

SEQ ID NO: 10 - DNA, 100 bp, Mus musculus
  source 1..100: mol_type = genomic DNA
gtaagtaagagcaagtcttgtgtctatagaacaagaaggccaggtgggtaggaaggctaggatggaaaagctttattgcactgaaatggcttcccaaaaa

SEQ ID NO: 11 - DNA, 100 bp, Mus musculus
  source 1..100: mol_type = genomic DNA
atgtcttagaagaaagcttgtcagcatattttctatctcagtctctatgttgtcaaaaagcttctgactattgtttgtgtctctctgtttgtttctacag

SEQ ID NO: 12 - DNA, 100 bp, Homo sapiens
  source 1..100: mol_type = genomic DNA
gtgagtgggaccgccgtggccgcgggcggggacccttgccggggggcgggggtcaggggcgggacgtggcgcgggaggggcccgcggggtcggacgacac

SEQ ID NO: 13 - DNA, 100 bp, Homo sapiens
  source 1..100: mol_type = genomic DNA
ggggccttgcatgtggtgggggtccaagcctgcctctgctcacttgttctgcagactggcatgttctctgtgatacttacatacttgtttaacacttcag

SEQ ID NO: 14 - DNA, 100 bp, Homo sapiens
  source 1..100: mol_type = genomic DNA
gtaagtaagagcaactcctatctctacagggcagggagggcagggacaaggatccctcatggagcaggaaaatgtatgtgcccagggtggggtcgggggg

SEQ ID NO: 15 - DNA, 100 bp, Homo sapiens
  source 1..100: mol_type = genomic DNA
tttgtcagagcgttgtcaccaccatccgctctgccctatctctgacattgctatggagagcctctaattgttccttgtgtctttctgtttgtccccacag

SEQ ID NO: 16 - RNA, 407 nt, Homo sapiens
  source 1..407: mol_type = other RNA
ctttacacggagcggattgcaaagcaaaccaacaagaataaaggcagctgttgtctcttctcggccagaagtagctttgcacttttctaaactagaatccagaaacttgttcttccaaagcagagagcattatgaccaaatatcaccaggactgttactcaatgtgtgccgaaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttctttta

SEQ ID NO: 17 - RNA, 208 nt, Homo sapiens
  source 1..208: mol_type = other RNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 18 - RNA, 45 nt, Homo sapiens
  source 1..45: mol_type = unassigned RNA
annaatttttggagcaggttttctgacttcggtcggaaaacccct

SEQ ID NO: 19 - DNA, 255 bp, Mus musculus
  source 1..255: mol_type = genomic DNA
aacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 20 - DNA, 411 bp, Mus musculus
  source 1..411: mol_type = genomic DNA
taaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 21 - DNA, 297 bp, Homo sapiens
  source 1..297: mol_type = genomic DNA
aacaacataggagctgtgattggctgttttcagccaatcagcactgactcagggcgacttctatgtagatgaggcagcgcagaggctgctgcctttacaagcggtcacaaactcaagaaacgagcggttttaatagtcttttagaatattgtttatcgaaccgaataaggaactgtgctttgtgattcacatatcagtggaggggtgtggaaatggcaccttgatgggagtgcgcgaggcaagtgaccgtgtgtggagttgatgtccttccctggctcgctacagacgcacttccgc

SEQ ID NO: 22 - DNA, 286 bp, Mus musculus
  source 1..286: mol_type = other DNA
aacaacataggagctgtgattggctgttttcagccaatcagcactgactcagggcgacttctatgtagatgaggcagcgcagaggctgctgcctttacaagcggtcacaaactcaagaaacgagcggttttaatagtcttttagaatattgtttatcgaaccgaataaggaactgtgctttgtgattcacatatcagtggaggggtgtggaaatggcaccttgatttaccgtaactatgaaatggagttgatgtccttccctggctcgctacagacgcacttccgc

SEQ ID NO: 23 - DNA, 104 bp, Mus musculus
  source 1..104: mol_type = unassigned DNA
cccaatttcactggtctacaatgaaagcaaaacagttctcttccccgctccccggtgtgtgagaggggctttgatccttctctggtttcctaggaaacgcgtat

SEQ ID NO: 24 - DNA, 35 bp, Homo sapiens
  source 1..35: mol_type = unassigned DNA
actttctggagtttcaaaagtagactgtacgctaa

SEQ ID NO: 25 - DNA, 141 bp, unidentified
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 26 - RNA, 20 nt, Mus musculus
  source 1..20: mol_type = unassigned RNA
gcccctggctcgcctcctct

SEQ ID NO: 27 - RNA, 20 nt, Mus musculus
  source 1..20: mol_type = unassigned RNA
ggcggaggaaaccatcatat

SEQ ID NO: 28 - RNA, 20 nt, Mus musculus
  source 1..20: mol_type = unassigned RNA
gagacaatagaccttagtaa

SEQ ID NO: 29 - RNA, 20 nt, Mus musculus
  source 1..20: mol_type = unassigned RNA
ggcccacttcgcggtgtaat

SEQ ID NO: 30 - RNA, 20 nt, Mus musculus
  source 1..20: mol_type = unassigned RNA
tcctcctcctccgctcggcg

SEQ ID NO: 31 - RNA, 20 nt, Mus musculus
  source 1..20: mol_type = unassigned RNA
agtctctcctcctcgcctcc

SEQ ID NO: 32 - RNA, 20 nt, Mus musculus
  source 1..20: mol_type = unassigned RNA
gccttggcggtcccactcac

SEQ ID NO: 33 - RNA, 20 nt, Mus musculus
  source 1..20: mol_type = unassigned RNA
agcaagggtccccgcccgcg

SEQ ID NO: 34 - RNA, 24 nt, Mus musculus
  source 1..24: mol_type = unassigned RNA
tcctcctcctccgctcggcgcggc

SEQ ID NO: 35 - RNA, 24 nt, Mus musculus
  source 1..24: mol_type = unassigned RNA
agtctctcctcctcgcctcctcct

SEQ ID NO: 36 - RNA, 24 nt, Mus musculus
  source 1..24: mol_type = unassigned RNA
gccttggcggtcccactcacagtc

SEQ ID NO: 37 - RNA, 24 nt, Mus musculus
  source 1..24: mol_type = unassigned RNA
agcaagggtccccgcccgcggcct

SEQ ID NO: 38 - RNA, 26 nt, Mus musculus
  source 1..26: mol_type = unassigned RNA
tcctcctcctccgctcggcgcggcgg

SEQ ID NO: 39 - RNA, 26 nt, Mus musculus
  source 1..26: mol_type = unassigned RNA
agtctctcctcctcgcctcctcctcc

SEQ ID NO: 40 - RNA, 26 nt, Mus musculus
  source 1..26: mol_type = unassigned RNA
gccttggcggtcccactcacagtctc

SEQ ID NO: 41 - RNA, 26 nt, Mus musculus
  source 1..26: mol_type = unassigned RNA
agcaagggtccccgcccgcggccttg

SEQ ID NO: 42 - RNA, 30 nt, Mus musculus
  source 1..30: mol_type = unassigned RNA
tcctcctcctccgctcggcgcggcggcggc

SEQ ID NO: 43 - RNA, 30 nt, Mus musculus
  source 1..30: mol_type = unassigned RNA
agtctctcctcctcgcctcctcctcctcct

SEQ ID NO: 44 - RNA, 30 nt, Mus musculus
  source 1..30: mol_type = unassigned RNA
gccttggcggtcccactcacagtctctcct

SEQ ID NO: 45 - RNA, 30 nt, Mus musculus
  source 1..30: mol_type = unassigned RNA
agcaagggtccccgcccgcggccttggcgg

SEQ ID NO: 46 - RNA, 20 nt, Mus musculus
  source 1..20: mol_type = unassigned RNA
agttgaaagtccccattgag

SEQ ID NO: 47 - RNA, 20 nt, Mus musculus
  source 1..20: mol_type = unassigned RNA
ctaaaacaaagaaaatcgta

SEQ ID NO: 48 - RNA, 20 nt, Mus musculus
  source 1..20: mol_type = unassigned RNA
agtctgtatttttatggagc

SEQ ID NO: 49 - RNA, 20 nt, Mus musculus
  source 1..20: mol_type = unassigned RNA
catcaaagcaggaactggtg

SEQ ID NO: 50 - RNA, 24 nt, Mus musculus
  source 1..24: mol_type = unassigned RNA
agttgaaagtccccattgagctgc

SEQ ID NO: 51 - RNA, 24 nt, Mus musculus
  source 1..24: mol_type = unassigned RNA
ctaaaacaaagaaaatcgtaagtt

SEQ ID NO: 52 - RNA, 24 nt, Mus musculus
  source 1..24: mol_type = unassigned RNA
agtctgtatttttatggagcctaa

SEQ ID NO: 53 - RNA, 24 nt, Mus musculus
  source 1..24: mol_type = unassigned RNA
catcaaagcaggaactggtgagtc

SEQ ID NO: 54 - RNA, 26 nt, Mus musculus
  source 1..26: mol_type = unassigned RNA
agttgaaagtccccattgagctgcac

SEQ ID NO: 55 - RNA, 26 nt, Mus musculus
  source 1..26: mol_type = unassigned RNA
ctaaaacaaagaaaatcgtaagttga

SEQ ID NO: 56 - RNA, 26 nt, Mus musculus
  source 1..26: mol_type = unassigned RNA
agtctgtatttttatggagcctaaaa

SEQ ID NO: 57 - RNA, 26 nt, Mus musculus
  source 1..26: mol_type = unassigned RNA
catcaaagcaggaactggtgagtctg

SEQ ID NO: 58 - RNA, 30 nt, Mus musculus
  source 1..30: mol_type = unassigned RNA
agttgaaagtccccattgagctgcaccttt

SEQ ID NO: 59 - RNA, 30 nt, Mus musculus
  source 1..30: mol_type = unassigned RNA
ctaaaacaaagaaaatcgtaagttgaaagt

SEQ ID NO: 60 - RNA, 30 nt, Mus musculus
  source 1..30: mol_type = unassigned RNA
agtctgtatttttatggagcctaaaacaaa

SEQ ID NO: 61 - RNA, 30 nt, Mus musculus
  source 1..30: mol_type = unassigned RNA
catcaaagcaggaactggtgagtctgtatt

SEQ ID NO: 62 - RNA, 20 nt, Mus musculus
  source 1..20: mol_type = unassigned RNA
taccatggaatcctgttgga

SEQ ID NO: 63 - RNA, 20 nt, Mus musculus
  source 1..20: mol_type = unassigned RNA
ctgagccctaacatcccagc

SEQ ID NO: 64 - RNA, 20 nt, Mus musculus
  source 1..20: mol_type = unassigned RNA
ggaaggttacttacctgagc

SEQ ID NO: 65 - RNA, 24 nt, Mus musculus
  source 1..24: mol_type = unassigned RNA
taccatggaatcctgttggagctg

SEQ ID NO: 66 - RNA, 24 nt, Mus musculus
  source 1..24: mol_type = unassigned RNA
ctgagccctaacatcccagctacc

SEQ ID NO: 67 - RNA, 24 nt, Mus musculus
  source 1..24: mol_type = unassigned RNA
aggaaggttacttacctgagccct

SEQ ID NO: 68 - RNA, 26 nt, Mus musculus
  source 1..26: mol_type = unassigned RNA
taccatggaatcctgttggagctggt

SEQ ID NO: 69 - RNA, 26 nt, Mus musculus
  source 1..26: mol_type = unassigned RNA
ctgagccctaacatcccagctaccat

SEQ ID NO: 70 - RNA, 26 nt, Mus musculus
  source 1..26: mol_type = unassigned RNA
aggaaggttacttacctgagccctaa

SEQ ID NO: 71 - RNA, 30 nt, Mus musculus
  source 1..30: mol_type = unassigned RNA
taccatggaatcctgttggagctggtctac

SEQ ID NO: 72 - RNA, 30 nt, Mus musculus
  source 1..30: mol_type = unassigned RNA
ctgagccctaacatcccagctaccatggaa

SEQ ID NO: 73 - RNA, 30 nt, Mus musculus
  source 1..30: mol_type = unassigned RNA
aaggaaggttacttacctgagccctaacat

SEQ ID NO: 74 - RNA, 20 nt, Mus musculus
  source 1..20: mol_type = unassigned RNA
gtaagtattacagagaacac

SEQ ID NO: 75 - RNA, 20 nt, Mus musculus
  source 1..20: mol_type = unassigned RNA
ctgaagtattaaacaaatat

SEQ ID NO: 76 - RNA, 20 nt, Mus musculus
  source 1..20: mol_type = unassigned RNA
ggtcttctgacttttcctcc

SEQ ID NO: 77 - RNA, 20 nt, Mus musculus
  source 1..20: mol_type = unassigned RNA
tctgaggccctggagatcct

SEQ ID NO: 78 - RNA, 24 nt, Mus musculus
  source 1..24: mol_type = unassigned RNA
gtaagtattacagagaacacagct

SEQ ID NO: 79 - RNA, 24 nt, Mus musculus
  source 1..24: mol_type = unassigned RNA
ctgaagtattaaacaaatatgtaa

SEQ ID NO: 80 - RNA, 24 nt, Mus musculus
  source 1..24: mol_type = unassigned RNA
ggtcttctgacttttcctccctga

SEQ ID NO: 81 - RNA, 24 nt, Mus musculus
  source 1..24: mol_type = unassigned RNA
tctgaggccctggagatcctggtc

SEQ ID NO: 82 - RNA, 26 nt, Mus musculus
  source 1..26: mol_type = unassigned RNA
gtaagtattacagagaacacagctgt

SEQ ID NO: 83 - RNA, 26 nt, Mus musculus
  source 1..26: mol_type = unassigned RNA
ctgaagtattaaacaaatatgtaagt

SEQ ID NO: 84 - RNA, 26 nt, Mus musculus
  source 1..26: mol_type = unassigned RNA
ggtcttctgacttttcctccctgaag

SEQ ID NO: 85 - RNA, 26 nt, Mus musculus
  source 1..26: mol_type = unassigned RNA
tctgaggccctggagatcctggtctt

SEQ ID NO: 86 - RNA, 30 nt, Mus musculus
  source 1..30: mol_type = unassigned RNA
gtaagtattacagagaacacagctgtctgc

SEQ ID NO: 87 - RNA, 30 nt, Mus musculus
  source 1..30: mol_type = unassigned RNA
ctgaagtattaaacaaatatgtaagtatta

SEQ ID NO: 88 - RNA, 30 nt, Mus musculus
  source 1..30: mol_type = unassigned RNA
ggtcttctgacttttcctccctgaagtatt

SEQ ID NO: 89 - RNA, 30 nt, Mus musculus
  source 1..30: mol_type = unassigned RNA
tctgaggccctggagatcctggtcttctga

SEQ ID NO: 90 - RNA, 20 nt, Mus musculus
  source 1..20: mol_type = unassigned RNA
ctttccagcagatcggccag

SEQ ID NO: 91 - RNA, 20 nt, Mus musculus
  source 1..20: mol_type = unassigned RNA
ttgatcaaatatacatcata

SEQ ID NO: 92 - RNA, 20 nt, Mus musculus
  source 1..20: mol_type = unassigned RNA
caagacttgctcttacttac

SEQ ID NO: 93 - RNA, 20 nt, Mus musculus
  source 1..20: mol_type = unassigned RNA
gccttcttgttctatagaca

SEQ ID NO: 94 - RNA, 24 nt, Mus musculus
  source 1..24: mol_type = unassigned RNA
ctttccagcagatcggccagactt

SEQ ID NO: 95 - RNA, 24 nt, Mus musculus
  source 1..24: mol_type = unassigned RNA
ttgatcaaatatacatcatacttt

SEQ ID NO: 96 - RNA, 24 nt, Mus musculus
  source 1..24: mol_type = unassigned RNA
caagacttgctcttacttacttga

SEQ ID NO: 97 - RNA, 24 nt, Mus musculus
  source 1..24: mol_type = unassigned RNA
gccttcttgttctatagacacaag

SEQ ID NO: 98 - RNA, 26 nt, Mus musculus
  source 1..26: mol_type = unassigned RNA
ctttccagcagatcggccagacttcc

SEQ ID NO: 99 - RNA, 26 nt, Mus musculus
  source 1..26: mol_type = unassigned RNA
ttgatcaaatatacatcatactttcc

SEQ ID NO: 100 - RNA, 26 nt, Mus musculus
  source 1..26: mol_type = unassigned RNA
caagacttgctcttacttacttgatc

SEQ ID NO: 101 - RNA, 26 nt, Mus musculus
  source 1..26: mol_type = unassigned RNA
gccttcttgttctatagacacaagac

SEQ ID NO: 102 - RNA, 30 nt, Mus musculus
  source 1..30: mol_type = unassigned RNA
ctttccagcagatcggccagacttcctttg

SEQ ID NO: 103 - RNA, 30 nt, Mus musculus
  source 1..30: mol_type = unassigned RNA
ttgatcaaatatacatcatactttccagca

SEQ ID NO: 104 - RNA, 30 nt, Mus musculus
  source 1..30: mol_type = unassigned RNA
caagacttgctcttacttacttgatcaaat

SEQ ID NO: 105 - RNA, 30 nt, Mus musculus
  source 1..30: mol_type = unassigned RNA
gccttcttgttctatagacacaagacttgc

SEQ ID NO: 106 - RNA, 20 nt, Mus musculus
  source 1..20: mol_type = unassigned RNA
acacaaacaatagtcagaag

SEQ ID NO: 107 - RNA, 20 nt, Mus musculus
  source 1..20: mol_type = unassigned RNA
ctgtagaaacaaacagagag

SEQ ID NO: 108 - RNA, 20 nt, Mus musculus
  source 1..20: mol_type = unassigned RNA
gaaaagcttttccctgggga

SEQ ID NO: 109 - RNA, 20 nt, Mus musculus
  source 1..20: mol_type = unassigned RNA
aatcaattctactttagagc

SEQ ID NO: 110 - RNA, 24 nt, Mus musculus
  source 1..24: mol_type = unassigned RNA
acacaaacaatagtcagaagcttt

SEQ ID NO: 111 - RNA, 24 nt, Mus musculus
  source 1..24: mol_type = unassigned RNA
ctgtagaaacaaacagagagacac

SEQ ID NO: 112 - RNA, 24 nt, Mus musculus
  source 1..24: mol_type = unassigned RNA
gaaaagcttttccctggggactgt

SEQ ID NO: 113 - RNA, 24 nt, Mus musculus
  source 1..24: mol_type = unassigned RNA
aatcaattctactttagagcgaaa

SEQ ID NO: 114 - RNA, 26 nt, Mus musculus
  source 1..26: mol_type = unassigned RNA
acacaaacaatagtcagaagcttttt

SEQ ID NO: 115 - RNA, 26 nt, Mus musculus
  source 1..26: mol_type = unassigned RNA
ctgtagaaacaaacagagagacacaa

SEQ ID NO: 116 - RNA, 26 nt, Mus musculus
  source 1..26: mol_type = unassigned RNA
gaaaagcttttccctggggactgtag

SEQ ID NO: 117 - RNA, 26 nt, Mus musculus
  source 1..26: mol_type = unassigned RNA
aatcaattctactttagagcgaaaag

SEQ ID NO: 118 - RNA, 30 nt, Mus musculus
  source 1..30: mol_type = unassigned RNA
acacaaacaatagtcagaagctttttgaca

SEQ ID NO: 119 - RNA, 30 nt, Mus musculus
  source 1..30: mol_type = unassigned RNA
ctgtagaaacaaacagagagacacaaacaa

SEQ ID NO: 120 - RNA, 30 nt, Mus musculus
  source 1..30: mol_type = unassigned RNA
gaaaagcttttccctggggactgtagaaac

SEQ ID NO: 121 - RNA, 30 nt, Mus musculus
  source 1..30: mol_type = unassigned RNA
aatcaattctactttagagcgaaaagcttt

SEQ ID NO: 122 - RNA, 20 nt, Homo sapiens
  source 1..20: mol_type = unassigned RNA
gcccctggctcgcctcctct

SEQ ID NO: 123 - RNA, 20 nt, Homo sapiens
  source 1..20: mol_type = unassigned RNA
ggcggaggaaaccatcatat

SEQ ID NO: 124 - RNA, 20 nt, Homo sapiens
  source 1..20: mol_type = unassigned RNA
gagacaatagaccttagtaa

SEQ ID NO: 125 - RNA, 20 nt, Homo sapiens
  source 1..20: mol_type = unassigned RNA
ggcccacttcgcggtgtaat

SEQ ID NO: 126 - RNA, 20 nt, Homo sapiens
  source 1..20: mol_type = unassigned RNA
tcctcctcctccgctcggcg

SEQ ID NO: 127 - RNA, 20 nt, Homo sapiens
  source 1..20: mol_type = unassigned RNA
agtctctcctcctcgcctcc

SEQ ID NO: 128 - RNA, 20 nt, Homo sapiens
  source 1..20: mol_type = unassigned RNA
gccacggcggtcccactcac

SEQ ID NO: 129 - RNA, 20 nt, Homo sapiens
  source 1..20: mol_type = unassigned RNA
ggcaagggtccccgcccgcg

SEQ ID NO: 130 - RNA, 24 nt, Homo sapiens
  source 1..24: mol_type = unassigned RNA
tcctcctcctccgctcggcgcggc

SEQ ID NO: 131 - RNA, 24 nt, Homo sapiens
  source 1..24: mol_type = unassigned RNA
agtctctcctcctcgcctcctcct

SEQ ID NO: 132 - RNA, 24 nt, Homo sapiens
  source 1..24: mol_type = unassigned RNA
gccacggcggtcccactcacagtc

SEQ ID NO: 133 - RNA, 24 nt, Homo sapiens
  source 1..24: mol_type = unassigned RNA
ggcaagggtccccgcccgcggcca

SEQ ID NO: 134 - RNA, 26 nt, Homo sapiens
  source 1..26: mol_type = unassigned RNA
tcctcctcctccgctcggcgcggcgg

SEQ ID NO: 135 - RNA, 26 nt, Homo sapiens
  source 1..26: mol_type = unassigned RNA
agtctctcctcctcgcctcctcctcc

SEQ ID NO: 136 - RNA, 26 nt, Homo sapiens
  source 1..26: mol_type = unassigned RNA
gccacggcggtcccactcacagtctc

SEQ ID NO: 137 - RNA, 26 nt, Homo sapiens
  source 1..26: mol_type = unassigned RNA
ggcaagggtccccgcccgcggccacg

SEQ ID NO: 138 - RNA, 30 nt, Homo sapiens; identical to SEQ ID NO: 42
  source 1..30: mol_type = unassigned RNA

SEQ ID NO: 139 - RNA, 30 nt, Homo sapiens; identical to SEQ ID NO: 43
  source 1..30: mol_type = unassigned RNA

SEQ ID NO: 140 - RNA, 30 nt, Homo sapiens
  source 1..30: mol_type = unassigned RNA
gccacggcggtcccactcacagtctctcct

SEQ ID NO: 141 - RNA, 30 nt, Homo sapiens
  source 1..30: mol_type = unassigned RNA
ggcaagggtccccgcccgcggccacggcgg

SEQ ID NO: 142 - RNA, 20 nt, Homo sapiens
  source 1..20: mol_type = unassigned RNA
agttgaaagcccccattgag

SEQ ID NO: 143 - RNA, 20 nt, Homo sapiens
  source 1..20: mol_type = unassigned RNA
ctaaaacaaagaaaattgta

SEQ ID NO: 144 - RNA, 20 nt, Homo sapiens
  source 1..20: mol_type = unassigned RNA
agtctgtatttttatggagc

SEQ ID NO: 145 - RNA, 20 nt, Homo sapiens
  source 1..20: mol_type = unassigned RNA
catcaaagcaggaactggtg

SEQ ID NO: 146 - RNA, 24 nt, Homo sapiens
  source 1..24: mol_type = unassigned RNA
agttgaaagcccccattgagctgc

SEQ ID NO: 147 - RNA, 24 nt, Homo sapiens
  source 1..24: mol_type = unassigned RNA
ctaaaacaaagaaaattgtaagtt

SEQ ID NO: 148 - RNA, 24 nt, Homo sapiens
  source 1..24: mol_type = unassigned RNA
agtctgtatttttatggagcctaa

SEQ ID NO: 149 - RNA, 24 nt, Homo sapiens
  source 1..24: mol_type = unassigned RNA
catcaaagcaggaactggtgagtc

SEQ ID NO: 150 - RNA, 26 nt, Homo sapiens
  source 1..26: mol_type = unassigned RNA
agttgaaagcccccattgagctgcac

SEQ ID NO: 151 - RNA, 26 nt, Homo sapiens
  source 1..26: mol_type = unassigned RNA
ctaaaacaaagaaaattgtaagttga

SEQ ID NO: 152 - RNA, 26 nt, Homo sapiens
  source 1..26: mol_type = unassigned RNA
agtctgtatttttatggagcctaaaa

SEQ ID NO: 153 - RNA, 26 nt, Homo sapiens
  source 1..26: mol_type = unassigned RNA
catcaaagcaggaactggtgagtctg

SEQ ID NO: 154 - RNA, 30 nt, Homo sapiens
  source 1..30: mol_type = unassigned RNA
agttgaaagcccccattgagctgcacgacc

SEQ ID NO: 155 - RNA, 30 nt, Homo sapiens
  source 1..30: mol_type = unassigned RNA
ctaaaacaaagaaaattgtaagttgaaagc

SEQ ID NO: 156 - RNA, 30 nt, Homo sapiens; identical to SEQ ID NO: 60
  source 1..30: mol_type = unassigned RNA

SEQ ID NO: 157 - RNA, 30 nt, Homo sapiens; identical to SEQ ID NO: 61
  source 1..30: mol_type = unassigned RNA

SEQ ID NO: 158 - RNA, 20 nt, Homo sapiens
  source 1..20: mol_type = unassigned RNA
taccatggaatcctgttgga

SEQ ID NO: 159 - RNA, 20 nt, Homo sapiens
  source 1..20: mol_type = unassigned RNA
ctgagccctaacatcccagc

SEQ ID NO: 160 - RNA, 20 nt, Homo sapiens
  source 1..20: mol_type = unassigned RNA
ggaaggttacttacctgagc

SEQ ID NO: 161 - RNA, 24 nt, Homo sapiens
  source 1..24: mol_type = unassigned RNA
taccatggaatcctgttggagctg

SEQ ID NO: 162 - RNA, 24 nt, Homo sapiens
  source 1..24: mol_type = unassigned RNA
ctgagccctaacatcccagctacc

SEQ ID NO: 163 - RNA, 24 nt, Homo sapiens
  source 1..24: mol_type = unassigned RNA
aggaaggttacttacctgagccct

SEQ ID NO: 164 - RNA, 26 nt, Homo sapiens
  source 1..26: mol_type = unassigned RNA
taccatggaatcctgttggagctggt

SEQ ID NO: 165 - RNA, 26 nt, Homo sapiens
  source 1..26: mol_type = unassigned RNA
ctgagccctaacatcccagctaccat

SEQ ID NO: 166 - RNA, 26 nt, Homo sapiens
  source 1..26: mol_type = unassigned RNA
aggaaggttacttacctgagccctaa

SEQ ID NO: 167 - RNA, 30 nt, Homo sapiens; identical to SEQ ID NO: 71
  source 1..30: mol_type = unassigned RNA

SEQ ID NO: 168 - RNA, 30 nt, Homo sapiens; identical to SEQ ID NO: 72
  source 1..30: mol_type = unassigned RNA

SEQ ID NO: 169 - RNA, 30 nt, Homo sapiens; identical to SEQ ID NO: 73
  source 1..30: mol_type = unassigned RNA

SEQ ID NO: 170 - RNA, 20 nt, Homo sapiens
  source 1..20: mol_type = unassigned RNA
gtaagtatcacagagaacat

SEQ ID NO: 171 - RNA, 20 nt, Homo sapiens
  source 1..20: mol_type = unassigned RNA
ctgaagtgttaaacaagtat

SEQ ID NO: 172 - RNA, 20 nt, Homo sapiens
  source 1..20: mol_type = unassigned RNA
ggtcttctgacttttcttcc

SEQ ID NO: 173 - RNA, 20 nt, Homo sapiens
  source 1..20: mol_type = unassigned RNA
cttgaggccctggaggtcct

SEQ ID NO: 174 - RNA, 24 nt, Homo sapiens
  source 1..24: mol_type = unassigned RNA
gtaagtatcacagagaacatgcca

SEQ ID NO: 175 - RNA, 24 nt, Homo sapiens
  source 1..24: mol_type = unassigned RNA
ctgaagtgttaaacaagtatgtaa

SEQ ID NO: 176 - RNA, 24 nt, Homo sapiens
  source 1..24: mol_type = unassigned RNA
ggtcttctgacttttcttccctga

SEQ ID NO: 177 - RNA, 24 nt, Homo sapiens
  source 1..24: mol_type = unassigned RNA
cttgaggccctggaggtcctggtc

SEQ ID NO: 178 - RNA, 26 nt, Homo sapiens
  source 1..26: mol_type = unassigned RNA
gtaagtatcacagagaacatgccagt

SEQ ID NO: 179 - RNA, 26 nt, Homo sapiens
  source 1..26: mol_type = unassigned RNA
ctgaagtgttaaacaagtatgtaagt

SEQ ID NO: 180 - RNA, 26 nt, Homo sapiens
  source 1..26: mol_type = unassigned RNA
ggtcttctgacttttcttccctgaag

SEQ ID NO: 181 - RNA, 26 nt, Homo sapiens
  source 1..26: mol_type = unassigned RNA
cttgaggccctggaggtcctggtctt

SEQ ID NO: 182 - RNA, 30 nt, Homo sapiens
  source 1..30: mol_type = unassigned RNA
gtaagtatcacagagaacatgccagtctgc

SEQ ID NO: 183 - RNA, 30 nt, Homo sapiens
  source 1..30: mol_type = unassigned RNA
ctgaagtgttaaacaagtatgtaagtatca

SEQ ID NO: 184 - RNA, 30 nt, Homo sapiens
  source 1..30: mol_type = unassigned RNA
ggtcttctgacttttcttccctgaagtgtt

SEQ ID NO: 185 - RNA, 30 nt, Homo sapiens
  source 1..30: mol_type = unassigned RNA
cttgaggccctggaggtcctggtcttctga

SEQ ID NO: 186 - RNA, 20 nt, Homo sapiens
  source 1..20: mol_type = unassigned RNA
cttcccagcagagcggccag

SEQ ID NO: 187 - RNA, 20 nt, Homo sapiens
  source 1..20: mol_type = unassigned RNA
ttgatcaaatacacatcata

SEQ ID NO: 188 - RNA, 20 nt, Homo sapiens
  source 1..20: mol_type = unassigned RNA
taggagttgctcttacttac

SEQ ID NO: 189 - RNA, 20 nt, Homo sapiens
  source 1..20: mol_type = unassigned RNA
ccctccctgccctgtagaga

SEQ ID NO: 190 - RNA, 24 nt, Homo sapiens
  source 1..24: mol_type = unassigned RNA
cttcccagcagagcggccagattt

SEQ ID NO: 191 - RNA, 24 nt, Homo sapiens
  source 1..24: mol_type = unassigned RNA
ttgatcaaatacacatcatacttc

SEQ ID NO: 192 - RNA, 24 nt, Homo sapiens
  source 1..24: mol_type = unassigned RNA
taggagttgctcttacttacttga

SEQ ID NO: 193 - RNA, 24 nt, Homo sapiens
  source 1..24: mol_type = unassigned RNA
ccctccctgccctgtagagatagg

SEQ ID NO: 194 - RNA, 26 nt, Homo sapiens
  source 1..26: mol_type = unassigned RNA
cttcccagcagagcggccagatttcc

SEQ ID NO: 195 - RNA, 26 nt, Homo sapiens
  source 1..26: mol_type = unassigned RNA
ttgatcaaatacacatcatacttccc

SEQ ID NO: 196 - RNA, 26 nt, Homo sapiens
  source 1..26: mol_type = unassigned RNA
taggagttgctcttacttacttgatc

SEQ ID NO: 197 - RNA, 26 nt, Homo sapiens
  source 1..26: mol_type = unassigned RNA
ccctccctgccctgtagagataggag

SEQ ID NO: 198 - RNA, 30 nt, Homo sapiens
  source 1..30: mol_type = unassigned RNA
cttcccagcagagcggccagatttcctttg

SEQ ID NO: 199 - RNA, 30 nt, Homo sapiens
  source 1..30: mol_type = unassigned RNA
ttgatcaaatacacatcatacttcccagca

SEQ ID NO: 200 - RNA, 30 nt, Homo sapiens
  source 1..30: mol_type = unassigned RNA
taggagttgctcttacttacttgatcaaat

SEQ ID NO: 201 - RNA, 30 nt, Homo sapiens
  source 1..30: mol_type = unassigned RNA
ccctccctgccctgtagagataggagttgc

SEQ ID NO: 202 - RNA, 20 nt, Homo sapiens
  source 1..20: mol_type = unassigned RNA
acacaaggaacaattagagg

SEQ ID NO: 203 - RNA, 20 nt, Homo sapiens
  source 1..20: mol_type = unassigned RNA
ctgtggggacaaacagaaag

SEQ ID NO: 204 - RNA, 20 nt, Homo sapiens
  source 1..20: mol_type = unassigned RNA
gaaaggcttttccctgggga

SEQ ID NO: 205 - RNA, 20 nt, Homo sapiens
  source 1..20: mol_type = unassigned RNA
aatcaactccactttagagc

SEQ ID NO: 206 - RNA, 24 nt, Homo sapiens
  source 1..24: mol_type = unassigned RNA
acacaaggaacaattagaggctct

SEQ ID NO: 207 - RNA, 24 nt, Homo sapiens
  source 1..24: mol_type = unassigned RNA
ctgtggggacaaacagaaagacac

SEQ ID NO: 208 - RNA, 24 nt, Homo sapiens
  source 1..24: mol_type = unassigned RNA
gaaaggcttttccctggggactgt

SEQ ID NO: 209 - RNA, 24 nt, Homo sapiens
  source 1..24: mol_type = unassigned RNA
aatcaactccactttagagcgaaa

SEQ ID NO: 210 - RNA, 26 nt, Homo sapiens
  source 1..26: mol_type = unassigned RNA
acacaaggaacaattagaggctctcc

SEQ ID NO: 211 - RNA, 26 nt, Homo sapiens
  source 1..26: mol_type = unassigned RNA
ctgtggggacaaacagaaagacacaa

SEQ ID NO: 212 - RNA, 26 nt, Homo sapiens
  source 1..26: mol_type = unassigned RNA
gaaaggcttttccctggggactgtgg

SEQ ID NO: 213 - RNA, 26 nt, Homo sapiens
  source 1..26: mol_type = unassigned RNA
aatcaactccactttagagcgaaagg

SEQ ID NO: 214 - RNA, 30 nt, Homo sapiens
  source 1..30: mol_type = unassigned RNA
acacaaggaacaattagaggctctccatag

SEQ ID NO: 215 - RNA, 30 nt, Homo sapiens
  source 1..30: mol_type = unassigned RNA
ctgtggggacaaacagaaagacacaaggaa

SEQ ID NO: 216 - RNA, 30 nt, Homo sapiens
  source 1..30: mol_type = unassigned RNA
gaaaggcttttccctggggactgtggggac

SEQ ID NO: 217 - RNA, 30 nt, Homo sapiens
  source 1..30: mol_type = unassigned RNA
aatcaactccactttagagcgaaaggcttt

SEQ ID NO: 218 - DNA, 90 bp, Mus musculus
  source 1..90: mol_type = genomic DNA
gccgccgccgcgccgagcggaggaggaggaggaggcgaggaggagagactgtgagtgggaccgccaaggccgcgggcggggacccttgct

SEQ ID NO: 219 - DNA, 90 bp, Mus musculus
  source 1..90: mol_type = genomic DNA
aaaggtgcagctcaatggggactttcaacttacgattttctttgttttaggctccataaaaatacagactcaccagttcctgctttgatg

SEQ ID NO: 220 - DNA, 66 bp, Mus musculus
  source 1..66: mol_type = genomic DNA
gtagaccagctccaacaggattccatggtagctgggatgttagggctcaggtaagtaaccttcctt

SEQ ID NO: 221 - DNA, 90 bp, Mus musculus
  source 1..90: mol_type = genomic DNA
gcagacagctgtgttctctgtaatacttacatatttgtttaatacttcagggaggaaaagtcagaagaccaggatctccagggcctcaga

SEQ ID NO: 222 - DNA, 90 bp, Mus musculus
  source 1..90: mol_type = genomic DNA
caaaggaagtctggccgatctgctggaaagtatgatgtatatttgatcaagtaagtaagagcaagtcttgtgtctatagaacaagaaggc

SEQ ID NO: 223 - DNA, 90 bp, Mus musculus
  source 1..90: mol_type = genomic DNA
tgtcaaaaagcttctgactattgtttgtgtctctctgtttgtttctacagtccccagggaaaagcttttcgctctaaagtagaattgatt

SEQ ID NO: 224 - DNA, 90 bp, Homo sapiens
  source 1..90: mol_type = genomic DNA
gccgccgccgcgccgagcggaggaggaggaggaggcgaggaggagagactgtgagtgggaccgccgtggccgcgggcggggacccttgcc

SEQ ID NO: 225 - DNA, 90 bp, Homo sapiens
  source 1..90: mol_type = genomic DNA
ggtcgtgcagctcaatgggggctttcaacttacaattttctttgttttaggctccataaaaatacagactcaccagttcctgctttgatg

SEQ ID NO: 226 - DNA, 66 bp, Homo sapiens; identical to SEQ ID NO: 220
  source 1..66: mol_type = genomic DNA

SEQ ID NO: 227 - DNA, 90 bp, Homo sapiens
  source 1..90: mol_type = genomic DNA
gcagactggcatgttctctgtgatacttacatacttgtttaacacttcagggaagaaaagtcagaagaccaggacctccagggcctcaag

SEQ ID NO: 228 - DNA, 90 bp, Homo sapiens
  source 1..90: mol_type = genomic DNA
caaaggaaatctggccgctctgctgggaagtatgatgtgtatttgatcaagtaagtaagagcaactcctatctctacagggcagggaggg

SEQ ID NO: 229 - DNA, 90 bp, Homo sapiens
  source 1..90: mol_type = genomic DNA
ctatggagagcctctaattgttccttgtgtctttctgtttgtccccacagtccccagggaaaagcctttcgctctaaagtggagttgatt

SEQ ID NO: 230 - DNA, 200 bp, Mus musculus
  source 1..200: mol_type = genomic DNA
ctgtggtaaaacccgtccggaaaatggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggc

SEQ ID NO: 231 - DNA, 324 bp, Mus musculus
  source 1..324: mol_type = genomic DNA
acatagtatgttgtctgtttatcttcagaatgttccaaaaagccttgggaaaaggtgcagctcaatggggactttcaacttacgattttctttgttttaggctccataaaaatacagactcaccagttcctgctttgatgtgacatgtgactccccagaatacaccttgcttctgtagaccagctccaacaggattccatggtagctgggatgttagggctcaggtaagtaaccttcctttttttttttttagtatatgtcctggtttggccatctgtttttaaaaaaaaaaaaaaggaaaaaaagatatactattcttggaca

SEQ ID NO: 232 - DNA, 200 bp, Mus musculus
  source 1..200: mol_type = genomic DNA
agccaggggtctcatgtgtggcactcaagctcacctatactcactactgtgcagacagctgtgttctctgtaatacttacatatttgtttaatacttcagggaggaaaagtcagaagaccaggatctccagggcctcagagacaagccactgaagtttaagaaggcgaagaaagacaagaaggaggacaaagaaggcaag

SEQ ID NO: 233 - DNA, 200 bp, Mus musculus
  source 1..200: mol_type = genomic DNA
ctatgtatgatgaccccaccttgcctgaaggttggacacgaaagcttaaacaaaggaagtctggccgatctgctggaaagtatgatgtatatttgatcaagtaagtaagagcaagtcttgtgtctatagaacaagaaggccaggtgggtaggaaggctaggatggaaaagctttattgcactgaaatggcttcccaaaaa

SEQ ID NO: 234 - DNA, 200 bp, Mus musculus
  source 1..200: mol_type = genomic DNA
atgtcttagaagaaagcttgtcagcatattttctatctcagtctctatgttgtcaaaaagcttctgactattgtttgtgtctctctgtttgtttctacagtccccagggaaaagcttttcgctctaaagtagaattgattgcatactttgaaaaggtgggagacacctccttggaccctaatgattttgacttcacggta

SEQ ID NO: 235 - DNA, 200 bp, Homo sapiens
  source 1..200: mol_type = genomic DNA
ctctcggagagagggctgtggtaaaagccgtccggaaaatggccgccgccgccgccgccgcgccgagcggaggaggaggaggaggcgaggaggagagactgtgagtgggaccgccgtggccgcgggcggggacccttgccggggggcgggggtcaggggcgggacgtggcgcgggaggggcccgcggggtcggacgacac

SEQ ID NO: 236 - DNA, 324 bp, Homo sapiens
  source 1..324: mol_type = genomic DNA
ctttgtctatgtgtttatcttcaaaatgtcccaaatagccctgggaaaaaggtcgtgcagctcaatgggggctttcaacttacaattttctttgttttaggctccataaaaatacagactcaccagttcctgctttgatgtgacatgtgactccccagaatacaccttgcttctgtagaccagctccaacaggattccatggtagctgggatgttagggctcaggtaagtaaccttcctttttttttttttagtatatgtcctggtttggccatctgttttttttttttttaaaaaaaaaaaaaaaaggaaaagaggaaaaaaa

SEQ ID NO: 237 - DNA, 200 bp, Homo sapiens
  source 1..200: mol_type = genomic DNA
ggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaag

SEQ ID NO: 238 - DNA, 200 bp, Homo sapiens
  source 1..200: mol_type = genomic DNA
ccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcgggggg

SEQ ID NO: 239 - DNA, 200 bp, Homo sapiens
  source 1..200: mol_type = genomic DNA
tttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggta

SEQ ID NO: 240 - DNA, 20 bp, Homo sapiens
  source 1..20: mol_type = other DNA
atgatagggacttagggtga

SEQ ID NO: 241 - DNA, 20 bp, Homo sapiens
  source 1..20: mol_type = other DNA
tttgttccgtgggtggttta

SEQ ID NO: 242 - DNA, 20 bp, Homo sapiens
  source 1..20: mol_type = other DNA
tggggggaggtaggtaggta

SEQ ID NO: 243 - RNA, 11 nt, Homo sapiens
  source 1..11: mol_type = unassigned RNA
aatttgtctag

SEQ ID NO: 244 - RNA, 11 nt, Homo sapiens
  source 1..11: mol_type = unassigned RNA
aatttttggag

SEQ ID NO: 245 - DNA, 4336 bp, synthetic construct
  source 1..4336: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtcggtaagtttagtctttttgtcttttatttcaggtcccggatccggtggtggtgcaaatcaaagaactgctcctcagtggatgttgcctttacttctaggcgcgcgcgctccctcctctcggagagagggctgtggtaaaagccgtccggaaaatgtacccatacgatgttccagattacgctcctaggggaggcggaggcatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgactttacacggagcggattgcaaagcaaaccaacaagaataaaggcagctgttgtctcttctcggccagaagtagctttgcacttttctaaactagaatccagaaacttgttcttccaaagcagagagcattatgaccaaatatcaccaggactgttactcaatgtgtgccgaaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttagcatgctaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaatgatagggacttagggtgaagcaagggtccccgcccgcggccttggcggaatttttggagcaggttttctgacttcggtcggaaaacccctcccaatttcactggtctacaatgaaagcaaaacagttctcttccccgctccccggtgtgctagggaaacgcgtatctgtctgagtcgaccaaggacacaatcgagctccgatccgtactgtcgagaaacttgtatccgtaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaatgatagggacttagggtgaagcaagggtccccgcccgcggccttggcggaatttttggagcaggttttctgacttcggtcggaaaacccctcccaatttcactggtctacaatgaaagcaaaacagttctcttccccgctccccggtgtgctagggaaacgcgtatctgtgagcgttcccagcacttcagccaaggaagctaccaatttttagtttccgagtgtcacgttaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaatgatagggacttagggtgaagcaagggtccccgcccgcggccttggcggaatttttggagcaggttttctgacttcggtcggaaaacccctcccaatttcactggtctacaatgaaagc

SEQ ID NO: 246 - DNA, 4323 bp, synthetic construct
  source 1..4323: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtcggtaagtttagtctttttgtcttttatttcaggtcccggatccggtggtggtgcaaatcaaagaactgctcctcagtggatgttgcctttacttctaggcgcgcgcgctccctcctctcggagagagggctgtggtaaaagccgtccggaaaatgatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctcggccagaagtagctttgcacttttctaaactagaatccagaaacttgttcttccaaagcagagagcattatgaccaaatatcaccaggactgttactcaatgtgtgccgaaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttagcatgctggggagcacgcacgcgggatatccttaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaatgatagggacttagggtgaaaacaagtatgtaagtatcacagagaacataatttttggagcaggttttctgacttcggtcggaaaacccctactttctggagtttcaaaagtagactgtacgctaagggtcatatctttttttgttttggtttgttctgagtcgaccaaggacacaatcgagctccgatccgtactgtcgagaaacttgtatccgtaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaatgatagggacttagggtgaaaacaagtatgtaagtatcacagagaacataatttttggagcaggttttctgacttcggtcggaaaacccctactttctggagtttcaaaagtagactgtacgctaagggtcatatctttttttgttttggtttgttgagcgttcccagcacttcagccaaggaagctaccaatttttagtttccgagtgtcacgttaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcaatgatagggacttagggtgaaaacaagtatgtaagtatcacagagaacataatttttggagcaggttttctgacttcggtcggaaaacccctactttctggagtttcaaaagtagactgtacgctaagggtcatatctttttttgttttggtttgtacatgga

SEQ ID NO: 247 - DNA, 4302 bp, synthetic construct
  source 1..4302: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtcggtaagtttagtctttttgtcttttatttcaggtcccggatccggtggtggtgcaaatcaaagaactgctcctcagtggatgttgcctttacttctaggcgcgcgcgctccctcctctcggagagagggctgtggtaaaagccgtccggaaaatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctcggccagaagtagctttgcacttttctaaactagaatccagaaacttgttcttccaaagcagagagcattatgaccaaatatcaccaggactgttactcaatgtgtgccgaaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttagcatgctggggagcacgcacgcgggatatccttaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatggggggaggtaggtaggtatttcttccctgaagtgttaaacaaaatttttggagcaggttttctgacttcggtcggaaaacccctactttctggagtttcaaaagtagactgtacgctaagggtcatatctttttttgttttggtttgttctgagtcgaccaaggacacaatcgagctccgatccgtactgtcgagaaacttgtatccgtaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatggggggaggtaggtaggtatttcttccctgaagtgttaaacaaaatttttggagcaggttttctgacttcggtcggaaaacccctactttctggagtttcaaaagtagactgtacgctaagggtcatatctttttttgttttggtttgttgagcgttcccagcacttcagccaaggaagctaccaatttttagtttccgagtgtcacgttaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatggggggaggtaggtaggtatttcttccctgaagtgttaaacaaaatttttggagcaggttttctgacttcggtcggaaaacccctactttctggagtttcaaaagtagactgtacgctaagggtcatatctttttttgttttggtttgtacatgga

SEQ ID NO: 248 - DNA, 1497 bp, Homo sapiens; identical to SEQ ID NO: 7
  source 1..1497: mol_type = genomic DNA

SEQ ID NO: 249 - DNA, 64 bp, Homo sapiens
  source 1..64: mol_type = genomic DNA
actttctggagtttcaaaagtagactgtacgctaagggtcatatctttttttgttttggtttgt

SEQ ID NO: 250 - RNA, 20 nt, Homo sapiens
  source 1..20: mol_type = unassigned RNA
gcaccccatcccccactaca

SEQ ID NO: 251 - RNA, 30 nt, Homo sapiens
  source 1..30: mol_type = unassigned RNA
gggatcgaatcagagtcatgcggaggtgta

SEQ ID NO: 252 - RNA, 30 nt, Homo sapiens
  source 1..30: mol_type = unassigned RNA
gacagtgctctaggattattctggtagtta

SEQ ID NO: 253 - RNA, 24 nt, Homo sapiens
  source 1..24: mol_type = unassigned RNA
cggcggtcccactcacagtctctc

SEQ ID NO: 254 - RNA, 24 nt, Homo sapiens
  source 1..24: mol_type = unassigned RNA
ccgcccgcggccacggcggtccca

SEQ ID NO: 255 - RNA, 24 nt, Homo sapiens
  source 1..24: mol_type = unassigned RNA
ccggcaagggtccccgcccgcggc

SEQ ID NO: 256 - RNA, 30 nt, Homo sapiens
  source 1..30: mol_type = unassigned RNA
cggccacggcggtcccactcacagtctctc

SEQ ID NO: 257 - RNA, 30 nt, Homo sapiens
  source 1..30: mol_type = unassigned RNA
tccccgcccgcggccacggcggtcccactc

SEQ ID NO: 258 - RNA, 30 nt, Homo sapiens
  source 1..30: mol_type = unassigned RNA
ccggcaagggtccccgcccgcggccacggc

SEQ ID NO: 259 - RNA, 24 nt, Homo sapiens
  source 1..24: mol_type = unassigned RNA
cggcggtcccactcacagtctctc

SEQ ID NO: 260 - RNA, 24 nt, Homo sapiens
  source 1..24: mol_type = unassigned RNA
ccgcccgcggccacggcggtccca

SEQ ID NO: 261 - RNA, 24 nt, Homo sapiens
  source 1..24: mol_type = unassigned RNA
ccggcaagggtccccgcccgcggc

SEQ ID NO: 262 - RNA, 30 nt, Homo sapiens; identical to SEQ ID NO: 256
  source 1..30: mol_type = unassigned RNA

SEQ ID NO: 263 - RNA, 30 nt, Homo sapiens; identical to SEQ ID NO: 257
  source 1..30: mol_type = unassigned RNA

SEQ ID NO: 264 - RNA, 30 nt, Homo sapiens; identical to SEQ ID NO: 258
  source 1..30: mol_type = unassigned RNA

SEQ ID NO: 265 - RNA, 24 nt, Homo sapiens
  source 1..24: mol_type = unassigned RNA
aagtgagcagaggcaggcttggac

SEQ ID NO: 266 - RNA, 24 nt, Homo sapiens
  source 1..24: mol_type = unassigned RNA
gaacatgccagtctgcagaacaag

SEQ ID NO: 267 - RNA, 24 nt, Homo sapiens
  source 1..24: mol_type = unassigned RNA
caagtatgtaagtatcacagagaa

SEQ ID NO: 268 - RNA, 24 nt, Homo sapiens
  source 1..24: mol_type = unassigned RNA
tttcttccctgaagtgttaaacaa

SEQ ID NO: 269 - RNA, 30 nt, Homo sapiens
  source 1..30: mol_type = unassigned RNA
agaacaagtgagcagaggcaggcttggacc

SEQ ID NO: 270 - RNA, 30 nt, Homo sapiens
  source 1..30: mol_type = unassigned RNA
cagagaacatgccagtctgcagaacaagtg

SEQ ID NO: 271 - RNA, 30 nt, Homo sapiens
  source 1..30: mol_type = unassigned RNA
aaacaagtatgtaagtatcacagagaacat

SEQ ID NO: 272 - RNA, 30 nt, Homo sapiens
  source 1..30: mol_type = unassigned RNA
cttttcttccctgaagtgttaaacaagtat

SEQ ID NO: 273 - RNA, 24 nt, Homo sapiens
  source 1..24: mol_type = unassigned RNA
aagtgagcagaggcaggcttggac

SEQ ID NO: 274 - RNA, 24 nt, Homo sapiens
  source 1..24: mol_type = unassigned RNA
gaacatgccagtctgcagaacaag

SEQ ID NO: 275 - RNA, 24 nt, Homo sapiens
  source 1..24: mol_type = unassigned RNA
caagtatgtaagtatcacagagaa

SEQ ID NO: 276 - RNA, 24 nt, Homo sapiens
  source 1..24: mol_type = unassigned RNA
tttcttccctgaagtgttaaacaa

SEQ ID NO: 277 - RNA, 30 nt, Homo sapiens; identical to SEQ ID NO: 269
  source 1..30: mol_type = unassigned RNA

SEQ ID NO: 278 - RNA, 30 nt, Homo sapiens; identical to SEQ ID NO: 270
  source 1..30: mol_type = unassigned RNA

SEQ ID NO: 279 - RNA, 30 nt, Homo sapiens; identical to SEQ ID NO: 271
  source 1..30: mol_type = unassigned RNA

SEQ ID NO: 280 - RNA, 30 nt, Homo sapiens; identical to SEQ ID NO: 272
  source 1..30: mol_type = unassigned RNA

SEQ ID NO: 281 - RNA, 20 nt, Homo sapiens
  source 1..20: mol_type = unassigned RNA
gcaccccatcccccactaca

SEQ ID NO: 282 - RNA, 30 nt, Homo sapiens; identical to SEQ ID NO: 251
  source 1..30: mol_type = unassigned RNA

SEQ ID NO: 283 - RNA, 30 nt, Homo sapiens; identical to SEQ ID NO: 252
  source 1..30: mol_type = unassigned RNA

SEQ ID NO: 284 - RNA, 24 nt, Homo sapiens
  source 1..24: mol_type = unassigned RNA
agttgctcttacttacttgatcaa

SEQ ID NO: 285 - RNA, 24 nt, Homo sapiens
  source 1..24: mol_type = unassigned RNA
tccctgccctgtagagataggagt

SEQ ID NO: 286 - RNA, 24 nt, Homo sapiens
  source 1..24: mol_type = unassigned RNA
gagggatccttgtccctgccctcc

SEQ ID NO: 287 - RNA, 24 nt, Homo sapiens
  source 1..24: mol_type = unassigned RNA
acatacattttcctgctccatgag

SEQ ID NO: 288 - RNA, 30 nt, Homo sapiens; identical to SEQ ID NO: 200
  source 1..30: mol_type = unassigned RNA

SEQ ID NO: 289 - RNA, 30 nt, Homo sapiens; identical to SEQ ID NO: 201
  source 1..30: mol_type = unassigned RNA

SEQ ID NO: 290 - RNA, 30 nt, Homo sapiens
  source 1..30: mol_type = unassigned RNA
atgagggatccttgtccctgccctccctgc

SEQ ID NO: 291 - RNA, 30 nt, Homo sapiens
  source 1..30: mol_type = unassigned RNA
cacatacattttcctgctccatgagggatc

SEQ ID NO: 292 - RNA, 24 nt, Homo sapiens
  source 1..24: mol_type = unassigned RNA
tggtggtgacaacgctctgacaaa

SEQ ID NO: 293 - RNA, 24 nt, Homo sapiens
  source 1..24: mol_type = unassigned RNA
tcagagatagggcagagcggatgg

SEQ ID NO: 294 - RNA, 24 nt, Homo sapiens
  source 1..24: mol_type = unassigned RNA
tagaggctctccatagcaatgtca

SEQ ID NO: 295 - RNA, 24 nt, Homo sapiens
  source 1..24: mol_type = unassigned RNA
cagaaagacacaaggaacaattag

SEQ ID NO: 296 - RNA, 24 nt, Homo sapiens
  source 1..24: mol_type = unassigned RNA
cctggggactgtggggacaaacag

SEQ ID NO: 297 - RNA, 30 nt, Homo sapiens
  source 1..30: mol_type = unassigned RNA
caatgtcagagatagggcagagcggatggt

SEQ ID NO: 298 - RNA, 30 nt, Homo sapiens
  source 1..30: mol_type = unassigned RNA
caattagaggctctccatagcaatgtcaga

SEQ ID NO: 299 - RNA, 30 nt, Homo sapiens
  source 1..30: mol_type = unassigned RNA
aaacagaaagacacaaggaacaattagagg

SEQ ID NO: 300 - RNA, 30 nt, Homo sapiens
  source 1..30: mol_type = unassigned RNA
tccctggggactgtggggacaaacagaaag

SEQ ID NO: 301 - RNA, 24 nt, Homo sapiens
  source 1..24: mol_type = unassigned RNA
agttgctcttacttacttgatcaa

SEQ ID NO: 302 - RNA, 24 nt, Homo sapiens
  source 1..24: mol_type = unassigned RNA
tccctgccctgtagagataggagt

SEQ ID NO: 303 - RNA, 24 nt, Homo sapiens
  source 1..24: mol_type = unassigned RNA
gagggatccttgtccctgccctcc

SEQ ID NO: 304 - RNA, 24 nt, Homo sapiens
  source 1..24: mol_type = unassigned RNA
acatacattttcctgctccatgag

SEQ ID NO: 305 - RNA, 30 nt, Homo sapiens; identical to SEQ ID NO: 200
  source 1..30: mol_type = unassigned RNA

SEQ ID NO: 306 - RNA, 30 nt, Homo sapiens; identical to SEQ ID NO: 201
  source 1..30: mol_type = unassigned RNA

SEQ ID NO: 307 - RNA, 30 nt, Homo sapiens; identical to SEQ ID NO: 290
  source 1..30: mol_type = unassigned RNA

SEQ ID NO: 308 - RNA, 30 nt, Homo sapiens; identical to SEQ ID NO: 291
  source 1..30: mol_type = unassigned RNA

SEQ ID NO: 309 - RNA, 24 nt, Homo sapiens
  source 1..24: mol_type = unassigned RNA
tggtggtgacaacgctctgacaaa

SEQ ID NO: 310 - RNA, 24 nt, Homo sapiens
  source 1..24: mol_type = unassigned RNA
tcagagatagggcagagcggatgg

SEQ ID NO: 311 - RNA, 24 nt, Homo sapiens
  source 1..24: mol_type = unassigned RNA
tagaggctctccatagcaatgtca

SEQ ID NO: 312 - RNA, 24 nt, Homo sapiens
  source 1..24: mol_type = unassigned RNA
cagaaagacacaaggaacaattag

SEQ ID NO: 313 - RNA, 24 nt, Homo sapiens
  source 1..24: mol_type = unassigned RNA
cctggggactgtggggacaaacag

SEQ ID NO: 314 - RNA, 30 nt, Homo sapiens; identical to SEQ ID NO: 297
  source 1..30: mol_type = unassigned RNA

SEQ ID NO: 315 - RNA, 30 nt, Homo sapiens; identical to SEQ ID NO: 298
  source 1..30: mol_type = unassigned RNA

SEQ ID NO: 316 - RNA, 30 nt, Homo sapiens; identical to SEQ ID NO: 299
  source 1..30: mol_type = unassigned RNA

SEQ ID NO: 317 - RNA, 30 nt, Homo sapiens; identical to SEQ ID NO: 300
  source 1..30: mol_type = unassigned RNA

SEQ ID NO: 318 - DNA, 500 bp, Homo sapiens
  source 1..500: mol_type = genomic DNA
tttcttttgtaatccgaaacattctagtctgcgaattaaaagccattatttgaagaaggatgccccggctccatctggccaccgaaagattgctccttaacacaggctaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 319 - DNA, 500 bp, Homo sapiens
  source 1..500: mol_type = genomic DNA
gtttcttctgtaatccgaaacattctagtctgcgaattaaaagccattatttgaagaaggatgccccggctccatctggccaccgaaaggttgctccttaacacaggctaaggaccagcttttttgggagagaacagacgcaggggcgggagggaaaaagggagaggcagacgtcacttcctcttggcggctctggcagcagattggtcagttgagtggcagaaaagcagacggggactgggcaaggcactgtcggtgacatcacggacagggcgacttctatgtagatgaggcagcgcagaggctgctgcttcgccacttgctgcttcgccacgaaggagttcccctgccctgggagcgggttcaggaccgcggatcggaagagagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 320 - DNA, 500 bp, Homo sapiens
  source 1..500: mol_type = genomic DNA
gtttcttttgtaatccgaaacattctagtctgcgaattaaaagccattatttgaagaaggatgccccggctccatctggccaccgaaaggttgctccttaacacaggctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 321 - DNA, 500 bp, Homo sapiens
  source 1..500: mol_type = genomic DNA
tctccccgcccgcgggcggcgaagtaaaggcccagcgcagcccgcgctcctgccctggagcctcgtctttctccaggaaaacgtggaccgctctccgccgacaggtctcttccacagacccctgtcgccttcgcccccggtctcttccggttctgtcttttcgctggctcgatacgaacaaggaagtcgcccccagcggagccccggctcccccaggcagaggcggccccgggggcggagtcaacggcggaggccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 322 - DNA, 500 bp, Homo sapiens
  source 1..500: mol_type = genomic DNA
ccaacaagagagaccctcatctctacaaaatattttttaaaataactgggcctcatggtgcattcctgtaatctgagctagttgggaggatagctagagcccaggagatcgaggctgcagagaaccgtgatcacacgactgcactccagcctgggtgacagagcaagaccctgtctcaatagatagatagatagatagatagatagatagatagatagatagatagataaaaacagagaggtgaaaaatatggttttaagggttctgtttaagaatactctgttccaacatggagtttgtcaactctaaagtggtaaaaaacaagacagattgtagacattttcataaaaggcaggattatttggtgtcaccaaattaaatatgttaatggtcacagcggaggccgactcaatcagccttttattctgccaggctatttgatactgctgagggcttacctatttcctgaggagaaaaatagtcattaaaatgaccttata

SEQ ID NO: 323 - DNA, 500 bp, Homo sapiens
  source 1..500: mol_type = genomic DNA
gggcatgaaagataaagttctggaggtaagtagtgatgatggttacacaacaatgtgaacatattctatgccagtgaatttacacttaaggttaaaatggtaaattttatgtatactttggcataatttaaaaaaaagtttgaaaaagaaaaaagacaaaacaataatcgtctttggagaagtctgttacactgaatctggagccaagtgaacagaaccctgattttgatcccttttctctcaaaagcccttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 324 - DNA, 494 bp, Homo sapiens
  source 1..494: mol_type = genomic DNA
gagacagggcttaaaagtcctgggcaattagctgggcacagtgtcgtgcgcctgtaatcccacctagttgggaggctgaggcagaagaatctcttgaacccgaggtagaggttgcagtgagccgagatcgcgccactgcactccagcctggatgacagagtgagaccctgtctcaaaaaaaaaaaaagtcctgggcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 325 - DNA, 500 bp, Homo sapiens
  source 1..500: mol_type = genomic DNA
cgactcgcaaccttttcggggtcccgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 326 - DNA, 500 bp, Homo sapiens
  source 1..500: mol_type = genomic DNA
caaagccggttatgaagcgggggtggggtgggctagttttaataggtccaggcgattagtacctggcatccttaaccacctacagtttgagaagggagtgggtgataaaagcctggaagggaagggaaatcgggccgggcattaggctccatcgctcatcaatagacaaggcctttaggaaactgcgacaacggcttttgctctgggcctttactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcag

SEQ ID NO: 327 - DNA, 500 bp, Homo sapiens
  source 1..500: mol_type = genomic DNA
cacgcctgcctgggctgctccaaggtggtggctaggggtggcacaagttacacattttcaatttggataggttgtgattcttgtcttccccagtcagatacctacagccgctgagagccggtattccaacgccatgagacgccttctgtcgtgagcagttgacaagaaagacccagcacttactgcaaccggaaggagcggttcctccctcgctcgcgcagccgctcttccccgccactccctcggtgcccgccagcacattcccagcaagccctgagtatatttgcatatcaactcactacatttttttcttctaactaaaaaatcgaaaggacaaattccagattctccttgtgaagtcttcctttcagttcagaagaaatggaattcgctcttcaacttcaggaagttgaaataaagagttgcttggattttgtgttcacctttaccaaaaaatggatttggtaacactgccaccctgctttggtgacagagaaagc

SEQ ID NO: 328 - DNA, 500 bp, Homo sapiens
  source 1..500: mol_type = genomic DNA
tcgagctccactaggccgaggaccctaaaccccgaagaaaggaaaggcctctcaccgtcctttcgccgccgccccgcgcttcctgatgagttcgtccagggagatgtccgccatcttgctccgccgagcaagccgaaagcagtcgagaccccgcgagcccgccccttcggccgctagggagcgacgaactacttccggcgtccgcggaccaactctcgcgacagccagctcaaagcaggcaagaaccggaaggggcggggacgttccccgtgagccttcgcggtgctggctgctcatctgcatacggaagttcggcacattatgaattatttattttcctcgagggaaaaaattaaatgaaaagcaacaaaatacattattaacaagtgagacaaacttcaatggaactggatcatgacctcaacagtcaactacgatagtcatcatacgcctaatgagaatagaattcattacctaggaaataaactaaaaacgtcctt

SEQ ID NO: 329 - DNA, 500 bp, Homo sapiens
  source 1..500: mol_type = genomic DNA
gacaagagaaggactgaactgggggagtgagggaagaatgcgagagtgagactcagagagtaaaggggagggaaaaaaaagaagagagaaagggcgaggaagggggcgaccgcaggagggagacagacctggagtccactcggaggcctggagccgcacaaagcgccaggtcagcggtcccggctgggtgagaccagcaggcggctctagcgcgcgggagctgggcgaggctccgggacgacctcaccaatggagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcg

SEQ ID NO: 330 - DNA, 500 bp, Mus musculus
  source 1..500: mol_type = genomic DNA
cacaagtaatgtacttttaaaattaacctttctataccggttttcacagtgcggctgtttttgttgttcctcttagtgttaattcacactaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 331 - DNA, 500 bp, Mus musculus
  source 1..500: mol_type = genomic DNA
agtccttgctcttaactactgaaccaactctcccgtcctataagtaaattttcagagcactaatctattctacttcaagtttctcctgcatcagatccaggccagctggagcgagctacacggttaccccccctcctcccaaaggggaaaataaggatcaggtgaaaatgtttggaaattacaatagtagaatataaagaaagaatatgaaggcggccatgaaggggtagaaaggtagatgaccaggcgtcatccctgatgtcacgggtatgggcgtggtgtatgcagataagcgcgcagggtctgccgggtccgcgctccgtgcagaaatgtgaggggtttctcccgcggcgagggagggagcgcggatacgcgggtgtcagcagagcgcaagaatgtcgtgtgcgacgagggagacccgagatcggccagggctaagtgaccgtgtgttaagagtgtagaggcgacgggtgtgagccggggccgggcagtgggaaagc

SEQ ID NO: 332 - DNA, 500 bp, Mus musculus
  source 1..500: mol_type = genomic DNA
cggtggggctggctcctgtgctgcccgactcctgtgctccgacttggagaagacgggcgtccgttcgtccatacccaaaagagtcggccacaaggatgttggcacctgggggtgggccacgagcggccagttaggggtaagagacggtgcaaggtgacttgtccctggtccttcaccgctccagcactgcgccagtgagaaagtccaacaggccccgggatcgggcggcgaaaggaaggggcgtgatgtcactcctgggtggctgacggggcggggtgtatgcagatgagggtcgcgcaggggctgcggcgtccgcagcggccggggaaacgcgagagatttctcgcgcggcgtgggagagaacgcgggtcgccagatgtcagcggagcgccgagacggcgtgtgcgacgagggagacccgagatcgggcggggctaagtgaccgtgtgttaagagtgcaggggcggcgggtgtgagccggggccgggcgctgggagagc

SEQ ID NO: 333 - DNA, 500 bp, Mus musculus
  source 1..500: mol_type = genomic DNA
gctcctcctaggagctaacactggagccagggggatcaaagctgtgtagtgagtgtcacaaaacatcctagcactaaggagactgagacgggaacattgtagtaagttctagcctgggctacagtaagagacgtagtctcaaacaaggaaatgcgctataaataaataagatggagcttcaatggtctgtgcataactggggggttgaaggagccttccaaaggctgggttccggctccgttcagtcgttgacttgagctgggcggggcatgcaaataaggacgccgcctggtctccgcggaactctgggaacgaaaagcgcaacaaaatcctctcggcccccttggattcctcgggctttccctggcgtaaaggtggtggacaggatagaagaggagtttcggcgtcgggcgcgtgtttgcggtcgcgcgagtgttggcgaccgtgagttgaaggtgtgtgtggtgttgagtgtatggggcgtgtgggcgcagttcggt

SEQ ID NO: 334 - DNA, 501 bp, Mus musculus
  source 1..501: mol_type = genomic DNA
acagaatttactaaatagagagtgcagtcatggaattgccattttccccacgtggtttttcgtttgttcctcaccggaggcctccggtttgagcccgaagtctaaaccggtgcgcgtctgcgagctcgcgtgcgcctccgcggccggagcacagtcgcgggcgggcatcagggcggtgcgtggccgtcagtgacgcatgacccttcgcaggcagaagtgggacgggtcaagagaggactgtgatccgattggctgttcggcgaccgtatgctaatcgcggtgacagtctgcgtggtgtagggacacggtagcgactcaaaatgccccgtggacggggtctggagaggtgtcggtctgtcagagaaggtgtccccgcgagggcagacgcggtgtgagatagaggcgcggacagggatgtcacgggtcgtctgtgcgtcagtcgccgtgactaagatgtgggggcggccggtgcagtgcggcgcggtgtggagcgtgaggttt

SEQ ID NO: 335 - DNA, 500 bp, Mus musculus
  source 1..500: mol_type = genomic DNA
ttatctacgttggttttactacagaaactactgttctcagtaaaagcagagagagtacaatgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 336 - DNA, 500 bp, Mus musculus
  source 1..500: mol_type = genomic DNA
ggcatgtgattttgttttgtttaggtttttttttaactttatttgatcaacatgaaataatacaataagctatgtcttctaaccagcaggcagccagccaggagccatgtgtagcagtgttagaagctgcagcaggcatgtgggtaattgtggggagcttttaggaacagctagggagctgtgtggagctgcagagatgcagaggagcctggaggaacagaccagtgctgtgggctgggaggagcccccctcacaggtacttgctcaacggctccacgaccataagccttgaataagttacatggacattttacaaatggcacaggattgattatgtctgctgccagaagcctcatgtcgataaggagagagctggccaaaaggtaaagtatgctagctccatacagtgcaatagccacgtagtctcacagagaaagaggcaggcctgcatcatatccaaatatggccaagaattttattaaattaaaatgcaaacagca

SEQ ID NO: 337 - DNA, 500 bp, Mus musculus
  source 1..500: mol_type = genomic DNA
tatttttaatgaagcagggccatggagcccaagctggccttgaacttctcagcttttgtgctggtatcacagaaatgtggcgctatgcccggtcgggcctgatgttccagagtctttgcagtgtggggaacttgtaaggatgagactacaagacatcgggccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 338 - DNA, 500 bp, Mus musculus
  source 1..500: mol_type = genomic DNA
gttctttgcttttacagaccgtctcctgtgtgtccctgtcagagacctgaattcggttcctagcacccatatgttagaacataacgtccaagttggaggctacaagtggcactaaattacacattatcagttttgacaggttggggcaccgtgtaaccctagtaacttagcgaaaggctcctcgaacccaacatcccacctctagctgggagcagctgttttgcaccagcgccgcctacctctccctccgcgtcggccagagcattcccagcaagcactgctctgatttgcatattgactcacggactttcctttactgctatacaaaaaacgacacaacagttctctttgtgaagagctttccctcagcccgagaagaatcgatagtttgccttttagcttcgacaatatacagttgaaagttggttagactttattttcacctttaccgtaagatgggtggaccgacaatgccactgtgcttccgaacgcgctcgagc

SEQ ID NO: 339 - DNA, 500 bp, Mus musculus
  source 1..500: mol_type = genomic DNA
gaagcagcgaggcccctacgagcctcgaggaaaagggaaagggccgctcaccgtcccttggccgccgtcccgcgcttccggatgagctcgtccagcgagagatcggccatcttgcggcgcgcagctagcggagaacggaggagaagccgactgcggcgagcccgccccgcggccggagtcaagagcggcttccggcaacccggacggccaactctcgcgacagctcaaacgaaaagctcgagacggaaggggtggagcctctctccccgtgagcctttgcggtgctcgcttctaatttgcatggaggagtccggagccttatgggctctctctcccccctcctccttttgctaatgaggaattagaattatatcgatcataaaccattaaaaagcattatcaagaggacaagtcttttagtcatacagccgatcggacactcaccatacttgtaatgagaatataatttcttacccctgaggaacgtagacacgttcctc

SEQ ID NO: 340 - DNA, 500 bp, Mus musculus
  source 1..500: mol_type = genomic DNA
cgcttcggtgtgtcacttcggccacagagggagacagacgattacggcccacggaaggccgcgtgagtctcgtgaagctggagatgcgctgggtctacctttcgccgtccgctccgccttggcagaggcgccgacacgcaccgaaggcagagaacccatctcgtatgggaccgcaccggacacgggcgcctgggccaggagcagagccgggccgtagaatagacatggccgtcgggggcggggcttcggaaggtttaaccaatccaaactgttgtattttgcatagccccaaagcattttggttaacagtaaaaacatcctaaatttaagtattttaatttaaacttagaacgaagcgagtataaaaaggattatttaaccctaaaacggattcaggatttgttataatatcaagtacagtcggctacataaggtcaccacatgtgtaaagttacaaaattctatggccttatatacctaccaagagcctgcttactctc

SEQ ID NO: 341 - DNA, 500 bp, Homo sapiens
  source 1..500: mol_type = genomic DNA
actttctggagtttcaaaaacagactgtacgccaagggtcatatctttttttgtattggtttgtgtcttggttggtgtcttaggtgttaatcctacagtggagggtttatgggaataggaagtaacatgtcgcctgcatgtcataggagaaaaagcgagcatcagccgtatcggctttgtaacacaaattagctatcgtgaagtccgctcagctcttccctttctaccctggctgctttttgcagggattggtccgtggtctccagtctcttgggttctcaccctgtgtgaaaatcttcgtgtttttccctaccccccagtcacctcttacacagcctctgcttccaagcgcagcccccacaggagtttgtaggatttctgtgctagcggggaatgtgttctcacctcatagagccaggtagaaattatgcagatgggcagttctctgggaagaaagcagggcctttggggctctcagtgtccccgttgggttgtagaca

SEQ ID NO: 342 - DNA, 500 bp, Homo sapiens
  source 1..500: mol_type = genomic DNA
actttctggagtttctaaaagtagactgtacgctaagggtcatatctttttttgttttggtttgtgtcttggttggcgtcttaaatgttaatcctacagtggagggctggcgaataggaagtaacatgtcgcctgcacgccataggagaaaaagcgagcatcagccgtatcggctttgtaacacaaattagctatcgtgaagtccgctcagctcttccctttctaccctggctgctttttgcagggattggtccgtggtctccagtctcttgggttctcaccctgtgtgaaaatcttcctgtttttccctaccccccaagtcacctcttacacagcctctgcttccaagcgcagcccccacaggagtttgtaggatttctgtgctagcggggagtgtgttctcacctcatagagccaggtagaaactacgcagatgggtgctgttctccaggaagaaagcagggcctttggggctctcagtgtccccgttgggttgtaga

SEQ ID NO: 343 - DNA, 500 bp, Homo sapiens
  source 1..500: mol_type = genomic DNA
actttctggagtttcaaaaacagaccgtacgccaagggtcatgtcttttttcgtattggtttgtgtcttagttgttaatcctacagtggaggcctggggaataagaagtaacatgtggcctgcacgccataggagaaaaagcgagcatcagccgtatcggctttgtaacacaaattagctatcgtgaagtccgctcagctcttccatttctaccctggctgctttttgcagggattggtccgtggtctccagtctcttgggttctcaccctgtgtgaaaatcttcgtgtttttccctaccccccaagtcacctcttacacagcctctgcttccaagcgcagcccccacaggagtttgtaggatttctgtgctagcggggagtgtgttctcacctcatagagccaggtagaaattatgcagatgggcgctgttctctgggaagaaagcagggcctttggggctctcagtgtccccgttgggttgtagacataacactctta

SEQ ID NO: 344 - DNA, 304 bp, Homo sapiens
  source 1..304: mol_type = genomic DNA
cctccggggatacaacgtgtttcctaaaagtagagggaggtaagagacggtagcacctgcggggcggcttgcacgccgagtgcctgtgacgcgccggcttaacttaactgcttccctgaagtaccttgaggttcctgatgtgcgggcggtagacggtaggcttatgcggcacgctgtcgtttccaccgtggctactgcgctttgggaaggccacgacctcctcctttggggaggtccttaggatctcagcttggcagtcgagtgggtggcgaccttttaaaggaatgggacccacccggagttc

SEQ ID NO: 345 - DNA, 492 bp, Homo sapiens
  source 1..492: mol_type = genomic DNA
ttcccttcttgtaatacaaaataataacaaccacagaggacatcgcaagtaaaatgtgtataacaaccccatctaaagaaccctatcatctttctactatctgtctacctaaatatgtatagtataaataaccctatatagcactctaagcaacttaagttaggtaacttagaaaactcttttttttttttgagactgagtctcactctgtcgcacccaggctggagtgcagtggcgtgatctcggctcactgcaacctccaccttctgggttcaagtgattctcccatctcagtctcccaagtagctgggattacaggcacgccaccatgccctgctacatttttttttttttagtagagatgaggtttcaccacattggccaggctgttctcgaactcctgacctcaggtgatccgcccaccttggcctcatgaagtgctgggattacaggtgtgagccactgtacccagccagaaaactcttaaaaaac

SEQ ID NO: 346 - DNA, 382 bp, Homo sapiens
  source 1..382: mol_type = genomic DNA
ctgaattttcttgcagttgaacaacagaggctttttttgtgtgtgtgggggtgcttggttttgggaggttgaagagtacttgttcgcaaactctctaaatgagagatggggaagtgtcttaccagctgattcgcgagagcacctgcaagtttacctgttttagtgatttgcgtttatgaaagttggagttgctgattttgtgatctctacttttaaggtaaaagcagggcggtgttgacttttcttgccagttggctgtcctgttttctttctcgcactgttttctttttgagacgaagtctgtcgctcaggctggagtgcactggcgtgatctcggctcgctgcaaactttgccgaacccctgtttagcgatttccgtttg

SEQ ID NO: 347 - DNA, 500 bp, Homo sapiens
  source 1..500: mol_type = genomic DNA
atatgtggtaatccaacaatagaaattatttttaagtttgtgtgttcctttttctgttcaatggtgcttttgatattgttgtaaagcagtgactagcagattctgtgtggtaaaagcactgaaccctgtttagttcttttctccctaccttattggtaattatttctgtgtatatatgttccaaagaaatgcggcttgtatattcatgcttttcaactttttgtgatagacagttgatacacaagtagtacctcctccgggatcttcttagtcaagactgtttggtagatgcatgttttttgttacttgtaatgtacagtattcagttcattttctttttgcagcactgtccggttgaaacacaacgcgaggatcgggcgtggtggctcacgcctgtaatcccagcactttgggaggccgaggcaggacaatcaagaccaatcctggtcatcaaggcaggagaatcaagaccatcgaggtcaggagatcaagaccatcct

SEQ ID NO: 348 - DNA, 498 bp, Homo sapiens
  source 1..498: mol_type = genomic DNA
acatcaggttgtttttctgtttttacatcaggttgtttttctgtttggttttttttttacaccacgtttatacgccggtgcacggtttaccactgaaaacacctttcatctacaggtgatatcttttaacacaaataaaatgtagtagtcctaggagacggaatagaaggaggtggggcctaggcagattcatctctgcggtgcattttgcctctggccctcgggccctagtttgcgatctttcccatcttgcctctgcttccctctgcacaatcctgagtattgcagtaccttacacttaaggaaatttcgtcttgccaaagttgatccatttcagctccgaaacataacaggaactagtgcaacgataaacgtatgaggaacttgcaataagagtgacagaatcctttgaatcaaaaaaaaaaaaagccaccacttaatgagtatctaccatatgtcaagggcttcaccaatatttcattgaaatattccagacca

SEQ ID NO: 349 - DNA, 500 bp, Homo sapiens
  source 1..500: mol_type = genomic DNA
cttatgatgtttgttgccaatgatagattgttttcactgtgcaaaaattatgggtagttttggtggtcttgatgcagttgtaagcttggggtatgaaggtttgggccacgcctgggcgcttccggctgcgccggatgctgtttcctttccgctcccaggggcgttgggaacggttgtaggacgtggctctttattcgtgagttttccatttacctccgctgaacctagagcttcagacgccctatggcgtccgcctcgacccaaccggcggccttgagcgctgagcaagcaaagggtgagaatcgtcctagtcaaggcataggctgctggcctggggtagtcaaggcatgggctgctggtctggggaggatgcgggcggaggatgtggggcgacaaacctggctacgtccgccgggaaaatggggtaggggacgccgggtaccgtccttctaagtggggcgcttgcccccaagacttgggatcttattgggttacctaca

SEQ ID NO: 350 - DNA, 498 bp, Homo sapiens
  source 1..498: mol_type = genomic DNA
tctttactgttatatgttaggcgaaatattacgcgtttggagtaagtggtgctttttgtaactgaaaagagattctgtgtgtgtttttttttttttagaggctgcatagttatttgtatgaaccgcaggtgacctaattagcaaggtaattcagacctgtatcctagcacttaagaagtggaagtgggataaccctgcgtgttctcgtgtgggaggctagcttaagcaacaaagcaggaaggccccaccaacaaaagaatttaaaaattatacgtgcgtggtggcgcgcgtatgaggaaattatttggcaactgtgttacagtatagcagcaaatggaatagacctagtccctgactttaggagacctgcctcagccccccgaatggctgggactacaggcgtgcgcctccgcgcccagctaatttttgcatttttggtagagacgggttcaccatgttggccaggatggtctccatctcttcatctggtgatccgcc

SEQ ID NO: 351 - DNA, 500 bp, Homo sapiens
  source 1..500: mol_type = genomic DNA
accttattcacgcctaaaaagtagactgactgtggggtggtcgtgttttttgtttcttgttggtaggtggtgaatgcgtttttttcgttgttttctccgttactcaggctgccagttgcttggcagtcttgtcgctggctgtggacgctctgcacttcatcaccgtccccagctgcgctttgagccggtctaaggttgcccacagtattgacgttgggacccacgaaactggcaatttccatgtgtgcggccttacccagttctcccaaacttctgcgtataaccgcttctctgtgcttcagaaagataagtccggccccggcctcacacagcagctcagttcgtgacagcgtttaatatgtaaaatggagcaaataatttatagggcgttgggagagttaaagagactaattcatttacgtgttaaatacagggaatggcttttgcattgatataaccccagcctccagcatgcgccagacctgacgtaggtgttcggc

SEQ ID NO: 352 - DNA, 500 bp, Homo sapiens
  source 1..500: mol_type = genomic DNA
tttgacccattacccatctaagttagatgcttttttaaatgttttttaatttttaaatttttaatttttttcattatttattttttatttttgagacggatctcggctcactgtaacctccacctcccgggttcaagcgattctcctgactcagcctcccgagtagctgggattacaggcgcgcgccaccatgcccggctaatttttctatttttagtaaagacggggtttcgccatgttgaccagggtggtcttgaactcctgacgtcaggtgatcctcctgcctcgtgctgggattgcaggcgtgagccaccgctcccggcccttagatgctttttaatcagcaagtacgcaatgctttcctgacaggaatgtcaagtccgtcaggaaatccacataagggtgttaagcaggcaaagtaccacatacagattaaaatcaaaagcactgtcaacgcttatttaatttatacagtagaccctaacttttctaattttgtg

SEQ ID NO: 353 - DNA, 500 bp, Mus musculus
  source 1..500: mol_type = genomic DNA
gtttacttggttttaaaaatagcttgcactagcgataccgcgaatatggttattaggtttgttaggcacagtcgtgtcttactatagaaaaataacgtagtgttcattttgctgcctgtatgtgttaatttgtccttattgcgccattgttcttgttaagtcttctgtaaggagttgcgggtttcaaactgtcagtctgagagcataagctcacttttcagtcagttgaattgaatgttacattcgattggttctggatattgatattacccaaggcttgtcctgtttgtaagtcttttcctacacctgacaatatgattatgtttacaatgtccctagaactctgggtgtatcgttcccactgaataaaatttgatctctaagttgacctataaatttaagtaccaaaaaactggtttgtaagattttggaaggcagaggcagggaagagaccaacgtggtctacatataaagtaccaggtacatataaagtaccaggt

SEQ ID NO: 354 - DNA, 496 bp, Mus musculus
  source 1..496: mol_type = genomic DNA
atttttgtggtgctaaaagttagattgcattccgcccttctcacgtctctttacgtgttgtttgcgaggcgtggcctcggaggtaaatggtctccttttacctcagataagcttacctttgctctgtgagtaagaatgtagctacacttccaaatttagctattgtcctttgcggtccaaatgaaaatattaccttccgatctaacaatgttaaactagtcccaggcccttctatcgattgccagaatagactagactactacacagacacaccctctcaaatcaggccagctcctgcaacctgcacctacagtttgtcccctgggacaaatgaaaggacaggccctctttcccccccccccccaaatcagagcacacacagatcttggggtctggggcagaaatttcgttcatggaaaaaacaaacaaacaaacaaacacgatcaaaggaagctcgttccctttggcctcagggtttcttagacgccgtgttccc

SEQ ID NO: 355 - DNA, 497 bp, Mus musculus
  source 1..497: mol_type = genomic DNA
ataactggtaaccaaaagcagattactttcccccttgtggtcttcgcgtttgcggacgggagagaactttcggccattttccttttagaaatttaacttcttgaaggccaccaggtgacccttacttccctctcacctttccttctacctgtactttctctattaaaaagcctgcagaagtacccttgacaaattgccaaaaatgacacctaaaaaaaaaaaaaggaaaaacgacaccttaggtaattataggttgtaagaaaaagatttgacctcttggagtttttatttcttaataccaaaggacacccaaggaacgtatctctcaaaattgggtttcaaacctcagccgtttccttcttagttccagtgcttaacacgcaacagacctgcacaccactacactgcatacgagttttaaagttaataaatcagtcacccctccatagtcttctacctacttgaagacagaaacggctgctaggaagcaattcttc

SEQ ID NO: 356 - DNA, 500 bp, Mus musculus
  source 1..500: mol_type = genomic DNA
ctggggaataagagtggtttaaagaaagaacaatttgagagatctgcttagatctaataaaacccaagtatagtttctttagacgtcttatatctcacgtaggacctcttatttcagactcactgcaagaacaagctaaagaactatatacagtcaggaacaaggtgagccatcgcggttctcagtcccatgatcttaagagtttttcctcttcccctcttccctggctgtcctggagctcactctgtagaccaggctggtctcaaactcagaaatccacttacctctgcctcccaagggctgggattaaaggcatgcaccaccattgcccggcagggtatgtggactatgctgtccttcaactcagagatcgtggtactttaaatctcattttccgaaagaaaagaaaagaaaggaaggggggaaataaagccagtatattttgcacccacaatctgaagttctacaaatattgttagaaaatgcaaaggacccaagtc

SEQ ID NO: 357 - DNA, 500 bp, Mus musculus
  source 1..500: mol_type = genomic DNA
ctcggtttatcttaaagggttttaaatatagaaagcttccttagtcttatgggtgagagtgctgtttaagccttacctaggtatgtctttgaacatttattacagattgttatttgtgactatgaccactcaggtgttctgttcttgtgaatttagacacactcagaaaggctacacactcaaagaccacaccaaagtgaagtactatctgggccgtgaaactggtcacatatcagttattttgcagtaaaacaaggaagtcaatcatactgcttcagtgggtcagggagtcaagaatgcacagtacaagagctggtgagattcctcagcaggtaagagcactgaatgctcttccaaagattctgagttcaaatcccagcaaccacgtggtggatttgaacaaaggatgaaaacaaccatccataaccagatctgatgccctcttctggagtgtcaaaagacagctatggtgtacttacataaataaataagtaaaaaca

SEQ ID NO: 358 - DNA, 500 bp, Mus musculus
  source 1..500: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttgagccaccatgtggttgctgggatttgaactcaggacctttagaaaagtagttgggtgctcttacccgctgagccatctcaccagccctttttttttcttttttccagttcgggtttctctgtgtgtagccctggctgtcctggaactcattctgtagaccgggctggcctcgaactcagaaatctgcctgcctctgcctcccaagtgctgggattaaaggaatgtgccttcattgcccggctctagggctttttgttgttgtttgtttgtttgtttgtttgtttgcttttgtggatggcct

SEQ ID NO: 359 - DNA, 500 bp, Mus musculus
  source 1..500: mol_type = genomic DNA
aaaaccagtaagggagaaaggtcaagtaacatataaaggaaggcctatcagaattacaccagacttttcaccagagactatgaaagccagaagagcctggacagatgttatacagacactaagagaacacaaatgccagcccaggctactatacctggccaaactctcaattaccatagatggagaaaccaaagtattccacgacaaaaacaaattcacacaatatctttccacgaatccagcccttcaaaggataatgacagaaaagaagcaatacaaggacggaaatcacgccctagaacaaccaagaaactaatcattcaacaaaccaaaaagaagacagccacaagaacagaatgccaactctaacaacaaaaataaaaggaagcaacaattacttttccttaatatctcttaatatcaatggactcaattccccaataaaaagacatagactaacagactggctacacaaacaggacccaacattctgctgctta

SEQ ID NO: 360 - DNA, 500 bp, Mus musculus
  source 1..500: mol_type = genomic DNA
cccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcctgcatccgtgggggaggtggctggctgcagggttctccggatctcttctgtgttcccactactcaagcaccgagtggcgttctatggcgtccgcctcggctcagcccgcggccctgagcgcggagcaggccaagggtgaggaaagagcctagtctgggtgtgggccgtcttctggggggagggcgcgatactattctttgcctctccaagtggggcccttgcccattagggcgaagggttgggaggacaagcttatcgggttgcttgcaggcctcggcccactaattccctgcctcttccctcctctgggctcgcccgccgtcctccgcg

SEQ ID NO: 361 - DNA, 500 bp, Mus musculus
  source 1..500: mol_type = genomic DNA
gaatttgttaaattacaggaggaagcttttttagttcattttacaaatactttcttgttaggtagataataccttctaaggttattatctggatcttctaagtctttttatcgctagcaagtctaagtttctggactttggaattagaagttaatgtcgtaggcgggcatggtggtccacgcctttaatcccagagtttgggaggcagacgcaggtggttttctgagttcgaggccatcctgctcttcaatgttagttccaggacagccagggttatacaaagaaaccctgtctcgagaaacaaaacaaacaaaaaaagagtagctgagtgctctgagttgtctctccagccccttagctatttattttcatttaaattatttttgttttattcgcttgtgtgcctgagtttgaatttcccgggattaacagttgaagactagggctggtgagatggctcagcggttaagattgccgactgctcttccaaaggtcctgag

SEQ ID NO: 362 - DNA, 500 bp, Mus musculus
  source 1..500: mol_type = genomic DNA
accccctaattctacaagaatagaaagcttagcagctcctctgaatggttgctctcaggtgctagtggttgcaagtcgtatgttgatgcctgcctttgccctacctggtgtgtgagcgtgagcgcgccccacctctccagcctctctcttactgtttttcagacaagatgggactgttgttctcctactgctatgtacctcaatttcaattgatcatgcctggacataactgcccatgttgtaaggtcgtctacgttgtaaggtcgtgctcagtttctgatgcctagactagtcacctttccgtttgcctcctgactgtgtaaatgaggcaaatgaaggtggtctcttaactgacatgttagtgtttgacctatgggatagtttctcattgttgagacagaatttctagaagctggtccgctgagcggctaagagcacttgctgatattgcaaaggatctacctgggtttggttctcagcactcacatggaagttcacat

SEQ ID NO: 363 - DNA, 67 bp, Mus musculus
  source 1..67: mol_type = genomic DNA
tgtagttcctgcaatttaattttcgtttaaaactttattttattctttaacttaaaaaaaatatata

SEQ ID NO: 364 - protein, 486 aa, Homo sapiens
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 365 - DNA, 77 bp, Mus musculus
  source 1..77: mol_type = genomic DNA
atggccgccgctgccgccaccgccgccgccgccgccgcgccgagcggaggaggaggaggaggcgaggaggagagact

SEQ ID NO: 366 - DNA, 351 bp, Mus musculus
  source 1..351: mol_type = genomic DNA
ggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaa

SEQ ID NO: 367 - DNA, 1078 bp, Mus musculus
  source 1..1078: mol_type = genomic DNA
tccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctga

SEQ ID NO: 368 - DNA, 62 bp, Homo sapiens
  source 1..62: mol_type = genomic DNA
atggccgccgccgccgccgccgcgccgagcggaggaggaggaggaggcgaggaggagagact

SEQ ID NO: 369 - DNA, 351 bp, Homo sapiens
  source 1..351: mol_type = genomic DNA
ggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaa

SEQ ID NO: 370 - DNA, 1084 bp, Homo sapiens
  source 1..1084: mol_type = genomic DNA
tccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga